(19) United States
(12) Patent Application Publication
(10) Pub. No.: US 2004/0146907 A1
(43) Pub. Date: Jul. 29, 2004

(54) Methods and compositions for detecting dysplasia

(75) Inventor: Victoria Smith, Burlingame, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 10/712,124

(22) Filed: Nov. 13, 2003

Related U.S. Application Data
(60) Provisional application No. 60/425,813, filed on Nov. 13, 2002.

(51) Int. Cl. C12Q 1/68

(52) U.S. Cl. 435/6

(57) ABSTRACT

Methods and compositions are disclosed for detecting dysplasia in a tissue sample, screening candidate compounds for the ability to inhibit growth of a cancer cell, predicting predisposition to adenocarcinoma and treating cancer based on gene expression profiles.

This application is a non-provisional application filed under 37 CFR 1.53(b), claiming priority under 35 USC 119(e) to provisional application No. 60/425,813 filed Nov. 13, 2002, the contents of which application is incorporated herein by reference.

### TECHNICAL FIELD

The present invention relates to nucleic acid sequences, and compositions and uses therefore, which have been shown to be differentially expressed in high-grade dysplasia and which are useful as markers for the detection of high-grade dysplasia in a patient, and are implicated in the development of adenocarcinoma.

### BACKGROUND OF THE INVENTION

The incidence of esophageal adenocarcinoma is rising in Western Countries, replacing squamous cell carcinoma as the most common neoplasm of the esophagus in white males and increasing in other ethnic groups (Devesa et al., Cancer 83:2049-2053 (1998); and Bollschweiler et al., Cancer 92:549-555 (2001)). Barrett's esophagus (BE) is the primary recognized risk factor for esophageal adenocarcinoma. BE results from repeated injury to the esophageal mucosa and develops in a subset of patients with chronic gastrointestinal reflux disease. It is characterized by a metaplastic change of squamous esophageal epithelium to intestinalized columnar mucosa (Csendes et al., Dis. Esoph 13:5-11 (2000); Cameron et al., New Eng. J. Med. 313:857-859 (1985); and Drewitz et al., Amer. J. Gastroenterol 92:212-215 (1997)).

Barrett's esophagus is found in 6% -16% of patients undergoing upper gastrointestinal endoscopy for gastroesophageal reflux, and it is estimated that a substantial patient population remains undiagnosed (Sarr et al., Amer. J. Surgery 149:187-193 (1985); Winters et al., Gastroenterology 92:118-124 (1985); Cameron et al., Gastroenterology 99:918-922 (1990); and Cameron et al., Gastroenterology 103:1241-1245 (1992)). The risk of developing esophageal carcinoma is 30-150 times greater in patients with BE. The outlook for patients diagnosed with adenocarcinoma is poor, with a 5 year survival rate of 10-15% (Streitz et al., Ann. Surg. 213:122-125 (1991); Menke-Pluymers et al., Gut 33:1454-1458 (1992); and Lerut et al., J. Thorac. Cariovasc. Surg. 107:1059-1066 (1994)). Patients with BE are placed on surveillance programs, although the absolute risk of developing adenocarcinoma in the context of BE remains relatively low, estimated at approximately 0.5% per patient year (Drewitz et al., Amer. J. Gastroenterol 92:212-215; O'Connor et al., Am. J. Gastroenterol 94:2037-2042 (1999); Spechler et al., JAMA 285:2331-2338 (2001); and Shaheen et al., Gastroenterology 119:333-338 (2000)). The value and cost-effectiveness of surveillance programs continue to be debated due to lack of understanding of the natural history of BE, the difficulty in obtaining representative biopsies by random sampling due to the heterogeneous nature of intestinal metaplasia, and inter-observer variability in endoscopic and histopathologic diagnosis (Falk, Gastroenterology 122:1569-1591 (2002); Sampliner, Am. J Gastroenterol. 93:1028-1032 (1998); and Alikhan et al., Gastrointest. Endosc. 50:23-26 (1999)). A metaplasia-dysplasia-carcinoma sequence has been described for BE and genetic changes involving cell cycle abnormalities, DNA ploidy, mutations, and amplification and expression of oncogenes have been identified (al-Kasspooles et al., Internat. J. Cancer 54:213-219 (1993); Vissers et al., Anticancer Res. 21:3813-3820 (2001); Bani-Hani et al., J. Natl. Cancer Inst. 92:1316-1321 (2000); Walch et al., Am. J. Pathol. 156:555-566 (2000); Wong et al., Cancer Res. 61:8284-8289 (2001); and Romagnoli et al., Laboratory Investigation 81:241-247 (2001)). There is a need for reliable detection of high-grade dysplasia and diagnosis of patients, such as BE patients, likely to develop adenocarcinoma, thereby allowing the disease to be monitored and treated early in its progression.

### SUMMARY OF THE INVENTION

Generally, the present invention is based on the discovery that it is possible to detect high-grade dysplasia in a patient suspected of experiencing dysplasia, such as dysplasia associated with gastrointestinal reflux disease, such as Barrett's esophagus, or colon tissue dysplasia, by determining expression is an esophageal or colon biopsy from the patient wherein at least eight genes selected from a group of genes are expressed at a level of at least 1.5 fold over expression in a control sample. The control sample may comprise an esophageal or colon biopsy from a normal patient (i.e. one not experiencing gastrointestinal reflux disease) or from pooled samples of normal epithelial tissue (such as from normal liver, lung and kidney tissue). The group of high-grade dysplasia (HGD) gene markers, and their encoded polypeptides, comprise ET-1 (endothelin-1, NM_{—}001955) (SEQ ID NO:1 or 2); AGR2 (anterior gradient 2 (Xenepus laevis) homolog, NM_{—}006408) (SEQ ID NO:3 or 4); ADAM8 (NM_{—}001109) (SEQ ID NO:5 or 6); PRSS8 (Prostasin precursor, serine protease, NM_{—}002773) (SEQ ID NO:7 or 8); AXO1 (Axonin-1 precursor, NM_{—}005076) (SEQ ID NO:9 or 10); NROB2 (Nuclear hormone receptor, NM_{—}021969) (SEQ ID NO:11 or 12); TM7SF1 (NM_{—}003272) (SEQ ID NO:13 or 14); DLDH (dihydrolipamide dehydrogenase, NM_{—}000108) (SEQ ID NOS:15 or 16); MAT2B (methionine adenosyltransferase II, beta, NM_{—}013283) (SEQ ID NO:17 or 18); STC-2 (stanniocalcin-2, NM_{—}003714) (SEQ ID NO:19 or 20); PPBI (alkaline phosphatase, intestinal precursor, NM_{—}001631) (SEQ ID NO:21 or 22); SLNAC1 (sodium channel receptor SLNAC1, NM_{—}004769) (SEQ ID NO:23 or 24); CAH4 (carbonic anhydrase iv precursor, NM_{—}000717) (SEQ ID NO:25 or 26); PA21 (phopholipase a2 precursor, NM_{—}000928) (SEQ ID NO:27 or 28); PAR2 (proteinase activated receptor 2 precursor, NM_{—}005242) (SEQ ID NO:29 or 30); IDE (insulin-degrading enzyme, NM_{—}004969) (SEQ ID NO:31 or 32); MYO1A (myosin-1A, NM_{—}005379) (SEQ ID NO:33 or 34); CYP2J2 (cytochrome P450 monooxygenase, NM_{—}000775) (SEQ ID NO:35 or 36); PHYH (phytanoyl-CoA-hydroxylase (Refsum disease), NM_{—}006214) (SEQ ID NO:37 or 38); CYB5 (cytochrome b5, 3′ end, NM_{—}001914) (SEQ ID NO:39 or 40); COXVIb (coxVIb gene, last exon and flanking sequence, NM_{—}001863) (SEQ ID NO:41 or 42); and TCF4 (NM_{—}030756) (SEQ ID NO:43 or 44). HGD marker polypeptides refer to the polypeptides encoded by the HGD gene markers.

In an aspect, the invention involves a method for the diagnosis of esophageal high-grade dysplasia (HGD) in a patient, comprising establishing increased expression of at least eight genes (listed here with the polypeptide encoded by the gene) selected from the group consisting of ET-1 (endothelin-1, NM_{—}001955) (SEQ ID NO:1 or 2); AGR2 (anterior gradient 2 (Xenepus laevis) homolog, NM_{—}006408) (SEQ ID NO:3 or 4); ADAM8 (NM_{—}001109) (SEQ ID NO:5 or 6); PRSS8 (Prostasin precursor, serine protease, NM_{—}002773) (SEQ ID NO:7 or 8); AXO1 (Axonin-1 precursor, NM_{—}005076) (SEQ ID NO:9 or 10); NROB2 (Nuclear hormone receptor, NM_{—}021969) (SEQ ID NO:11 or 12); TM7SF1 (NM_{—}003272) (SEQ ID NO:13 or 14); DLDH (dihydrolipamide dehydrogenase, NM_{—}000108) (SEQ ID NOS:15 or 16); MAT2B (methionine adenosyltransferase II, beta, NM_{—}013283) (SEQ ID NO:17 or 18); STC-2 (stanniocalcin-2, NM_{—}003714) (SEQ ID NO:19 or 20); PPBI (alkaline phosphatase, intestinal precursor, NM_{—}001631) (SEQ ID NO:21 or 22); SLNAC1 (sodium channel receptor SLNAC1, NM_{—}004769) (SEQ ID NO:23 or 24); CAH4 (carbonic anhydrase iv precursor, NM_{—}000717) (SEQ ID NO:25 or 26); PA21 (phopholipase a2 precursor, NM_{—}000928) (SEQ ID NO:27 or 28); PAR2 (proteinase activated receptor 2 precursor, NM_{—}005242) (SEQ ID NO:29 or 30); IDE (insulin-degrading enzyme, NM_{—}004969) (SEQ ID NO:31 or 32); MYO1A (myosin-1A, NM_{—}005379) (SEQ ID NO:33 or 34); CYP2J2 (cytochrome P450 monooxygenase, NM_{—}000775) (SEQ ID NO:35 or 36); PHYH (phytanoyl-CoA-hydroxylase (Refsum disease), NM_{—}006214) (SEQ ID NO:37 or 38); CYB5 (cytochrome b5, 3′ end, NM_{—}001914) (SEQ ID NO:39 or 40); COXVIb (coxVIb gene, last exon and flanking sequence, NM_{—}001863) (SEQ ID NO:41 or 42); and TCF4 (NM_{—}030756) (SEQ ID NO:43 or 44); and comparing expression of the genes to a baseline expression of the genes in normal tissue controls; wherein an increase of at least 1.5-fold in expression (and/or p value <0/07) of the genes from the group relative to the baseline indicates that the patient is experiencing esophageal high-grade dysplasia. In an embodiment of the invention, the tissue is human tissue.

In another embodiment, the invention involves a method of identifying a patient susceptable to esophageal adenocarcoma, comprising diagnosing esophageal high-grade dysplasia in a patient by establishing increased expression of at least eight genes selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43); and comparing expression of the genes to a baseline expression of the genes in normal tissue controls; wherein an increase of at least 1.5-fold in expression of the genes from the group relative to the baseline indicates that the patient is experiencing esophageal high-grade dysplasia. Alternatively, the patient may be susceptible to colon carcinoma and the diagnosing of high-grade dysplasia is by similarly determining expression of at least eight genes of the above group in a test colon tissue sample compared to a normal colon tissue sample.

In still another embodiment, the invention involves a method for determining whether an esophageal tissue is predisposed to a neo-plastic transformation, comprising determining whether in a cell from the esophageal tissue at least eight nucleic acid sequences selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756):(SEQ ID NO:43) is expressed at least 1.5-fold above baseline expression in a normal tissue control. In an embodiment, the tissue is human tissue.

In another aspect, the invention involves a method for the diagnosis of esophageal high-grade dysplasia in a patient, comprising establishing the level of expression a polypeptide encoded by at least eight genes selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43); and comparing expression of the at least eight genes from the group to a baseline expression of the genes in normal tissue controls; wherein an increase of at least 1.5-fold in expression of the polypeptide encoded by the genes from the group relative to the baseline indicates that the patient has esophageal dysplasia.

In an embodiment, the method involves contacting a HGD cell or a cancer cell with an antibody that binds specifically to a polypeptide, or fragment thereof, encoded by a gene selected from the group of HGD marker genes or cancer marker genes as disclosed herein.

In an embodiment, the method involves determining expression of at least 8 of the genes of the group of HGD marker genes using by nucleic acid miroarray analysis. In further embodiment, the microarray comprises nucleic acid sequences of at least 20 nucleotides derived from at least eight of the genes from the following group: ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43).

In another embodiment, the invention involves analysis using a microarray comprising nucleic acid probe sequences comprising at least 20 contiguous nucleotides from at least 8 genes selected from the group of HGD marker genes: ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43).

In a further embodiment, the methods of detecting high-grade dysplasia, diagnosing high-grade dysplasia, or prognosing development of cancer from detected high-grade dysplasia involves determining expression of at least eight genes from the group of HGD markers disclosed herein above as determined by an analysis method including, but not limited to polymerase chain reaction analysis, real-time polymerase chain reaction analysis, Taqman® polymerase chain reaction analysis, nucleic acid hybridization, fluorescent in situ hybridization and non-fluorescent in situ hybridization (e.g. radioactive, calorimetric, enzymatic or enzyme-linked detection methods for in situ hybridization). Where the method of the invention involves determining increased expression of polypeptides encoded by at least eight HGD marker genes as disclosed herein above, an embodiment of the method involves analysis using an antibody capable of specifically binding to a polypeptide, or a fragment thereof, encoded by a HGD marker gene.

In an alternative embodiment, the analytical methods of the invention involve probes or targets labelled with radionuclides or enzymatic labels such that expression of a gene or polypeptide is determinable.

In an embodiment of any of the methods or compositions of the invention, the dysplasia is high-grade dysplasia of esophagus tissue and the cancer is esophageal adenocarcinoma. Alternatively the patient is a human patient.

In another aspect, the invention involves a method of treating high-grade esophageal dysplasia or inhibiting or preventing cancer in a patient in need of such treatment, the method comprising administering to the patient a compound capable of decreasing expression of a gene selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43).

In still another aspect, the invention involves a method of treating high-grade esophageal dysplasia or inhibiting or preventing cancer in a patient in need of such treatment, the method comprising administering to the patient a compound capable of decreasing expression of a polypeptide encoded by a gene selected from the HGD marker genes.

In still another aspect, the invention involves a method of treating high-grade esophageal dysplasia or inhibiting or preventing cancer in a patient in need of such treatment, the method comprising administering to the patient a compound capable of inhibiting activity of a polypeptide encoded by a gene which is one of at least eight genes selected from the group of HGD marker genes as disclosed herein. In an embodiment, the compound is an antagonist of the polypeptide. In a further embodiment, the antagonist is an antibody, such as a monoclonal antibody or a humanized monoclonal antibody.

In a further aspect, the invention involves a method of screening for candidate drugs which inhibits or prevents progression from dysplasia to adenocarcinoma, the method comprising contacting a cell with a candidate drug, and assaying inhibition of progression from high-grade dysplasia to cancer in the cell, wherein the cell, prior to contacting with the candidate drug, expresses at least eight genes at a level at least 1.5-fold increased relative to a normal tissue baseline level, wherein the genes are selected from group of HGD marker genes as disclosed herein.

In another aspect, the invention involves a method of inhibiting or preventing progression from high-grade dysplasia to cancer in a patient by administering a drug identified by screening for candidate drugs which inhibits or prevents progression from dysplasia to adenocarcinoma, the method comprising contacting a cell with a candidate drug, and assaying inhibition of progression from high-grade dysplasia to cancer in the cell, wherein the cell, prior to contacting with the candidate drug, expresses at least eight genes at a level at least 1.5-fold increased relative to a normal tissue baseline level, wherein the genes are selected from group of HGD marker genes as disclosed herein.

In another aspect, the invention involves a compound capable of inhibiting or preventing the progression from high-grade dysplasia to cancer in a patient. In an embodiment of the invention the compound is identified by screening for a candidate drug which inhibits or prevents progression from dysplasia to adenocarcinoma, the method comprising contacting a cell expressing at least 1.5-fold relative to a normal tissue baseline level at least eight genes selected from the group of HGD marker genes as disclosed herein, with a candidate drug, and assaying inhibition of progression from high-grade dysplasia to cancer in the cell. In an embodiment, the invention involves a pharmaceutical composition comprising a compound capable of inhibiting or preventing the progression from high-grade dysplasia to cancer in a patient, and a pharmaceutically acceptable carrier.

In still another aspect, the invention involves detecting cancer in a patient by determining that a gene, or the polypeptide it encodes, selected from the group consisting of CAD17 (liver-intestine cadherin, NM_{—}004063) (SEQ ID NO:45 or 46), CLDN15 (claudin 15, NM_{—}014343) (SEQ ID NO:47 or 48), SLNAC1 (sodium channel, NM_{—}004769) (SEQ ID NO:23 or 24), CFTR (chloride channel, NM_{—}000492) (SEQ ID NO:49 or 50), H2R (histamine H2 receptor, NM_{—}022304) (SEQ ID NO:51 or 52), PRSS8 (serine protease, NM_{—}002773) (SEQ ID NO:7 or 8), PA21 (phospholipase A2 group IB, NM_{—}000928) (SEQ ID NO:27 or 28), AGR2 (anterior gradient 2 homolog, (NM_{—}006408) (SEQ ID NO:3 or 4), EGFR (NM_{—}005228) (SEQ ID NO:53 or 54), EPHB2 (NM_{—}004442) (SEQ ID NO:55 or 56), CRIPTO CR-1 (NM_{—}003212) (SEQ ID NO:57 or 58), Eprin B1 (NM_{—}004429) (SEQ ID NO:59 or 60), MMP-17/MT4-MMP (NM_{—}016155) (SEQ ID NO:61 or 62), MMP26 (NM_{—}021801) (SEQ ID NO:63 or 64), ADAM10 (NM_{—}001110) (SEQ ID NO:65 or 66), ADAM8 (NM_{—}001109) (SEQ ID NO:5 or 6), ADAM1 (XM_{—}132370) (SEQ ID NO:67 or 68), TIM1 (NM_{—}003254) (SEQ ID NO:69 or 70), MUC1 (XM_{—}053256) (SEQ ID NO:71 or 72), CEA (NM_{—}004363) (SEQ ID NO:73 or 74), NCA (NM_{—}002483) (SEQ ID NO:75 or 76), Follistatin (NM_{—}006350) (SEQ ID NO:77 or 78), Claudin 1 (NM_{—}021101) (SEQ ID NO:79 or 80), Claudin 14 (NM_{—}012130) (SEQ ID NO:81 or 82), tenascin-R (NM_{—}003285) (SEQ ID NO:83 or 84), CAD3 (NM_{—}001793) (SEQ ID NO:85 or 86), AXO1 (NM_{—}005076) (SEQ ID NO:9 or 10), CONT (NM_{—}001843) (SEQ ID NO:87 or 88), Osteopontin (NM_{—}000582) (SEQ ID NO:89 or 90), Galectin 8 (NM_{—}006499) (SEQ ID NO:91 or 92), PGS1 (bihlycan, NM_{—}001711) (SEQ ID NO:93 or 94), Frizzled 2 (NM_{—}001466) (SEQ ID NO:95 or 96), ISLR (NM_{—}005545) (SEQ ID NO:97 or 98), FLJ23399 (NM_{—}022763) (SEQ ID NO:99 or 100), TEM1 (NM_{—}020404) (SEQ ID NO:101 or 102), Tie2 ligand2 (NM_{—}001147) (SEQ ID NO:103 or 104), STC-2 (NM_{—}003714) (SEQ ID NO:19 or 20), VEGFC (NM_{—}005429) (SEQ ID NO:105 or 106), tPA (NM_{—}000930) (SEQ ID NO:107 or 108), Endothelin 1 (NM_{—}001955) (SEQ ID NO:1 or 2), Thrombomodulin (NM_{—}000361) (SEQ ID NO:109 or 110), TF (NM_{—}001993) (SEQ ID NO:111 or 112), GPR4 (NM_{—}005282) (SEQ ID NO:113 or 114), GPR66 (NM_{—}006056) (SEQ ID NO:115 or 116), SLC22A2 (NM_{—}003058) ((SEQ ID NO:117 or 118), MLSN1 (NM_{—}002420) (SEQ ID NO:119 or 120), and ATN2 (Na/K transport, NM_{—}000702) (SEQ ID NO:121 or 122) is expressed at a level of about 1.5-fold in a test sample above the level of expression in a normal tissue sample of the same tissue type. The test sample is generally from a patient suspected of experiencing cancer, including, but not limited to, adenocarcinoma, esophageal adenocarcinoma, or colon cancer. The test sample is generally from the esophagus or colon of the patient. In an embodiment, at least two, alternatively at least three, alternatively at least five, and alternatively at least eight genes selected from the above group is upregulated in cancer tissue at 1.5-fold relative to normal tissue. Detection of the up-regulation of these genes is determined by, for example, hybridization analysis as standard in the and disclosed herein, as well as through antibody binding analysis of the level polypeptides expressed by the up-regulated gene or genes.

In an embodiment, the invention involves treatment by contacting a cancer cell with a compound that inhibits expression of at least one, optionally at least two, at least three, at least five, or at least eight genes, or the polypeptides encoded by the genes, selected from the group consisting of CAD17 (liver-intestine cadherin, NM_{—}004063) (SEQ ID NO:45 or 46), CLDN15 (claudin 15, NM_{—}014343) (SEQ ID NO:47 or 48), SLNAC1 (sodium channel, NM_{—}004769) (SEQ ID NO:23 or 24), CFTR (chloride channel, NM_{—}000492) (SEQ ID NO:49 or 50), H2R (histamine H2 receptor, NM_{—}022304) (SEQ ID NO:51 or 52), PRSS8 (serine protease, NM_{—}002773) (SEQ ID NO:7 or 8), PA21 (phospholipase A2 group IB, NM_{—}000928) (SEQ ID NO:27 or 28), AGR2 (anterior gradient 2 homolog, (NM_{—}006408) (SEQ ID NO:3 or 4), EGFR (NM_{—}005228) (SEQ ID NO:53 or 54), EPHB2 (NM_{—}004442) (SEQ ID NO:55 or 56), CRIPTO CR-1 (NM_{—}003212) (SEQ ID NO:57 or 58), Eprin B1 (NM_{—}004429) (SEQ ID NO:59 or 60), MMP-17/MT4-MMP (NM_{—}016155) (SEQ ID NO:61 or 62), MMP26 (NM_{—}021801) (SEQ ID NO:63 or 64), ADAM10 (NM_{—}001110) (SEQ ID NO:65 or 66), ADAM8 (NM_{—}001109) (SEQ ID NO:5 or 6), ADAM1 (XM_{—}132370) (SEQ ID NO:67 or 68), TIM1 (NM_{—}003254) (SEQ ID NO:69 or 70), MUC1 (XM_{—}053256) (SEQ ID NO:71 or 72), CEA (NM_{—}004363) (SEQ ID NO:73 or 74), NCA (NM_{—}002483) (SEQ ID NO:75 or 76), Follistatin (NM_{—}006350) (SEQ ID NO:77 or 78), Claudin 1 (NM_{—}021101) (SEQ ID NO:79 or 80), Claudin 14 (NM_{—}012130) (SEQ ID NO:81 or 82), tenascin-R (NM_{—}003285) (SEQ ID NO:83 or 84), CAD3 (NM_{—}001793) (SEQ ID NO:85 or 86), AXO1 (NM_{—}005076) (SEQ ID NO:9 or 10), CONT (NM_{—}001843) (SEQ ID NO:87 or 88), Osteopontin (NM_{—}000582) (SEQ ID NO:89 or 90), Galectin 8 (NM_{—}006499) (SEQ ID NO:91 or 92), PGS1 (bihlycan, NM_{—}001711) (SEQ ID NO:93 or 94), Frizzled 2 (NM_{—}001466) (SEQ ID NO:95 or 96), ISLR (NM_{—}005545) (SEQ ID NO:97 or 98), FLJ23399 (NM_{—}022763) (SEQ ID NO:99 or 100), TEM1 (NM_{—}020404) (SEQ ID NO:101 or 102), Tie2 ligand2 (NM_{—}001147) (SEQ ID NO:103 or 104), STC-2 (NM_{—}003714) (SEQ ID NO:19 or 20), VEGFC (NM_{—}005429) (SEQ ID NO:105 or 106), tPA (NM_{—}000930) (SEQ ID NO:107 or 108), Endothelin 1 (NM_{—}001955) (SEQ ID NO:1 or 2), Thrombomodulin (NM_{—}000361) (SEQ ID NO:109 or 110), TF (NM_{—}001993) (SEQ ID NO:111 or 112), GPR4 (NM_{—}005282) (SEQ ID NO:113 or 114), GPR66 (NM_{—}006056) (SEQ ID NO:115 or 116), SLC22A2 (NM_{—}003058) ((SEQ ID NO:117 or 118), MLSN1 (NM_{—}002420) (SEQ ID NO:119 or 120), and ATN2 (Na/K transport, NM_{—}000702) (SEQ ID NO:121 or 122). In another embodiment, treatment is by contacting the cancer cell with a compound that inhibits the production or activity of a polypeptide of the above group and/or encoded by a gene of the above group. Where inhibition of a polypeptide is desired, the compound is often an antibody specific for the polypeptide, is often a monoclonal antibody such as a humanized antibody.

In yet another aspect, the invention involves a method of screening a candidate compound for the ability to inhibit cancer cell growth or cause cancer cell death by contacting the candidate compound with a cancer cell expressing a gene or polypeptide selected from the following group: CAD17 (liver-intestine cadherin, NM_{—}004063) (SEQ ID NO:45 or 46), CLDN15 (claudin 15, NM_{—}014343) (SEQ ID NO:47 or 48), SLNAC1 (sodium channel, NM_{—}004769) (SEQ ID NO:23 or 24), CFTR (chloride channel, NM_{—}000492) (SEQ ID NO:49 or 50), H2R (histamine H2 receptor, NM_{—}022304) (SEQ ID NO:51 or 52), PRSS8 (serine protease, NM_{—}002773) (SEQ ID NO:7 or 8), PA21 (phospholipase A2 group IB, NM_{—}000928) (SEQ ID NO:27 or 28), AGR2 (anterior gradient 2 homolog, (NM_{—}006408) (SEQ ID NO:3 or 4), EGFR (NM_{—}005228) (SEQ ID NO:53 or 54), EPHB2 (NM_{—}004442) (SEQ ID NO:55 or 56), CRIPTO CR-1 (NM_{—}003212) (SEQ ID NO:57 or 58), Eprin B1 (NM_{—}004429) (SEQ ID NO:59 or 60), MMP-17/MT4-MMP (NM_{—}016155) (SEQ ID NO:61 or 62), MMP26 (NM_{—}021801) (SEQ ID NO:63 or 64), ADAM10 (NM_{—}001110) (SEQ ID NO:65 or 66), ADAM8 (NM_{—}001 109) (SEQ ID NO:5 or 6), ADAM1 (XM_{—}132370) (SEQ ID NO:67 or 68), TIM1 (NM_{—}003254) (SEQ ID NO:69 or 70), MUC1 (XM_{—}053256) (SEQ ID NO:71 or 72), CEA (NM_{—}004363) (SEQ ID NO:73 or 74), NCA (NM_{—}002483) (SEQ ID NO:75 or 76), Follistatin (NM_{—}006350) (SEQ ID NO:77 or 78), Claudin 1 (NM_{—}021101) (SEQ ID NO:79 or 80), Claudin 14 (NM_{—}012130) (SEQ ID NO:81 or 82), tenascin-R (NM_{—}003285) (SEQ ID NO:83 or 84), CAD3 (NM_{—}001793) (SEQ ID NO:85 or 86), AXO1 (NM_{—}005076) (SEQ ID NO:9 or 10), CONT (NM_{—}001843) (SEQ ID NO:87 or 88), Osteopontin (NM_{—}000582) (SEQ ID NO:89 or 90), Galectin 8 (NM_{—}006499) (SEQ ID NO:91 or 92), PGS1 (bihlycan, NM_{—}001711) (SEQ ID NO:93 or 94), Frizzled 2 (NM_{—}001466) (SEQ ID NO:95 or 96), ISLR (NM_{—}005545) (SEQ ID NO:97 or 98), FLJ23399 (NM_{—}022763) (SEQ ID NO:99 or 100), TEM1 (NM_{—}020404) (SEQ ID NO:101 or 102), Tie2 ligand2 (NM_{—}001147) (SEQ ID NO:103 or 104), STC-2 (NM_{—}003714) (SEQ ID NO:19 or 20), VEGFC (NM_{—}005429) (SEQ ID NO:105 or 106), tPA (NM_{—}000930) (SEQ ID NO:107 or 108), Endothelin 1 (NM_{—}001955) (SEQ ID NO:1 or 2), Thrombomodulin (NM_{—}000361) (SEQ ID NO:109 or 110), TF (NM_{—}001993) (SEQ ID NO:111 or 112), GPR4 (NM_{—}005282) (SEQ ID NO:113 or 114), GPR66 (NM_{—}006056) (SEQ ID NO:115 or 116), SLC22A2 (NM_{—}003058) ((SEQ ID NO:117 or 118), MLSN1 (NM_{—}002420) (SEQ ID NO:119 or 120), and ATN2 (Na/K transport, NM_{—}000702) (SEQ ID NO:121 or 122), wherein gene expression of at least one, at least two, at least three, at least five, or at least eight genes selected from the group are expressed at a level at least about 1.5-fold above the level in normal control tissue. Where the candidate compound is an antibody, the antibody is alternatively a polyclonal, monoclonal, humanized antibody, a Fab, a F(ab′)2, or a binding fragment of any one of these compounds.

In an embodiment, the sequences which are used to determine sequence identity or similarity are selected from the sequences described herein. Optionally, sequence variants are naturally occurring allelic variants, sequence variants or splice variants of these sequences. Sequence identity is typically calculated using the BLAST algorithm, described in Altschul et al Nucleic Acids Res. 25,3389-3402 (1997) with the BLOSUM62 default matrix.

In one embodiment, nucleic acid homology can be determined through hybridisation studies. Nucleic acids which hybridise under stringent conditions to the nucleic acids of the invention are considered high-grade esophageal dysplasia sequences. Under stringent conditions, hybridisation will most preferably occur at 42° C. in 750 mM NaCl, 75 mM trisodium citrate, 2% SDS, 50% formamide, 1× Denhart's, 10% (w/v) dextran sulphate and 100 pg/ml denatured salmon sperm DNA. Useful variations on these conditions will be readily apparent to those skilled in the art. The washing steps which follow hybridization most preferably occur at 65° C. in 15 mM NaCl, 1.5 mM trisodium citrate, and 1% SDS. Additional variations on these conditions will be readily apparent to those skilled in the art.

As a result of the degeneracy of the genetic code, a number of polynucleotide sequences encoding polypeptides of the invention, some that may have minimal similarity to the polynucleotide sequences of any known and naturally occurring gene, may be produced. Thus, the invention includes each and every possible variation of polynucleotide sequence that could be made by selecting combinations based on possible codon choices. These combinations are made in accordance with the standard triplet genetic code as applied to the polynucleotide sequence of naturally occurring high-grade esophageal dysplasia sequences, and all such variations are to be considered as being specifically disclosed.

The polynucleotides of this invention include RNA, cDNA, genomic DNA, synthetic forms, and mixed polymers, both sense and antisense strands, and may be chemically or biochemically modified, or may contain non-natural or derivatised nucleotide bases as will be appreciated by those skilled in the art. Such modifications include labels, methylation, intercalators, alkylators and modified linkages. In some instances it may be advantageous to produce nucleotide sequences encoding high-grade esophageal dysplasia sequences of the invention, or their derivatives, possessing a substantially different codon usage than that of the naturally occurring gene. For example, codons may be selected to increase the rate of expression of the peptide in a particular prokaryotic or eukaryotic host corresponding with the frequency that particular codons are utilized by the host. Other reasons to alter the nucleotide sequence encoding high-grade esophageal dysplasia sequences of the invention, or their derivatives, without altering the encoded amino acid sequences include the production of RNA transcripts having more desirable properties, such as a greater half-life, than transcripts produced from the naturally occurring sequence.

In some instances, useful nucleic acid sequences up-regulated in high-grade esophageal dysplasia of the invention are fragments of larger genes and may be used to identify and obtain corresponding full-length genes. Full-length sequences of the genes selected from the HGD gene marker group or cancer gene marker group of the invention can be obtained using a partial gene sequence using methods known per se to those skilled in the art. For example,“restriction-site PCR” may be used to retrieve unknown sequence adjacent to a portion of DNA whose sequence is known. In this technique universal primers are used to retrieve unknown sequence. Inverse PCR may also be used, in which primers based on the known sequence are designed to amplify adjacent unknown sequences. These upstream sequences may include promoters and regulatory elements. In addition, various other PCR-based techniques may be used, for example a kit available from Clontech (Palo Alto, Calif.) allows for a walking PCR technique, the 5′RACE kit (Gibco-BRL) allows isolation of additional sequence while additional 3′sequence can be obtained using practised techniques.

The present invention allows for the preparation of purified high-grade dysplasia polypeptide (i.e. a polypeptide encoded by a gene disclosed herein as up-regulated in high-grade esophageal dysplasia) or protein, from the polynucleotides of the present invention or variants thereof. In order to do this, host cells may be transfected with a nucleic acid molecule as described above. Typically said host cells are transfected with an expression vector comprising a nucleic acid encoding a high-grade esophageal dysplasia protein according to the invention. Cells are cultured under the appropriate conditions to induce or cause expression of the high-grade esophageal dysplasia protein. The conditions appropriate for high-grade esophageal dysplasia protein expression will vary with the choice of the expression vector and the host cell, and will be easily ascertained by one skilled in the art.

A variety of expression vector/hosi systems may be utilized to contain and express the high-grade dysplasia sequences of the invention and are well known in the art. These include, but are not limited to, microorganisms such as bacteria transformed with plasmid or cosmid DNA expression vectors; yeast transformed with yeast expression vectors; insect cell systems infected with viral expression vectors (e. g., baculovirus); or mouse or other animal or human tissue cell systems. In a preferred embodiment the high-grade esophageal dysplasia proteins of the invention are expressed in mammalian cells using various expression vectors including plasmid, cosmid and viral systems such as adenoviral, retroviral or vaccinia virus expression systems. The invention is not limited by the host cell employed.

The polynucleotide sequences, or variants thereof, of the present invention can be stably expressed in cell lines to allow long term production of recombinant proteins in mammalian systems. These sequences can be transformed into cell lines using expression vectors which may contain viral origins of replication and/or endogenous expression elements and a selectable marker gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

The protein produced by a transformed cell may be secreted or retained intracellularly depending on the sequence and/or the vector used. As will be understood by those of skill in the art, expression vectors containing polynucleotides which encode a protein of the invention may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of the protein may also be used to specify protein targeting, folding, and/or activity. Different host cells having specific cellular machinery and characteristic mechanisms for post- translational activities (e. g., CHO or HeLa cells), are available from the American Type Culture Collection (ATCC) and may be chosen to ensure the correct modification and processing of the foreign protein.

When large quantities of protein are needed such as for antibody production, vectors which direct high levels of high-grade esophageal dysplasia gene expression may be used such as those containing the T5 or T7 inducible bacteriophage promoter.

The present invention also includes the use of the expression systems described above in generating and isolating fusion proteins which contain important functional domains of the protein. These fusion proteins are used for binding, structural and functional studies as well as for the generation of appropriate antibodies.

In order to express and purify the protein as a fusion protein, the appropriate cDNA sequence is inserted into a vector which contains a nucleotide sequence encoding another peptide (for example, glutathionine succinyl transferase). The fusion protein is expressed and recovered from prokaryotic or eukaryotic cells. The fusion protein can then be purified by affinity chromatography based upon the fusion vector sequence. The relevant protein can subsequently be obtained by enzymatic cleavage of the fusion protein.

In one embodiment, a fusion protein may be generated by the fusion of a high-grade dysplasia polypeptide with a tag polypeptide which provides an epitope to which an anti-tag antibody can selectively bind. The epitope tag is generally placed at the amino-or carboxy-terminus of the high-grade esophageal dysplasia polypeptide. The presence of such epitope-tagged forms of a high-grade esophageal dysplasia polypeptide can be detected using an antibody against the tag polypeptide. Also, provision of the epitope tag enables the high-grade dysplasia polypeptide to be readily purified by affinity purification using an anti-tag antibody or another type of affinity matrix that binds to the epitope tag.

Various tag polypeptides and their respective antibodies are well known in the art. Examples include poly-histidine or poly-histidine-glycine tags and the c-myc tag and antibodies thereto. Fragments of high-grade dysplasia polypeptide may also be produced by direct peptide synthesis using solid-phase techniques. Automated synthesis may be achieved by using the ABI 433A Peptide Synthesizer (Applied Biosystems, Foster City, Calif.). Various fragments of high-grade dysplasia polypeptide may be synthesized separately and then combined to produce the full-length molecule.

In a further aspect of the invention there is provided a method of preparing a polypeptide as described above, comprising the steps of: (1) culturing the host cells under conditions effective for production of the polypeptide; and (2) harvesting the polypeptide.

Substantially purified high-grade dysplasia polypeptide or fragments thereof can then be used in further biochemical analyses to establish secondary and tertiary structure for example by x-ray crystallography of the protein or by nuclear magnetic resonance (NMR). Determination of structure allows for the rational design of pharmaceuticals to interact with the protein, alter protein charge configuration or charge interaction with other proteins, or to alter its function in the cell.

With the identification of the high-grade esophageal dysplasia marker gene nucleotide sequences and the polypeptide sequences encoded by them, probes and antibodies raised to the genes can be used in a variety of hybridisation and immunological assays to screen for and detect the presence of either a normal or mutated gene or gene product.

In addition the nucleotide and protein sequences of the high-grade dysplasia genes provided in this invention enable therapeutic methods for the treatment of cancer, such as adenocarcinoma associated with one or more of these genes, enable screening of compounds for therapeutic intervention, and also enable methods for the diagnosis or prognosis of cancer associated with the these genes. Examples of such cancers include, but are not limited to, esophageal adenocarcinoma.

Transducing retroviral vectors are often used for producing a cell line expressing a gene above the level of expression in a cell lacking the additional copy of the gene. Such a cell is useful according to the invention for the production of a cell line useful for screening candidate compounds capable of reducing expression of a gene associated with high-grade esophageal dysplasia, reducing expression of a polypeptide encoded by the gene, or inhibiting activity of the polypeptide, such that the cell does not progress from dysplasia to cancer. The full-length high-grade dysplasia gene, or portions thereof, can be cloned into a retroviral vector and expression can be driven from its endogenous promoter or from the retroviral long terminal repeat or from a promoter specific for the target cell type of interest. Other viral vectors can be used and include, as is known in the art, adenoviruses, adeno-associated virus, vaccinia virus, papovaviruses, lentiviruses and retroviruses of avian, murine and human origin.

The viral vector described herein above is also useful for gene therapy to reduce the activity of the high-grade dysplasia genes of the invention, such as by antisense expression inhibition or RNA interference (see, for example, Paddison, P. J. et al., Genes & Development 16:948-958 (2002) and Brummelkamp, T. R. et al., Science 296:550-553 (2002)). Gene therapy would be carried out according to established methods (Friedman, 1991; Culver, 1996). A vector containing a copy of a high-grade esophageal dysplasia gene linked to expression control elements and capable of replicating inside the cells is prepared. Alternatively the vector may be replication deficient and may require helper cells or helper virus for replication and virus production and use in gene therapy.

Gene transfer using non-viral methods of infection can also be used. These methods include direct injection of DNA, uptake of naked DNA in the presence of calcium phosphate, electroporation, protoplast fusion or liposome delivery. Gene transfer can also be achieved by delivery as a part of a human artificial chromosome or receptor-mediated gene transfer. This involves linking the DNA to a targeting molecule that will bind to specific cell-surface receptors to induce endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes.

Inhibiting high-grade esophageal dysplasia gene or polypeptide function that are up-regulated in cancer can be achieved in a variety of ways as would be appreciated by those skilled in the art. Typically, a vector expressing the complement of a polynucleotide encoding a high-grade dysplasia gene of the invention may be administered to a subject to treat or prevent a disorder associated with increased activity and/or expression of the gene including, but not limited to, those described above.

Antisense strategies may use a variety of approaches including the use of antisense oligonucleotides, ribozymes, DNAzymes, injection of antisense RNA and transfection of antisense RNA expression vectors. Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (see, for example, Goldman, C K. et al., Nature Biotechnology 15: 462-466 (1997))

Where purified protein or polypeptide is used to produce antibodies which specifically bind a high-grade dysplasia protein, the antibody(ies) are used directly as an antagonist or indirectly as a targeting or delivery mechanism for bringing a pharmaceutical agent to cells or tissues that express the protein. Such antibodies may include, but are not limited to, polyclonal, monoclonal, chimeric and single chain antibodies as would be understood by the person skilled in the art.

For the production of antibodies, various hosts including rabbits, rats, goats, mice, humans, and others may be immunized by injection with a protein of the invention or with any fragment or oligopeptide thereof, which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum.

It is preferred that the oligopeptides, peptides, or fragments used to induce antibodies to the high-grade dysplasia of the invention have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of amino acids from these proteins may be fused with those of another protein, such as KLH, and antibodies to the chimeric molecule may be produced.

Monoclonal antibodies to high-grade dysplasia polypeptides or proteins of the invention may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler, G. and Milstein, C., Nature 256:495-497 (1975); Kozbor, D. et al., Immunol. Methods 81:31-42 (1985); and Cole, S. P. et al., Mol. Cell Biol. 62:109-120 (1984)).

Antibodies may also be produced by inducing in vivo production in the lymphocyte population or by screening immunoglobulin libraries or panels of highly specific binding reagents as disclosed in the literature.

Antibody fragments which contain specific binding sites for the high-grade esophageal dysplasia proteins may also be generated. For example, such fragments include fragments produced by pepsin digestion of the antibody molecule and Fab fragments generated by reducing the disulfide bridges of the F(AB)2 fragments. Alternatively, Fab expression libraries may be constructed to allow rapid and easy identification of monoclonal Fab fragments with the desired specificity. (For example, see Huse, W. D. et al., Science 246:1275-1281 (1989)). Various immunoassays well known in art may be used for screening to identify antibodies having the desired specificity.

Numerous protocols for competitive binding or immunoradiometric assays using either polyclonal or monoclonal antibodies with established specificities are well known in the art. Such immunoassays typically involve the measurement of complex formation between a protein and its specific antibody. A two-site, monoclonal-based immunoassay utilizing monoclonal antibodies reactive to two non-interfering epitopes is preferred, but a competitive binding assay may also be employed.

Candidate pharmaceutical agents or compounds encompass numerous chemical classes, though typically they are organic molecules, preferably small organic compounds having molecular weight of more than 100 and less than about 2,500 daltons. Candidate agents are also found among biomolecules including peptides, saccharides, fatty acids and steroids and peptides.

Agent screening techniques include, but are not limited to, utilising eukaryotic or prokaryotic host cells that are stably transformed with recombinant molecules expressing a particular high-grade dysplasia polypeptide of the invention, or fragment thereof, preferably in competitive binding assays. Binding assays will measure for the formation of complexes between the high-grade esophageal dysplasia polypeptide, or fragments thereof, and the agent being tested, or will measure the degree to which an agent being tested will interfere with the formation of a complex between the high-grade esophageal dysplasia polypeptide, or fragment thereof, and a known ligand.

Another technique for drug screening provides high- throughput screening for compounds having suitable binding affinity to a high-grade dysplasia polypeptide. In such a technique, large numbers of small peptide test compounds are synthesised on a solid substrate and can be assayed through high-grade esophageal dysplasia polypeptide binding and washing. Bound high-grade dysplasia polypeptide is then detected by methods well known in the art. In a variation of this technique, purified polypeptides can be coated directly onto plates to identify interacting test compounds.

An additional method for drug screening involves the use of host eukaryotic cell lines which carry mutations in a particular high-grade dysplasia gene. The host cell lines are also defective at the polypeptide level. Other cell lines may be used where the gene expression of the high-grade esophageal dysplasia gene can be switched off or up-regulated. The host cell lines or cells are grown in the presence of various drug compounds and the rate of growth of the host cells is measured to determine if the compound is capable of regulating the growth of defective cells.

A high-grade esophageal dysplasia polypeptide encoded by an HGD marker gene may also be used for screening compounds developed as a result of combinatorial library technology. This provides a way to test a large number of different substances for their ability to modulate activity of a polypeptide. The use of peptide libraries is preferred with such libraries and their use known in the art.

A substance identified as a modulator of polypeptide function may be peptide or non-peptide in nature. Non-peptide “small molecules” are often preferred for many in vivo pharmaceutical applications. In addition, a mimic or mimetic of the substance may be designed for pharmaceutical use. The design of mimetics based on a known pharmaceutically active compound (i.e., a “lead compound”) is a common approach to the development of novel pharmaceuticals. This is often desirable where the original active compound is difficult or expensive to synthesise or where it provides an unsuitable method of administration. In the design of a mimetic, particular parts of the original active compound that are important in determining the target property are identified. These parts or residues constituting the active region of the compound are known as its pharmacophore. Once found, the pharmacophore structure is modelled according to its physical properties using data from a range of sources including x-ray diffraction data and NMR. A template molecule is then selected onto which chemical groups which mimic the pharmacophore can be added. The selection can be made such that the mimetic is easy to synthesise, is likely to be pharmacologically acceptable, does not degrade in vivo and retains the biological activity of the lead compound. Further optimisation or modification can be carried out to select one or more final mimetics useful for in vivo or clinical testing.

It is also possible to isolate a target-specific antibody and then solve its crystal structure. In principle, this approach yields a pharmacophore upon which subsequent drug design can be based as described above. It may be possible to avoid protein crystallography altogether by generating anti-idiotypic antibodies (anti-ids) to a functional, pharmacologically active antibody.

As a mirror image of a mirror image, the binding site of the anti-ids would be expected to be an analogue of the original binding site. The anti-id could then be used to isolate peptides from chemically or biologically produced peptide banks.

In further embodiments, any of the genes, proteins, antagonists, antibodies, complementary sequences, or vectors of the invention may be administered in combination with other appropriate therapeutic agents.

Selection of the appropriate agents may be made by those skilled in the art, according to conventional pharmaceutical principles. The combination of therapeutic agents may act synergistically to effect the treatment or prevention of the various disorders described above. Using this approach, therapeutic efficacy with lower dosages of each agent may be possible, thus reducing the potential for adverse side effects.

In a further aspect a pharmaceutical composition and a pharmaceutically acceptable carrier may be administered to a patient diagnosed as experiencing high-grade esophageal dysplasia for the inhibition or prevention of progression of the disease to adenocarcinoma.

The pharmaceutical composition may comprise any one or more of a polypeptide as described above, typically a substantially purified high-grade esophageal dysplasia polypeptide, an antibody to a high-grade esophageal dysplasia polypeptide, a vector capable of expressing a high-grade esophageal dysplasia polypeptide, a compound which increases or decreases expression of a high-grade esophageal dysplasia gene, a candidate drug that restores wild-type activity to a high-grade esophageal dysplasia gene or an antagonist of a high-grade esophageal dysplasia gene.

The pharmaceutical composition may be administered to a subject to treat or prevent a cancer associated with decreased activity and/or expression of a high-grade esophageal dysplasia gene including, but not limited to, those provided above.

Pharmaceutical compositions in accordance with the present invention are prepared by mixing a polypeptide of the invention, or active fragments or variants thereof, having the desired degree of purity, with acceptable carriers, excipients, or stabilizers which are well known.

Acceptable carriers, excipients or stabilizers are nontoxic at the dosages and concentrations employed, and include buffers such as phosphate, citrate, and other organic acids; antioxidants including absorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitrol or sorbitol; salt-forming counterions such as sodium; and/or nonionic surfactants such as Tween, Pluronics or polyethylene glycol (PEG).

Any of the therapeutic methods described above may be applied to any subject in need of such therapy, including, for example, mammals such as dogs, cats, cows, horses, rabbits, monkeys, and most preferably, humans.

Polynucleotide sequences encoding the high-grade esophageal dysplasia genes of the invention may be used for the diagnosis or prognosis of cancers associated with their dysfunction, or a predisposition to such cancers. Examples of such cancers include, but are not limited to, adenocarcinoma, such as in patients having Barrett's esophagus. Diagnosis or prognosis may be used to determine the severity, type or stage of the disease state in order to initiate an appropriate therapeutic intervention.

In another embodiment of the invention, the polynucleotides that may be used for diagnostic or prognostic purposes include oligonucleotide sequences, genomic DNA and complementary RNA and DNA molecules. The polynucleotides may be used to detect and quantitate gene expression in biopsied tissues in which mutations or abnormal expression of the relevant high-grade esophageal dysplasia gene may be correlated with disease. Genomic DNA used for the diagnosis or prognosis may be obtained from body cells, such as those present in the blood, tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, direct nucleotide sequencing, reverse transcriptase PCR (RT-PCR), hybridization using specific oligonucleotides, restriction enzyme digest and mapping, PCR mapping, RNAse protection, and various other methods may be employed.

Oligonucleotides specific to particular sequences can be chemically synthesized and labelled radioactively or non-radioactively and hybridised to individual samples immobilized on membranes or other solid-supports or in solution. The presence, absence or excess expression of a particular high-grade esophageal dysplasia gene may then be visualized using methods such as autoradiography, fluorometry, or colorimetry.

In a particular aspect, the nucleotide sequences encoding a high-grade esophageal dysplasia gene of the invention may be useful in assays that detect the presence of associated disorders, particularly those mentioned previously. The nucleotide sequences encoding the relevant high-grade esophageal dysplasia gene may be labelled by standard methods and added to a fluid or tissue sample from a patient under conditions suitable for the formation of hybridization complexes.

After a suitable incubation period, the sample is washed and the signal is quantitated and compared with a standard value. If the amount of signal in the patient sample is significantly altered in comparison to a control sample then the presence of altered levels of nucleotide sequences encoding the high-grade esophageal dysplasia gene in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis or prognosis of a disorder associated with a mutation in a particular high-grade esophageal dysplasia gene of the invention, the nucleotide sequence of the relevant gene can be compared between normal tissue and diseased tissue in order to establish whether the patient expresses a mutant gene.

In order to provide a basis for the diagnosis or prognosis of a disorder associated with abnormal expression of a particular high-grade esophageal dysplasia gene of the invention, a normal or standard profile for expression is established. This may be accomplished by combining body fluids or cell extracts taken from normal subjects, either animal or human, with a sequence, or a fragment thereof, encoding the relevant high-grade esophageal dysplasia gene, under conditions suitable for hybridization or amplification. Standard hybridization may be quantified by comparing the values obtained from normal subjects with values from an experiment in which a known amount of a substantially purified polynucleotide is used.

Another method to identify a normal or standard profile for expression of a particular high-grade esophageal dysplasia gene is through quantitative RT-PCR studies. RNA isolated from body cells of a normal individual, particularly RNA isolated from tumour cells, is reverse transcribed and real-time PCR using oligonucleotides specific for the relevant high-grade esophageal dysplasia gene is conducted to establish a normal level of expression of the gene.

Standard values obtained in both these examples may be compared with values obtained from samples from patients who are symptomatic for a disorder. Deviation from standard values is used to establish the presence of a disorder.

Once the presence of a disorder is established and a treatment protocol is initiated, hybridization assays or quantitative RT-PCR studies may be repeated on a regular basis to determine if the level of expression in the patient begins to approximate that which is observed in the normal subject. The results obtained from successive assays may be used to show the efficacy of treatment over a period ranging from several days to months.

In one aspect, hybridization with PCR probes which are capable of detecting polynucleotide sequences, including genomic sequences, encoding a particular high-grade esophageal dysplasia gene, or closely related molecules, may be used to identify nucleic acid sequences which encode the gene. The specificity of the probe, whether it is made from a highly specific region, e. g., the 5′regulatory region, or from a less specific region, e.g., a conserved motif, and the stringency of the hybridization or amplification will determine whether the probe identifies only naturally occurring sequences encoding the high-grade esophageal dysplasia gene, allelic variants, or related sequences.

Probes may also be used for the detection of related sequences, and should preferably have at least 50% sequence identity to any of the high-grade esophageal dysplasia encoding sequences. The hybridization probes of the subject invention may be DNA or RNA and may be derived from the sequence of HGD marker genes disclosed in Table 4 or from genomic sequences including promoters, enhancers, and introns of the genes.

Means for producing specific hybridization probes for DNAs encoding the high-grade esophageal dysplasia genes of the invention include the cloning of polynucleotide sequences encoding these genes or their derivatives into vectors for the production of mRNA probes. Such vectors are known in the art, and are commercially available. Hybridization probes may be labelled by radionuclides such as 32p or 35S, or by enzymatic labels, such as alkaline phosphatase coupled to the probe via avidin/biotin coupling systems, or other methods known in the art.

According to a further aspect of the invention there is provided the use of a polypeptide as described above in the diagnosis or prognosis of a cancer associated with a high-grade esophageal dysplasia gene of the invention, or a predisposition to such cancers.

When a diagnostic or prognostic assay is to be based upon a high-grade esophageal dysplasia protein, a variety of approaches are possible. For example, diagnosis or prognosis can be achieved by monitoring differences in the electrophoretic mobility of normal and mutant proteins. Such an approach will be particularly useful in identifying mutants in which charge substitutions are present, or in which insertions, deletions or substitutions have resulted in a significant change in the electrophoretic migration of the resultant protein. Alternatively, diagnosis may be based upon differences in the proteolytic cleavage patterns of normal and mutant proteins, differences in molar ratios of the various amino acid residues, or by functional assays demonstrating altered function of the gene products.

In another aspect, antibodies that specifically bind a high-grade esophageal dysplasia gene of the invention may be used for the diagnosis or prognosis of cancers characterized by abnormal expression of the gene, or in assays to monitor patients being treated with the gene or agonists, antagonists, or inhibitors of the gene. Antibodies useful for diagnostic purposes may be prepared in the same manner as described above for therapeutics. Diagnostic or prognostic assays include methods that utilize the antibody and a label to detect a high-grade esophageal dysplasia gene of the invention in human body fluids or in extracts of cells or tissues. The antibodies may be used with or without modification, and may be labelled by covalent or non-covalent attachment of a reporter molecule.

A variety of protocols for measuring a high-grade esophageal dysplasia gene of the invention, including ELISA, RIAs, and FACS, are known in the art and provide a basis for diagnosing altered or abnormal levels of their expression. Normal or standard values for their expression are established by combining body fluids or cell extracts taken from normal mammalian subjects, preferably human, with antibody to the high-grade esophageal dysplasia protein under conditions suitable for complex formation. The amount of standard complex formation may be quantitated by various methods, preferably by photometric means. Quantities of any of the high-grade esophageal dysplasia genes expressed in subject, control, and disease samples from biopsied tissues are compared with the standard values. Deviation between standard and subject values establishes the parameters for diagnosing disease.

Once an individual has been diagnosed with a cancer, effective treatments can be initiated. These may include administering a selective agonist to the relevant mutant high-grade esophageal dysplasia gene so as to restore its function to a normal level or introduction of the wild-type gene, particularly through gene therapy approaches as described above. Typically, a vector capable of expressing the appropriate full-length high-grade esophageal dysplasia gene or a fragment or derivative thereof may be administered. In an alternative approach to therapy, a substantially purified high-grade esophageal dysplasia polypeptide and a pharmaceutically acceptable carrier may be administered, as described above, or drugs which can replace the function of or mimic the action of the relevant high-grade esophageal dysplasia gene may be administered.

In the treatment of cancers associated with increased high-grade esophageal dysplasia gene expression and/or activity, the affected individual may be treated with a selective antagonist such as an antibody to the relevant protein or an antisense (complement) probe to the corresponding gene as described above, or through the use of drugs which may block the action of the relevant high-grade esophageal dysplasia gene.

In further embodiments, complete cDNAs, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to detect or prognose a disorder, and to develop and monitor the activities of therapeutic agents. Microarrays may be prepared, used, and analyzed using methods known in the art (for example, see Schena, M. et al. PNAS USA 93:10614-10619 (1996); Heller, R. A. et al., PNAS USA 94:2150-2155 (1997); and Heller, M. J., Annual Review of Biomedical Engineering 4:129-53 (2002)).

The present invention also provides for the production of genetically modified (knock-out, knock-down, knock-in and transgenic), non-human animal models transformed with the DNA molecules of the invention. These animals are useful for the study of high-grade esophageal dysplasia gene function, to study the mechanisms of cancer as related to the high-grade esophageal dysplasia genes, for the screening of candidate pharmaceutical compounds, for the creation of explanted mammalian cell cultures which express the protein or mutant protein and for the evaluation of potential therapeutic interventions.

One of the high-grade esophageal dysplasia genes of the invention may have been inactivated by knock-out deletion, and knock-out genetically modified non-human animals are therefore provided.

Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, genetically modified mice and rats are highly desirable due to their relative ease of maintenance and shorter life spans. For certain studies, transgenic yeast or invertebrates may be suitable and preferred because they allow for rapid screening and provide for much easier handling. For longer term studies, non-human primates may be desired due to their similarity with humans.

To create an animal model for a mutated high-grade esophageal dysplasia gene of the invention several methods can be employed. These include generation of a specific mutation in a homologous animal gene, insertion of a wild type human gene and/or a humanized animal gene by homologous recombination, insertion of a mutant (single or multiple) human gene as genomic or minigene cDNA constructs using wild type or mutant or artificial promoter elements or insertion of artificially modified fragments of the endogenous gene by homologous recombination. The modifications include insertion of mutant stop codons, the deletion of DNA sequences, or the inclusion of recombination elements (lox p sites) recognized by enzymes such as Cre recombinase.

To create a transgenic mouse, which is preferred, a mutant version of a particular high-grade esophageal dysplasia gene of the invention can be inserted into a mouse germ line using standard techniques of oocyte microinjection or transfection or microinjection into embryonic stem cells. Alternatively, if it is desired to inactivate or replace the endogenous high-grade esophageal dysplasia gene, homologous recombination using embryonic stem cells may be applied. For oocyte injection, one or more copies of the mutant or wild type high-grade esophageal dysplasia gene can be inserted into the pronucleus of a just-fertilized mouse oocyte. This oocyte is then reimplanted into a pseudo-pregnant foster mother. The liveborn mice can then be screened for integrants using analysis of tail DNA for the presence of human high-grade esophageal dysplasia gene sequences. The transgene can be either a complete genomic sequence injected as a YAC, BAC, PAC or other chromosome DNA fragment, a cDNA with either the natural promoter or a heterologous promoter, or a minigene containing all of the coding region and other elements found to be necessary for optimum expression. The genetically modified non-human animals as described above are useful for the screening of candidate pharmaceutical compounds.

### BRIEF DESCRIPTION OF THE DRAWINGS

FIGS. 1A and 1B are graphs showing a distribution of expression of IL-1H1 (FIG. 1A) and CYP2J2 (FIG. 1B) in the dysplasia-carcinoma sequence in BE. Expression in normal epithelium and in esophageal epithelia from samples of Barrett's esophagus (BE), dysplasia (D), BE adjacent to andenocarcinoma (BE-CA); and adenocarcinoma (CA) are plotted. The vertical line denotes the average Z score in each disease group. Normal refers to the normal esophagus group. Dysplasia includes low- and high-grade dysplasia samples.

FIGS. 2A and 2B are graphs showing a distribution of expression of AGR2 (FIG. 2A) and NROB2 (FIG. 2B) in the dysplasia-carcinoma sequence in BE. Expression in esophageal epithelia from samples of Barrett's esophagus (BE), dysplasia (D), BE adjacent to andenocarcinoma (BE-CA); and adenocarcinoma (CA) are plotted. The vertical line denotes the average Z score in each disease group. Normal refers to pooled epithelia samples. Dysplasia includes low- and high-grade dysplasia samples.

FIGS. 3A and 3B are graphs showing a distribution of expression of TCF4 (FIG. 3A) and FLJ23399 (FIG. 3B) in the dysplasia-carcinoma sequence in BE. Expression in esophageal epithelia from samples of Barrett's esophagus (BE), dysplasia (D), BE adjacent to andenocarcinoma (BE-CA); and adenocarcinoma (CA) are plotted. The vertical line denotes the average Z score in each disease group. Normal refers to pooled epithelia samples. Dysplasia includes low- and high-grade dysplasia samples.

FIGS. 4A and 4B show the nucleic acid sequence (SEQ ID NO:1) and the amino acid sequence (SEQ ID NO:2) of ET-1 (endothelin-1, NM_{—}001955).

FIGS. 5A and 5B show the nucleic acid sequence (SEQ ID NO:3) and the amino acid sequence (SEQ ID NO:4) of AGR2 (anterior gradient 2 (Xenepus laevis) homolog, NM_{—}006408).

FIGS. 6A and 6B show the nucleic acid sequence (SEQ ID NO:5) and the amino acid sequence (SEQ ID NO:6) of ADAM8 (NM_{—}001109).

FIGS. 7A and 7B show the nucleic acid sequence (SEQ ID NO:7) and the amino acid sequence (SEQ ID NO:8) of PSS8 (Prostasin precursor, serine protease, NM_{—}002773).

FIGS. 8A-8C show the nucleic acid sequence (SEQ ID NO:9) and FIG. 8D shows the amino acid sequence (SEQ ID NO:10) of AXO1 (Axonin-1 precursor, NM_{—}005076).

FIGS. 9A and 9B show the nucleic acid sequence (SEQ ID NO:11) and the amino acid sequence (SEQ ID NO:12) of NROB2 (Nuclear hormone receptor, NM_{—}021969).

FIGS. 10A and 10B show the nucleic acid sequence (SEQ ID NO:13) and the amino acid sequence (SEQ ID NO:14) of TM7SF1 (NM_{—}003272).

FIGS. 11A and 11B show the nucleic acid sequence (SEQ ID NO:15) and the amino acid sequence (SEQ ID NO:16) of DLDH (dihydrolipamide dehydrogenase, NM_{—}000108).

FIGS. 12A and 12B show the nucleic acid sequence (SEQ ID NO:17) and the amino acid sequence (SEQ ID NO:18) of MAT2B (methionine adenosyltransferase II, beta, NM_{—}013283).

FIGS. 13A and 13B show the nucleic acid sequence (SEQ ID NO:19) and the amino acid sequence (SEQ ID NO:20) of STC-2 (stanniocalcin-2, NM_{—}003714).

FIGS. 14A and 14B show the nucleic acid sequence (SEQ ID NO:21) and the amino acid sequence (SEQ ID NO:22) of PPBI (alkaline phosphatase, intestinal precursor, NM_{—}001631).

FIGS. 15A and 15B show the nucleic acid sequence (SEQ ID NO:23) and the amino acid sequence (SEQ ID NO:24) of SLNAC1 (sodium channel receptor SLNAC1, NM_{—}004769).

FIGS. 16A and 16B show the nucleic acid sequence (SEQ ID NO:25) and the amino acid sequence (SEQ ID NO:26) of CAH4 (carbonic anhydrase iv precursor, NM_{—}000717).

FIGS. 17A and 17B show shows the nucleic acid sequence (SEQ ID NO:27) and the amino acid sequence (SEQ ID NO:28) of PA21 (phopholipase a2 precursor, NM_{—}000928).

FIGS. 18A and 18B show the nucleic acid sequence (SEQ ID NO: 29) and the amino acid sequence (SEQ ID NO:30) of PAR2 (proteinase activated receptor 2 precursor, NM_{—}005242).

FIGS. 19A and 19B show the nucleic acid sequence (SEQ ID NO:31) and the amin acid sequence (SEQ ID NO:32) of IDE (insulin-degrading enzyme, NM_{—}004969).

FIGS. 20A-20B show the nucleic acid sequence (SEQ ID NO:33) and FIG. 20C shows the amino acid sequence (SEQ ID NO:34) of MYO1A (myosin-1A, NM_{—}005379).

FIGS. 21A and 21B the nucleic acid sequence (SEQ ID NO:35) and the amin acid sequence (SEQ ID NO:36) of CYP2J2 (cytochrome P450 monooxygenase, NM_{—}000775).

FIGS. 22A and 22B show the nucleic acid sequence (SEQ ID NO:37) and the amin acid sequence (SEQ ID NO:38) of PHYH (phytanoyl-CoA-hydroxylase (Refsum disease), NM_{—}006214).

FIGS. 23A and 23B show the nucleic acid sequence (SEQ ID NO:39) and the amin acid sequence (SEQ ID NO:40) of CYB5 (cytochrome b5, 3′ end, NM_{—}001914).

FIGS. 24A and 24B show the nucleic acid sequence (SEQ ID NO:41) and the amin acid sequence (SEQ ID NO:42) of COXVIb (coxVIb gene, last exon and flanking sequence, NM_{—}001863).

FIGS. 25A and 25B show the nucleic acid sequence (SEQ ID NO:43) and the amin acid sequence (SEQ ID NO:44) of TCF4 (NM_{—}030756).

FIGS. 26A-26B show the nucleic acid sequence (SEQ ID NO:45) and FIG. 26C shows the amino acid sequence (SEQ ID NO:46) of CAD17 (liver-intestine cadherin, NM_{—}004063).

FIGS. 27A and 27B show the nucleic acid sequence (SEQ ID NO:47) and the amino acid sequence (SEQ ID NO:48) of CLDN15 (claudin 15, NM_{—}014343).

FIGS. 28A-28B show the nucleic acid sequence (SEQ ID NO:49) and FIG. 28C shows the amino acid sequence (SEQ ID NO:50) of CFTR (chloride channel, NM_{—}000492).

FIGS. 29A and 29B show the nucleic acid sequence (SEQ ID NO:51) and the amino acid sequence (SEQ ID NO:52) of H2R (histamine H2 receptor, NM_{—}022304).

FIGS. 30A-30B show the nucleic acid sequence (SEQ ID NO:53) and FIG. 30C shows the amino acid sequence (SEQ ID NO:54) of EGFR (epidermal growth factor receptor, NM_{—}005228).

FIGS. 31A-31B show the nucleic acid sequence (SEQ ID NO:55) and FIG. 31C shows the amino acid sequence (SEQ ID NO:56) of EPHB2, NM_{—}004442).

FIGS. 32A and 32B show the nucleic acid sequence (SEQ ID NO:57) and the amino acid sequence (SEQ ID NO:58) of CRIPTO CR-1 (NM_{—}003212).

FIGS. 33A and 33B show the nucleic acid sequence (SEQ ID NO:59) and the amino acid sequence (SEQ ID NO:60) of Eprin B1 (NM_{—}004429).

FIGS. 34A and 34B show the nucleic acid sequence (SEQ ID NO:61) and the amino acid sequence (SEQ ID NO:62) of MMP-17/MT4-MMP (matrix metalloproteinase 17, NM_{—}016155).

FIGS. 35A and 35B show the the nucleic acid sequence (SEQ ID NO:63) and the amino acid sequence (SEQ ID NO:64) of MMP26 (matrix metalloproteinase 26, NM_{—}021801).

FIGS. 36A and 36B show the nucleic acid sequence (SEQ ID NO:65) and the amino acid sequence (SEQ ID NO:66) of ADAM10 (NM_{—}001110).

FIGS. 37A and 37B show the nucleic acid sequence (SEQ ID NO:67) and the amino acid sequence (SEQ ID NO:68) of ADAM1 (XM_{—}132370).

FIGS. 38A and 38B show the nucleic acid sequence (SEQ ID NO:69) and the amino acid sequence (SEQ ID NO:70) of TIM1(NM_{—}003254).

FIGS. 39A and 39B show the nucleic acid sequence (SEQ ID NO:71) and the amino acid sequence (SEQ ID NO:72) of MUC1 (XM_{—}053256).

FIGS. 40A and 40B show the nucleic acid sequence (SEQ ID NO:73) and the amino acid sequence (SEQ ID NO:74) of CEA (NM_{—}004363).

FIGS. 41A and 41B show the nucleic acid sequence (SEQ ID NO:75) and the amino acid sequence (SEQ ID NO:76) of NCA (NM_{—}002483).

FIGS. 42A and 42B show the nucleic acid sequence (SEQ ID NO:77) and the amino acid sequence (SEQ ID NO:78) of Follistatin (NM_{—}006350).

FIGS. 43A and 43B show the nucleic acid sequence (SEQ ID NO:79) and the amino acid sequence (SEQ ID NO:80) of Claudin 1 (NM_{—}021101).

FIGS. 44A and 44B show the nucleic acid sequence (SEQ ID NO:81) and the amino acid sequence (SEQ ID NO:82) of Claudin 14 (NM_{—}012130).

FIGS. 45A-45B show the nucleic acid sequence (SEQ ID NO:83) and FIG. 45C show the amino acid sequence (SEQ ID NO:84) of Tenascin-R (NM_{—}003285).

FIGS. 46A and 46B show the nucleic acid sequence (SEQ ID NO:85) and the amino acid sequence (SEQ ID NO:86) of CAD3 (NM_{—}001793).

FIGS. 47A and 47B show the nucleic acid sequence (SEQ ID NO:87) and the amino acid sequence (SEQ ID NO:88) of CONT (NM_{—}001843).

FIGS. 48A and 48B show the nucleic acid sequence (SEQ ID NO:89) and the amino acid sequence (SEQ ID NO:90) of Osteopontin (NM_{—}000582).

FIGS. 49A and 49B show the nucleic acid sequence (SEQ ID NO:91) and the amino acid sequence (SEQ ID NO:92) of Galectin 8 (NM_{—}006499).

FIGS. 50A and 50B show the nucleic acid sequence (SEQ ID NO:93) and the amino acid sequence (SEQ ID NO:94) of GS1 (bihlycan, NM_{—}001711).

FIGS. 51A and 51B show the nucleic acid sequence (SEQ ID NO:95) and the amino acid sequence (SEQ ID NO:96) of Fizzled 2 (NM001466).

FIGS. 52A and 52B show the nucleic acid sequence (SEQ ID NO:97) and the amino acid sequence (SEQ ID NO:98) of ISLR (NM_{—}005545).

FIGS. 53A-53B show the nucleic acid sequence (SEQ ID NO:) and FIG. 53C shows the amino acid sequence (SEQ ID NO:2) of

FIGS. 54A and 54B show the nucleic acid sequence (SEQ ID NO:1) and the amino acid sequence (SEQ ID NO:2) of

FIGS. 55A and 55B show the nucleic acid sequence (SEQ ID NO:103) and the amino acid sequence (SEQ ID NO:104) of Tie2 ligand2 (NM_{—}001147).

FIGS. 56A and 56B show the nucleic acid sequence (SEQ ID NO:105) and the amino acid sequence (SEQ ID NO:106) of VEGFC (NM_{—}005429).

FIGS. 57A and 57B show the nucleic acid sequence (SEQ ID NO:107) and the amino acid sequence (SEQ ID NO:108) of tPA (NM_{—}000930).

FIGS. 58A-58B show the nucleic acid sequence (SEQ ID NO:109) and FIG. 58C shows the amino acid sequence (SEQ ID NO:110) of thrombomodulin (NM_{—}000361).

FIGS. 59A and 59B show the nucleic acid sequence (SEQ ID NO:111) and the amino acid sequence (SEQ ID NO:112) of TF (coagulation factor III, thromboplastin, tissue factor, NM_{—}0001993).

FIGS. 60A and 60B show the nucleic acid sequence (SEQ ID NO:113) and the amino acid sequence (SEQ ID NO:114) of GPR4 (G-coupled protein receptor-4, NM_{—}005282).

FIGS. 61A and 61B show the nucleic acid sequence (SEQ ID NO:115) and the amino acid sequence (SEQ ID NO:116) of GPR66 (G-coupled protein receptor 66).

FIGS. 62A and 62B show the nucleic acid sequence (SEQ ID NO:117) and the amino acid sequence (SEQ ID NO:118) of SLC22A2 (NM_{—}003058).

FIGS. 63A-63B show the nucleic acid sequence (SEQ ID NO:119) and FIG. 63C shows the amino acid sequence (SEQ ID NO:120) of MLSN1 (NM_{—}002420).

FIGS. 64A-64B show the nucleic acid sequence (SEQ ID NO:121) and FIG. 64C shows the amino acid sequence (SEQ ID NO:122) of ATN2 (Na/K transport, NM_{—}000702).

### DESCRIPTION OF THE INVENTION

Barrett's esophagus, a complication of gastrointestinal reflux disease, is the primary risk factor for esophageal adenocarcinoma. Biopsy specimens representing disease progression through Barrett's esophagus, dysplasia and adenocarcinoma, were collected and analyzed using cDNA microarrays to identify genes expressed in the different disease stages. It was discovered that the expression of particular genes increased with the progression of the disease through dysplasia, especially high grade dysplasia, suggestive of a differentiated small intestinal enterocyte lineage. The present invention defines a collection of markers that assist in identifying patients with highest risk of developing cancer, especially the development of esophageal adenocarcinoma.

The progression of Barrett's esophagus through dysplasia to adenocarcinoma was examined, identifying specific genes associated with increasing risk of carcinogenesis. These data provide insight into the potential role of progressive intestinal metaplasia in generating the colon tumor-like expression profiles disclosed herein for esophageal adenocarcinoma. Genes that define early stages of this process, progression of BE to dysplasia, serve as markers to permit targeting of surveillance to those patients at most risk of developing esophageal carcinoma.

DNA microarray technology has been used to characterize and cluster Barrett's metaplasia from normal mucosa, and esophageal adenocarcinoma and squamous cell carcinoma (Barrett et al., Neoplasia 4:121-128 (2002); and Selaru et al., Oncogene 21:475-478 (2002)). The authors do not, however, describe HGD markers or dysplasia markers of any kind useful for predicting patients likely to develop adenocarcinoma.

The present invention provides nucleic acid and protein sequences that are differentially expressed in high-grade esophageal dysplasia when compared to normal tissue controls, here-in termed “high-grade dysplasia genes,” “high-grade dysplasia nucleic acid sequences,” “HGD marker genes” and the like. As outlined below, high-grade esophageal dysplasia sequences that are differentially expressed include those that are up-regulated in high-grade esophageal dysplasia). The differential expression of these sequences in high-grade esophageal dysplasia combined with the fact they have been identified in patients likely to develop cancer, such as adenocarcinoma, they are contributory factors in cancer. The high-grade esophageal dysplasia nucleic acid sequences, or the polypeptides encoded by the nucleic acids, of the invention are disclosed in Table 4 as HGD marker genes, or polypeptides, as follows: ET-1 (endothelin-1, NM_{—}001955) (SEQ ID NO:1 or 2); AGR2 (anterior gradient 2 (Xenepus laevis) homolog, NM_{—}006408) (SEQ ID NO:3 or 4); ADAM8 (NM_{—}001109) (SEQ ID NO:5 or 6); PRSS8 (Prostasin precursor, serine protease, NM_{—}002773) (SEQ ID NO:7 or 8); AXO1 (Axonin-1 precursor, NM_{—}005076) (SEQ ID NO:9 or 10); NROB2 (Nuclear hormone receptor, NM_{—}021969) (SEQ ID NO:11 or 12); TM7SF1 (NM_{—}003272) (SEQ ID NO:13 or 14); DLDH (dihydrolipamide dehydrogenase, NM_{—}000108) (SEQ ID NOS:15 or 16); MAT2B (methionine adenosyltransferase II, beta, NM_{—}013283) (SEQ ID NO:17 or 18); STC-2 (stanniocalcin-2, NM_{—}003714) (SEQ ID NO:19 or 20); PPBI (alkaline phosphatase, intestinal precursor, NM_{—}001631) (SEQ ID NO:21 or 22); SLNAC1 (sodium channel receptor SLNAC1, NM_{—}004769) (SEQ ID NO:23 or 24); CAH4 (carbonic anhydrase iv precursor, NM_{—}000717) (SEQ ID NO:25 or 26); PA21 (phopholipase a2 precursor, NM_{—}000928) (SEQ ID NO:27 or 28); PAR2 (proteinase activated receptor 2 precursor, NM_{—}005242) (SEQ ID NO:29 or 30); IDE (insulin-degrading enzyme, NM_{—}004969) (SEQ ID NO:31 or 32); MYO1A (myosin-1A, NM_{—}005379) (SEQ ID NO:33 or 34); CYP2J2 (cytochrome P450 monooxygenase, NM_{—}000775) (SEQ ID NO:35 or 36); PHYH (phytanoyl-CoA-hydroxylase (Refsum disease), NM_{—}006214) (SEQ ID NO:37 or 38); CYB5 (cytochrome b5, 3′ end, NM_{—}001914) (SEQ ID NO:39 or 40); COXVIb (coxVIb gene, last exon and flanking sequence, NM_{—}001863) (SEQ ID NO:41 or 42); and TCF4 (NM_{—}030756) (SEQ ID NO:43 or 44).

### Definitions

The phrases “gene amplification” and “gene duplication” are used interchangeably and refer to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

“Tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, carcinoma, adenocarcinoma; lymphoma, blastoma, sarcoma, and leukemia. More particular examples of such cancers include esophageal cancer, breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, liver cancer, vulval cancer, thyroid cancer, hepatic carcinoma and various types of head and neck cancer.

The term “diagnosis” or “diagnosing” as used herein shall refer to the determination of the nature of a case of a disease, such as by determining a gene expression profile or polypeptide expression profile unique to the disease or a stage of the disease.

A “normal” tissue sample refers to tissue or cells that are not diseased as defined herein, such as tissue from a mammal that is not experiencing a particular disease of interest. The term “normal cell” or “normal tissue” as used herein refers to a state of a cell or tissue in which the cell or tissue is apparently free of an adverse biological condition when compared to a diseased cell or tissue having that adverse biological condition. The normal cell or normal tissue may be from any prokaryotic or eukaryotic organism including, but not limited to, bacteria, yeast, insect, bird, reptile, and any mammal including human. Where the normal tissue or cell is used as a normal control sample, it is generally from the same species as the test sample. Where the cell or tissue is mammalian, the cell or tissue is any cell or tissue including, but not limited to blood, muscle, nerve, brain, breast, heart, lung, liver, pancreas, spleen, thymus, esophagus, stomach, intestine, kidney, testis, ovary, uterus, hair follicle, skin, bone, bladder, and spinal cord.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

A “pharmaceutical composition” as used herein refers to a composition comprising a chemotherapeutic agent for treatment of a disease combined with physiologically acceptable materials such as carriers, excepients, stabilzers, buffers, salts, antioxidants, hydrophilic polymers, amino acids, carbohydrates, ionic or nonionic uurfactants, and/or polyethylene or propylene glycol. The pharmaceutical composition may be in aqueous form, tablet, capsule, microcapsules, liposomes, trandermal patches, and the like.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, etc.

“Mammal” for purposes of treatment refers to any animal classified as a mammal, including humans, domestic and farm animals, and zoo, sports, or pet animals, such as dogs, horses, cats, cattle, pigs, sheep, etc. Preferably, the mammal is human.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Administration “in combination with” one or more further therapeutic agents includes simultaneous (concurrent) and consecutive administration in any order.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (Taxotere, Rhône-Poulenc Rorer, Antony, Rnace), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), 5-FU, 6-thioguanine, 6-mercaptopurine, actinomycin D, VP-16, chlorambucil, melphalan, and other related nitrogen mustards. Also included in this definition are hormonal agents that act to regulate or inhibit hormone action on tumors such as tamoxifen and onapristone. In an embodiment, the chemotherapeutic agent of the invention is a chemical compound useful in the treatment of HGD, adenocarcinoma, or for inhibiting or preventing progression from the HGD to adenocarcinoma in a patient.

A “growth inhibitory agent” when used herein refers to a compound or composition which inhibits growth of a cell, especially cancer cell overexpressing any of the genes identified herein, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al., (W B Saunders: Philadelphia, 1995), especially p. 13.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon -α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL- 1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy”, Biochemical Society Transactions, 14:375-382, 615th Meeting, Belfast (1986), and Stella et al., “Prodrugs: A Chemical Approach to Targeted Drug Delivery”, Directed Drug Delivery, Borchardt et al., (ed.), pp. 147-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glysocylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrugs form for use in this invention include, but are not limited to, those chemotherapeutic agents described above.

An “effective amount” or therapeutically effective amount” of a polypeptide disclosed herein or an antagonist thereof, in reference to inhibition of neoplastic cell growth, tumor growth or cancer cell growth, is an amount capable of inhibiting, to some extent, the growth of target cells. The term includes an amount capable of invoking a growth inhibitory, cytostatic and/or cytotoxic effect and/or apoptosis of the target cells. An “effective amount” is an amount of an antagonist of ET-1 (endothelin-1, NM_{—}001955) (SEQ ID NO:1 or 2); AGR2 (anterior gradient 2 (Xenepus laevis) homolog, NM_{—}006408) (SEQ ID NO:3 or 4); ADAM8 (NM_{—}001109) (SEQ ID NO:5 or 6); PRSS8 (Prostasin precursor, serine protease, NM_{—}002773) (SEQ ID NO:7 or 8); AXO1 (Axonin-1 precursor, NM_{—}005076) (SEQ ID NO:9 or 10); NROB2 (Nuclear hormone receptor, NM_{—}021969) (SEQ ID NO:11 or 12); TM7SF1 (NM_{—}003272) (SEQ ID NO:13 or 14); DLDH (dihydrolipamide dehydrogenase, NM_{—}000108) (SEQ ID NOS:15 or 16); MAT2B (methionine adenosyltransferase II, beta, NM_{—}013283) (SEQ ID NO:17 or 18); STC-2 (stanniocalcin-2, NM_{—}003714) (SEQ ID NO:19 or 20); PPBI (alkaline phosphatase, intestinal precursor, NM_{—}001631) (SEQ ID NO:21 or 22); SLNAC1 (sodium channel receptor SLNAC1, NM_{—}004769) (SEQ ID NO:23 or 24); CAH4 (carbonic anhydrase iv precursor, NM_{—}000717) (SEQ ID NO:25 or 26); PA21 (phopholipase a2 precursor, NM_{—}000928) (SEQ ID NO:27 or 28); PAR2 (proteinase activated receptor 2 precursor, NM_{—}005242) (SEQ ID NO:29 or 30); IDE (insulin-degrading enzyme, NM_{—}004969) (SEQ ID NO:31 or 32); MYO1A (myosin-1A, NM_{—}005379) (SEQ ID NO:33 or 34); CYP2J2 (cytochrome P450 monooxygenase, NM_{—}000775) (SEQ ID NO:35 or 36); PHYH (phytanoyl-CoA-hydroxylase (Refsum disease), NM_{—}006214) (SEQ ID NO:37 or 38); CYB5 (cytochrome b5, 3′ end, NM_{—}001914) (SEQ ID NO:39 or 40); COXVIb (coxVIb gene, last exon and flanking sequence, NM_{—}001863) (SEQ ID NO:41 or 42); and TCF4 (NM_{—}030756) (SEQ ID NO:43 or 44) gene or polypeptide for purposes of inhibiting neoplastic cell growth, tumor growth or cancer cell growth, may be determined empirically and in a routine manner. The terms further refer to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (ie., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of an antagonist of ET-1 (endothelin-1, NM_{—}001955) (SEQ ID NO:1 or 2); AGR2 (anterior gradient 2 (Xenepus laevis) homolog, NM_{—}006408) (SEQ ID NO:3 or 4); ADAM8 (NM_{—}001109) (SEQ ID NO:5 or 6); PRSS8 (Prostasin precursor, serine protease, NM_{—}002773) (SEQ ID NO:7 or 8); AXO1 (Axonin-1 precursor, NM_{—}005076) (SEQ ID NO:9 or 10); NROB2 (Nuclear hormone receptor, NM_{—}021969) (SEQ ID NO:11 or 12); TM7SF1 (NM_{—}003272) (SEQ ID NO:13 or 14); DLDH (dihydrolipamide dehydrogenase, NM_{—}000108) (SEQ ID NOS:15 or 16); MAT2B (methionine adenosyltransferase II, beta, NM_{—}013283) (SEQ ID NO:17 or 18); STC-2 (stanniocalcin-2, NM_{—}003714) (SEQ ID NO:19 or 20); PPBI (alkaline phosphatase, intestinal precursor, NM_{—}001631) (SEQ ID NO:21 or 22); SLNAC1 (sodium channel receptor SLNAC1, NM_{—}004769) (SEQ ID NO:23 or 24); CAH4 (carbonic anhydrase iv precursor, NM_{—}000717) (SEQ ID NO:25 or 26); PA21 (phopholipase a2 precursor, NM_{—}000928) (SEQ ID NO:27 or 28); PAR2 (proteinase activated receptor 2 precursor, NM_{—}005242) (SEQ ID NO:29 or 30); IDE (insulin-degrading enzyme, NM_{—}004969) (SEQ ID NO:31 or 32); MYO1A (myosin-1A, NM_{—}005379) (SEQ ID NO:33 or 34); CYP2J2 (cytochrome P450 monooxygenase, NM_{—}000775) (SEQ ID NO:35 or 36); PHYH (phytanoyl-CoA-hydroxylase (Refsum disease), NM_{—}006214) (SEQ ID NO:37 or 38); CYB5 (cytochrome b5, 3′ end, NM_{—}001914) (SEQ ID NO:39 or 40); COXVIb (coxVIb gene, last exon and flanking sequence, NM_{—}001863) (SEQ ID NO:41 or 42); or TCF4 (NM_{—}030756) (SEQ ID NO:43 or 44) gene or polypeptide for purposes of treatment of tumor may be determined empirically and in a routine manner.

A “growth inhibitory amount” of a compound that inhibits growth of a cell expressing genes, or polypeptides, from the following group: ET-1 (endothelin-1, NM_{—}001955) (SEQ ID NO:1 or 2); AGR2 (anterior gradient 2 (Xenepus laevis) homolog, NM_{—}006408) (SEQ ID NO:3 or 4); ADAM8 (NM_{—}001109) (SEQ ID NO:5 or 6); PRSS8 (Prostasin precursor, serine protease, NM_{—}002773) (SEQ ID NO:7 or 8); AXO1 (Axonin-1 precursor, NM_{—}005076) (SEQ ID NO:9 or 10); NROB2 (Nuclear hormone receptor, NM_{—}021969) (SEQ ID NO:11 or 12); TM7SF1 (NM_{—}003272) (SEQ ID NO:13 or 14); DLDH (dihydrolipamide dehydrogenase, NM_{—}000108) (SEQ ID NOS:15 or 16); MAT2B (methionine adenosyltransferase II, beta, NM_{—}013283) (SEQ ID NO:17 or 18); STC-2 (stanniocalcin-2, NM_{—}003714) (SEQ ID NO:19 or 20); PPBI (alkaline phosphatase, intestinal precursor, NM_{—}001631) (SEQ ID NO:21 or 22); SLNAC1 (sodium channel receptor SLNAC1, NM_{—}004769) (SEQ ID NO:23 or 24); CAH4 (carbonic anhydrase iv precursor, NM_{—}000717) (SEQ ID NO:25 or 26); PA21 (phopholipase a2 precursor, NM_{—}000928) (SEQ ID NO:27 or 28); PAR2 (proteinase activated receptor 2 precursor, NM_{—}005242) (SEQ ID NO:29 or 30); IDE (insulin-degrading enzyme, NM_{—}004969) (SEQ ID NO:31 or 32); MYO1A (myosin-1A, NM_{—}005379) (SEQ ID NO:33 or 34); CYP2J2 (cytochrome P450 monooxygenase, NM_{—}000775) (SEQ ID NO:35 or 36); PHYH (phytanoyl-CoA-hydroxylase (Refsum disease), NM_{—}006214) (SEQ ID NO:37 or 38); CYB5 (cytochrome b5, 3′ end, NM_{—}001914) (SEQ ID NO:39 or 40); COXVIb (coxVIb gene, last exon and flanking sequence, NM_{—}001863) (SEQ ID NO:41 or 42); and TCF4 (NM_{—}030756) (SEQ ID NO:43 or 44) is an amount of the compound capable of inhibiting the growth of a cell, especially tumor, e.g., cancer cell, either in vitro or in vivo. Optionally, the compound is an antagonist of the gene or polypeptide, such as an antagonist antibody or antagonist small organic molecule. A “growth inhibitory amount” of such a compound, for purposes of inhibiting neoplastic cell growth, may be determined empirically and in a routine manner.

A “cytotoxic amount” of an ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); or TCF4 (NM_{—}030756) (SEQ ID NO:44) polypeptide antagonist is an amount capable of causing the destruction of a cell, especially tumor, e.g., cancer cell, either in vitro or in vivo. A “cytotoxic amount” of a such a polypeptide antagonist for purposes of inhibiting neoplastic cell growth may be determined empirically and in a routine manner.

The terms ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); and TCF4 (NM_{—}030756) (SEQ ID NO:44) polypeptide or protein when used herein encompass native sequence ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); and TCF4 (NM_{—}030756) (SEQ ID NO:44) polypeptide variants (which are further defined herein). The ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); or TCF4 (NM_{—}030756) (SEQ ID NO:44) polypeptide may be isolated from a variety of sources, such as from human tissue types or from another source, or prepared by recombinant and/or synthetic methods.

A “native sequence polypeptide” of each HGD marker polypeptide has the same amino acid sequence or is a polypeptide variant having at least about 80% amino acid sequence identity, preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and most preferably at least about 99% amino acid sequence identity with a full-length native sequence polypeptide sequence, lacking the signal peptide as disclosed herein, as the ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); or TCF4 (NM_{—}030756) (SEQ ID NO:44) polypeptide as derived from nature. Such native sequence polypeptide can be isolated from nature or can be produced by recombinant and/or synthetic means. The term “native sequence polypeptide” specifically encompasses naturally-occurring truncated or secreted forms (e.g., an extracellular domain sequence), naturally-occurring variant forms (e.g., alternatively spliced forms) and naturally-occurring allelic variants of the polypeptides encoded by a HGD marker gene as disclosed herein. In one embodiment of the invention, the native sequence HGD marker polypeptide is a mature or full-length native sequence HGD marker polypeptide as encoded by the nucleic acid sequences of the GenBank accession numbers listed in Table 4A for the respective polypeptide. Also, the HGD marker polypeptides encoded by the nucleic acid sequences disclosed in the respective GenBank accession numbers listed in Table 4A, are shown to begin with the methionine residue designated therein as amino acid position 1, it is conceivable and possible that another methionine residue located either upstream or downstream from amino acid position 1 may be employed as the starting amino acid residue for HGD marker polypeptide.

The “extracellular domain” or “ECD” of a polypeptide disclosed herein refers to a form of the polypeptide which is essentially free of the transmembrane and cytoplasmic domains. Ordinarily, a polypeptide ECD will have less than about 1% of such transmembrane and/or cytoplasmic domains and preferably, will have less than about 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified and as shown in the appended figures. As such, in one embodiment of the present invention, the extracellular domain of a polypeptide of the present invention comprises amino acids 1 to X of the mature amino acid sequence, wherein X is any amino acid within 5 amino acids on either side of the extracellular domain/transmembrane domain boundary.

The approximate location of the “signal peptides” of the various PRO polypeptides disclosed herein are shown in the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng., 10:1-6 (1997) and von Heinje et al., Nucl. Acids. Res., 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

A “polypeptide variant” of any one of ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); or TCF4 (NM_{—}030756) (SEQ ID NO:44) polypeptide as defined above or below having at least about 80% amino acid sequence identity with a full-length native sequence polypeptide, with or without the signal peptide, as disclosed herein or any other fragment of a full-length HGD marker polypeptides wherein one or more amino acid residues are added, or deleted, at the N- or C-terminus of the full-length native amino acid sequence. Ordinarily, a HGD marker polypeptide variant will have at least about 80% amino acid sequence identity, preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and most preferably at least about 99% amino acid sequence identity with a full-length native sequence polypeptide sequence lacking the signal peptide as disclosed herein, an extracellular domain of a HGD marker polypeptide, with or without the signal peptide, as disclosed herein or any other fragment of a full-length HGD marker polypeptide sequence as disclosed herein. Ordinarily, a HGD marker polypeptide variant is at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 300 amino acids in length, or more.

“Percent (%) amino acid sequence identity” with respect to the amino acid sequence of any of the HGD marker polypeptides identified herein is defined as the percentage of amino acid residues in a candidate sequence that are identical with the amino acid residues in an ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); or TCF4 (NM 030756) (SEQ ID NO:44) polypeptide, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2, wherein the complete source code for the ALIGN-2 program is provided in Table 5. The ALIGN-2 sequence comparison computer program was authored by Genentech, Inc., and the source code shown in Table 5 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 5. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations, Tables 2A-2B demonstrate how to calculate the % amino acid sequence identity of the amino acid sequence designated “Comparison Protein” to the amino acid sequence designated “PRO”.

Unless specifically stated otherwise, all % amino acid sequence identity values used herein are obtained as described above using the ALIGN-2 sequence comparison computer program. However, % amino acid sequence identity may also be determined using the sequence comparison program NCBI-BLAST2 (Altschul et al., Nucleic Acids Res., 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

In addition, % amino acid sequence identity may also be determined using the WU-BLAST-2 computer program (Altschul et al., Methods in Enzymology, 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. For purposes herein, a % amino acid sequence identity value is determined by dividing (a) the number of matching identical amino acids residues between the amino acid sequence of the PRO polypeptide of interest having a sequence derived from the native PRO polypeptide and the comparison amino acid sequence of interest (i.e., the sequence against which the PRO polypeptide of interest is being compared which may be a PRO variant polypeptide) as determined by WU-BLAST-2 by (b) the total number of amino acid residues of the PRO polypeptide of interest. For example, in the statement “a polypeptide comprising an amino acid sequence A which has or having at least 80% amino acid sequence identity to the amino acid sequence B”, the amino acid sequence A is the comparison amino acid sequence of interest and the amino acid sequence B is the amino acid sequence of the PRO polypeptide of interest.

As used herein, a “HGD marker” or “cancer marker gene or polypeptide,” or “anti-[HGD marker]” or “anti-[cancer marker]” refers to any one of the genes, polypeptides encoded by the genes, or antibodies specific for the polypeptides described herein as diagnositic for HGD or cnacer. Thus, for example, “TCF4” refers to the gene marker or its encoded polypeptide, whereas anti-TCF4 refers to an antobidy to the TCF4-encoded polypeptide.

A “gene variant polynucleotide” as used herein refers to a nucleic acid sequence that varies from the native sequence of its respective HGD marker gene NCBI accession sequence as disclosed in Table 4A, and further refers to a nucleic acid molecule which encodes a biologically active polypeptide and which nucleic acid molecule has at least about 80% nucleic acid sequence identity with a nucleic acid sequence selected from the group of marker genes: ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43), which genes encode, respectively, the full-length native polypeptides of the group: ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); and TCF4 (NM_{—}030756) (SEQ ID NO:44) polypeptide sequence as disclosed herein, a full-length native sequence HGD marker polypeptide sequence lacking the signal peptide as disclosed herein, an extracellular domain of a HGD marker polypeptide, with or without the signal peptide, as disclosed herein or any other fragment of a full-length HGD marker polypeptide sequence as disclosed herein. Ordinarily, a HGD marker variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with the nucleic acid sequence encoding a full-length native sequence HGD marker polypeptide sequence as disclosed herein, a full-length native sequence HGD marker polypeptide sequence lacking the signal peptide as disclosed herein, an extracellular domain of a HGD marker polypeptide, with or without the signal sequence, as disclosed herein or any other fragment of a full-length HGD marker polypeptide sequence as disclosed herein. Variants do not encompass the native nucleotide sequence.

Ordinarily, HGD marker gene variant polynucleotides are at least about 20 nucleotides in length, frequently at least about 30 nucleotides in length, often at least about 60 nucleotides in length, more often at least about 90 nucleotides in length, more often at least about 120 nucleotides in length, more often at least about 150 nucleotides in length, more often at least about 180 nucleotides in length, more often at least about 210 nucleotides in length, more often at least about 240 nucleotides in length, more often at least about 270 nucleotides in length, more often at least about 300 nucleotides in length, more often at least about 450 nucleotides in length, more often at least about 600 nucleotides in length, more often at least about 900 nucleotides in length, or more.

“Percent (%) nucleic acid sequence identity” with respect to variant polypeptides of each of the HGD marker polypeptide-encoding nucleic acid sequences identified herein is defined as the percentage of nucleotides in a candidate sequence that are identical with the nucleotides in a HGD marker polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained as described below by using the sequence comparison computer program ALIGN-2, wherein the complete source code for the ALIGN-2 program is provided in Table 5. The ALIGN-2 sequence comparison computer program was authored by Genentech, Inc., and the source code shown in Table 5 has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 5. The ALIGN-2 program should be compiled for use on a UNIX operating system, preferably digital UNIX V4.0D. All sequence comparison parameters are set by the ALIGN-2 program and do not vary.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 2C-2D demonstrate how to calculate the % nucleic acid sequence identity of the nucleic acid sequence designated “Comparison DNA” to the nucleic acid sequence designated “PRO-DNA”.

Unless specifically stated otherwise, all % nucleic acid sequence identity values used herein are obtained as described above using the ALIGN-2 sequence comparison computer program. However, % nucleic acid sequence identity may also be determined using the sequence comparison program NCBI-BLAST2 (Altschul et al., Nucleic Acids Res., 25:3389-3402 (1997)). The NCBI-BLAST2 sequence comparison program may be downloaded from http:H/www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In addition, % nucleic acid sequence identity values may also be generated using the WU-BLAST-2 computer program (Altschul et al., Methods in Enzymology, 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. For purposes herein, a % nucleic acid sequence identity value is determined by dividing (a) the number of matching identical nucleotides between the nucleic acid sequence of the PRO polypeptide-encoding nucleic acid molecule of interest having a sequence derived from the native sequence PRO polypeptide-encoding nucleic acid and the comparison nucleic acid molecule of interest (i.e., the sequence against which the PRO polypeptide-encoding nucleic acid molecule of interest is being compared which may be a variant PRO polynucleotide) as determined by WU-BLAST-2 by (b) the total number of nucleotides of the PRO polypeptide-encoding nucleic acid molecule of interest. For example, in the statement “an isolated nucleic acid molecule comprising a nucleic acid sequence A which has or having at least 80% nucleic acid sequence identity to the nucleic acid sequence B”, the nucleic acid sequence A is the comparison nucleic acid molecule of interest and the nucleic acid sequence B is the nucleic acid sequence of the PRO polypeptide-encoding nucleic acid molecule of interest.

In other embodiments, variants of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:1 1); TM7SFI (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); or TCF4 (NM_{—}030756) (SEQ ID NO:43) HGD marker genes encode an active HGD marker polypeptide, and nucleic acid sequences useful for identifying the marker genes by, for example, nucleic acid hybridization assays or PCR assays are capable of hybridizing, preferably under stringent hybridization and wash conditions, to nucleotide sequences encoding the full-length ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43) gene or hybridizable fragments thereof, which nucleotide sequences are found in the NCBI accession numbers listed in Table 4A for the respective polypeptides. HGD variant polypeptides may be those that are encoded by a HGD marker gene variant polynucleotide.

The term “positives”, in the context of the amino acid sequence identity comparisons performed as described above, includes amino acid residues in the sequences compared that are not only identical, but also those that have similar properties. Amino acid residues that score a positive value to an amino acid residue of interest are those that are either identical to the amino acid residue of interest or are a preferred substitution (as defined in Table 4A below) of the amino acid residue of interest.

For purposes herein, the % value of positives of a given amino acid sequence A to, with, or against a given amino acid sequence B (which can alternatively be phrased as a given amino acid sequence A that has or comprises a certain % positives to, with, or against a given amino acid sequence B) is calculated as follows:

100 times the fraction X/Y

where X is the number of amino acid residues scoring a positive value as defined above by the sequence alignment program ALIGN-2 in that program's alignment of A and B, and where Y is the total number of amino acid residues in B. It will be appreciated that where the length of amino acid sequence A is not equal to the length of amino acid sequence B, the % positives of A to B will not equal the % positives of B to A.

“Isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); or TCF4 (NM_{—}030756) (SEQ ID NO:44) polypeptide's natural environment will not be present. Ordinarily, however, isolated polypeptide will be prepared by at least one purification step.

An “isolated” nucleic acid molecule encoding an ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); or TCF4 (NM_{—}030756) (SEQ ID NO:44) polypeptide or an “isolated” nucleic acid encoding an anti-[HGD marker polypeptide] antibody, is a nucleic acid molecule that is identified and separated from at least one contaminant nucleic acid molecule with which it is ordinarily associated in the natural source of the HGD marker genes or the anti-[HGD marker polypeptide]-encoding nucleic acid. Preferably, the isolated nucleic acid is free of association with all components with which it is naturally associated. An isolated polypeptide or nucleic acid sequence is other than in the form or setting in which it is found in nature. Isolated nucleic acid molecules therefore are distinguished from the nucleic acid molecule as it exists in natural cells. However, an isolated nucleic acid molecule encoding a HGD maker polypeptide or an anti-[HGD marker polypeptide] antibody includes HGD marker gene nucleic acid molecules and anti-[HGD marker polypeptide]-encoding nucleic acid molecules contained in cells that ordinarily express HGD marker polypeptides or express anti-[HGD maker polypeptide] antibodies where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-[HGD marker polypeptide] monoclonal antibodies (including antagonist, and neutralizing antibodies), anti-[HGD marker polypeptide] antibody compositions with polyepitopic specificity, single chain anti-[HGD marker polypeptide] antibodies, and fragments thereof (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 □g/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 35□C-50° C. The skilled artisan will recognize how to adjust the temperature, ionic strength, etc. as necessary to accommodate factors such as probe length and the like.

The term “epitope tagged” when used herein refers to a chimeric polypeptide comprising a HGD marker polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of ET-1 (endothelin-1, NM_{—}001955) (SEQ ID NO:2); AGR2 (anterior gradient 2 (Xenepus laevis) homolog, NM_{—}006408) (SEQ ID NO:4); ADAM8 (NM_{—}001109) (SEQ ID NO:6); PRSS8 (Prostasin precursor, serine protease, NM_{—}002773) (SEQ ID NO:8); AXO1 (Axonin-1 precursor, NM_{—}005076) (SEQ ID NO:10); NROB2 (Nuclear hormone receptor, NM_{—}021969) (SEQ ID NO:12); TM7SF1 (NM_{—}003272) (SEQ ID NO:14); DLDH (dihydrolipamide dehydrogenase, NM_{—}000108) (SEQ ID NO:16); MAT2B (methionine adenosyltransferase II, beta, NM_{—}013283) (SEQ ID NO:18); STC-2 (stanniocalcin-2, NM_{—}003714) (SEQ ID NO:20); PPBI (alkaline phosphatase, intestinal precursor, NM_{—}001631) (SEQ ID NO:22); SLNAC1 (sodium channel receptor SLNAC1, NM_{—}004769) (SEQ ID NO:24); CAH4 (carbonic anhydrase iv precursor, NM_{—}000717) (SEQ ID NO:26); PA21 (phopholipase a2 precursor, NM_{—}000928) (SEQ ID NO:28); PAR2 (proteinase activated receptor 2 precursor, NM_{—}005242) (SEQ ID NO:30); IDE (insulin-degrading enzyme, NM_{—}004969) (SEQ ID NO:32); MYO1A (myosin-1A, NM_{—}005379) (SEQ ID NO:34); CYP2J2 (cytochrome P450 monooxygenase, NM_{—}000775) (SEQ ID NO:36); PHYH (phytanoyl-CoA-hydroxylase (Refsum disease), NM_{—}006214) (SEQ ID NO:38); CYB5 (cytochrome b5, 3′ end, NM_{—}001914) (SEQ ID NO:40); COXVIb (coxVIb gene, last exon and flanking sequence, NM_{—}001863) (SEQ ID NO:42); or TCF4 (NM_{—}030756) (SEQ ID NO:44) polypeptides which retain a biological and/or an immunological activity/property of a native or naturally-occurring HGD marker polypeptide, wherein “biological” activity refers to a function (either inhibitory or stimulatory) caused by a native or naturally-occurring HGD marker polypeptide other than the ability to induce the production of an antibody against an antigenic epitope possessed by a native or naturally-occurring HGD marker polypeptide and an “immunological” activity refers to the ability to induce the production of an antibody against an antigenic epitope possessed by a native or naturally-occurring HGD marker polypeptide.

“Biological activity” in the context of an antibody or another antagonist molecule, or therapeutic compound that can be identified by the screening assays disclosed herein (e.g., an organic or inorganic small molecule, peptide, etc.) is used to refer to the ability of such molecules to bind or complex with the polypeptides encoded by the amplified genes identified herein, or otherwise interfere with the interaction of the encoded polypeptides with other cellular proteins or otherwise interfere with the transcription or translation of a HGD marker polypeptide. “Biological activity” in the context of an agonist molecule that enhances the activity of, for example, native anti-angiogenic molecules refers to the ability of such molecules to bind or complex with the polypeptides encoded by the amplified genes identified herein or otherwise modify the interaction of the encoded polypeptides with other cellular proteins or otherwise enhance the transcription or translation of a TIMP1 or thrombospondin 2 polypeptide. A preferred biological activity is growth inhibition of a target tumor cell. Another preferred biological activity is cytotoxic activity resulting in the death of the target tumor cell.

The term “biological activity” in the context of a HGD marker polypeptide means the typical activity of the HGD marker polypeptide in the cell.

The phrase “immunological activity” means immunological cross-reactivity with at least one epitope of a HGD marker polypeptide.

“Immunological cross-reactivity” as used herein means that the candidate polypeptide is capable of competitively inhibiting the qualitative biological activity of a HGD marker polypeptide having this activity with polyclonal antisera raised against the known active HGD marker polypeptide. Such antisera are prepared in conventional fashion by injecting goats or rabbits, for example, subcutaneously with the known active analogue in complete Freund's adjuvant, followed by booster intraperitoneal or subcutaneous injection in incomplete Freunds. The immunological cross-reactivity preferably is “specific”, which means that the binding affinity of the immunologically cross-reactive molecule (e.g., antibody) identified, to the corresponding HGD marker polypeptide is significantly higher (preferably at least about 2-times, more preferably at least about 4-times, even more preferably at least about 8-times, most preferably at least about 10-times higher) than the binding affinity of that molecule to any other known native polypeptide.

The term “antagonist” is used in the broadest sense, and includes any molecule that partially or fully blocks, inhibits, or neutralizes a biological activity of a native HGD marker polypeptide disclosed herein or the transcription or translation thereof, particularly when the HGD marker polypeptide is expressed about 1.5-fold above the level of expression in normal tissue controls. Suitable antagonist molecules specifically include antagonist antibodies or antibody fragments, binding fragments, peptides, small organic molecules, anti-sense nucleic acids, etc. Included are methods for identifying antagonists of an ET-1 (endothelin-1, NM_{—}001955) (SEQ ID NO:1 or 2); AGR2 (anterior gradient 2 (Xenepus laevis) homolog, NM_{—}006408) (SEQ ID NO:3 or 4); ADAM8 (NM_{—}001109) (SEQ ID NO:5 or 6); PRSS8 (Prostasin precursor, serine protease, NM_{—}002773) (SEQ ID NO:7 or 8); AXO1 (Axonin-1 precursor, NM_{—}005076) (SEQ ID NO:9 or 10); NROB2 (Nuclear hormone receptor, NM_{—}021969) (SEQ ID NO:11 or 12); TM7SF1 (NM_{—}003272) (SEQ ID NO:13 or 14); DLDH (dihydrolipamide dehydrogenase, NM_{—}000108) (SEQ ID NOS: 15 or 16); MAT2B (methionine adenosyltransferase II, beta, NM_{—}013283) (SEQ ID NO:17 or 18); STC-2 (stanniocalcin-2, NM_{—}003714) (SEQ ID NO:19 or 20); PPBI (alkaline phosphatase, intestinal precursor, NM_{—}001631) (SEQ ID NO:21 or 22); SLNAC1 (sodium channel receptor SLNAC1, NM_{—}004769) (SEQ ID NO:23 or 24); CAH4 (carbonic anhydrase iv precursor, NM_{—}000717) (SEQ ID NO:25 or 26); PA21 (phopholipase a2 precursor, NM_{—}000928) (SEQ ID NO:27 or 28); PAR2 (proteinase activated receptor 2 precursor, NM_{—}005242) (SEQ ID NO:29 or 30); IDE (insulin-degrading enzyme, NM_{—}004969) (SEQ ID NO:31 or 32); MYO1A (myosin-1A, NM_{—}005379) (SEQ ID NO:33 or 34); CYP2J2 (cytochrome P450 monooxygenase, NM_{—}000775) (SEQ ID NO:35 or 36); PHYH (phytanoyl-CoA-hydroxylase (Refsum disease), NM_{—}006214) (SEQ ID NO:37 or 38); CYB5 (cytochrome b5, 3′ end, NM_{—}001914) (SEQ ID NO:39 or 40); COXVIb (coxVIb gene, last exon and flanking sequence, NM_{—}001863) (SEQ ID NO:41 or 42); and TCF4 (NM_{—}030756) (SEQ ID NO:43 or 44) gene or polypeptide with a candidate antagonist molecule and measuring a detectable change in one or more biological activities normally associated with the ET-1 (endothelin-1, NM_{—}001955) (SEQ ID NO:1 or 2); AGR2 (anterior gradient 2 (Xenepus laevis) homolog, NM_{—}006408) (SEQ ID NO:3 or 4); ADAM8 (NM_{—}001109) (SEQ ID NO:5 or 6); PRSS8 (Prostasin precursor, serine protease, NM_{—}002773) (SEQ ID NO:7 or 8); AXO1 (Axonin-1 precursor, NM_{—}005076) (SEQ ID NO:9 or 10); NROB2 (Nuclear hormone receptor, NM_{—}021969) (SEQ ID NO:11 or 12); TM7SF1 (NM_{—}003272) (SEQ ID NO:13 or 14); DLDH (dihydrolipamide dehydrogenase, NM_{—}000108) (SEQ ID NOS:15 or 16); MAT2B (methionine adenosyltransferase II, beta, NM_{—}013283) (SEQ ID NO:17 or 18); STC-2 (stanniocalcin-2, NM_{—}003714) (SEQ ID NO:19 or 20); PPBI (alkaline phosphatase, intestinal precursor, NM_{—}001631) (SEQ ID NO:21 or 22); SLNAC1 (sodium channel receptor SLNAC1, NM_{—}004769) (SEQ ID NO:23 or 24); CAH4 (carbonic anhydrase iv precursor, NM_{—}000717) (SEQ ID NO:25 or 26); PA21 (phopholipase a2 precursor, NM_{—}000928) (SEQ ID NO:27 or 28); PAR2 (proteinase activated receptor 2 precursor, NM_{—}005242) (SEQ ID NO:29 or 30); IDE (insulin-degrading enzyme, NM_{—}004969) (SEQ ID NO:31 or 32); MYO1A (myosin-1A, NM_{—}005379) (SEQ ID NO:33 or 34); CYP2J2 (cytochrome P450 monooxygenase, NM_{—}000775) (SEQ ID NO:35 or 36); PHYH (phytanoyl-CoA-hydroxylase (Refsum disease), NM_{—}006214) (SEQ ID NO:37 or 38); CYB5 (cytochrome b5, 3′ end, NM_{—}001914) (SEQ ID NO:39 or 40); COXVIb (coxVIb gene, last exon and flanking sequence, NM_{—}001863) (SEQ ID NO:41 or 42); and TCF4 (NM_{—}030756) (SEQ ID NO:43 or 44) gene or polypeptide.

A “small molecule” is defined herein to have a molecular weight below about 500 Daltons.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas. The term “antibody” is used in the broadest sense and specifically covers, without limitation, intact monoclonal antibodies, polyclonal antibodies, multispecific antibodies (e.g., bispecific antibodies) formed from at least two intact antibodies, and antibody fragments so long as they exhibit the desired biological activity.

“Native antibodies” and “native immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR) regions. The variable domains of native heavy and light chains each comprise four FR regions, largely adopting a P-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., NIH Publ. No.91-3242, Vol. I, pages 647-669 (1991)). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institute of Health, Bethesda, Md. [1991]) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain ; Clothia and Lesk, J. Mol. Biol., 196:901-917 [1987]). “Framework” or “FR” residues are those variable domain residues other than the hypervariable region residues as herein defined.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 [1975], or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 [1991] and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 [1984]).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat or rabbit having the desired specificity, affinity, and capacity. In some instances, Fv FR residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see, Jones et al., Nature, 321:522-525 (1986); Reichmann et al., Nature, 332:323-329 [1988]; and Presta, Curr. Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest.

“Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et. al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition which is detectable. Radionuclides that can serve as detectable labels include, for example, I-131, I-123, I-125, Y-90, Re-188, Re-186, At-211, Cu-67, Bi-212, and Pd-109. The label may also be a non-detectable entity such as a toxin.

A “liposome” is a small vesicle composed of various types of lipids, phospholipids and/or surfactant which is useful for delivery of a drug (such as a CXCR4; Laminin alpha 4; TIMP1; Type IV collagen alpha 1; Laminin alpha 3; Adrenomedullin; Thrombospondin 2; Type I collagen alpha 2; Type VI collagen alpha 2; Type VI collagen alpha 3; Latent TGFbeta binding protein 2 (LTBP2); Serine or cystein protease inhibitor heat shock protein (HSP47); Procollagen-lysine, 2-oxoglutarate 5-dioxygenase; connexin 43; Type IV collagen alpha 2; Connexin 37; Ephrin A1; Laminin beta 2; Integrin alpha 1; Stanniocalcin 1; Thrombospondin 4; or CD36 polypeptide or antibody thereto and, optionally, a chemotherapeutic agent) to a mammal. The components of the liposome are commonly arranged in a bilayer formation, similar to the lipid arrangement of biological membranes.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Up-regulation,” “increased expression,” and “overexpression” are used interchangeably and, as used herein, mean at least about a 1.5-fold increase in expression, alternatively at least about a 2-fold increase in expression, alternatively with at least about a 2.5-fold or higher increase in expression of a gene measured as an increase in its DNA (amplification), its mRNA (increased transcription), or in the level of polypeptide encoded by the gene. Alternatively, up-regulation or increased expression is determined using a Z score as a p value <0.07 relative to a normal tissue control.

The term “package insert” is used to refer to instructions customarily included in commercial packages of therapeutic products, that contain information about the indications, usage, dosage, administration, contraindications and/or warnings concerning the use of such therapeutic products.

It will be clearly understood that, although a number of art publications are referred to herein, this reference does not constitute an admission that any of these documents forms part of the common general knowledge in the art, in Australia or in any other country.

Throughout this specification and the claims, the terms “comprise,” “comprises,” and “comprising” are used in a non-exclusive sense, except where the context requires otherwise.

### EXAMPLES

The following examples are offered by way of illustration and not by way of limitations. The examples are provided so as to provide those of ordinary skill in the art with a complete disclosure and description of how to make and use the compounds, compositions, and methods of the invention and are not intended to limit the scope of what the inventors regard as their invention. Efforts have been made to insure accuracy with respect to numbers used (e.g. amounts, temperature, etc. but some experimental errors and deviation should be accounted for. Unless indicated otherwise, parts are in parts by weight, temperature is in degrees C, and pressure is at or near atmospheric. The disclosures of all citations in the specification are expressly incorported herein by reference.

### Example 1

### Patients and Tissue Collection

Esophageal mucosal biopsies were obtained from patients undergoing surveillance endoscopy at the Western General Hospital and Royal Infirmary, Edinburgh during 2000-1. The study was approved by the Lothian Research and Ethics Committee and written, informed consent was obtained from all patients. All procedures were performed by one of two experienced endoscopists with expertise in Barrett's esophagus in a standard manner according to a local protocol for Barrett's surveillance. BE was defined as tongues or circumferential salmon pink mucosa extending for at least 3 cm above the gastro-esophageal junction. At endoscopy, careful note was made of the length of the CE segment, severity of any esophagitis if present and the presence of macroscopically visible abnormalities within the BE. Data on smoking history, use of acid-suppressing drugs and Helicobacter pylori status were also recorded.

Paired biopsies were taken. One sample was fixed in formalin for histology and the other stored fresh-frozen (−70° C.) for microarray analysis. Two gastrointestinal pathologists reviewed all specimens, which were categorized as: normal squamous esophagus, BE (columnar lined esophagus with intestinal metaplasia and the presence of goblet cells and alcian blue positive mucin), BE with changes indeterminate dysplasia, BE with low-grade dysplasia (LGD), BE with high-grade dysplasia (HGD) or BE with adenocarcinoma (CA). For some patients, 2 separate biopsy specimens for the same disease state were available for array analysis. Additional matched samples were also analyzed (e.g. biopsies of BE adjacent to carcinoma in BE from the same patient). Analyzed samples included 10 normal esophagus, 28 samples of BE from 20 patients, 6 samples of LGD from 3 patients, 3 samples indeterminate for dysplasia from 2 patients, 6 samples HGD from 3 patients, 10 samples of BE adjacent to CA (BE-CA) from 7 patients, 16 samples CA from 10 patients.

Microarrays containing 9031 genes were generated by printing PCR products derived from cDNA clones (Invitrogen, California and Genentech, Inc.) on glass slides coated with 3-aminopropyltriethoxysilane(Aldrich, Milwaukee Wis.) and 1,4-phenylenediisothiocyanate (Aldrich, Milwaukee Wis.) using a robotic arrayer (Norgren Systems, Mountain View, Calif.). RNA isolation was accomplished by CsCl step gradient, (Kingston, Current Protocols in Molecular Biology 1:4.2.5-4.2.6 (1998)) typically 0.1-2 μg of total RNA was obtained. Probes for array analysis were generated by conservative amplification and subsequent labelling as follows: double-stranded DNA generated from 0.1 μg of total RNA (Invitrogen, Carlsbad, Calif.) was amplified using a single round of a modified in vitro transcription protocol (MEGASCript T7 from Ambion, Austin, Tex. (Gelder et al., Proc. Natl. Acad. Sci. USA 87:1663-1667 (1990)). The resulting cRNA was used as a template to generate a sense DNA probe using random primers (9mers, 0.15 mg/ml), Alexa 488 dUTP or Alexa 546 dUTP (40 μM and 6 μM, respectively, Molecular Probes, Eugene, Oreg.) using MMLV-derived reverse transcriptase (Invitrogen, Carlsbad, Calif.). A reference probe to reflect general epithelial cell expression was generated from 0.1 μg of total RNA from a pool of liver, lung and kidney (Clontech, Palo Alto, Calif.). Probes were hybridized to arrays overnight in 50% formamide/5×SSC at 37° C. and washed the next day in 2×SSC, 0.2% SDS followed by 0.2×SSC, 0.2% SDS. Array images were collected using a CCD-camera based imaging system (Norgren Systems, Mountain View, Calif.) equipped with a Xenon light source and optical filters appropriate for each dye. Full dynamic-range images were collected (Autograb, Genentech Inc) and intensities and ratios extracted using automated gridding and data extraction software (gImage, Genentech Inc) built on a Matlab (the MathWorks, Natick, Mass.) platform.

### Example 3

### Data Analysis

Data were sorted to identify genes expressed above background (N intensity of >12 where background values range from 0-8) in the test sample such that only meaningful ratios were included. Ratio values were further normalized for experimental scatter at different intensity values within each experiment by plotting log ratio versus N intensity and by fitting a normal distribution at each intensity level. A measure of standard deviation (Z score) around a mean of zero was derived for each gene in each experiment and this value was used in data mining. Specifically, for each microarray, data were normalized by computing Z-scores, which were obtained from a scatterplot of the logarithm of the ratio of the test and reference data versus the logarithm of the minimum of the test and reference data. The median of the ratio as a function of intensity was estimated by applying the loess algorithm to the scatterplot. The standard error was estimated by applying loess to the square root of the absolute residuals, and squaring the result to obtain the median absolute deviation (MAD), and making a multiplicative correction to convert from MAD to a standard error. The Z scores were determined for each ratio by dividing its vertical distance from the median loess curve by the standard error at that intensity.

A computational process useful computing Z-scores may be written in a standard high-level statistical language, S-Plus, as follows:

pos.test→test[test>0 & ref>0]

pos.ref→ref[test>0 & ref>0]

minorder→order(pmin(pos.test,pos.ref))

y→log(pos.test[minorder]+10)−log(pos.ref[minorder]+10)

x→log(pmin(pos.test[minorder],pos.ref[minorder]))

residuals→loess(y˜x)$residuals

sqresiduals→sqrt(abs(residuals))

sqrt.mad→loess(sqresiduals˜x)$fitted

sigma→sqrt.mad*sqrt.mad/0.6745

zscore→ifelse(sigma>0,residuals/sigma,0)

This code may be executed in a commercially available S-Plus program such as, for example, (http://www.insightful.com), or in a freely available substituteprogram, R (http://www.r-project.org).

### Example 4

### Differential Expression in Barrett's Esophagus-to-Adenocarcinoma Disease Stages

Samples and Data Mining:

High-quality data were obtained from >90% of biopsy specimens, including those of poor RNA quality and very limited RNA quantity (eg. less than 200 ng total RNA). A data mining strategy was applied to identify genes specifically associated with the different stages of disease progression. Experiments were grouped into disease categories based on pathologic diagnosis, and these groups compared to identify genes with significant elevated expression for at least 25% of the samples within a disease group with respect to both the epithelial pool reference and the normal esophagus group. Typically, genes with elevated expression were identified as those with Z scores of >1.7 (p<0.05) in the disease group, corresponding to ratio values of 2-20 in most cases. A total of 460 genes satisfied these criteria across the disease groups BE, dysplasia, and carcinoma (some genes are associated with more than one disease group). Selected genes (117) are listed (Tables 1, 2, 3). All dysplasia samples (high-, low-grade and indeterminate) were combined into a single group to improve data analysis, and the genes identified were then further inspected to determine if they were more prevalent in low- or high-grade dysplasia. HGD sample data were independently analyzed to determine gene expression profiles diagnostic for high-grade dysplasia (Table 4A).

Inflammation:

Significant expression of proinflammatory, costimulatory and inducible cytokines and receptors was observed in BE, dysplasia and carcinoma, and the most prevalent genes are listed (Table 1). Some binding partners were detected, such as putative inflammatory cytokine IL-17 family member IL-17E and its receptor IL-17BR, and SCYA20/LARC and receptor CCR6 (Lee et al., J. Biol. Chem. 276:1660-1664 (2001); and Baba et al., J. Biol. Chem. 272:14893-14898 (1997)). SCYA20 is expressed in the epithelium of the small intestine and is chemotactic for lymphocytes and dendritic cells (Tanaka et al., Eur. J. Immunol. 29:644-642 (1999)). Activin A is a TGF beta superfamily member that can act as a potent mediator of cell growth and differentiation and may be involved in response to injury (Munz et al., EMBO J. 18:5205-5215 (1999)). It was co-expressed particularly in carcinoma in Barrett's samples with its serine-threonine kinase receptor AVRII (the type I receptor was also detected but less well correlated). Chemokine receptors CXCR4 and CCR7 have been detected on a variety of inflammatory cell types, but have also been described has highly expressed in breast tumor cells, with possible involvement in lymph node metastasis (Muller et al., Nature 410:50-56 (2001)). In this study, CXCR4 in particular was associated with high-grade dysplasia and detected in some samples of adenocarcinoma.

TABLE 1A
Cytokines and chemokines up-regulated in BE-to-Adenocarcinoma

| NCBI RefSeq | Gene | BE | D | BE-CA | CA |
|---|---|---|---|---|---|
| NM_000594 | TNF-a | * | | * | * |
| NM_002546 | Osteoprotegerin | * | | * | |
| NM_002993 | GCP-2 | (*) | *H | (*) | * |
| NM_025240 | B7-H3 | | *L | (*) | * |
| NM_002995 | Lymphotactin | (*) | * | | (*) |
| NM_005746 | PBEF | * | | | (*) |
| NM_004591 | SCYA20 | | (*) | * | |
| NM_004843 | WSX1 | | * | | |
| NM_019618 | IL1-H1 | (*) | | * | * |
| NM_000418 | IL-4R | | | | * |
| NM_022789 | IL-17E | (*) | * | * | * |
| NM_018725 | IL-17BR | | *H | | (*) |
| NM_014432 | IL-20Ra | | *L | | (*) |
| NM_021798 | IL-21R | (*) | | * | * |
| NM_002192 | Activin A | | (*) | (*) | * |
| NM_001616 | AVR2, type II activin receptor | | * | | * |
| NM_001105 | Activin A type I Receptor | | | | (*) |
| NM_031409 | CCR6 | (*) | | * | * |
| NM_003467 | CXCR4 | | *H | | (*) |
| NM_001838 | CKR7 | (*) | (*) | * | |

TABLE 1B
Prostaglandin synthesis-related genes up-regulated
in BE-to-Adenocarcinoma

| NCBI | | | | | |
|---|---|---|---|---|---|
| RefSeq | Gene | BE | D | BE-CA | CA |
| NM_000963 | COX-2, prostaglandin synthase 2 | (*) | *H | | * |
| NM_000962 | COX-1, prostaglandin synthase 1 | | | | * |
| NM_007366 | PLA2R phosphlipase A2 R1 | | * | (*) | * |
| NM_000953 | PD2R prostaglandin D2 R | (*) | | (*) | * |
| NM_000959 | PF2AR prostaglandin F2α R | | * | (*) | (*) |
| NM_000957 | PER3 prostaglandin E R 2 | | | (*) | * |
| NM_000960 | Prostaglindin IP (I2) R | * | * | (*) | |

Genes are associated with the disease states B3, dysplasia (D), BE adjacent to carcinoma (BE-CA), or carcinoma (CA) if present in at least 25% of samples tested.
(*) indicates gene expression changes associated with 15-25% of samples.

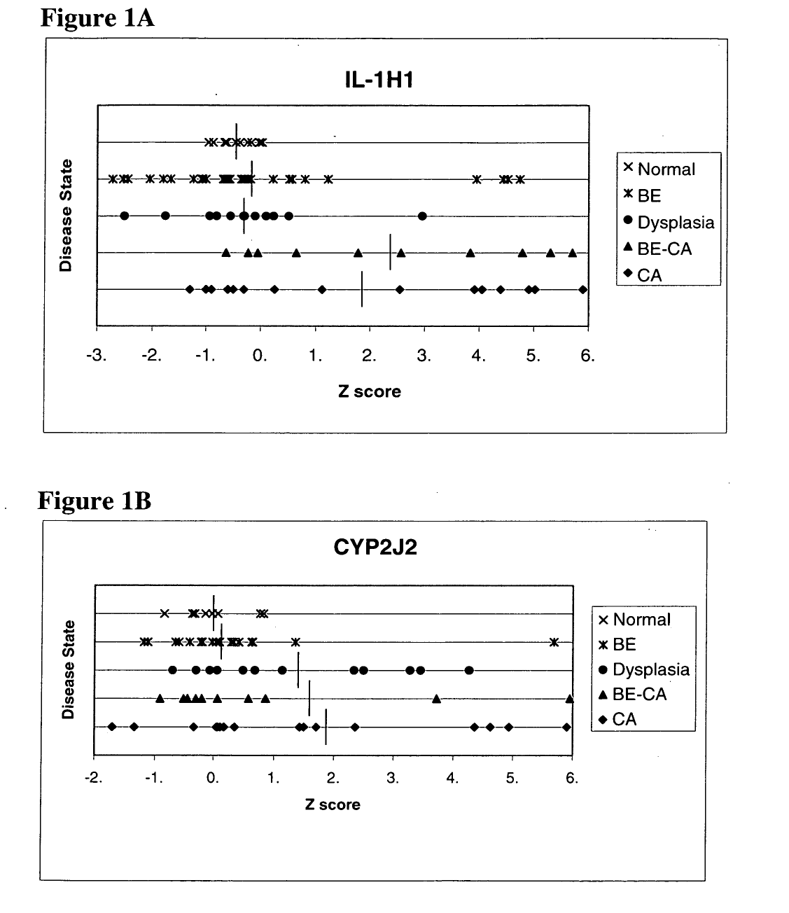

An otherwise rare IL-1 homolog, IL1-H1, was highly expressed in carcinoma in Barrett's, and also the matched adjacent BE tissue from the same patients (FIG. 1). A previous study of the murine Il-1H1 ortholog detected constitutive only in esophageal squamous mucosa. In addition, human IL1-H1 mRNA could be induced in TNF□ and IFN□ treated keratinocytes and squamous epithelial tumor cell line A431 (Kumar et al., J. Biol. Chem. 275:10308-10314 (2000)). This gene is one marker of a specific esophageal squamous cell type exhibiting a striking induction of expression in both adenocarcinoma and patient-matched BE, amidst primarily intestinal and tumor markers observed in this study (Tables 2 and 3). The high expression in BE matched with adenocarcinoma in addition to adenocarcinoma suggests a possible epigenetic association.

Cylooxyengase isoform 2 (COX-2), which catalyzes a rate-limiting step in conversion of arachidonate to inflammatory prostaglandins, has been implicated in Barrett's metaplasia and other cancers (Morris et al., Am. J. Gastroenterol. 96:990-996 (2001); Heasley et al., J. Biol. Chem. 272:14501-14504 (1997); and Tsujii et al., Cell 93:705-716 (1998)). Consistent with previous reports, a significant increase was observed in COX-2 gene expression with increasing dysplasia (high-grade dysplasia) and in adenocarcinoma (Table 1B). Smaller changes were also observed in COX-1 and several prostaglandin receptors. Arachidonic acid is released from the membrane by the action of phospholipases. Phospholipase A2 expression associated with increasing malignancy was also observed (Table 2) along with the M-type receptor (PLA2R, Table 1B), consistent with studies suggesting that COX-2, PA2 and PLA2R are coordinately expressed (Rys-Sikora et al., Am. Physiol. Cell Physiol. 278:822-833 (2000)).

Elevated expression was detected for another enzyme that generates a different class of biologically active eicosanoids from arachidonic acid, the epoxygenase CYP2J2 (FIG. 1B, Table 2). This cytochrome P450 enzyme is expressed in a variety of cell types in the small intestine, including epithelial cells, and may play a role in electrolyte transport, intestinal motility, and other processes (Wu et al., J. Biol. Chem. 271:3460-3468 (1996); Zeldin et al., Mol. Pharm. 51:931-943 (1997); and Node et al., Science 285:1276-1279 (1999)). Similar to COX-2, elevated expression is most apparent in samples of adenocarcinoma and dysplasia (both low-grade and high-grade dysplasia). The expression profile for CYP2J2 also reflects the progressive intestinal metaplasia observed in this study (Table 2).

Intestinal Metaplasia:

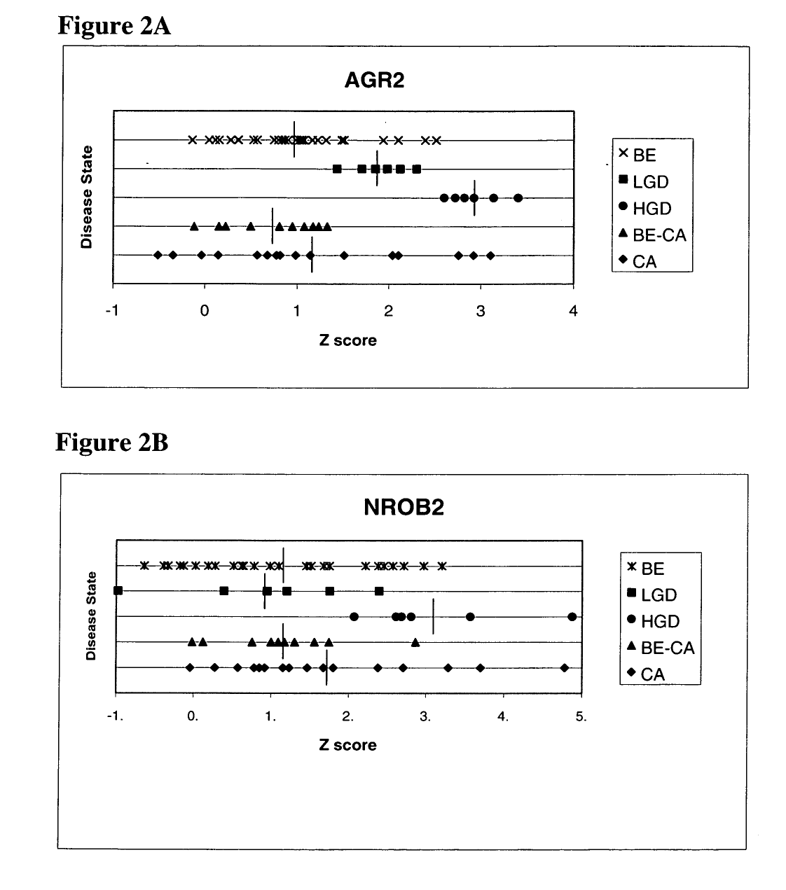

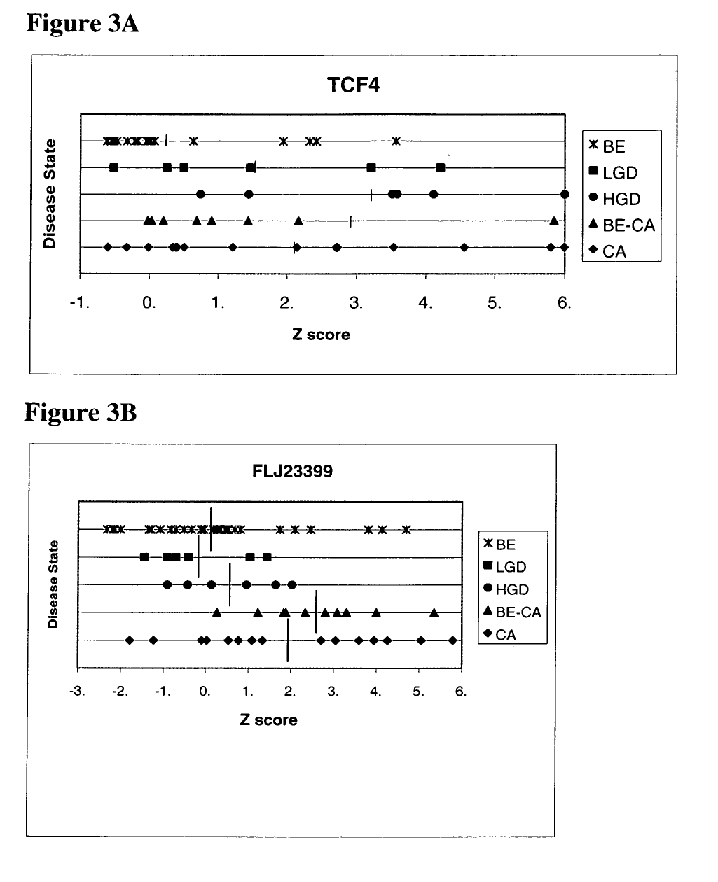

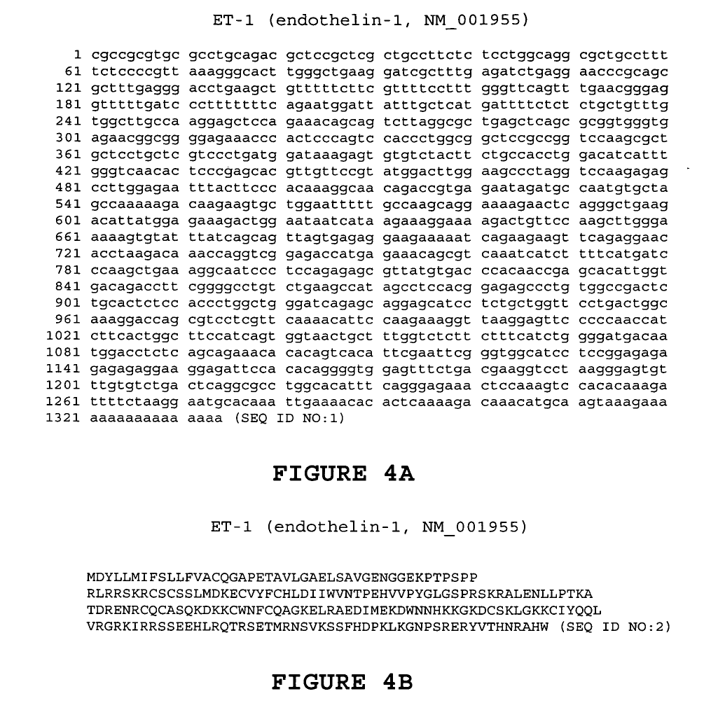

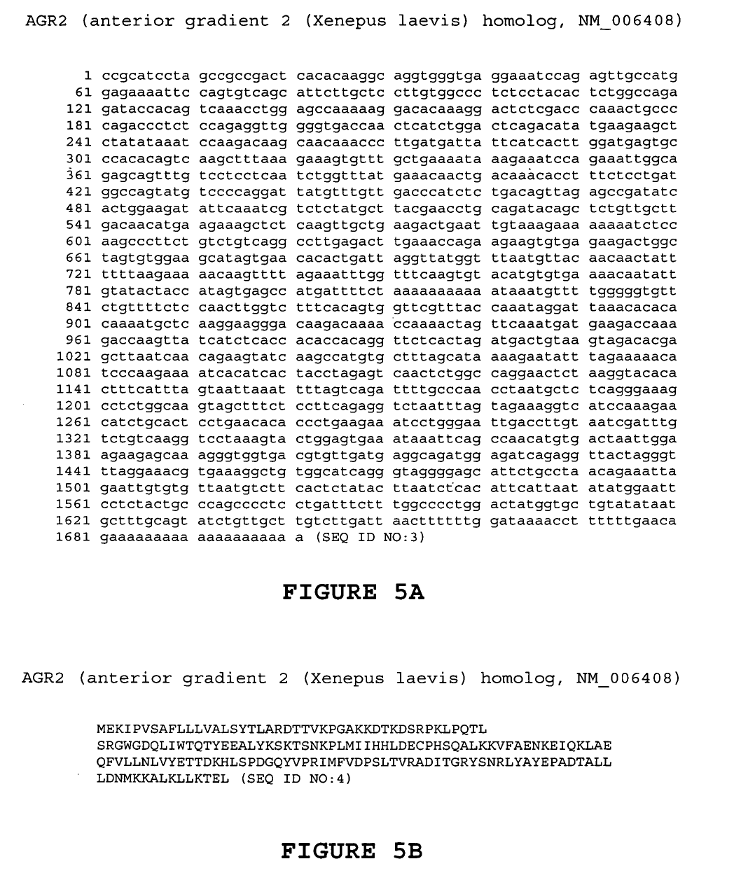

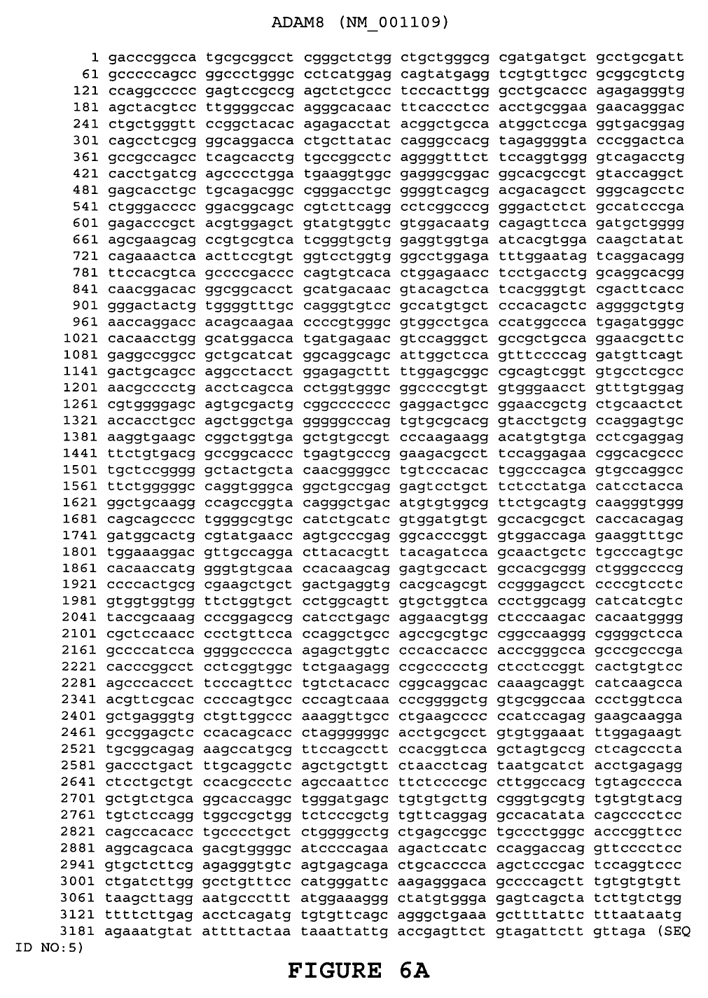

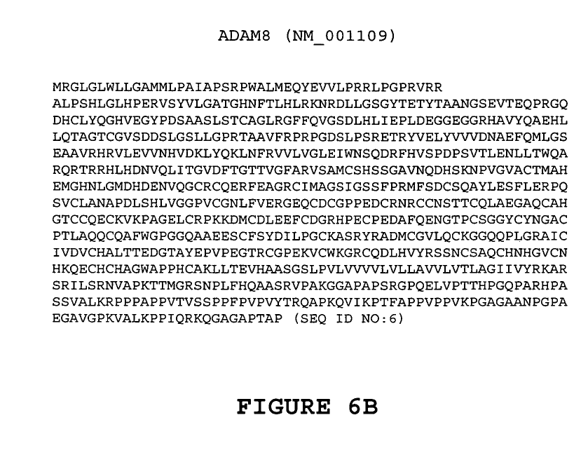

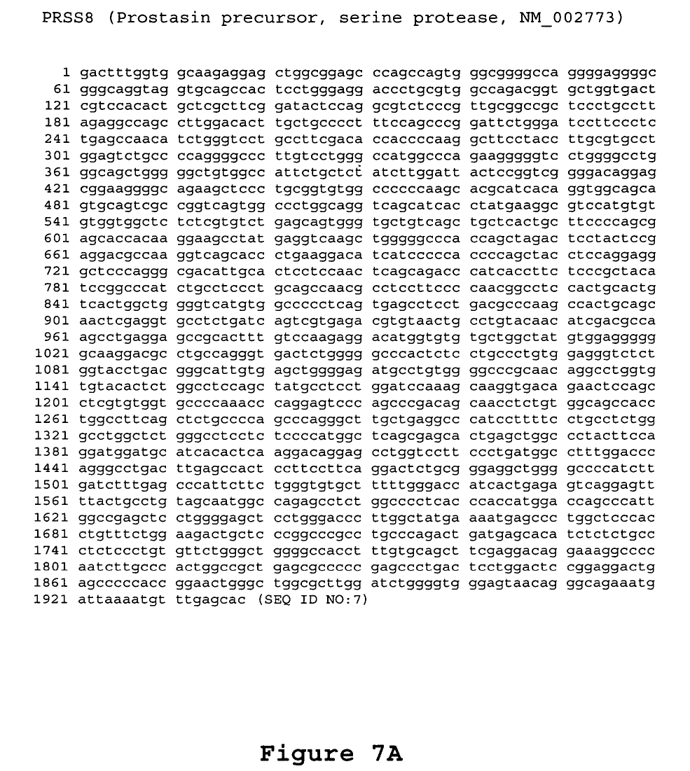

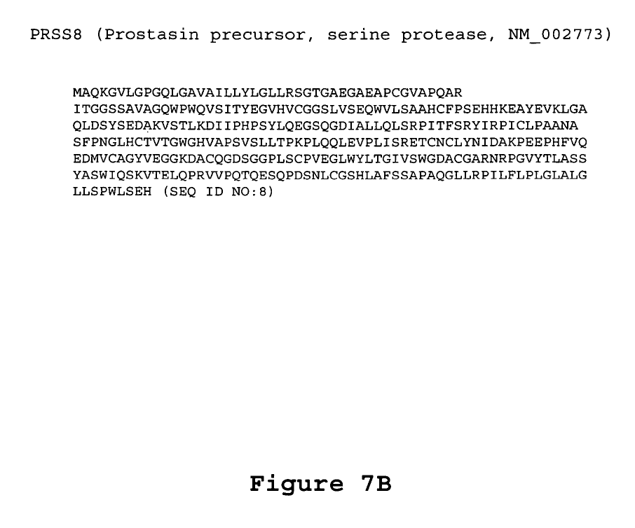

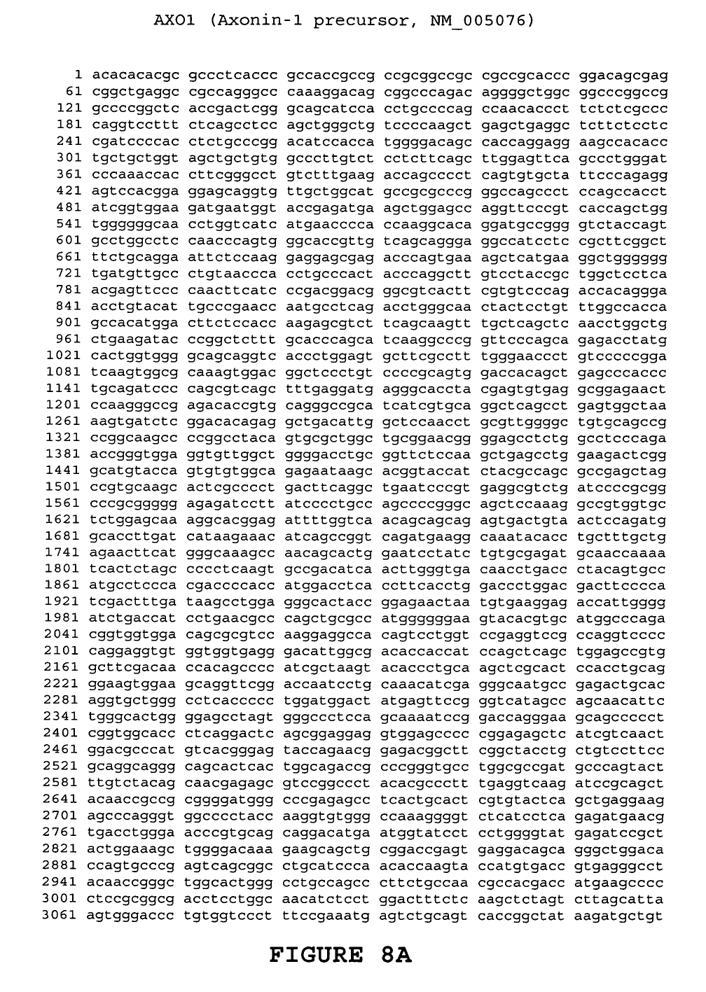

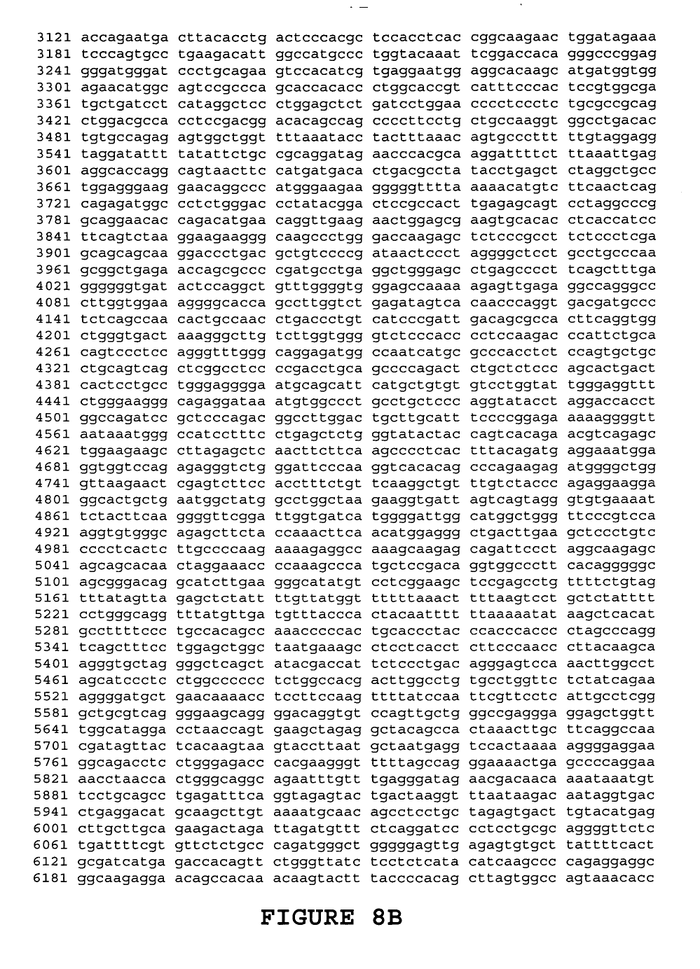

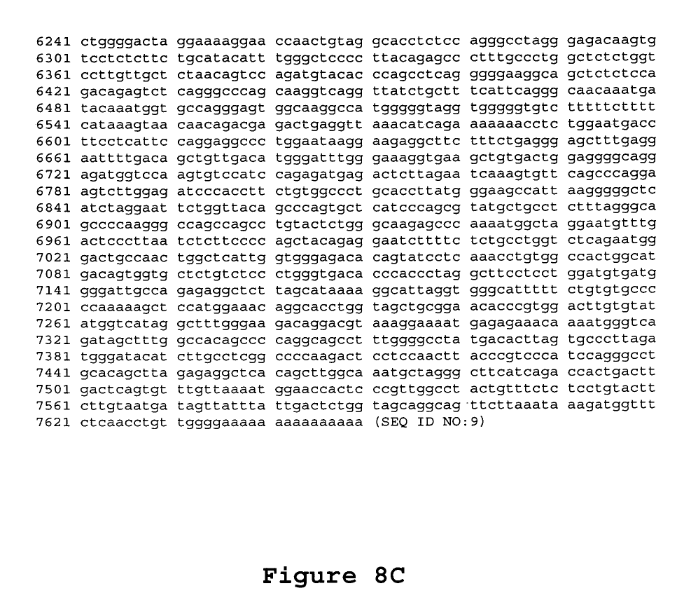

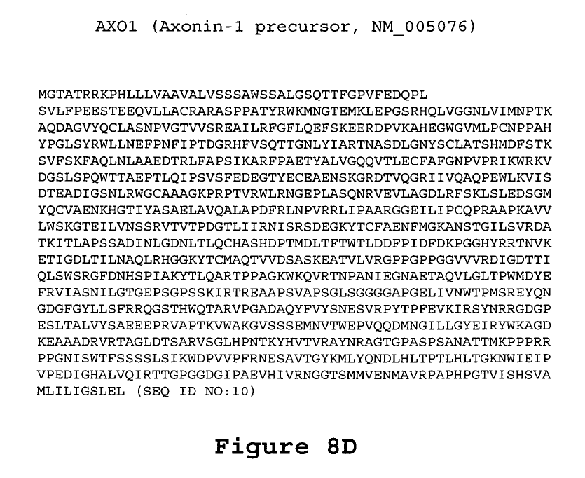

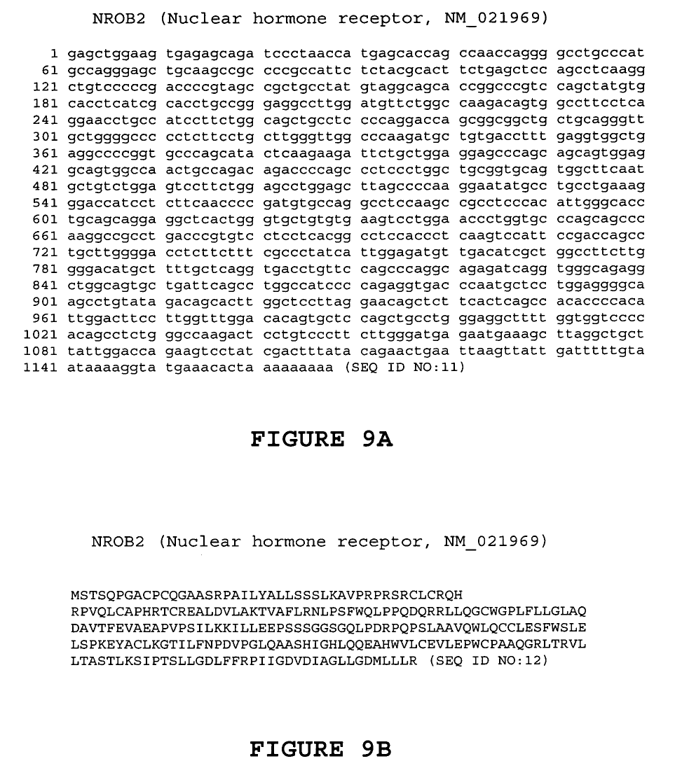

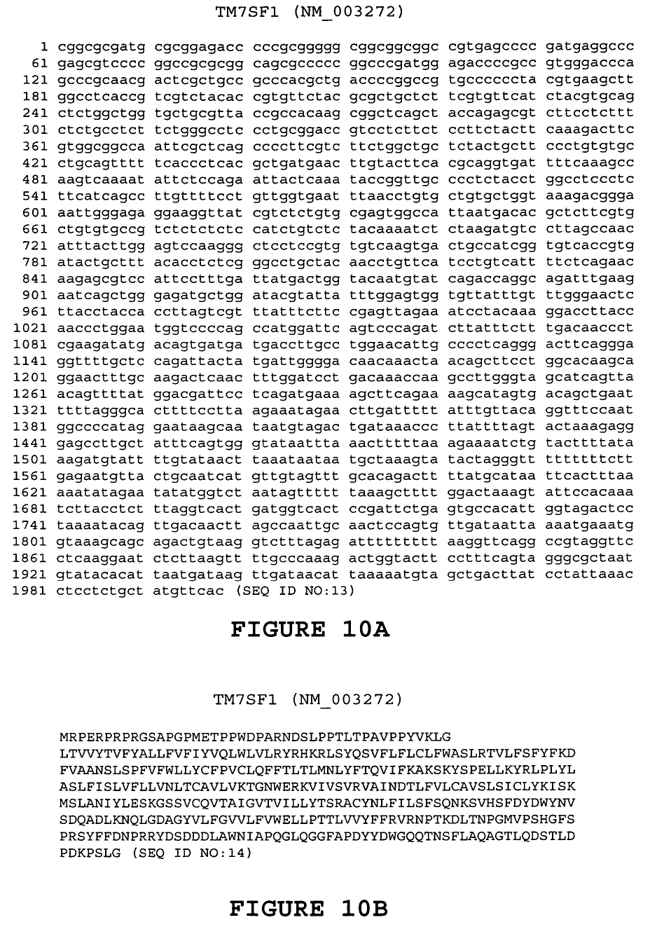

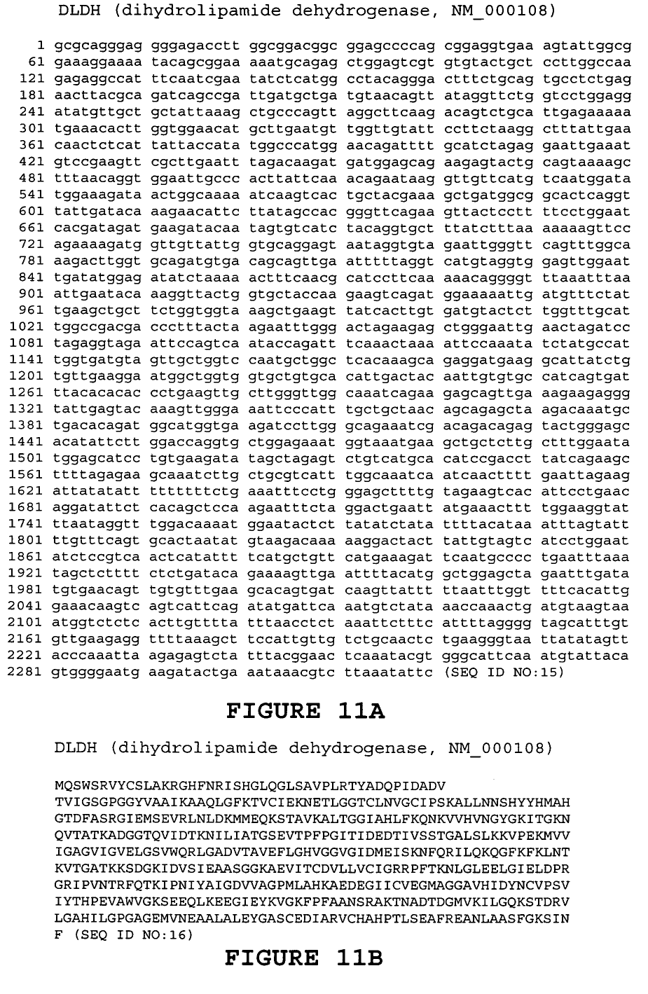

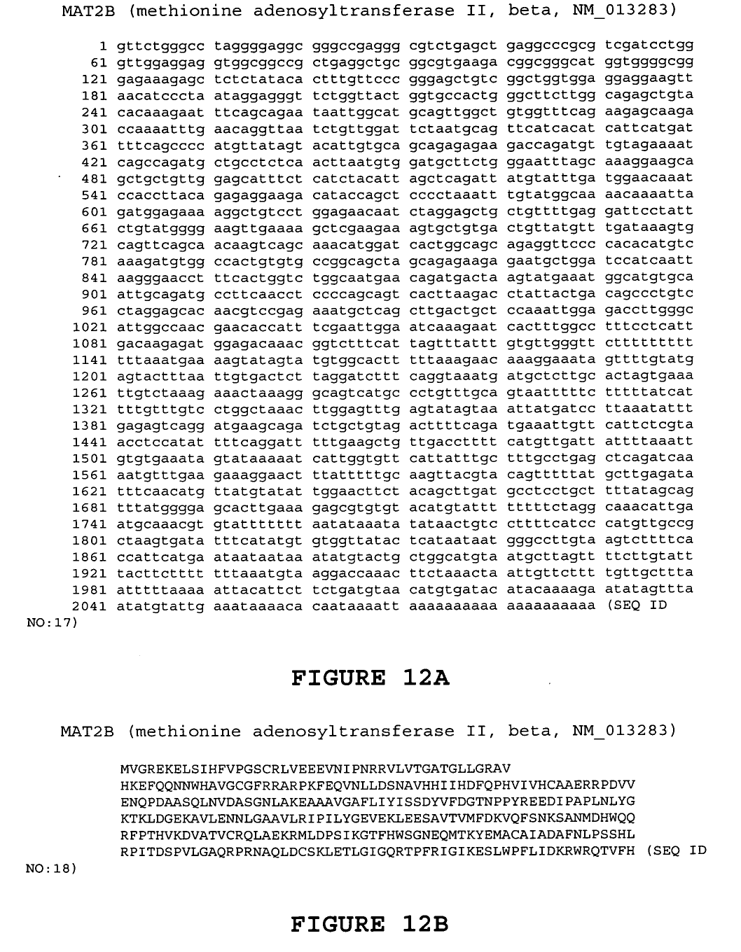

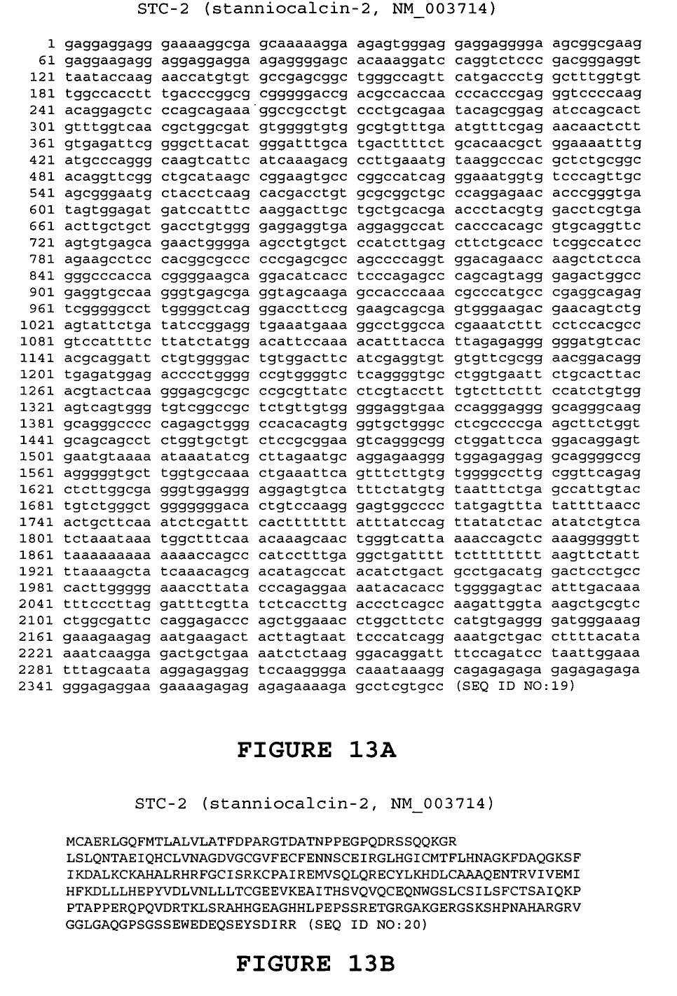

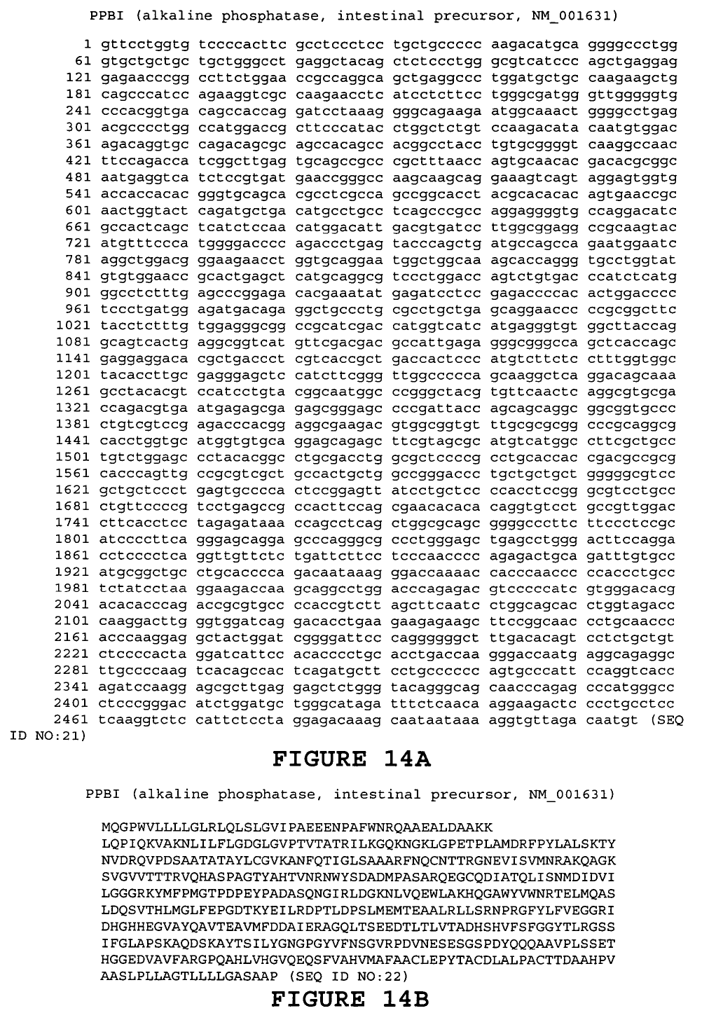

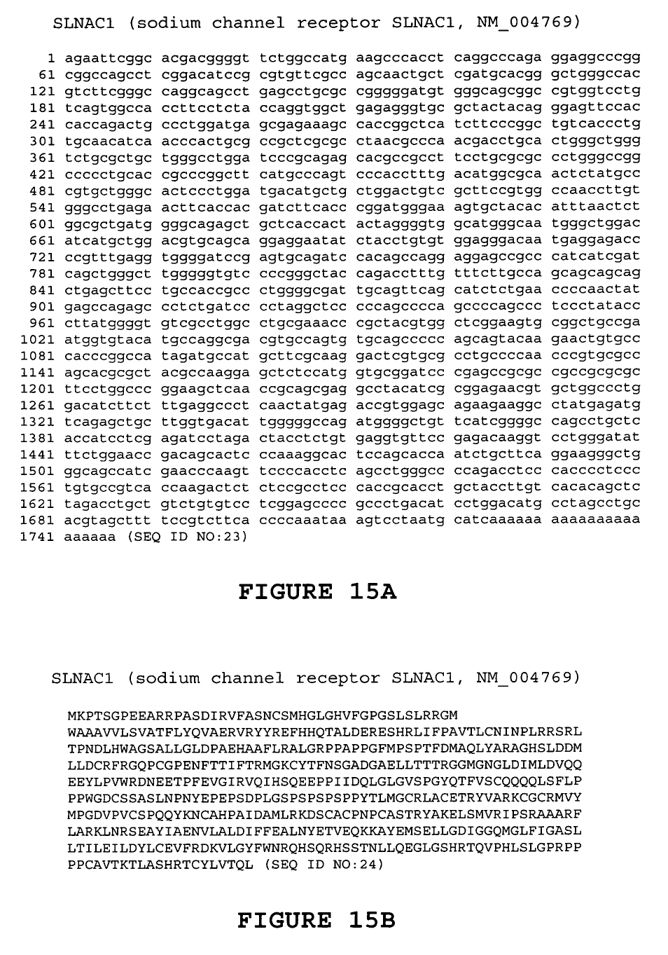

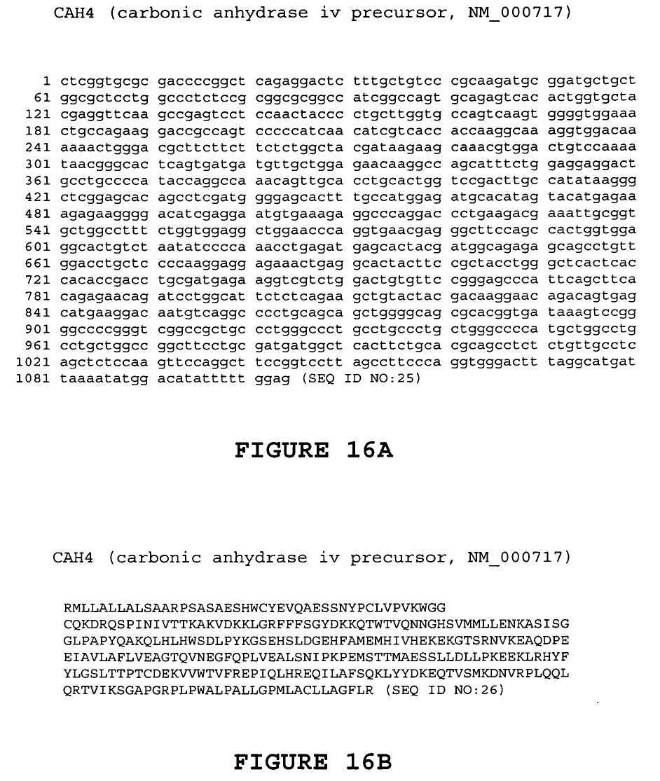

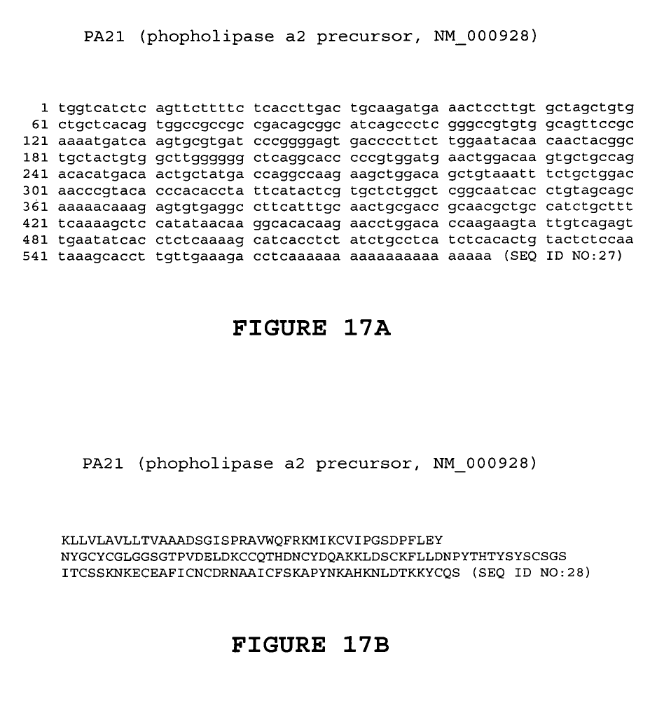

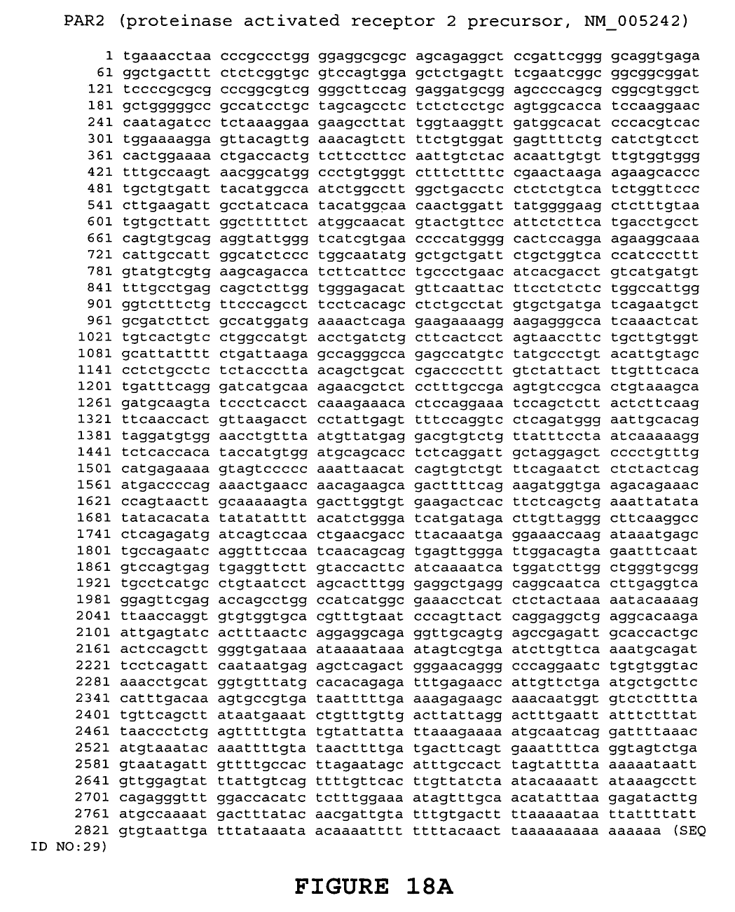

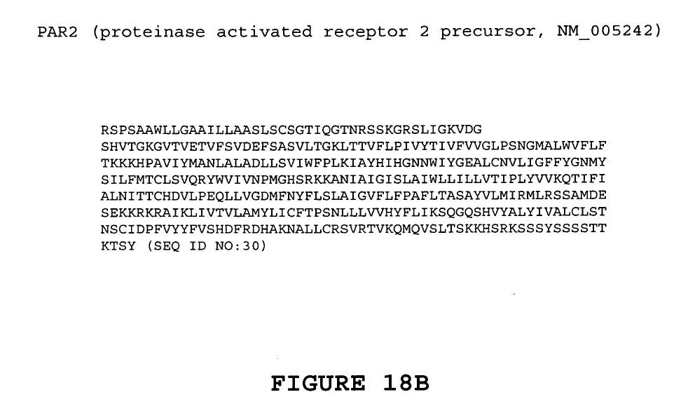

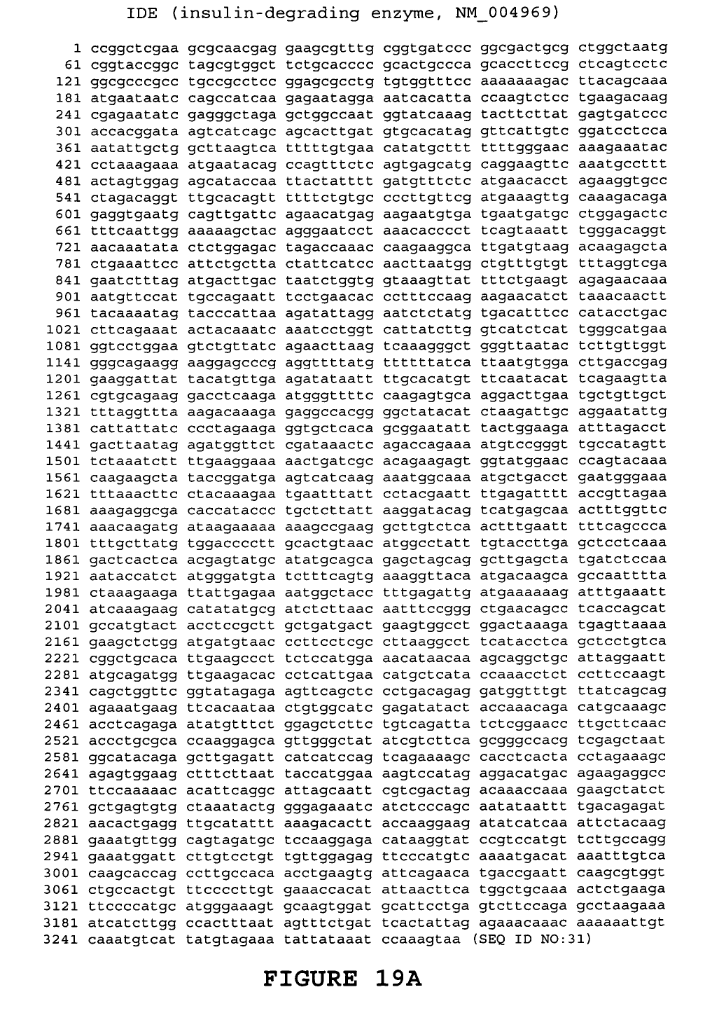

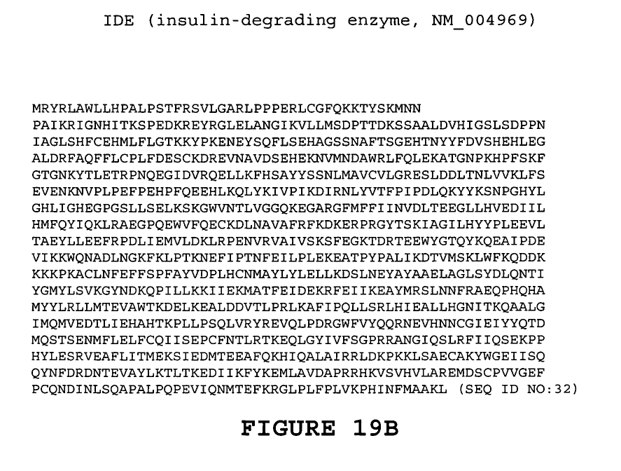

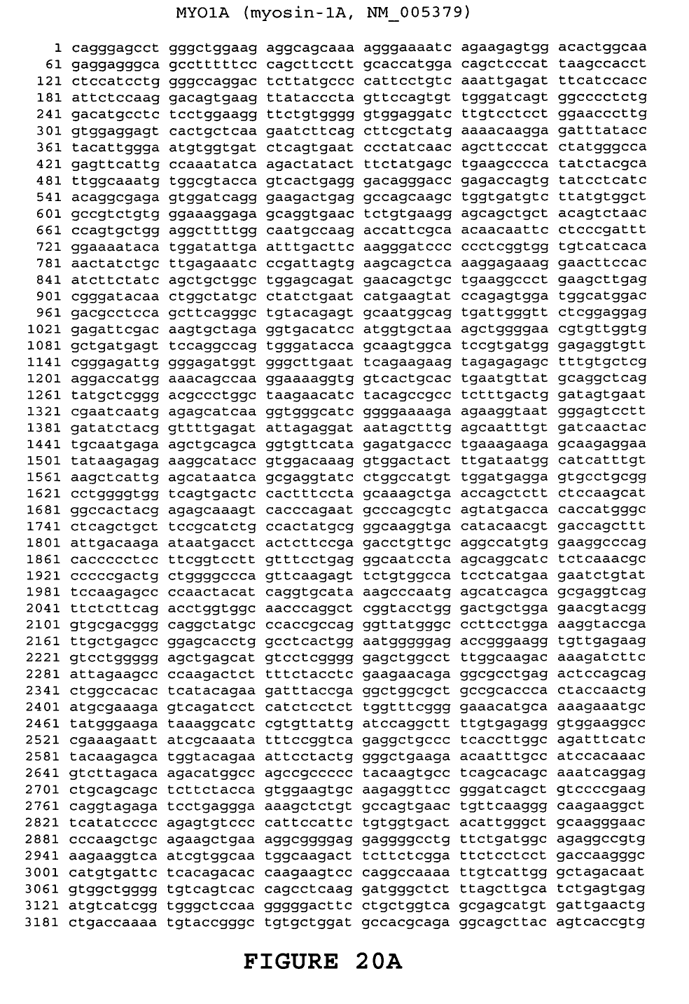

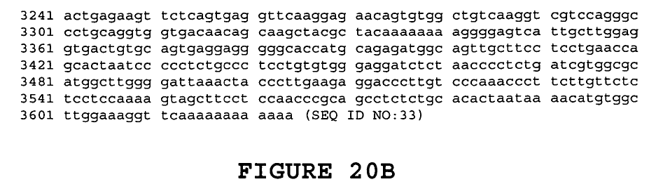

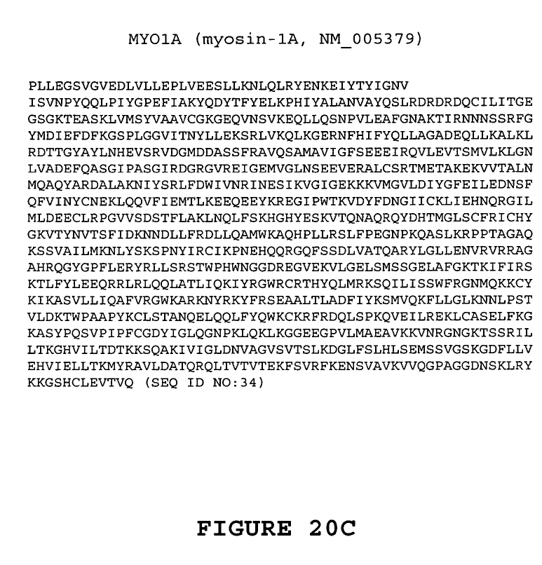

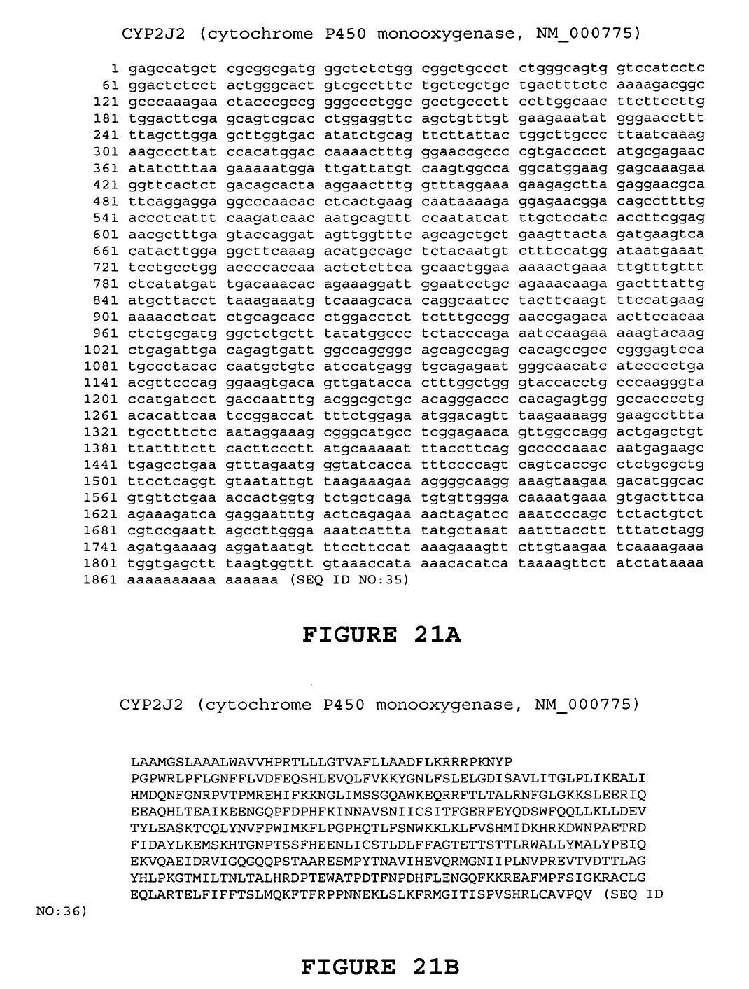

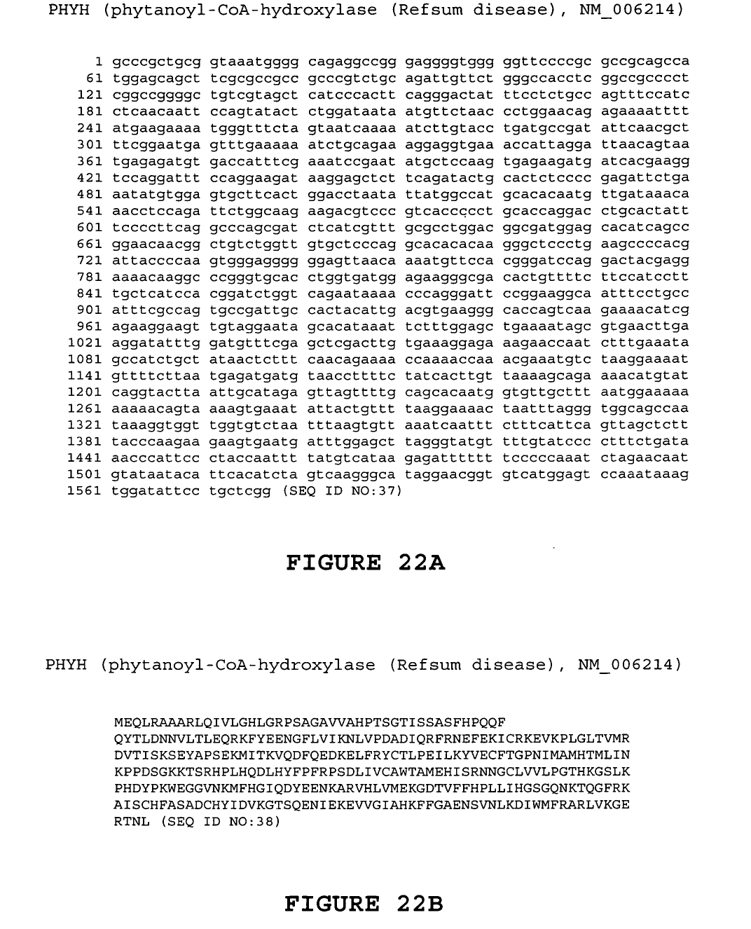

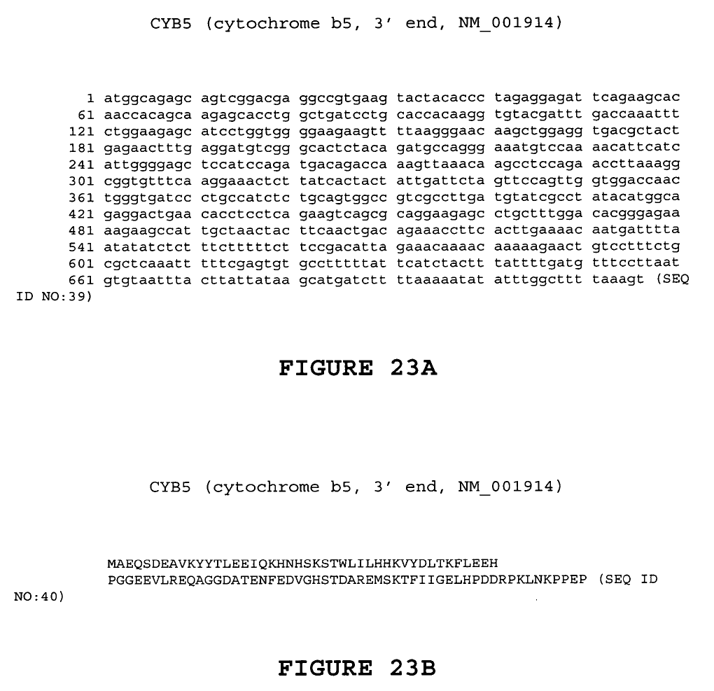

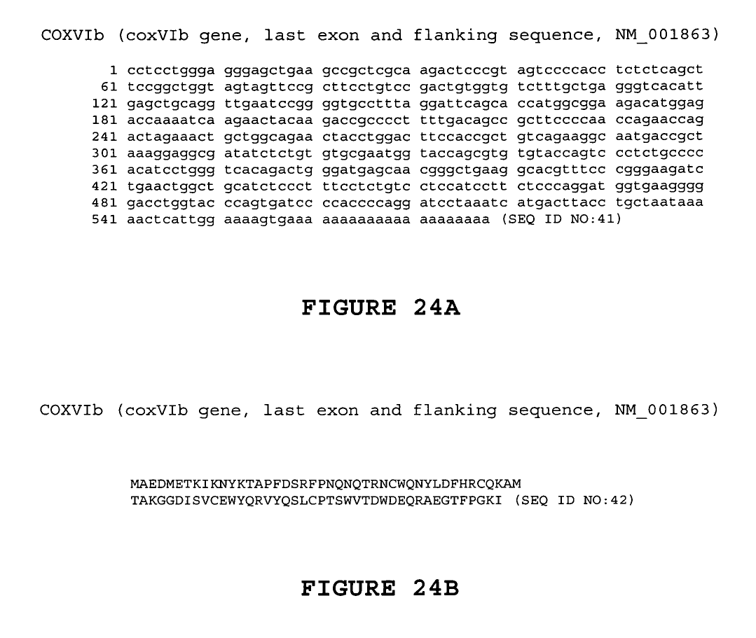

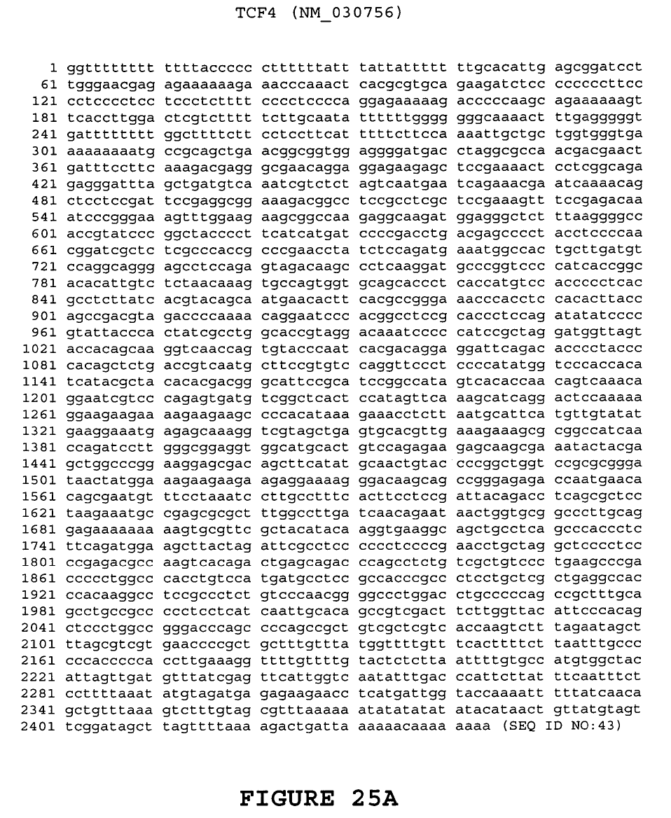

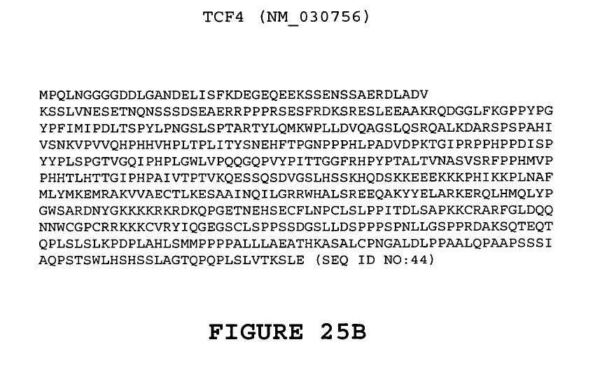

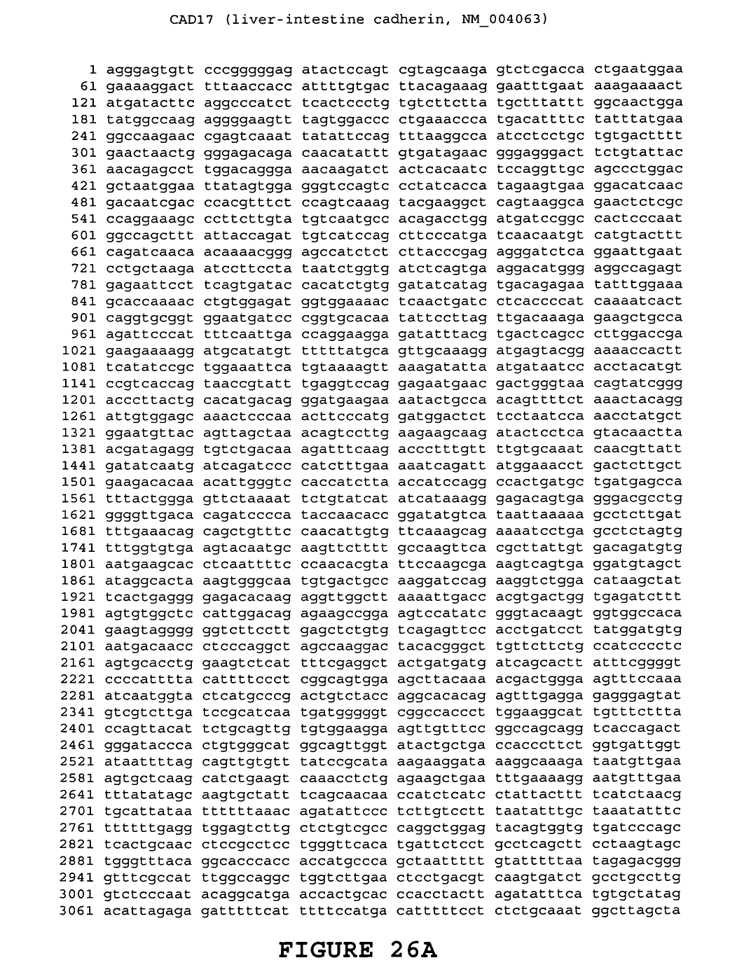

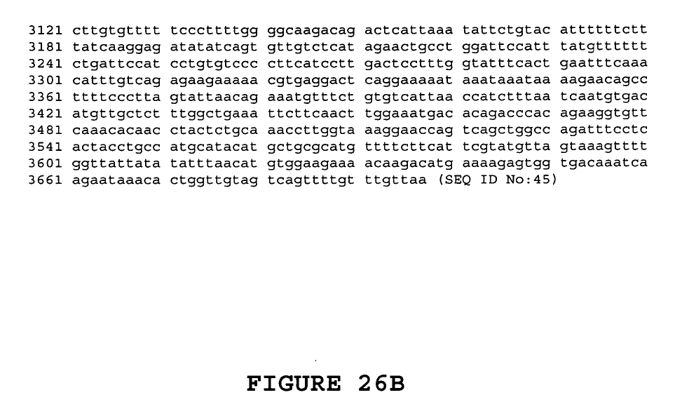

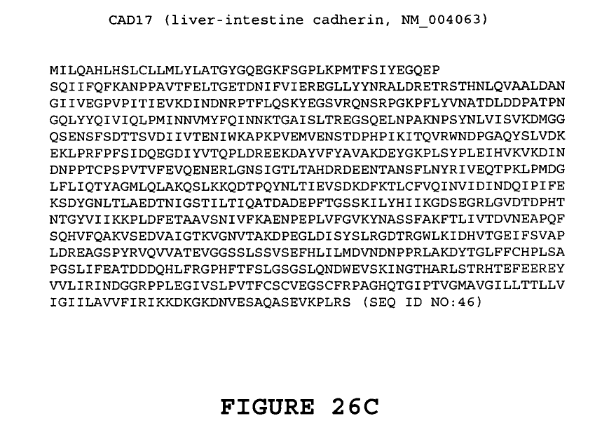

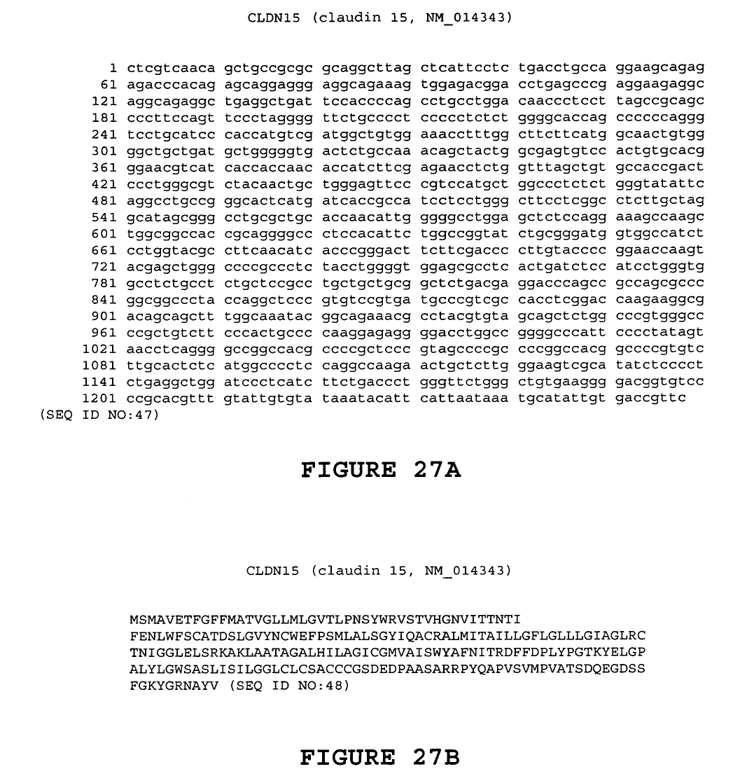

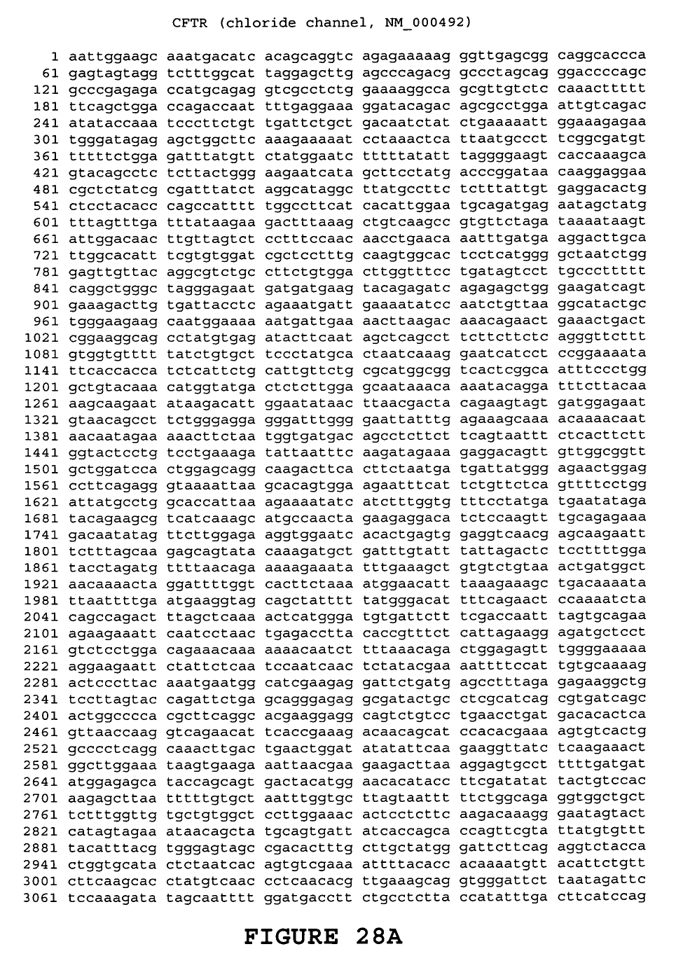

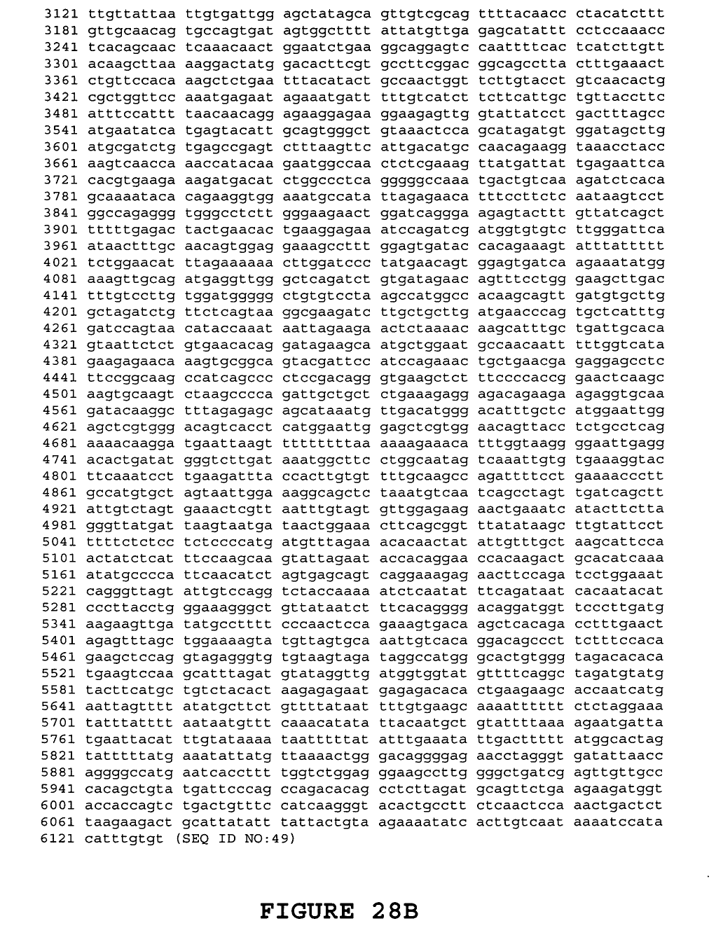

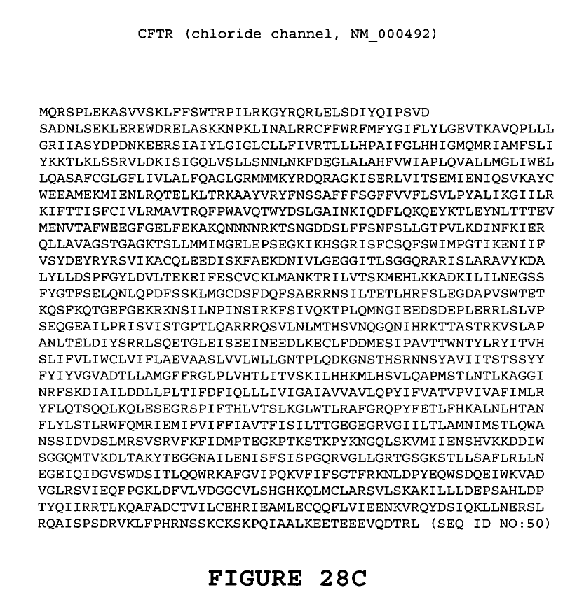

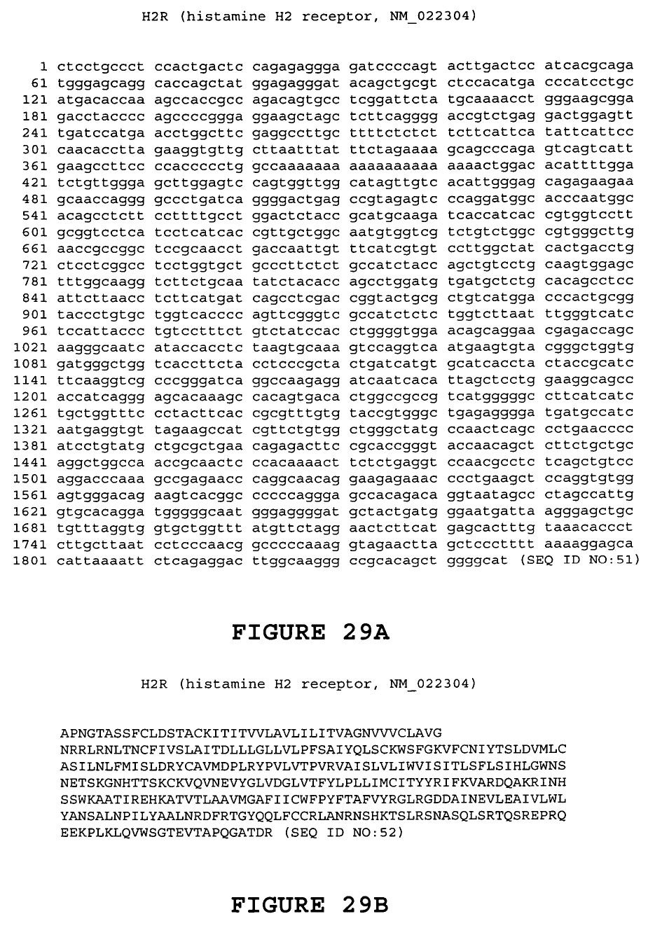

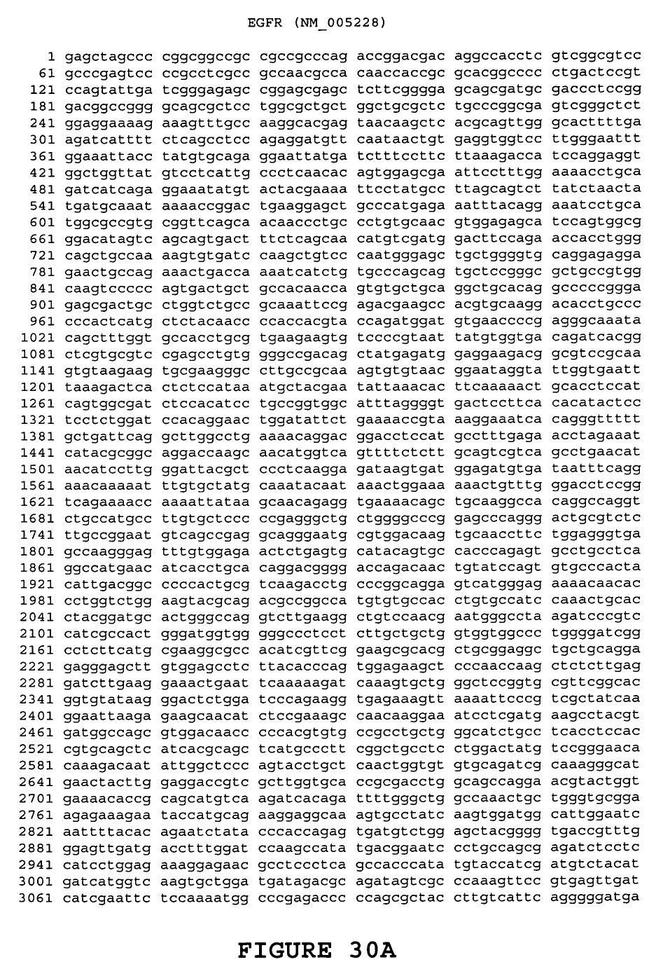

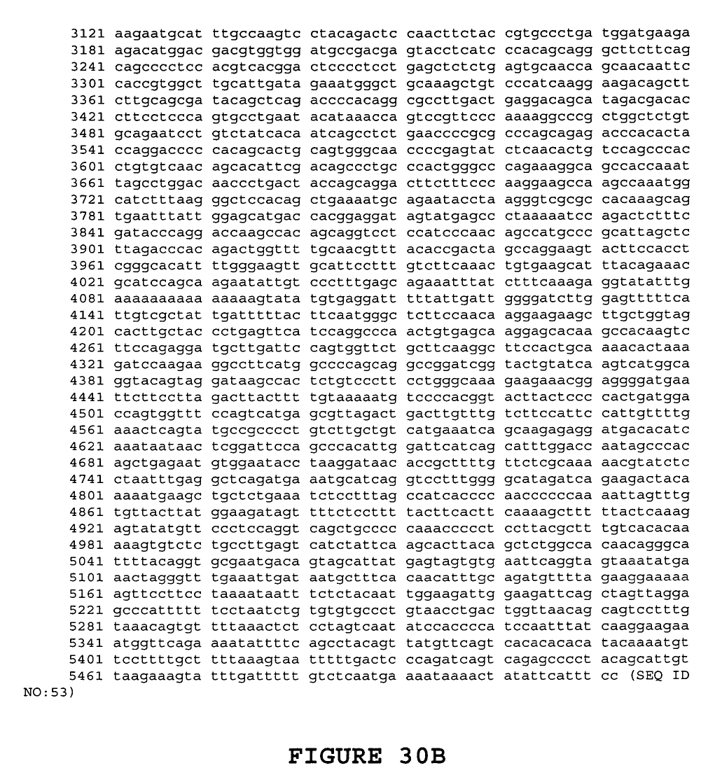

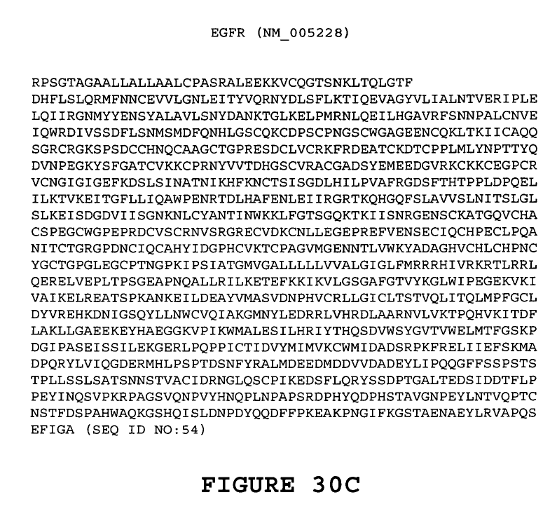

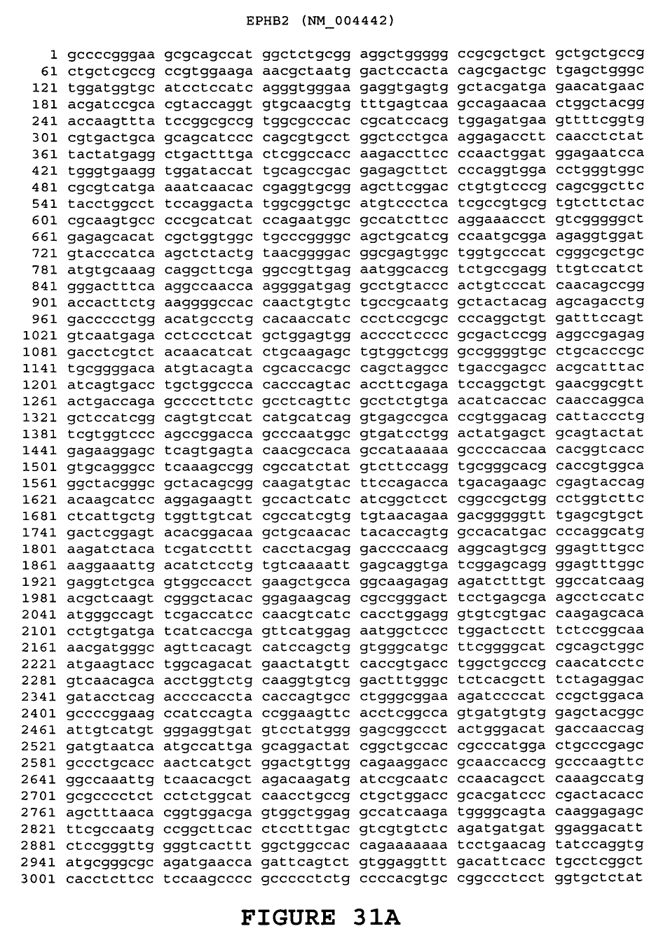

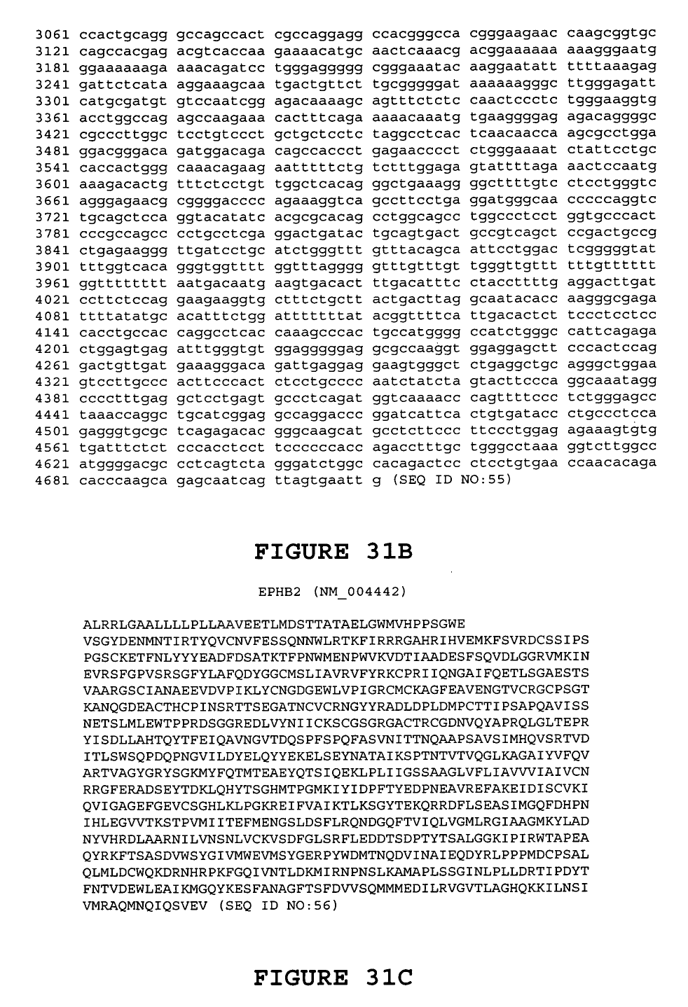

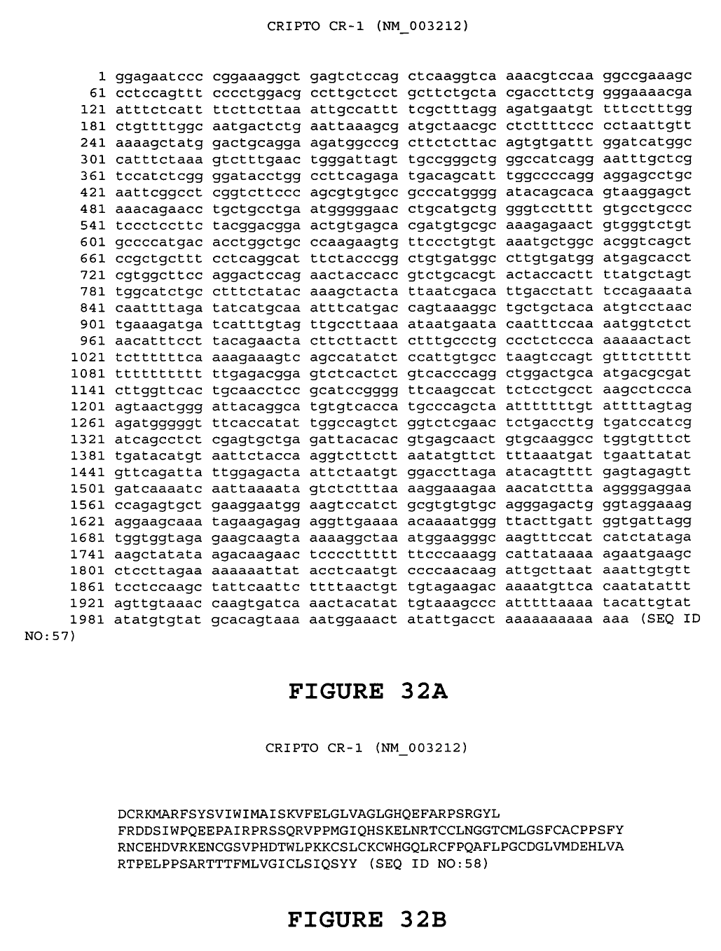

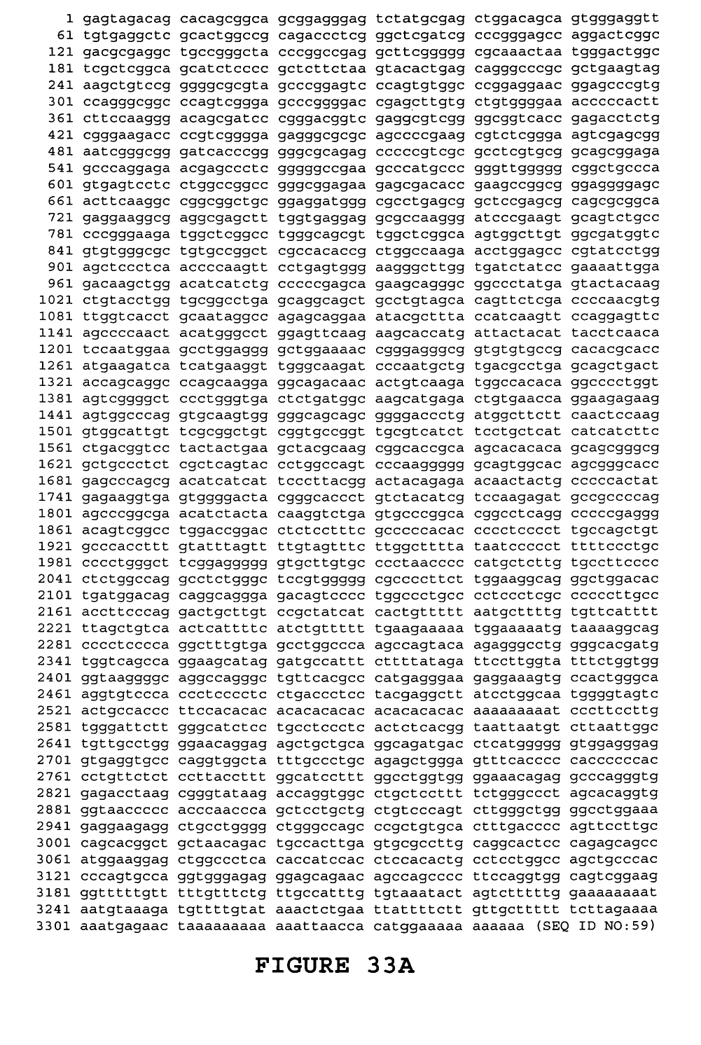

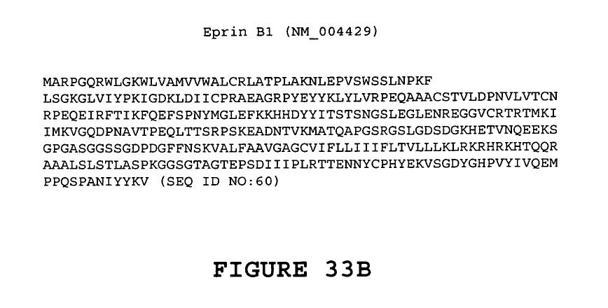

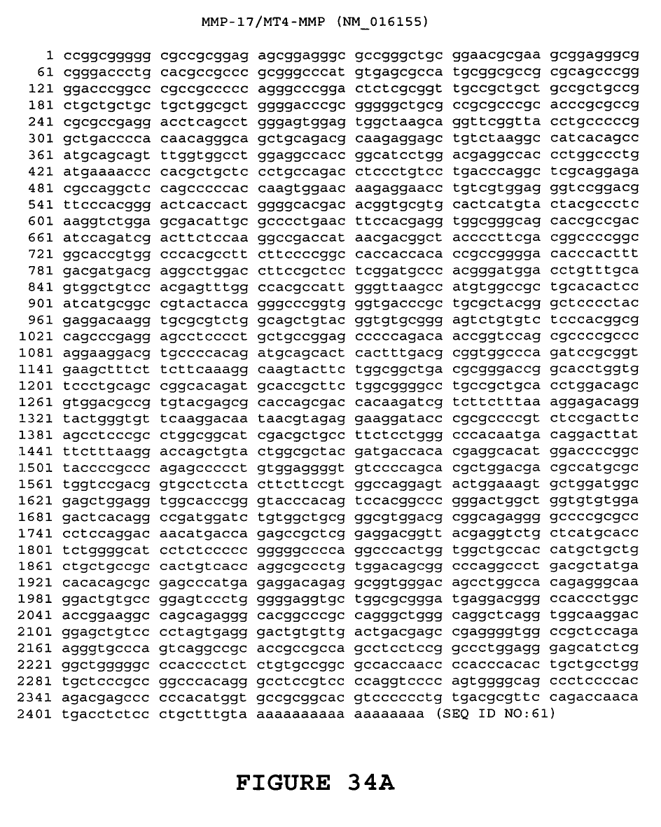

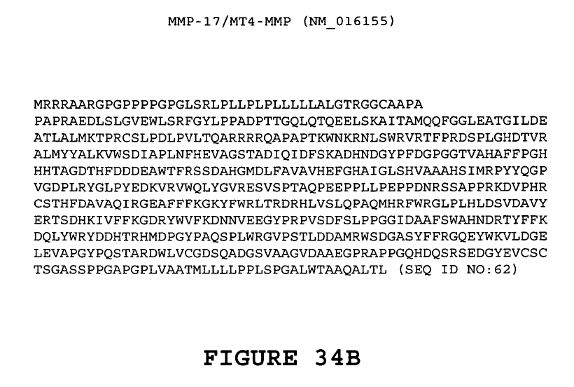

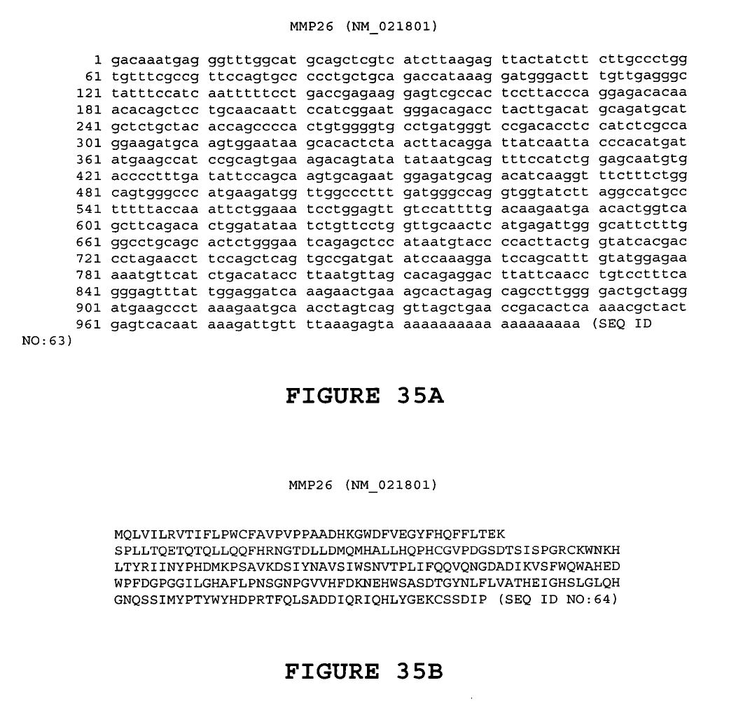

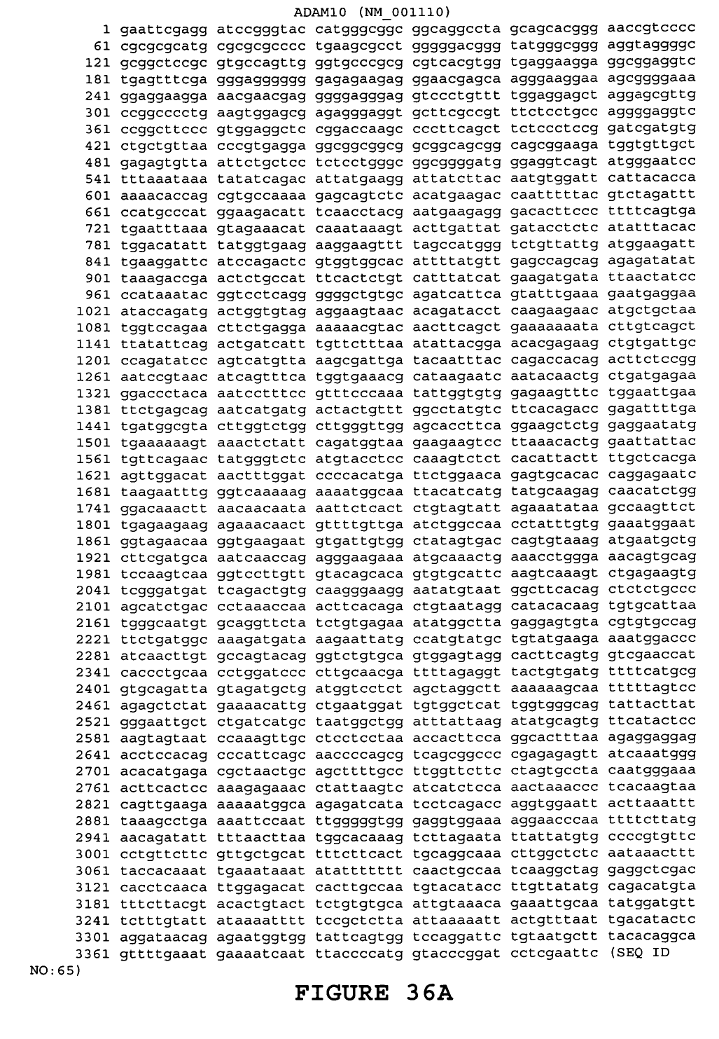

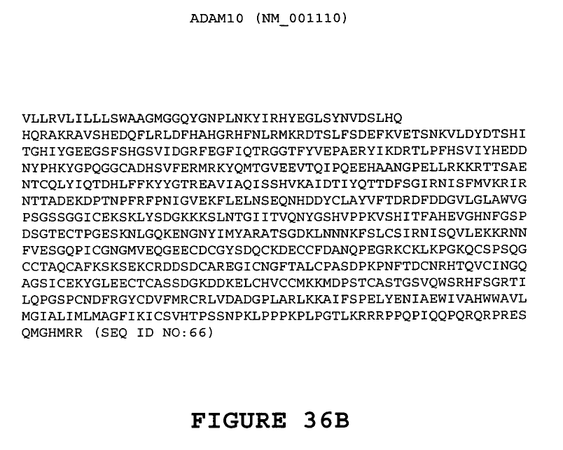

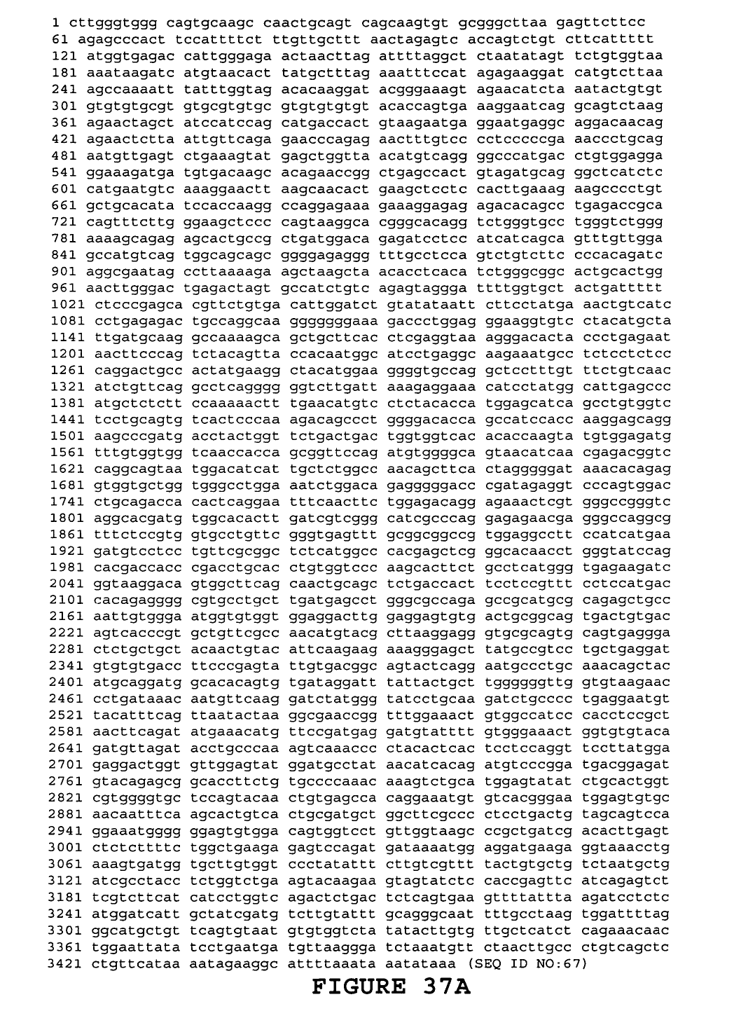

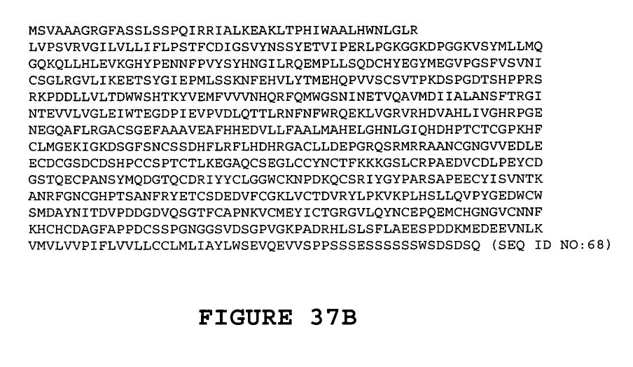

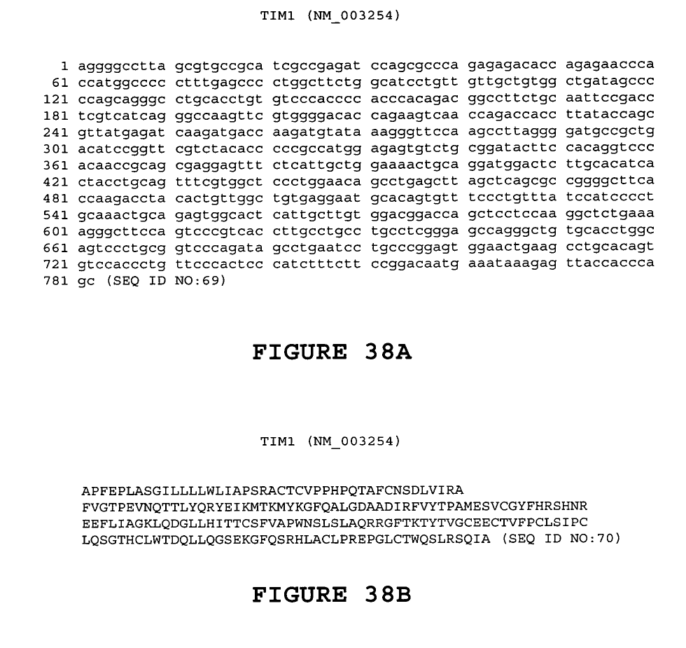

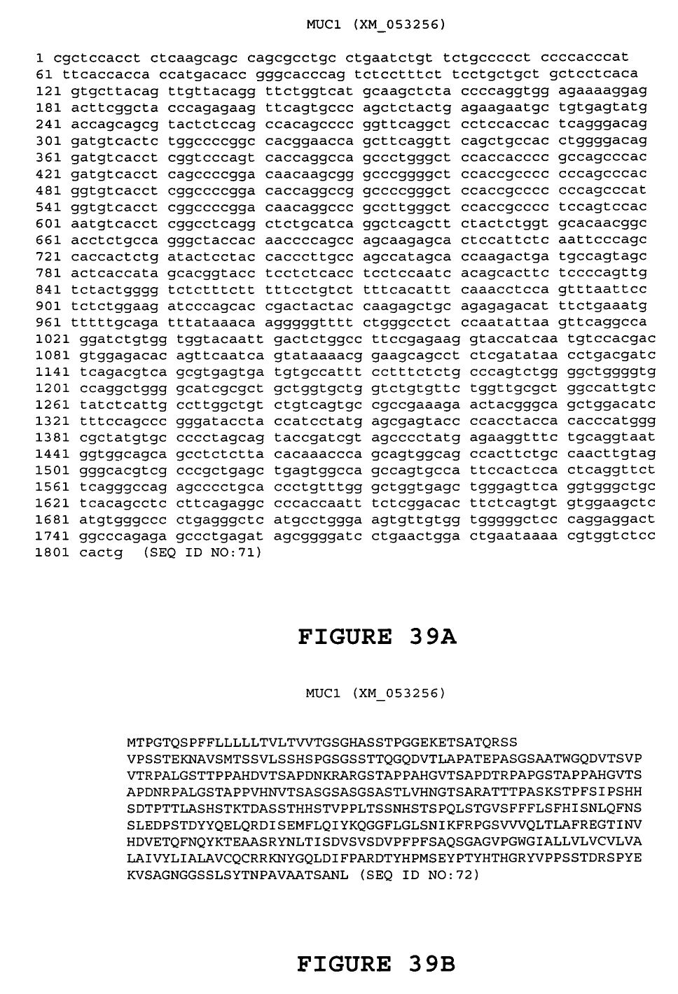

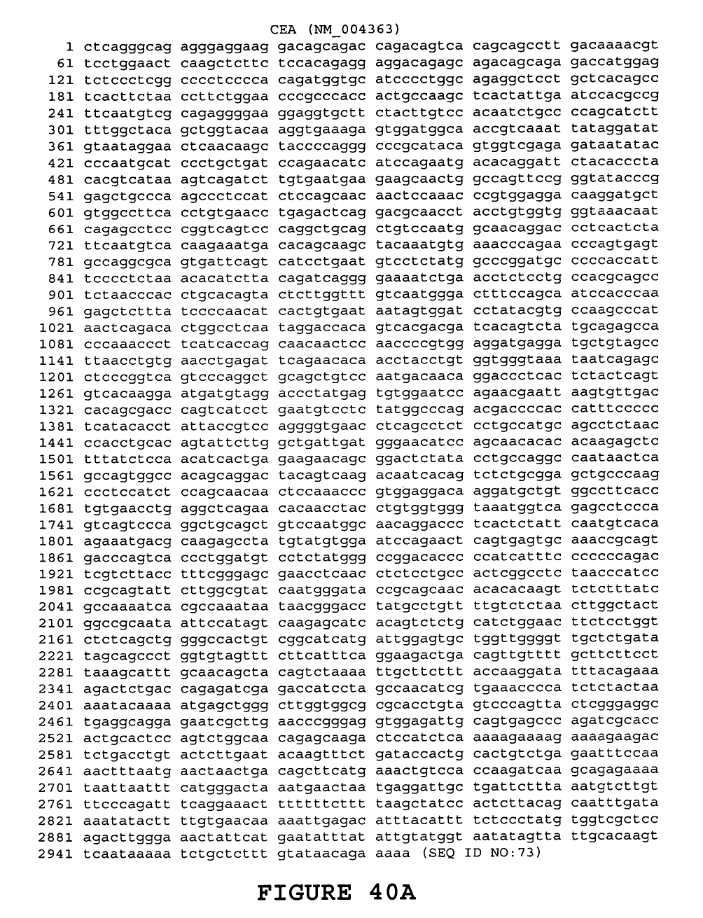

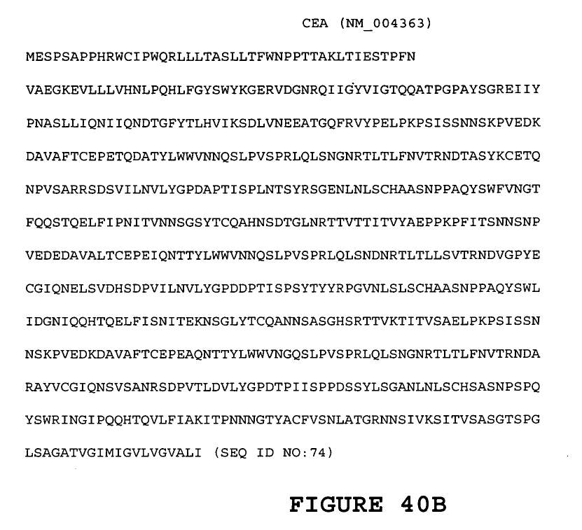

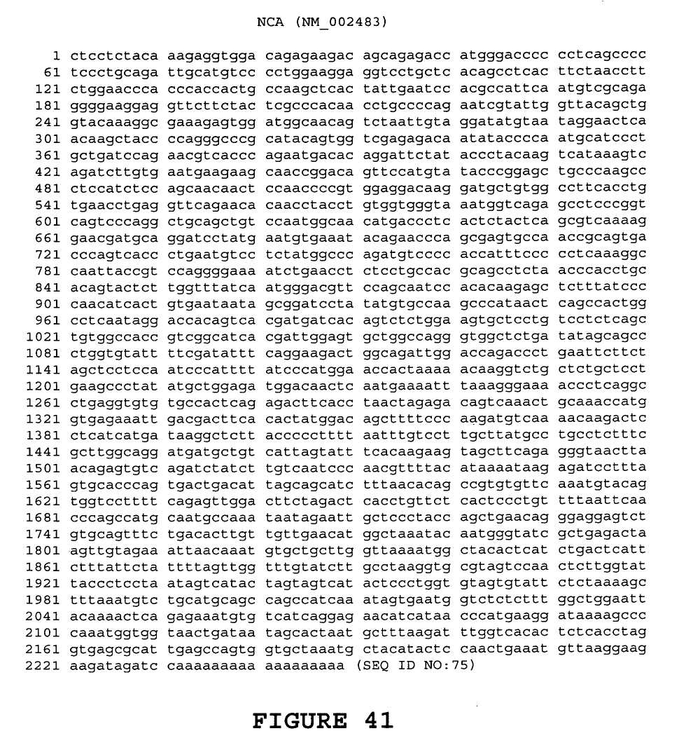

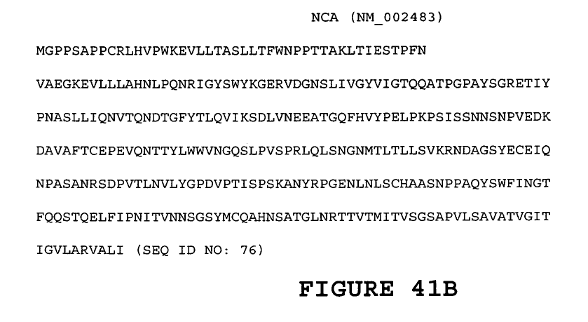

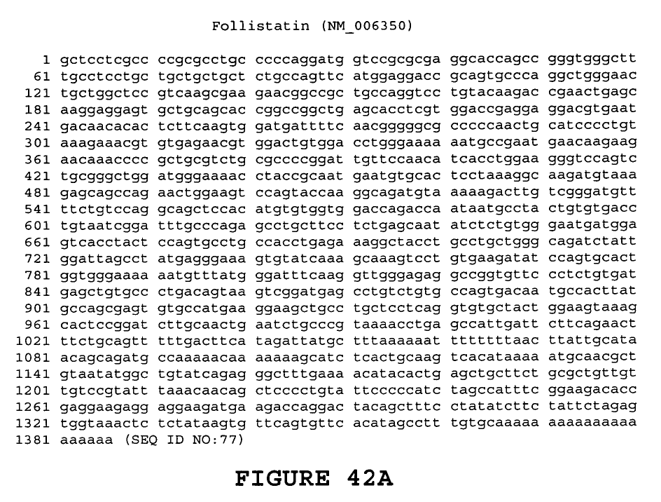

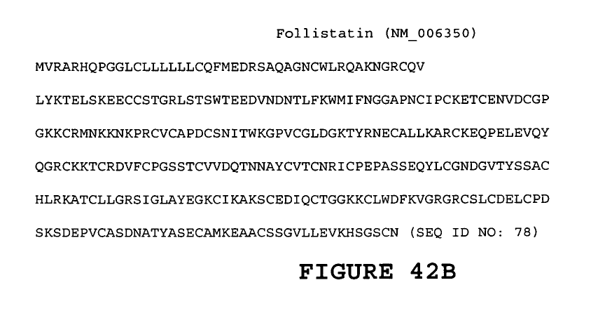

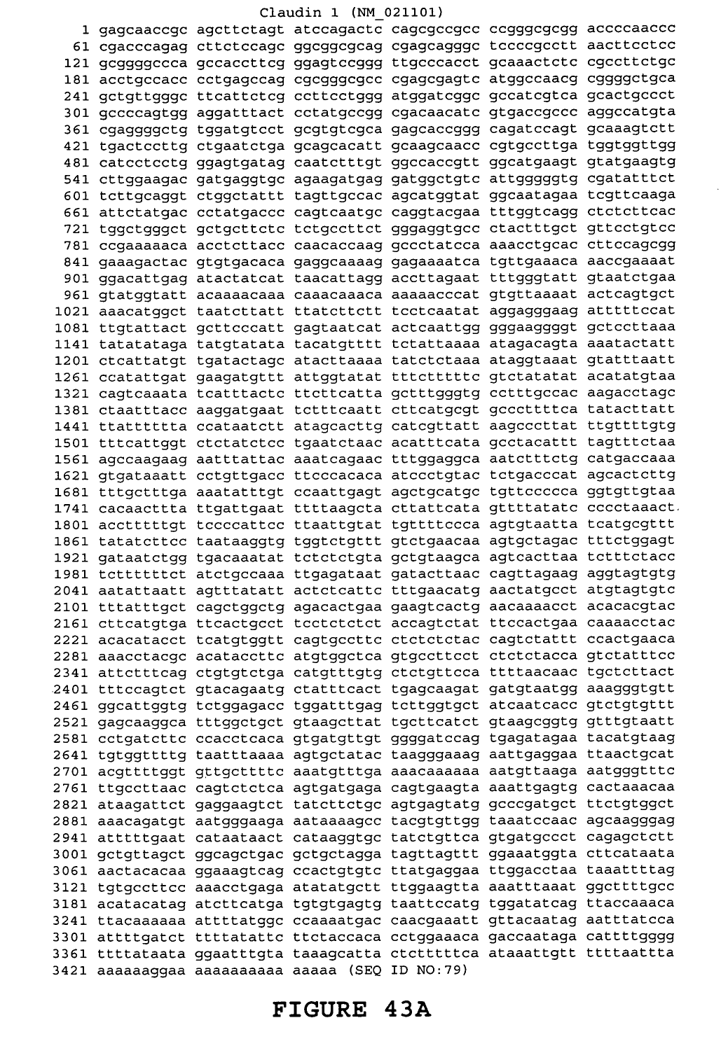

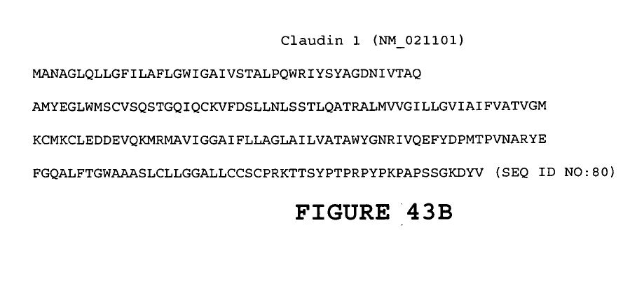

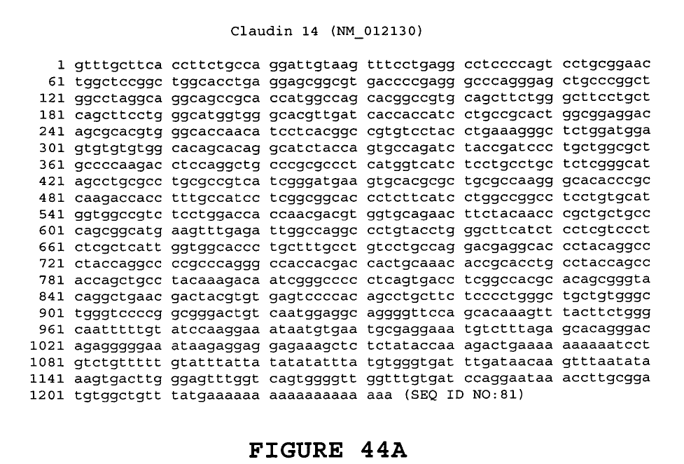

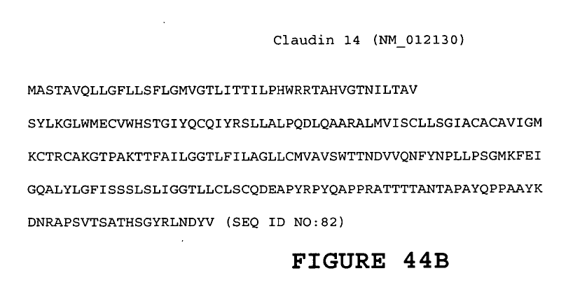

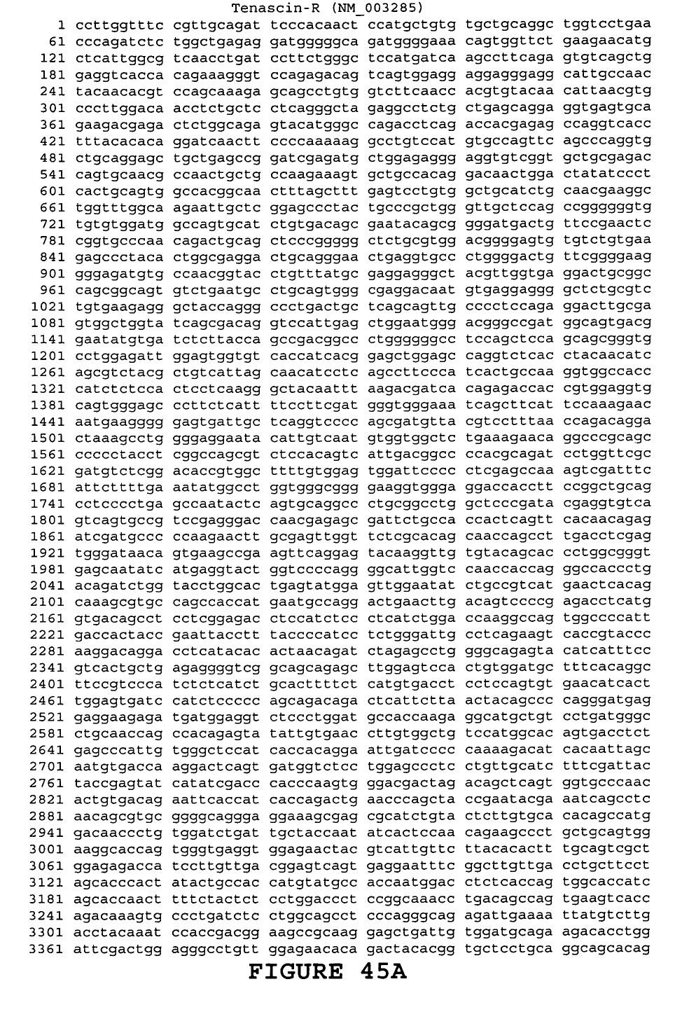

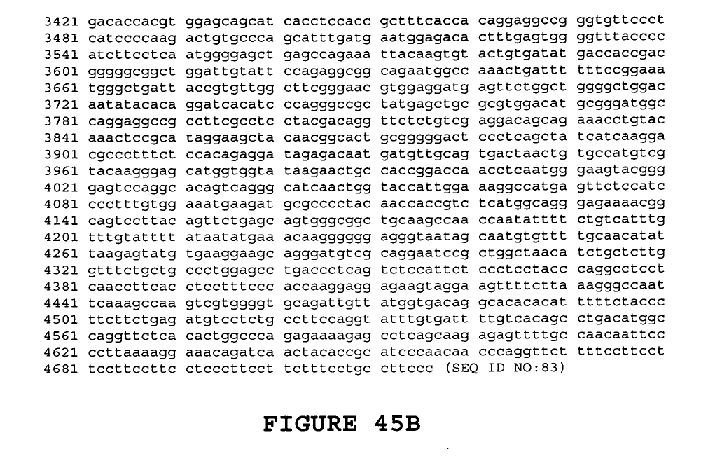

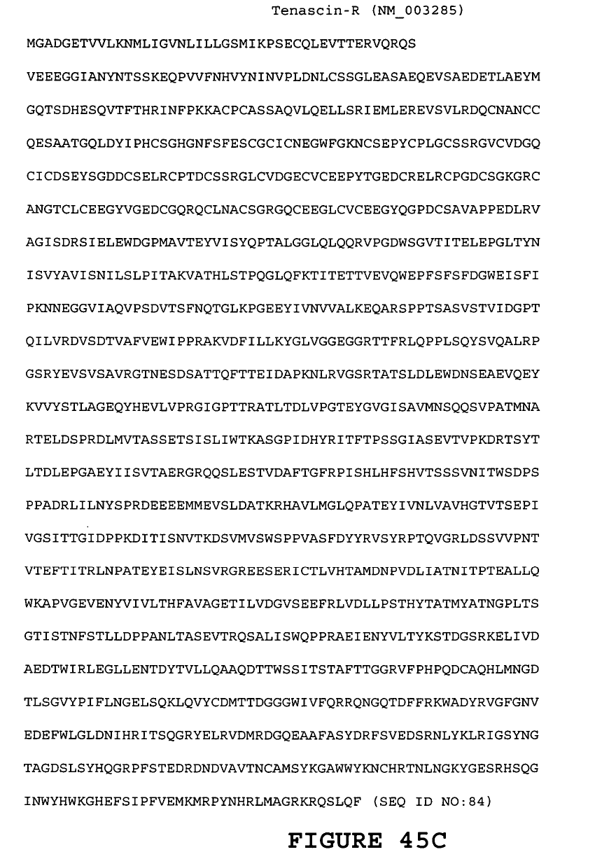

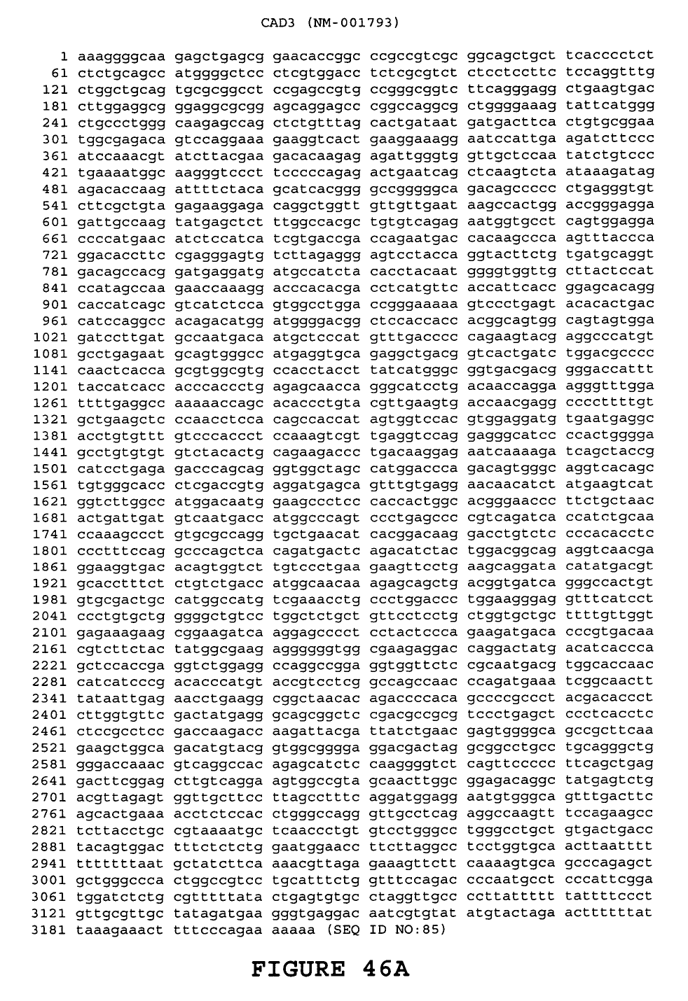

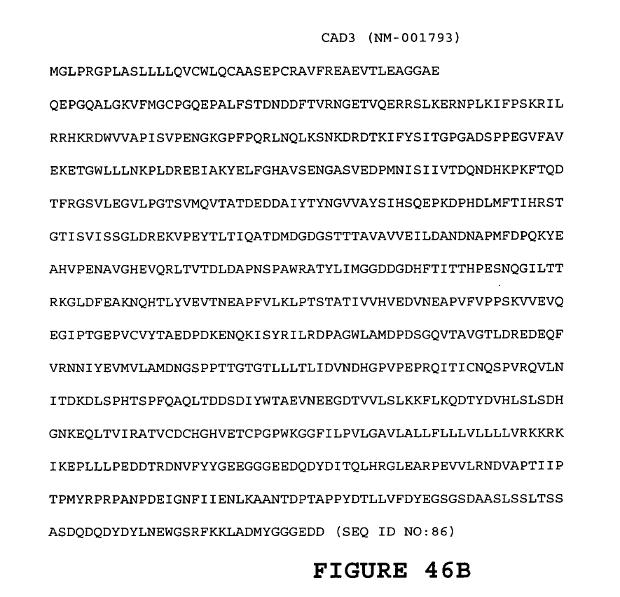

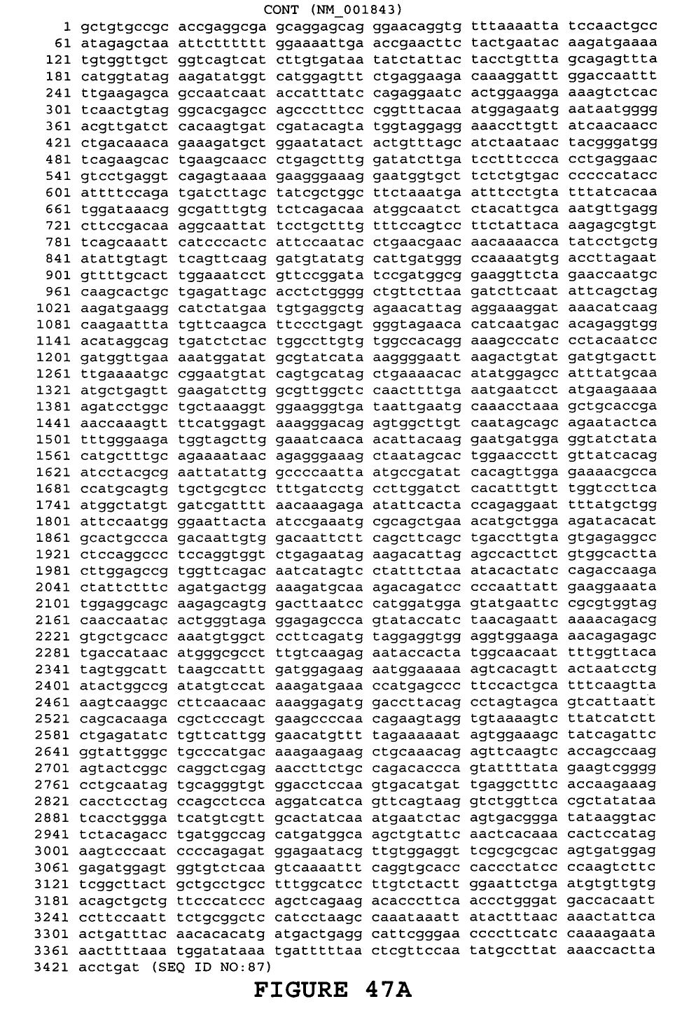

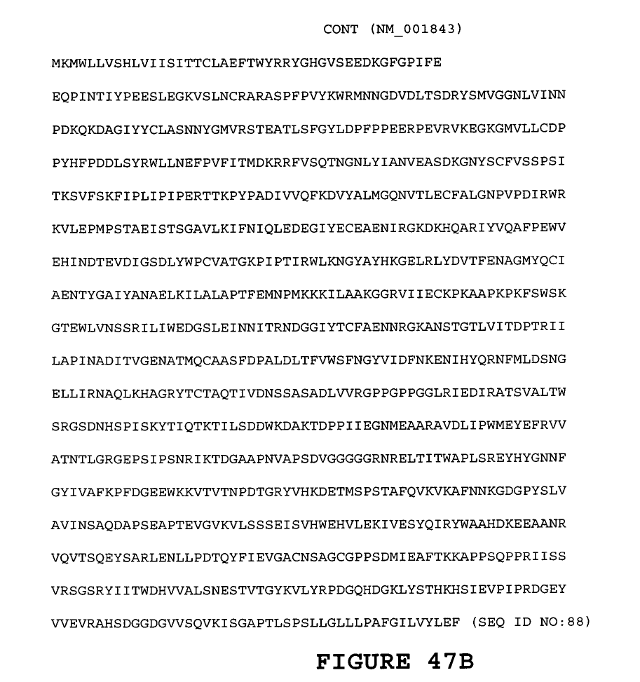

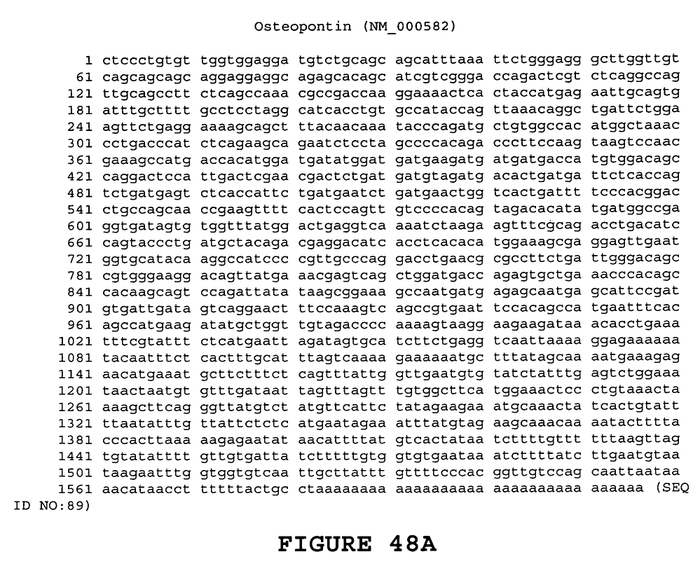

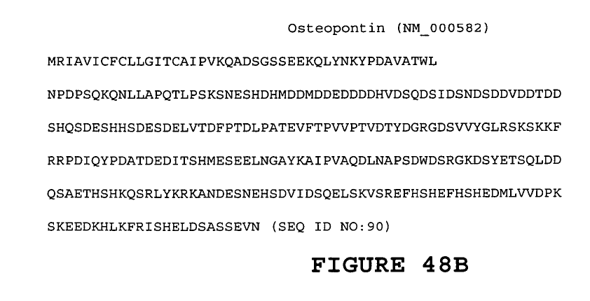

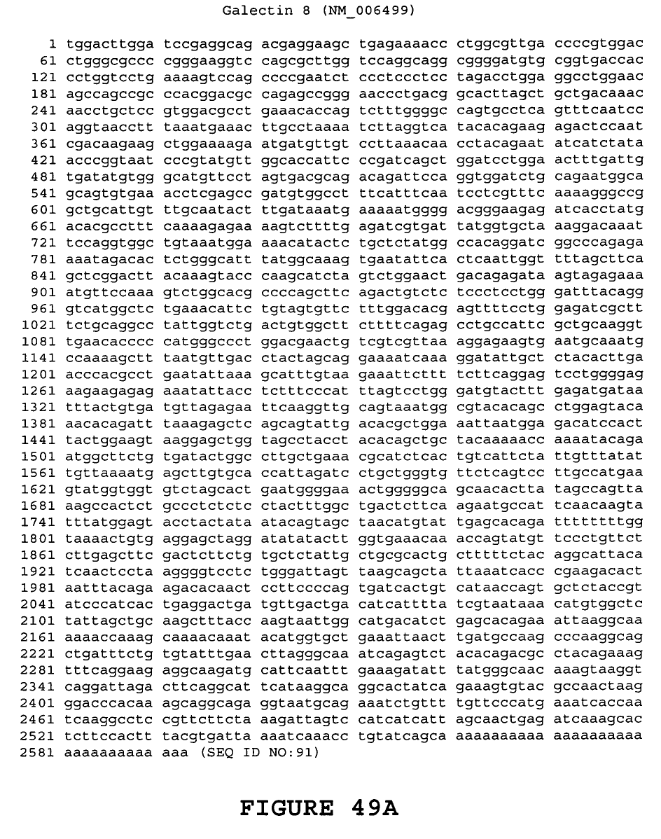

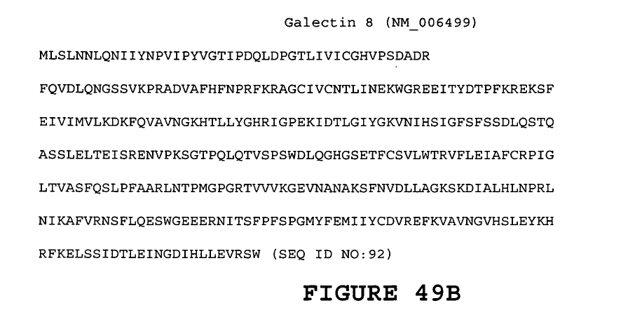

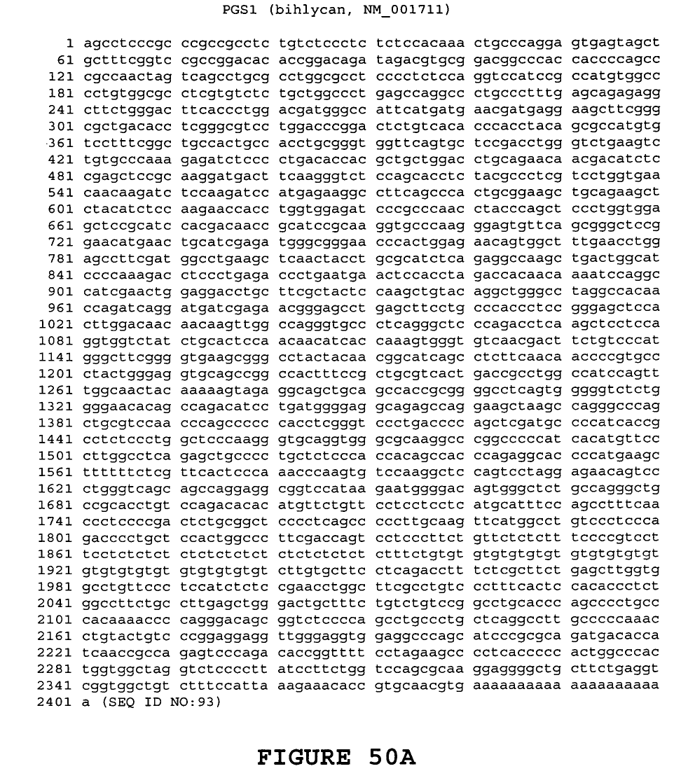

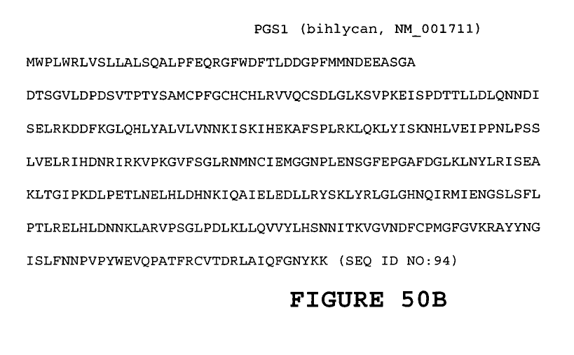

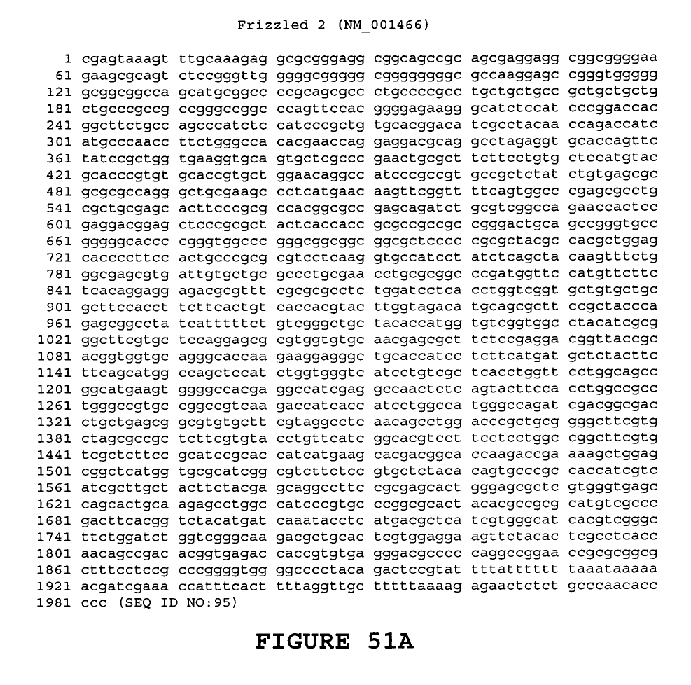

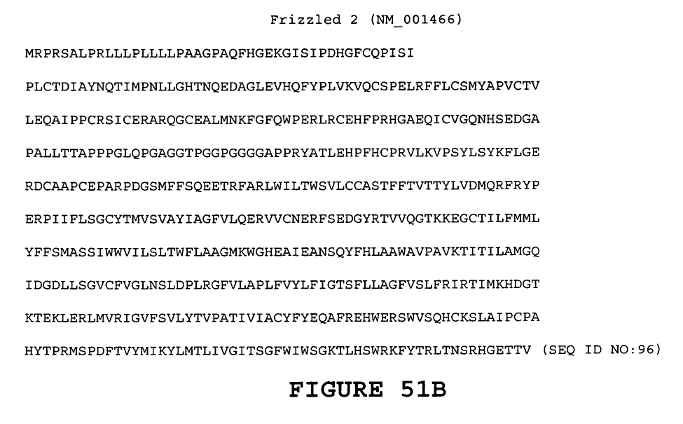

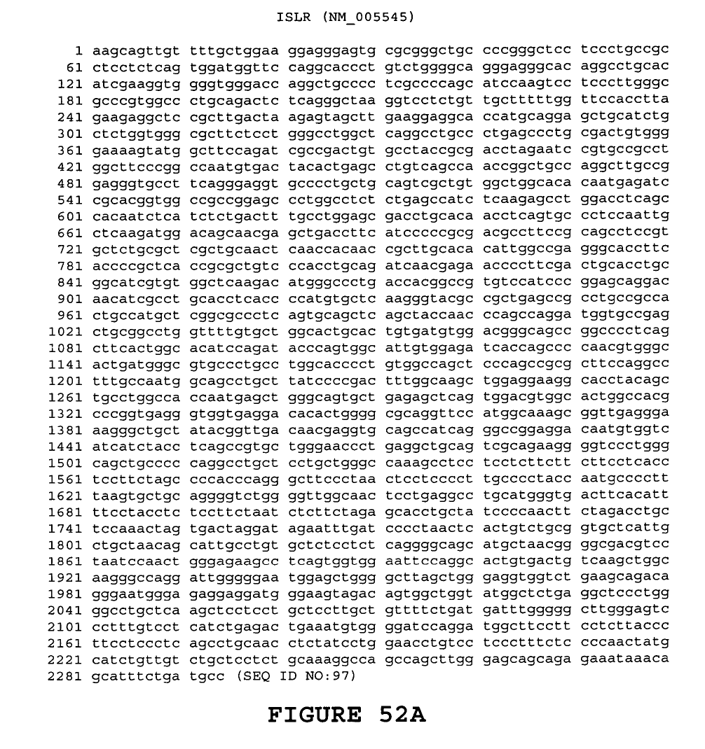

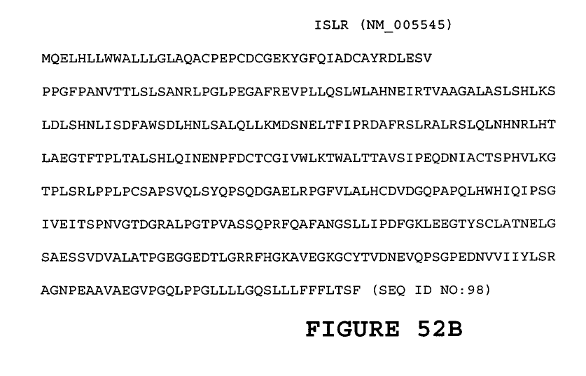

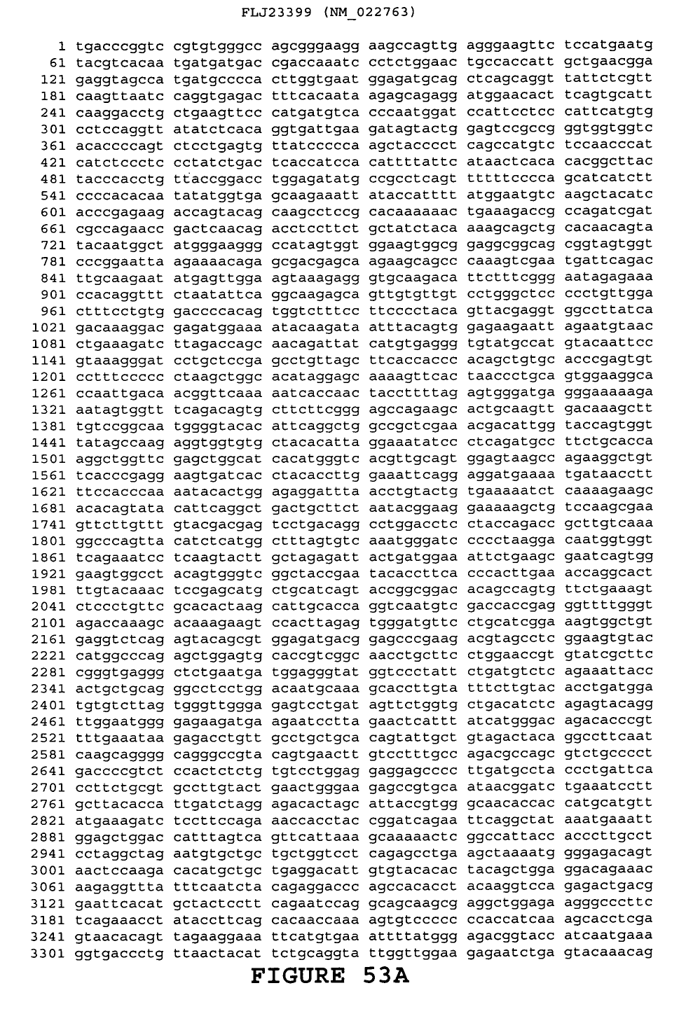

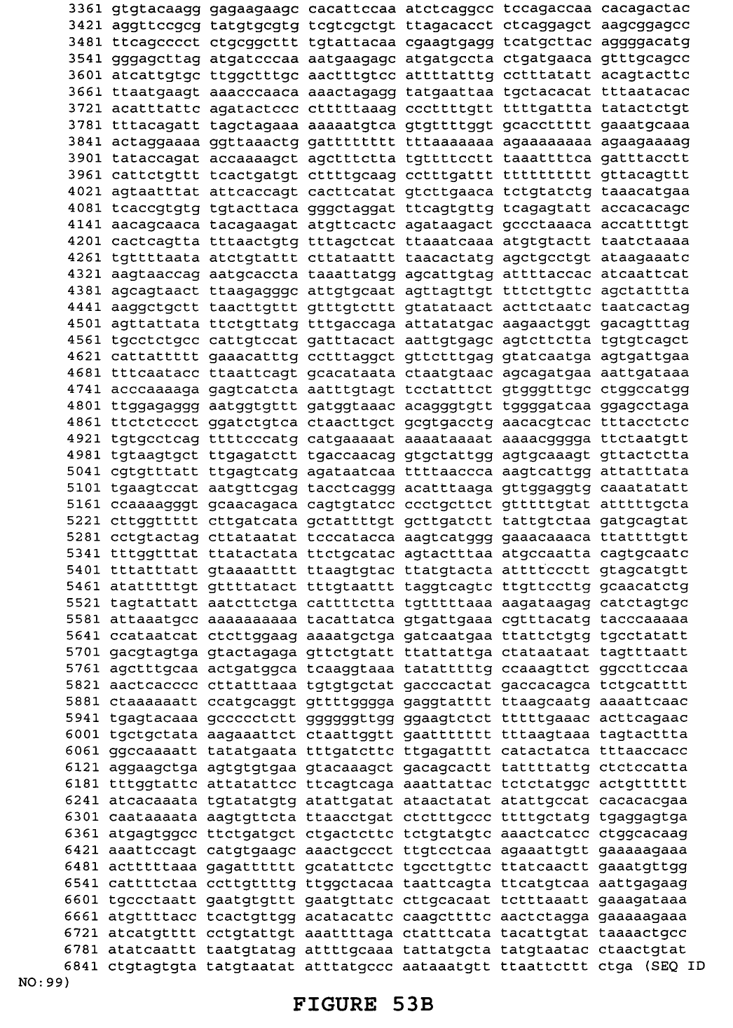

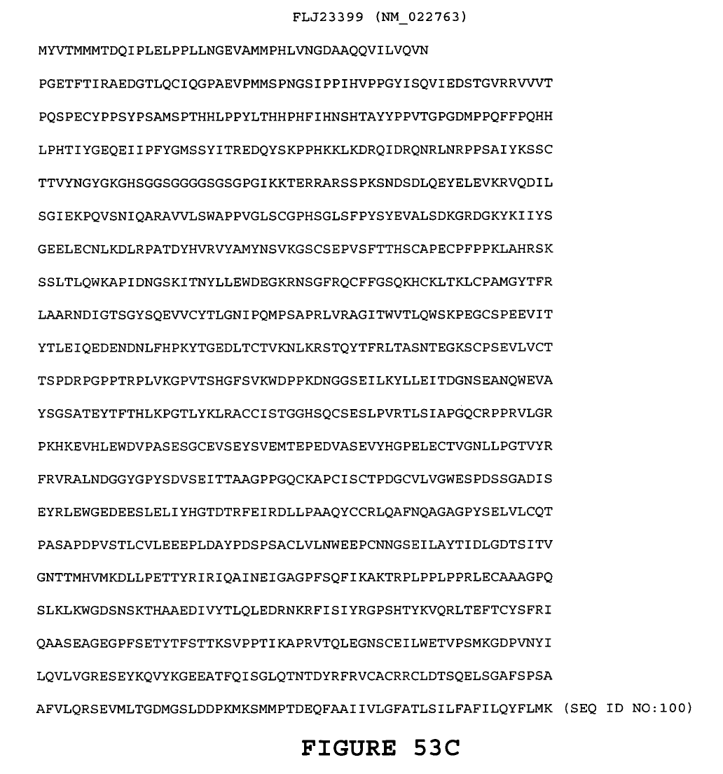

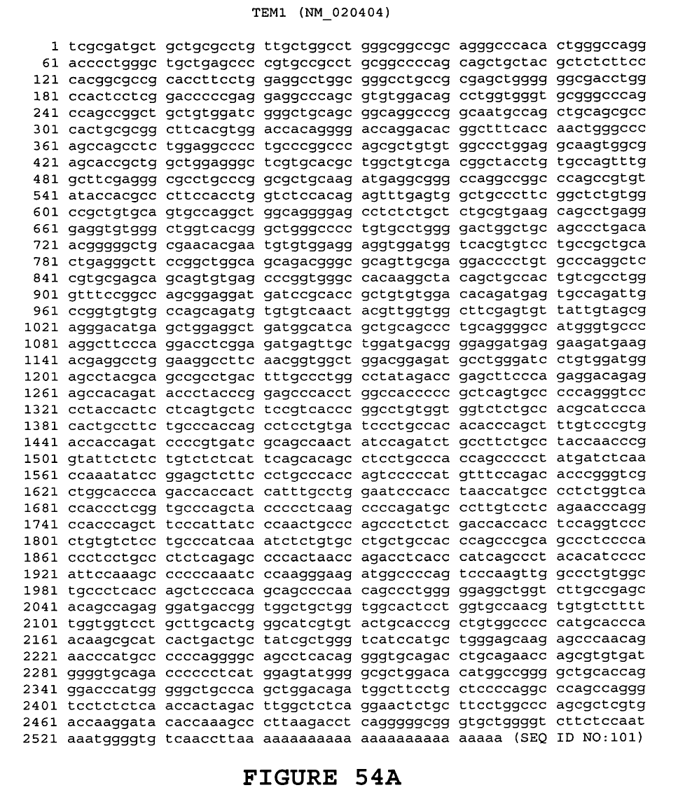

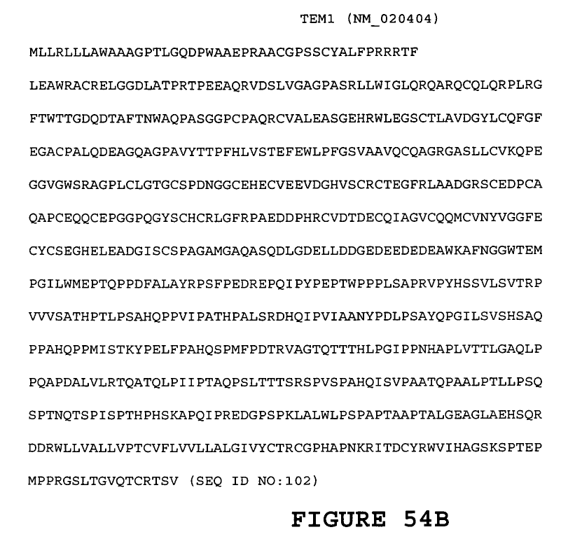

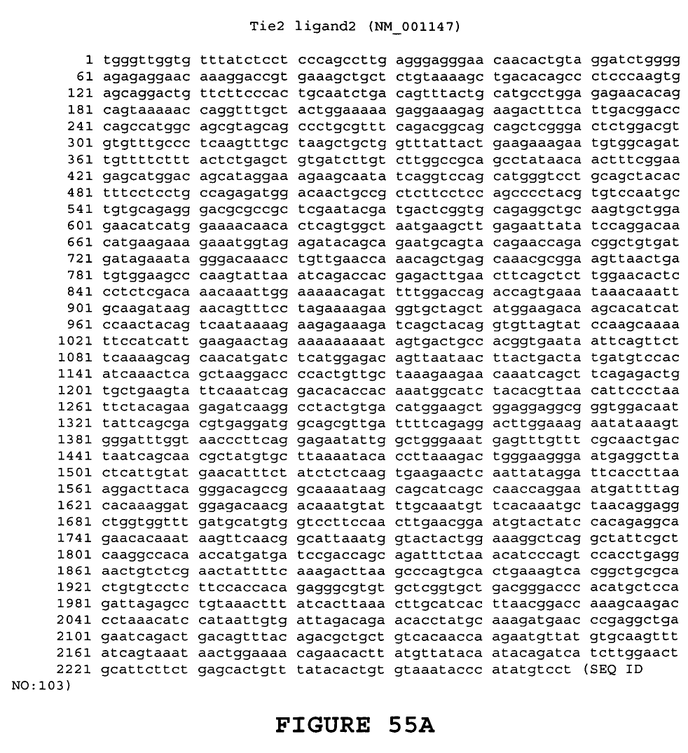

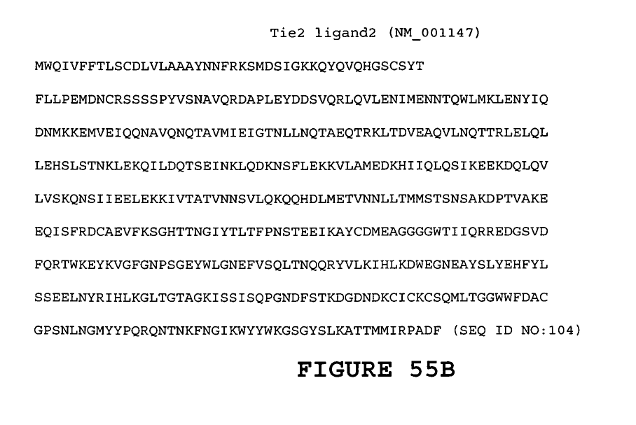

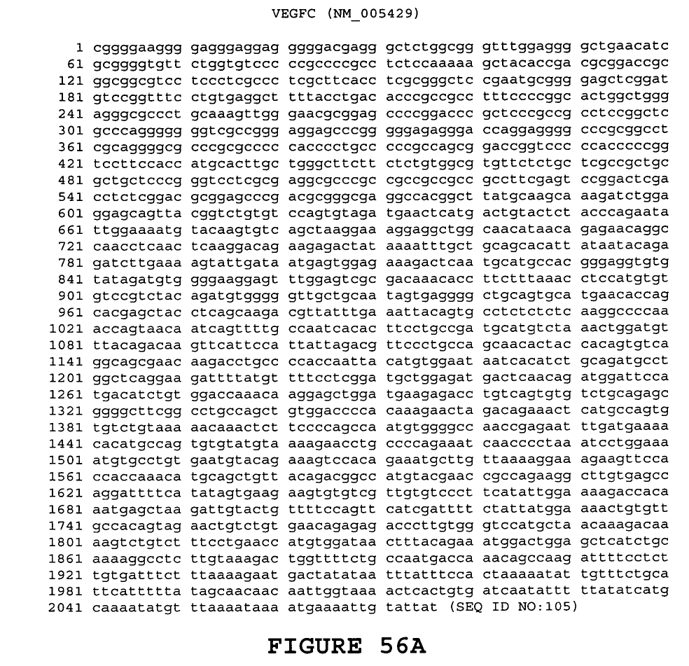

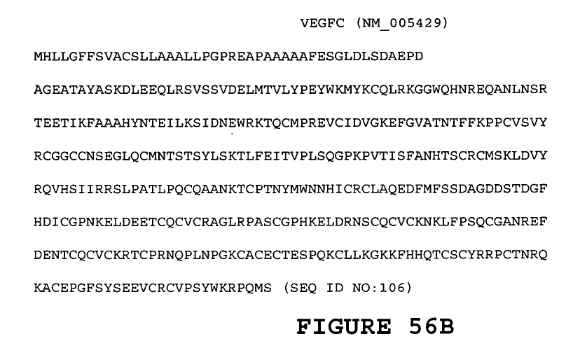

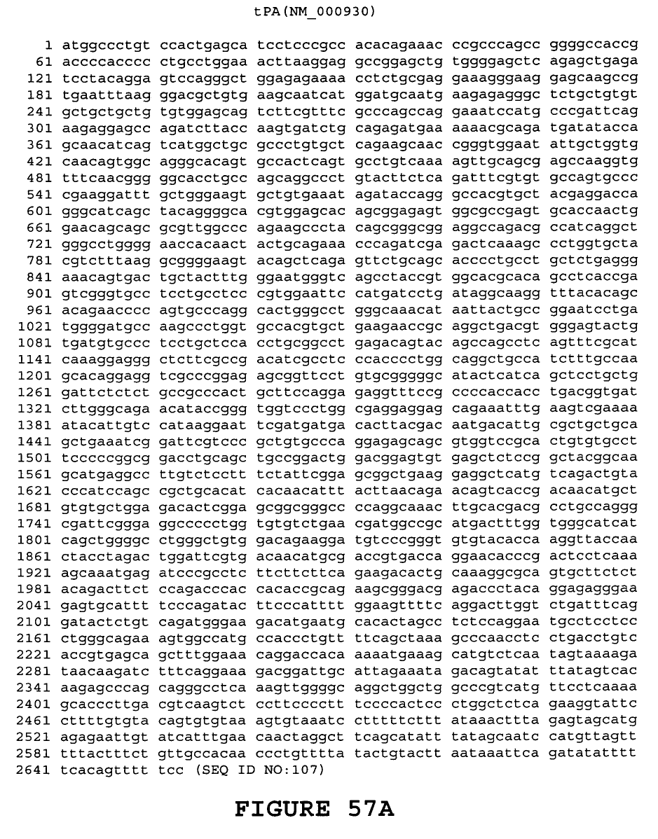

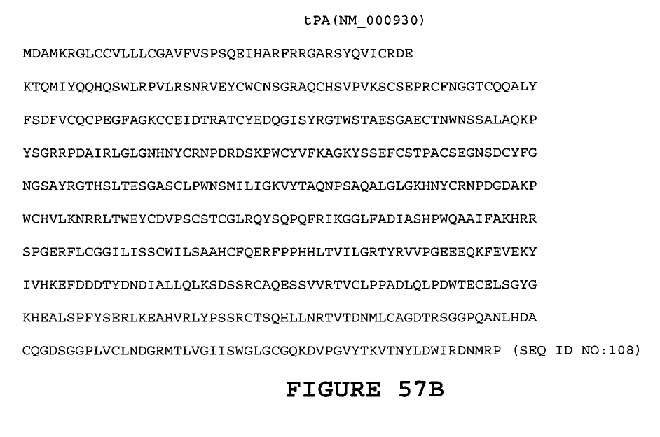

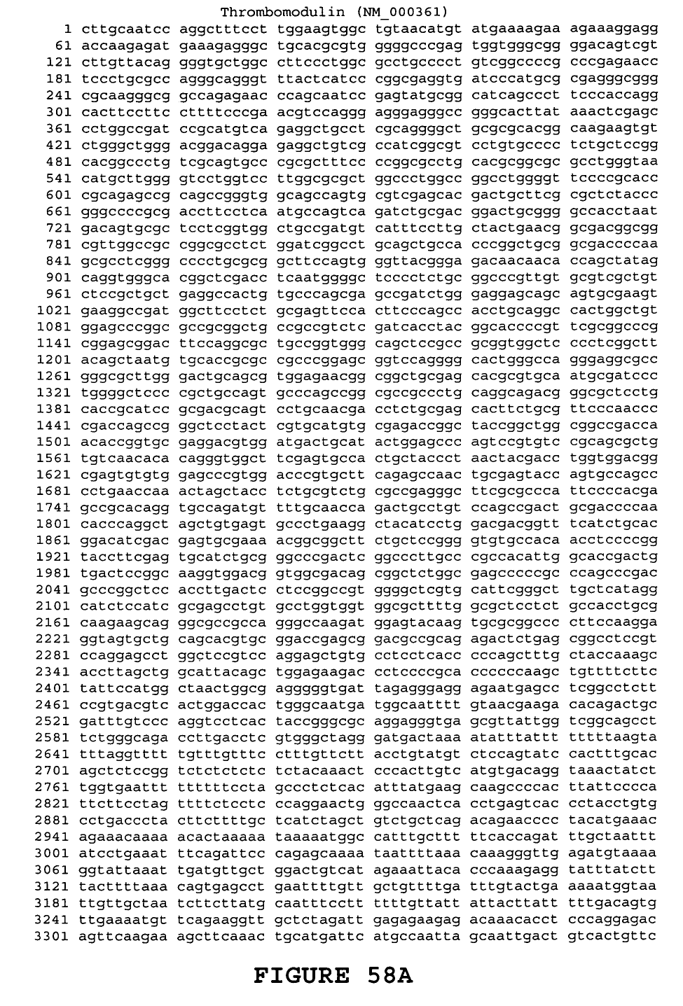

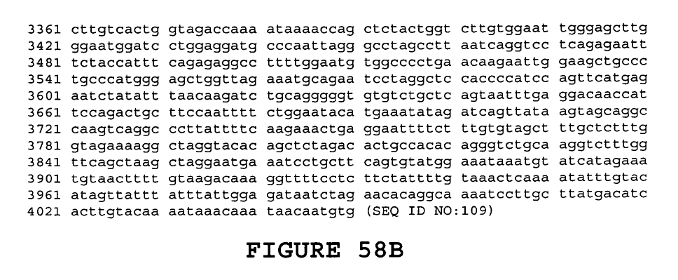

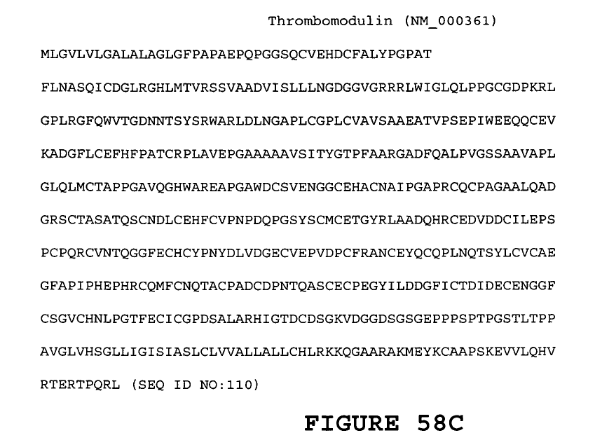

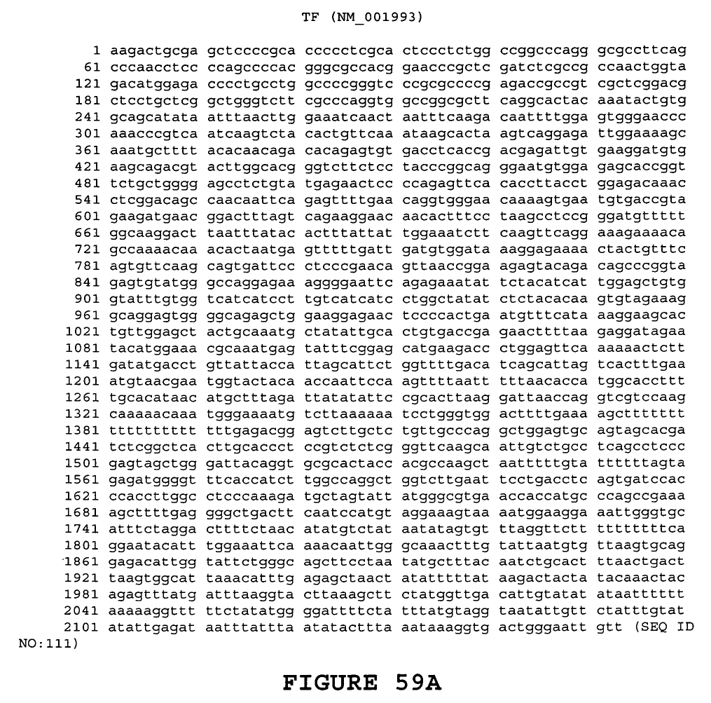

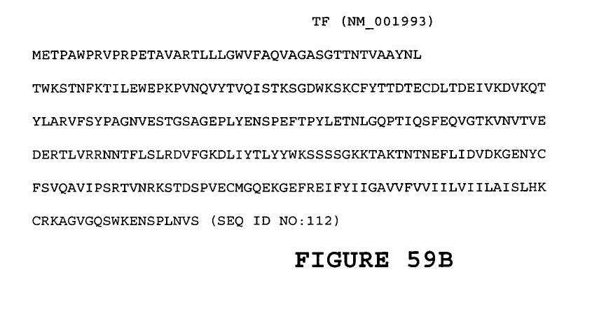

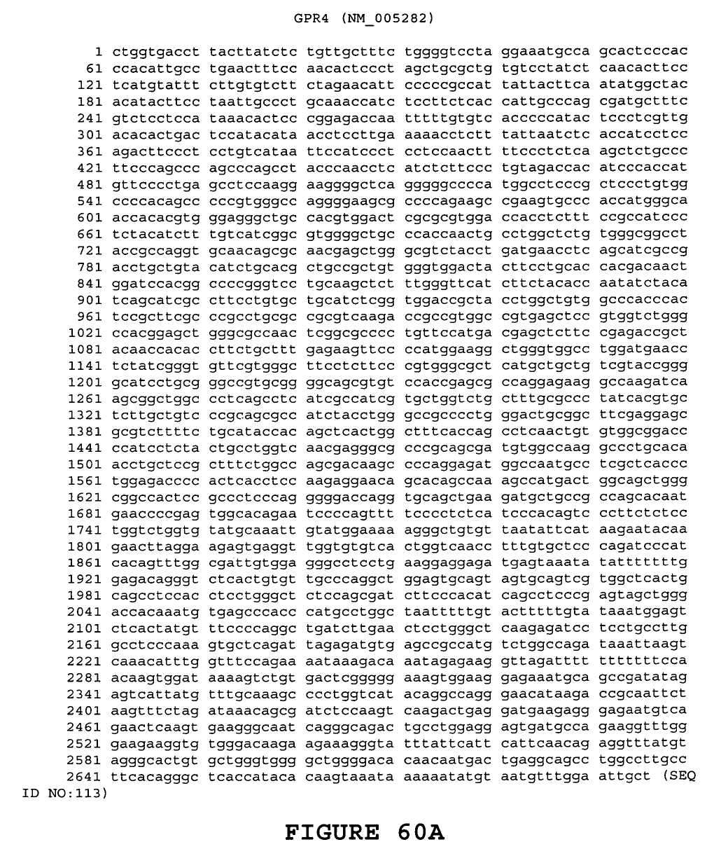

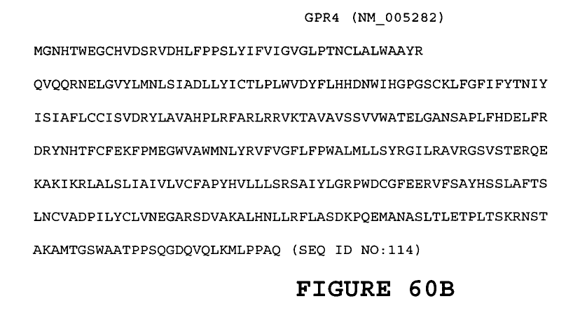

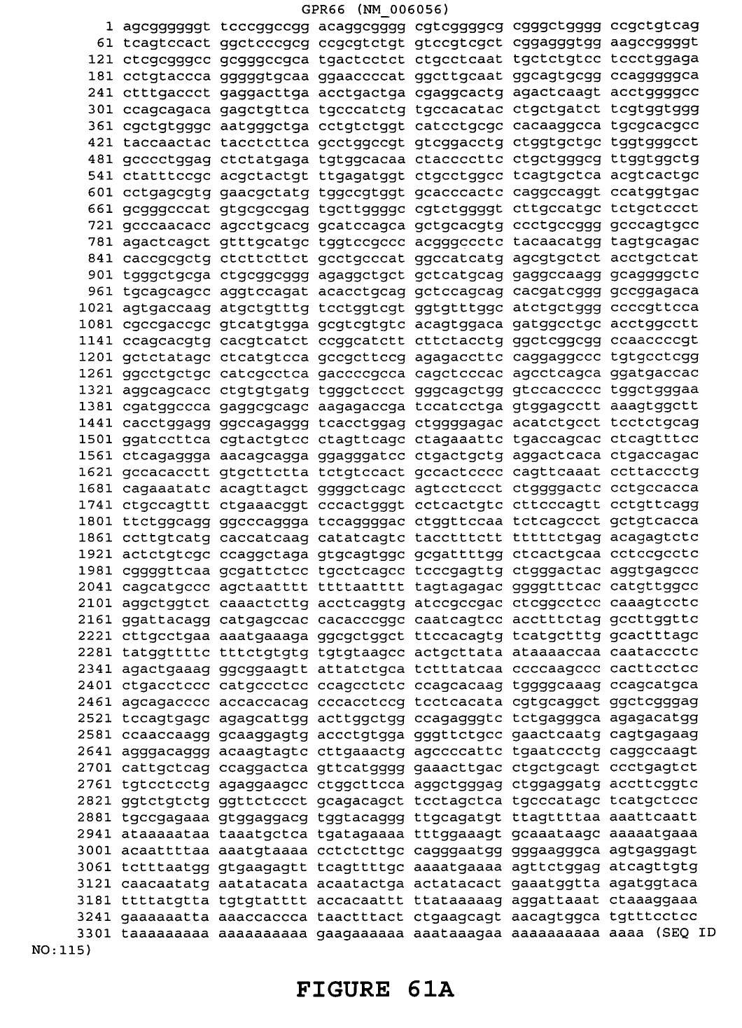

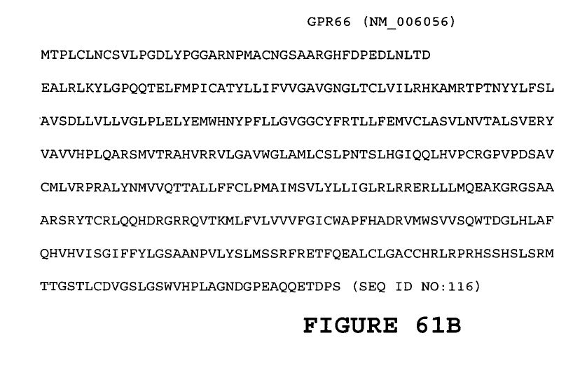

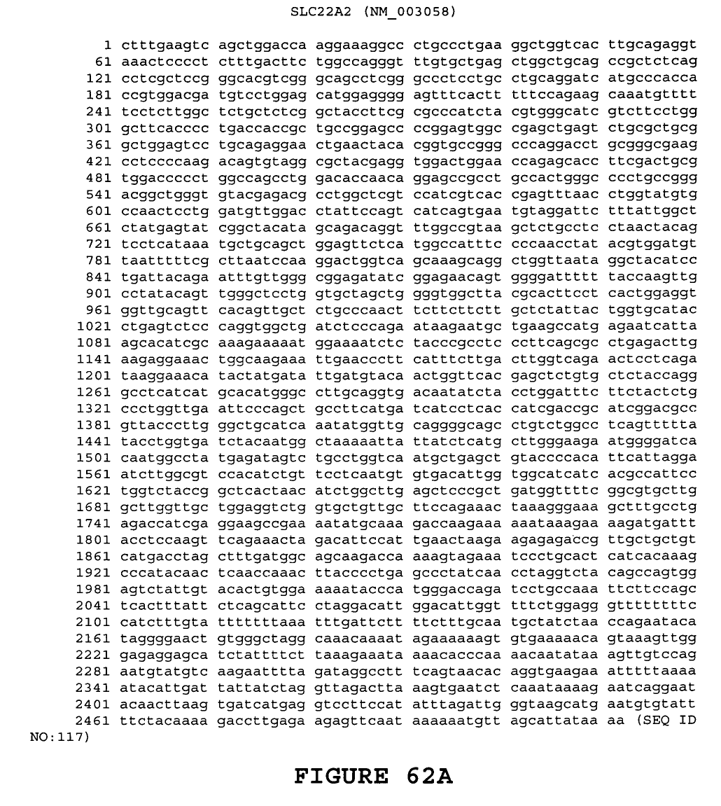

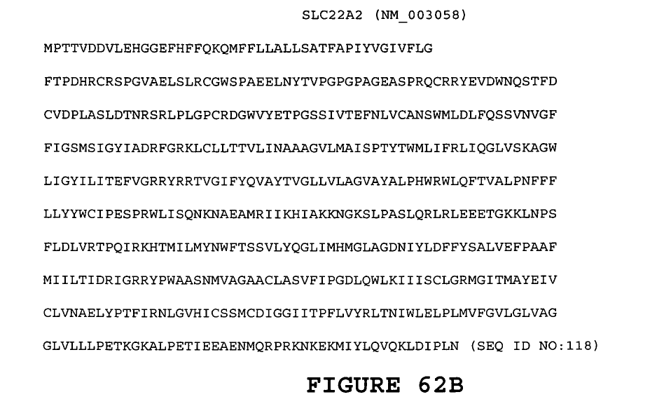

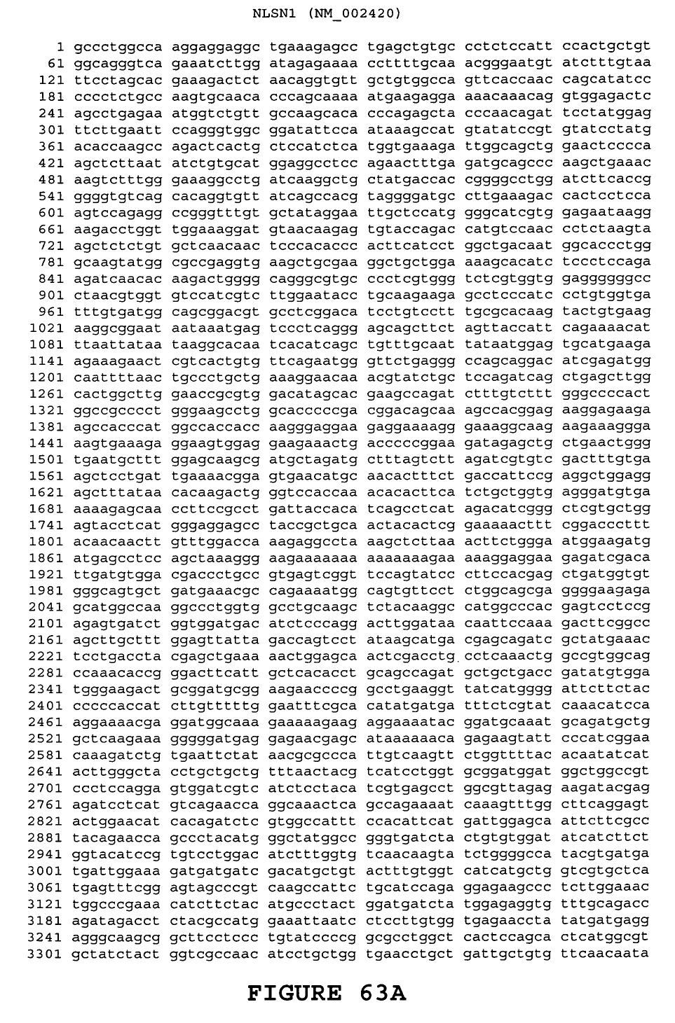

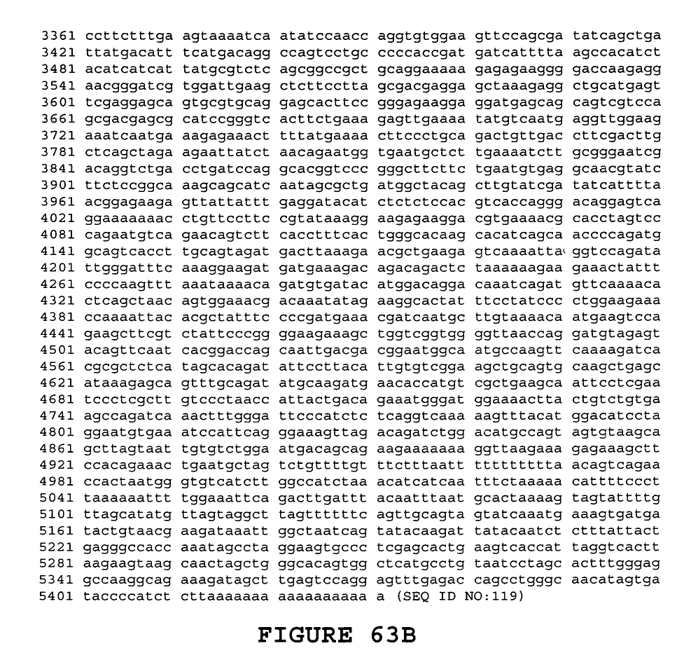

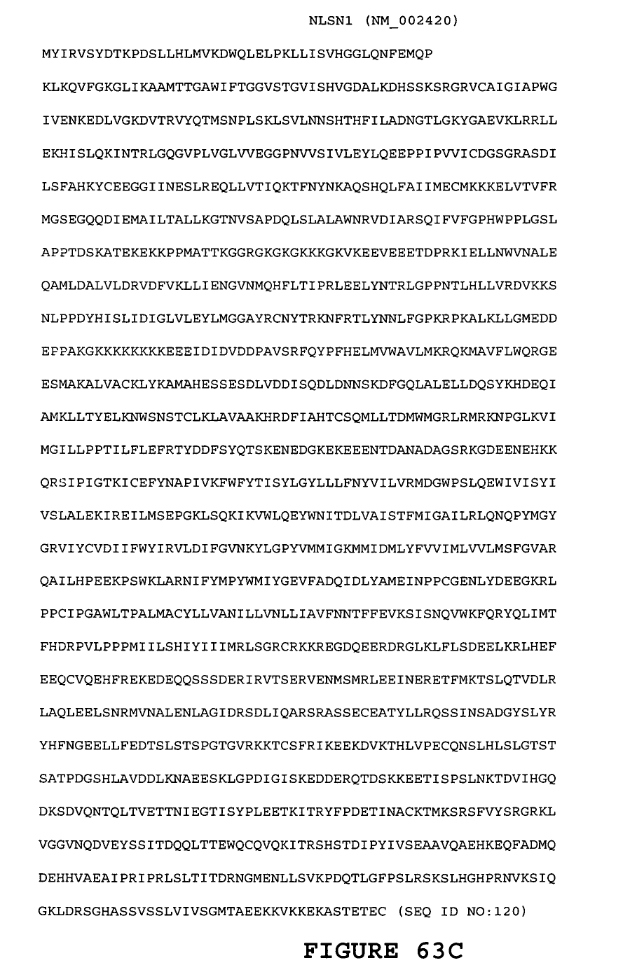

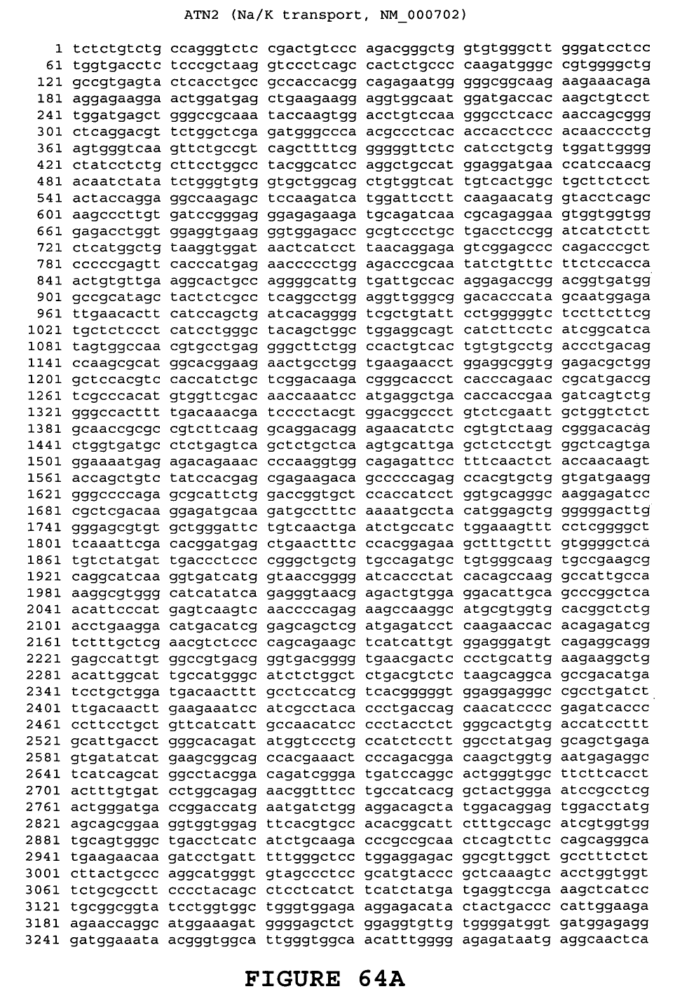

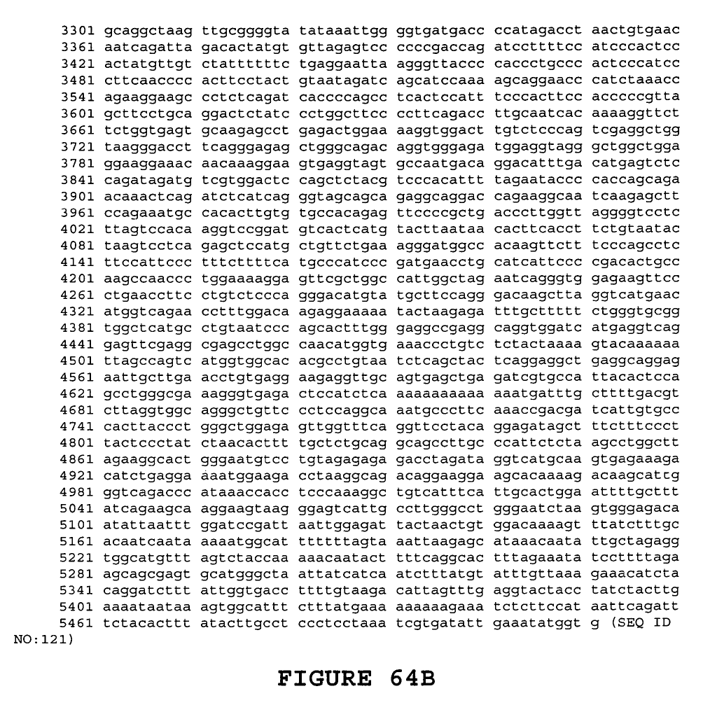

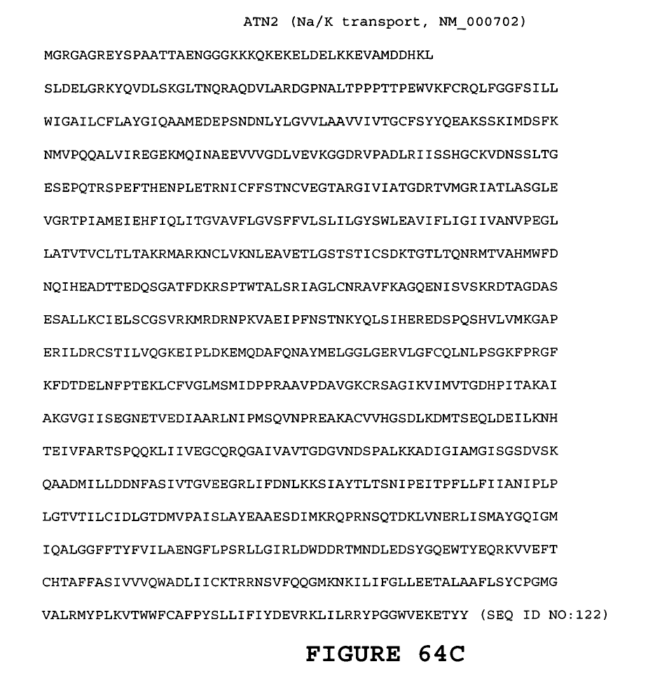

Analysis for gene expression changes associated with dysplasia revealed a large group of genes whose normal expression is primarily associated with the small intestine, and to a lesser extent, colon (Table 2). The previously described marker villin was detected, (Peterson and Moosekar, J. Cell Sci. 102:581-600 (1992)) along with a diverse set of genes including cell surface cadherins and claudins, ion channels and transporters, and enzymes, many of which are normally associated with structural and absorptive functions of small intestinal villi. Increased expression of many of these genes was associated with dysplasia and a significant subset of carcinoma samples, with differential expression also detected in a smaller subset of BE samples. Furthermore, expression of the majority of genes was less prevalent in matched BE samples taken from the carcinoma patients, even when expression was apparent in the tumor sample (FIGS. 2A, 2B, 3A; Table 2). This suggests that these gene expression changes are more specifically associated with the foci of dysplasia and developing carcinoma within the larger region of BE.

TABLE 2
Genes up-regulated in intestinal metaplasia

| | SEQ ID NOS | | | | | | | |
|---|---|---|---|---|---|---|---|---|
| NCBI RefSeq | (na and aa) | Gene | Gene Description | BE | D | BE-CA | CA | Normal Tissues |
| NM_007127 | | Villin 1 | actin binding protein | * | * | * | * | SI, C |
| NM_003379 | | Villin 2 | actin binding protein | * | | | | SI, St, C, O |
| NM_000775 | 35 and 36 | CYP2J2 | arachidonic acid epoxygenase | | * | (*) | * | SI, L, H |
| NM_005379 | 33 and 34 | MYO1A | myosin 1A | | *H | | * | SI (C) |
| NM_004063 | 45 and 46 | CAD17 | liver-intestine cadherin | (*) | (*H) | (*) | * | SI, C |
| NM_017717 | | MUCDHL | mucin and cadherin like | | | * | | SI (C, K) |
| NM_014343 | 47 and 48 | CLDN15 | claudin 15 | (*) | *L | (*) | * | SI |
| NM_012132 | | CLDN8 | claudin 8 | | * | | (*) | C, K |
| NM_005567 | | IR-95 | lectin-binding | | | (*) | * | C, SI, St, O |
| NM_000021 | | Presenilin-1 | beta-catenin binding | | *H | | (*) | SI, C |
| NM_003039 | | GLUT5 | glucose transporter | * | (*) | | (*) | SI |
| NM_001081 | | CUBN | transport (HDL, vit.B12, etc) | | *L | | | K, SI |
| NM_004769 | 23 and 24 | SLNAC1 | sodium channel | | *H | * | * | CNS, SI, O |
| NM_000492 | 49 and 50 | CFTR | chloride channel | * | (*H) | | * | P, SI, C |
| NM_003272 | 13 and 14 | TM7SF1 | novel GPCR | (*) | *H | | | K, C, SI, O |
| NM_005242 | 29 and 30 | PAR2/F2RL1 | GPCR, proteinase-activated | | *H | | | SI, C |
| NM_022304 | 51 and 52 | H2R | histamine H2 receptor | (*) | * | * | * | St-par |
| NM_004624 | | VIPR1 | intestinal peptide GPCR | | | * | | L, SI, C, CNS |
| NM_002773 | 7 and 8 | PRSS8 | serine protease | | | * | * | SI, C, St |
| NM_058186 | | RPLA320 | novel | | *L | (*) | | SI (St, C, P) |
| NM_003561 | | SPLA2 | phosphlipase A2 group X | | * | (*) | (*) | C, St, SI |
| NM_000928 | 27 and 28 | PA21 | phospholipase A2 group IB | | * | (*) | * | P, SI, C |
| NM_001631 | 21 and 22 | PPBI | intestinal alkaline phosphatase | (*) | * | | | SI |
| NM_000717 | 25 and 26 | CAH4 | carbonic anhydrase IV | | *H | | (*) | C, SI |
| NM_005763 | | LKR/SDH | lysine catabolism | (*) | *H | | * | SI, C, O |
| NM_004969 | 31 and 32 | IDE | insulin degrading enzyme | (*) | * | * | * | SI-ent., O |
| NM_001914 | 39 and 40 | CYB5 | cytochrome B5 | (*) | *H | | (*) | L, SI, K |
| NM_001863 | 41 and 42 | COX6B | cytochrome C oxidase subunit | (*) | *H | | * | H, M, SI, C, St |
| NM_000108 | 15 and 16 | DLDH | dihydrolipamide dehydrogenase | (*) | * | | | H, M, K; SI, C |
| NM_006214 | 37 and 38 | PHYH | phytanoyl-CoA hydroxylase | | *H | | | L, K, M; SI, C |
| NM_013283 | 17 and 18 | MAT2B | methionine adenosyltransferase | | *H | (*) | (*) | SI, C, O |
| NM_000414 | | BHSD | hydroxysteroid dehydrogenase | | | (*) | * | L, SI, O |
| NM_005038 | | cyclophilin-40 | peptidyl prolyl isomerase | | *L | | * | SI, C, L, M |
| NM_138393 | | DP1 | membrane trafficking | | (*) | * | * | L, SI |
| NM_006408 | 3 and 4 | AGR2 | anterior gradient 2 homolog | | *H | | * | St, SI, C |
| NM_021969 | 11 and 12 | NROB2 | nuclear hormone receptor | * | *H | | * | SI, L, St |
| NM_005524 | | Hes1 | transcriptional regulator | * | *H | * | * | SI-ent., O |
| NM_002054 | | GCG | proglucagon | | (*) | | * | P, SI, C |

Genes are assocaited withthe disease states B3, dysplasia (D), BE adjacent to carcinoma (BE-CA), or carcinoma (CA) if present in at lease 25% of samples tested.
(*) indicateds gene expression changes associated wiht 15-25% of samples.
Normal Tissues: highest normal tissue expression is listed. SI (small intestine); C (colon); St (stomach); K (kidney); P (pancreas); L (liver); M (muscle); H (heart); CNS (central nervous system); SI-ent (intestinal enterocytes); St-par (parietal cells; O (other tissues). In the dysplasia column, H or L denote expression associated with high-grade or low-grade dysplasia, respectively. GPCR (G protein coupled recepter.
“na” and “aa” refer to the nucleic acid and amino acid SEQ ID NO, respectively, for the associated markers.

Examples include MYO1A, an unconventional myosin that is differentially expressed along with crypt-villus axis, exhibiting low level cytosolic expression in immature crypts and high expression in villus cells with localization at the brush border (Skowron et al., Cell Motil Cytoskel. 41:308-324 (1998); and MacLennan et al., Molec. Carcinogen. 24:137-143 (1999)). Unlike villin, another marker of the brush border that was detected across all disease states, MYO1A was most associated with high-grade dysplasia and carcinoma. The novel secreted factor AGR2 gives one of the most striking profiles as a marker for high-grade dysplasia (FIG. 2A). AGR2 is a human homolog of the X. laevis cement gland gene XAG-2, which is implicated in ectodermal patterning (Aberger et al., Mech. Dev. 72:115-130 (1998)). Elevated expression of this gene is also associated with hormonally-responsive high-grade esophageal dysplasias (Thompson and Weigel, Biochem. Biophys. Res. Commun. 251:111-116 (1998)).

Expression of nuclear hormone receptor NROB2 is induced by bile acids, and NROB2 in turn participates in transcriptional repression of the rate-limiting enzyme (CYP7A1) in bile synthesis (Lu et al., Mol. Cell 6:507-515 (2000)). In this study, overexpression of NROB2 is detected in particularly in high-grade dysplasia, in addition to some carcinomas and a subset of BE samples (FIG. 2B). In addition to supporting the general pattern of intestinal metaplasia, expression of NROB2 may further reflect the response to the unnatural exposure of esophageal cells to bile, which is considered to be a contributing factor in Barrett's metaplasia (Bremner et al, Surgery 68:209-216 (1970); and Gillen et al., Br. J. Surg. 75:1352-1355 (1988)). Bile acids have also been shown to activate transcription of COX-2 (Zhang et al., J. Biol. Chem. 273:2424-2428 (1998)).

While these gene expression profiles are consistent with the observations of an increased columnar cell type in BE, the most consistent changes are associated with dysplasia, especially high-grade dypslasia (Table 2). These genes could serve as markers for progression in a clinical setting. For example, the number of genes which meet the described criteria for elevated expression in individual samples progressively increases through BE and dysplasia. The average of the number of markers detected per sample is 7.6 for BE, 11.7 for low-grade dysplasia, and 16.4 for high-grade dysplasia. Within the BE group, 3 samples have unusually high scores of 12, 12, and 14 markers detected. The two samples with 12 markers are different biopsies from the same patient: while the overall expression profiles vary between the 2 biopsies, they score identically in the marker analysis. Marker selection could be further refined to a subset associated with particular disease stages. This type of quantitative analysis may be of utility in identifying BE patients with greater risk of progression, and may be less sensitive to sampling and observer-related effects. Some of the secreted and processed factors listed (Table 1A, 2, 3) may even be detectable in the blood, which could further simplify screening.

Adenocarcinoma:

Many of the genes differentially expressed in adenocarcinoma in Barrett's, similar to other solid tumors, reflect the changes occurring as the cells acquire a more proliferative and invasive phenotype (Table 3). Included are genes involved with growth, cell adhesion, matrix invasion, vascularization, and intracellular remodeling. The majority of genes are most prevalent in adenocarinoma, but some are also detected at earlier stages. For example, genes likely to be involved in tumor angiogenesis showed significant upregulation in samples with dysplasia (eg. tumor endothelial marker 1 (TEM1), Tie2 ligand 2, VEGFC, endothelin 1).

TABLE 3
Genes up-regulated in esophageal adenocarcinoma

| | | | | | |
|---|---|---|---|---|---|
| NCBI RefSeq | Gene families/genes | BE | D | BE-CA | CA |

Growth factors/receptors

| | | | | |
|---|---|---|---|---|
| NM_005228 | EGFR | | (*H) | * |
| NM_004442 | EPHB2 | | | * |
| NM_003212 | CRIPTO CR-1 | (*) | * | * |
| NM_004429 | Ephrin B1 | | | *$ |

Metalloproteinases - related

| | | | | | |
|---|---|---|---|---|---|
| NM_016155 | MMP-17/MT4-MMP | | | | * |
| NM_021801 | MMP26 | (*) | (*) | (*) | *$ |
| NM_001110 | ADAM10 | | | * | * |
| NM_001109 | ADAM8 | | *H | | (*) |
| XM_132370# | ADAM1 | | * | | (*) |
| NM_003254 | TIM1 | * | * | * | * |

Intracellular cytoskeletal

| | | | | | |
|---|---|---|---|---|---|
| NM_001665 | rho G | (*) | | * | * |
| NM_006113 | VAV3 | | | * | * |
| NM_002086 | GRB2 | | * | * | (*) |
| NM_001666 | C1 | | *H | | |
| NM_007124 | Utrophin | | | | * |

Transcription/nuclear

| | | | | | |
|---|---|---|---|---|---|
| NM_030756 | Tcf4, DNA269446 | (*) | * | | * |
| NM_005252 | c-Fos | | * | * | * |
| NM_002592 | PCNA | | | * | * |
| NM_004060 | cyclin G | | * | | |
| NM_053056 | Cyclin D1 | | * | | (*) $ |
| NM_003401 | XRCC4 | | | | * |
| NM_007149 | Zinc finger protein | | | | * |

Cell surface adhesion/matrix

| | | | | | |
|---|---|---|---|---|---|
| XM_053256 | MUC1 | * | * | * | * |
| NM_004363 | CEA | | (*) | | * |
| NM_002483 | NCA | | | | * |
| NM_006350 | Follistatin | | *H | (*) | *$ |
| NM_021101 | Claudin 1 | | | | *$ |
| NM_012130 | Claudin 14 | | | | * |
| NM_003285 | tenascin-R | (*) | * | | * |
| NM_001793 | CAD3 | (*) | | * | * |
| NM_005076 | AXO1 | | *H | | |
| NM_001843 | CONT | | *H | | |
| NM_000582 | Osteopontin | (*) | | * | * |
| NM_006499 | Galectin 8 | (*) | | | * |
| NM_001711 | PGS1 (biglycan) | * | *L | | |
| NM_001466 | Frizzled 2 | | | | *$ |
| NM_005545 | ISLR | | | | *$ |
| NM_022763 | FLJ23399 | (*) | | * | * |

Vascularization

| | | | | | |
|---|---|---|---|---|---|
| NM_020404 | TEM1 | | *H | | (*) |
| NM_001147 | Tie2 ligand2 | | * | * | * |
| NM_003714 | STC-2 | | *H | | (*) |
| NM_005429 | VEGFC | | * | | (*) |
| NM_000930 | tPA | | | * | * |
| NM_001955 | Endothelin 1 | | *H | | (*) |
| NM_000361 | Thrombomodulin | | | (*) | * |
| NM_001993 | TF | (*) | * | | * |

Channel/transmembrane

| | | | | | |
|---|---|---|---|---|---|
| NM_005282 | GPR4 | | | * | * |
| NM_006056 | GPR66 | | | | * |
| NM_003058 | SLC22A2 | (*) | (*H) | * | * |
| NM_002420 | MLSN1 | | | | * |
| NM_000702 | ATN2, Na/K transport | | | | * |

Genes are associated with the disease states B3, dysplasia (D), BE adjacent to carcinoma (BE-CA), or carcinoma (CA) if present in at least 25% of samples tested.
(*) indicates geneexpression changes associated wiht 15-25% of samples.
$ indicates a target of the Wnt signallin pathway.

The gene expression profiles in Barrett's adenocarcinoma share many similarities with colon tumors. For example, epidermal growth factor receptor (EGFR; previously described in carcinoma in BE) (ak-Kasspooles et al., Internat. J. Cancer 54:213-219 (1993), along with other growth factor-related or cell-surface proteins such as Cripto CR1, EPHB2, MUC1, NCA/CEACAM6, CEA (Table 3), are often highly expressed in colon cancer (Ciardiello et al., Proc. Natl. Acad. Sci. USA 88:7792-7796 (1991); Liu et al., Cancer 94:934-939 (2002); Zimmerman et al., Proc. Natl. Acad. Sci. USA 84:2960-2964 (1987); Medina et al., Cancer Res. 59:1061-1070 (1999); and Ilantzis et al., Neoplasia 4:151-163 (2002)). The sodium channel associated with cystic fibrosis, CFTR, was upregulated in adenocarcinoma and can be detected in some cases of high-grade dysplasia (Table 2). This gene is also overexpressed in colon tumors. Furthermore, there is evidence that several genes listed are targets of Wnt signalling pathways (Table 3) (Tetsu and McCormick, Nature 398:422-426 (1999); Miwa et al., Oncol. Res. 12:469-476 (2000); Marchenko et al., Biochem. J. 363:253-262 (2002); Sagara et al., Biochem. and Biophys. Res. Comm. 252:117-122 (1998); Lescher et al., Dev. Dyn. 213:440-451 (1998); Willert et al., BMC Dev. Biol. 2:1-6 (2002); and Tice et al., J. Biol. Chem. 277:14329-14335 (2002)), and it is possible that COX-2, which is implicated in colon cancer as well as adenocarcinoma in Barrett's, is a Wnt pathway target (Howe et al., Cancer Res. 59:1572-1577 (1999)). An additional synergistic link is suggested by the recent finding that EGFR is activated by prostaglandin E2, a product of COX-2 (Tsujii et al., Cell 93:705-716 (1998); Tsujii et al., Proc. Natl. Acad. Sci. USA 94:3336-3340 (1997); and Pai et al., Nature Med. 8:289-293 (2002)).

More support for Wnt/beta catenin-like induction comes from the strong induction of transcription factor and TCF4 (TCF7L2) in several dysplasia and adenocarcinoma samples (FIG. 3A). Knockout studies in mice indicate that TCF4 is necessary for the maintenance of proliferative crypts in the small intestine, and constitutive acitivity of TCF4 in APC-deficient human epithelial cells may contribute to their malignant transformation (Korinek et al., Nature Gen. 19:379-383 (1998)). Given its role in colon carcinogenesis, TCF4 provides another key link between intestinal metaplasia and carcinoma in BE.

Most genes listed represent known genes, but the novel gene FLJ23399 was one of the genes most consistently observed in adenocarcinoma and patient-matched adjacent BE samples (FIG. 3B). Expression in BE adjacent to carcinoma suggests the induction may be epigenetic, or possibly reflect small foci of adencarcinoma that cannot be identified histologically. Increased expression of this gene was also discovered herein to be associated with colon tumors, and with metastatic prostate tumors (increased expression with metastasis as compared to primary tumors). Its function is unknown, but the presence of 4 type III fibronectin domains in the putative extracellular region suggest a possible role in cell adhesion and/or cell-matrix interactions.

Barrett's Esophagus-to-Adenocarcinoma Disease Progression:

Despite the difficulties associated with sampling and interpretation, the presence and degree of dysplasia is still the most predictive factor for risk of progression to adenocarinoma (Miros et al., Gut 32:1441-1446 (1991)). Foci of carcinoma typically appear adjacent to dysplasia, and esophageal resections of high-grade dysplasia frequently contain previously unrecognized adenocarcinoma (Falk et al., Gastrointest. Endosc. 49:170-176 (1999); and Cameron and Carpenter, Am. J. Gastroenterol. 92:586-591 (1997)). In this study, by the time dysplasia was apparent, there was evidence of progressive development toward a gene expression profile similar to a differentiated small intestinal enterocyte (along with a small group of genes representative of other intestinal cell types). A possible key contributing factor is the increased expression of TCF4 with advancing disease. Homozygous disruption of TCF4 in mice results in death shortly after birth, and the neonatal epithelium is composed only of non-dividing villus cells (Korinek, V. et al., Nature Gen. 19:379-383 (1998)). This suggests that the genetic program controlled by TCF4 maintains, and possibly establishes, the crypt stem cells of the small intestine. In humans, TCF4 is expressed strongly in the crypts in early fetal development, with increasing expression on the villi up to week 22 as the small intestine develops (Barker et al., Am. J. Pathol. 154:29-35 (1999)). TCF4 is also expressed along the crypt-villus axis of adult small intestine and along the epithelial lining of the crypts of adult colon. The TCF4 profile observed in dysplasia and carcinoma in BE may reflect the inappropriate activation of a developmental pathway with a possible underlying dynamic and differentiating stem cell-like population, or acquisition of some of these characteristics. The delicate cells of the small intestine, with their specialized absorptive and digestive functions and rapid turnover, would seem highly susceptible to damage in the context of the esophagus and gastrointestinal reflux disease.

The developing intestinal phenotype apparent by progression to dysplasia, associated with increased expression of TCF4, suggests some tantalizing links to the development of carcinoma and the similarities in gene expression between adenocarcinoma of the esophagus and colon. In the context of loss of APC function, association of beta catenin with TCF4 results in constitutive transcription of Tcf target genes, a proposed crucial event in the early transformation of colonic epithelia in colon cancer (Korinek et al., Science 275:1784-1787 (1997)). While there is not strong evidence of truncating mutations in APC or oncogenic beta catenin in esophageal adenocarcinoma, there is evidence of hypermethylation of the APC promoter (in 48/52 of adenocarcinoma patients and 17/43 patients with BE metaplasia) (Kawakami et al., J. Natl. Cancer Inst. 92:1805-1811 (2000)). APC hypermethylation has also been implicated in progression in colon cancer (Hiltunen et al., Int. J. Cancer 70:644-648 (1997)). In this context, it is interesting to note that elevated c-Fos expression was apparent in our study in both dysplasia and carcinoma (Table 3). This could perhaps be related to the presence of bile acids from reflux, overexpression of proglucagon-derived peptide GLP2 (Table 2), or of TNFa (Table 1), all of which have been shown to induce c-Fos expression (Bakin and Curran, Science 283:387-390 (1999); Di Toro et al., Eur. J. Pharm. Sci. 11:291-298 (2000); and Bjerknes and Cheng, Proc. Natl. Acad. Sci. USA 98:12497-12502 (2001)). One proposal for oncogenic transformation by c-Fos is hypermethylation resulting from induction of DNA 5-methylcytosine transferase (Goetze et al., Atherosclerosis 159:93-101 (2001)). These factors may contribute to a potential increased availability of beta catenin to combine with TCF4 and activate transcriptional pathways that contribute to carcinogenesis. c-Fos may play an earlier role in intestinal metaplasia as well: studies of intestinal development in mice indicate that GLP2-mediated induction of c-Fos in enteric neurons signals growth of columnar epithelial cell progenitors and stem cells (Di Toro et al., Eur. J. Pharm. Sci. 11:291-298 (2000)).

Gene expression profiling of esophageal biopsies has revealed several intriguing associations for the progression of malignancy in the context of Barrett's esophagus. Many of the genes may be involved in potentiating regulatory cycles, and there is potential synergy for the development of adenocarcinoma between exposure to damaging agents (eg. bile), inflammatory response and prostaglandin synthesis, intestinal metaplasia and TCF4 induction, along with induction of growth factors such as EGFR and oncogenes such as c-Fos. Subsets of the genes identified may also eventually serve as markers to identify patients at higher risk for adenocarcinoma. This could permit streamlining of expensive and time-consuming surveillance programs, along with earlier detection and associated improved survival chances for high-risk patients.

Diagnosis of High-grade Esophageal Dysplasia and Prognosis of Esophageal Adenocarcinoma:

Several HGD gene markers were discovered as being up-regulated at least 1.5-fold in many high-grade dysplasia samples but are up-regulated in relatively few Barrett's esophagus samples (see Table 4A compared to Table 4B). According to the invention, where at least eight of the twenty-two HGD gene markers are detected to be up-regulated at 1.5-fold in an esophageal tissue sample, cells of the tissue sample are said to exhibit HGD. In addition, the patient from whom the sample was taken may be diagnosed as experiencing high-grade esophageal dysplasia. Further, the prognosis for the patient includes the likely development of adenocarcinoma. Based on the detection of HGD, diagnosis and prognosis, the patient may be treated accordingly and at an earlier stage in the BE-to-cancer progression than would otherwise have occurred prior to disclosure of the instant invention. Alternatively, in a test esophageal tissue sample, where at least one of the at least eight up-regulated HGD marker genes is AGR2 (SEQ ID NO:3), TM7SF1 (SEQ ID NO:13), MAT2B (SEQ ID NO:17), SLNAC1 (SEQ ID NO:23), or TCF4 (SEQ ID NO:43), cells of the tissue sample exhibit HGD and the the patient is said to be diagnosed as experiencing dysplasia, particularly high-grade dysplasia, and is likely to develop adenocarcinoma.

TABLE 4A
High-grade Dysplasia Markers

| | | | | Sample ID # | | | | | | |
|---|---|---|---|---|---|---|---|---|---|---|
| | SEQ ID NO: | | | Z score* | | | | | | |
| NCBI # | (na and aa) | Gene name | | 2493 | 2955 | 2491 | 2958 | 3128 | 2493 | 3130 |
| NM_001955 | 1 and 2 | Endothelin 1 | ET-1 | 2.9 | | 1.9 | 2.7 | 2.2 | | |
| NM_006408 | 3 and 4 | anterior gradient 2 (Xenepus laevis) homolog | AGR2 | 3.1 | 2.7 | 2.6 | 2.7 | 3.4 | 2. | 2.9 |
| NM_001109 | 5 and 6 | ADAM8 | ADAM8 | 3.6 | | 1.8 | | 2.3 | | |
| NM_002773 | 7 and 8 | Prostasin precursor, serine protease | PRSS8 | 2.5 | 1.8 | 2.7 | | 3.1 | 2.3 | |
| NM_005076 | 9 and 10 | Axonin-1 precursor | AXO1 | 2. | | 1.6 | 2. | | 1.5 | |
| NM_021969 | 11 and 12 | Nuclear hormone receptor | NROB2 | 4.9 | | 2.1 | 2.8 | 3.6 | 2.6 | 2.7 |
| NM_003272 | 13 and 14 | TM7SF1 | TM7SF1 | 1.5 | 3.6 | 2.3 | 1.7 | 3. | 2.2 | 1.7 |
| NM_000108 | 15 and 16 | dihydrolipamide dehydrogenase | DLDH | 2.1 | 3.2 | 1.9 | 1.7 | | | |
| NM_013283 | 17 and 18 | methionine adenosyltransferase II, beta | MAT2B | 2.5 | 1.8 | 2.2 | 3. | 2.7 | | |
| NM_003714 | 19 and 20 | stanniocalcin-2 | STC-2 | 2.3 | | 1.7 | 1.9 | 1.6 | | 1.9 |
| NM_001631 | 21 and 22 | Alkaline phosphatase, intestinal precursor | PPBI | 2.3 | | 1.6 | 2. | 2.4 | ND | |
| NM_004769 | 23 and 24 | Sodium channel receptor SLNAC1 | SLNAC1 | 2.9 | 1.8 | 3.6 | 3. | 2.9 | ND | 2.5 |
| NM_000717 | 25 and 26 | Carbonic anhydrase iv precursor | CAH4 | | | | 1.7 | 1.8 | | 1.8 |
| NM_000928 | 27 and 28 | Phospholipase a2 precursor | PA21 | 2. | | | | 2.4 | 2.4 | |
| NM_005242 | 29 and 30 | Proteinase activated receptor 2 precursor | PAR2 | | | | 2.9 | | 2.7 | |
| NM_004969 | 31 and 32 | Insulin-degrading enzyme | IDE | | 1.6 | 2.5 | 4.4 | 1.8 | 1.9 | 1.8 |
| NM_005379 | 33 and 34 | Myosin IA (MYO1A) | MYO1A | | 1.8 | 2.3 | 1.5 | | | |
| NM_000775 | 35 and 36 | Cytochrome P450 monooxygenase CYP2J2 | CYP2J2 | | 2.4 | | 4.3 | 2.3 | | |
| NM_006214 | 37 and 38 | Phytanoyl-CoA hydroxylase (Refsum disease) | PHYH | | 2.9 | | 2.4 | | 1.9 | |
| NM_001914 | 39 and 40 | “Cytochrome b5, 3′ end” | CYB5 | | | 3. | | | 2.4 | |
| NM_001863 | 41 and 42 | “CoxVIb gene, last exon and flanking sequence” | coxVIb | 1.9 | | 2.2 | 2. | 1.9 | | 1.6 |
| NM_030756 | 43 and 44 | TCF4 | TCF4 | 3.6 | | 2.6 | 6.8 | 3.5 | 4.1 | |
| | | total number | | 15 | 10 | 17 | 18 | 16 | 12 | 8 |

Z score cut-off was 1.5 or above (p < 0.07).
“na” and “aa” refer to the nucleic acid and amino acid SEQ ID NO, respectively, for the associated markers

TABLE 4B
Low Prevalence of HGD Markers

| | | | | | Sample ID # | | | | | | |
|---|---|---|---|---|---|---|---|---|---|---|---|
| | SEQ ID NO: | | | | Z score* | | | | | | |
| NCBI # | (na and aa) | | Gene name | | B-15 | B-17 | B-18 | B | 3091 | 3131 | 3132 |
| NM_001955 | 1 and 2 | | ET-1 | | | | | | | | |
| NM_006408 | 3 and 4 | | AGR2 | | | | | 2.5 | | | |
| NM_001109 | 5 and 6 | | ADAM8 | | | 2.2 | | | | | |
| NM_002773 | 7 and 8 | | PRSS8 | | | | 3.4 | 1.5 | | | |
| NM_005076 | 9 and 10 | | AXO1 | | | | | | | | |
| NM_021969 | 11 and 12 | | NROB2 | | | | 3.2 | | | 2.4 | 2.4 |
| NM_003272 | 13 and 14 | | TM7SF1 | | | | 3.1 | | | | |
| NM_000108 | 15 and 16 | | DLDH | | 2. | | | | | | |
| NM_013283 | 17 and 18 | | MAT2B | | | | | 2.4 | | | |
| NM_003714 | 19 and 20 | | STC-2 | | | | | | | | |
| NM_001631 | 21 and 22 | | PPBI | | | | | | | 2. | |
| NM_004769 | 23 and 24 | | SLNAC1 | | 2.8 | | | | | | |
| NM_000717 | 25 and 26 | | CAH4 | | | 1.8 | 1.5 | | | | |
| NM_000928 | 27 and 28 | | PA21 | | | | | | | | |
| NM_005242 | 29 and 30 | | PAR2 | | | | | | | | |
| NM_004969 | 31 and 32 | | IDE | | | | | 1.5 | | | |
| NM_005379 | 33 and 34 | | MYO1A | | 1.5 | | | | | | 1.6 |
| NM_000775 | 35 and 36 | | CYP2J2 | | | | | | | | |
| NM_006214 | 37 and 38 | | PHYH | | | | | | | | |
| NM_001914 | 39 and 40 | | CYB5 | | 5.3 | 1.8 | | | | | |
| NM_001863 | 41 and 42 | | coxVIb | | 1.8 | 1.9 | | | | | |
| NM_030756 | 43 and 44 | | TCF4 | | | | 2.4 | | | | |
| | | | Total # | | 5 | 4 | 5 | 4 | 0 | 2 | 2 |
| | Sample ID # | | | | | | | | | | |
| | Z score* | | | | | | | | | | |
| NCBI # | 3142 | 3143 | 3088 | 2296 | 2554 | 2555 | 3134 | 3135 | 3140 | 3181 | 3141 |
| NM_001955 | | | | | | | | | | | |
| NM_006408 | | | | | | | | | | 1.5 | |
| NM_001109 | | | | | | | | | | | |
| NM_002773 | | | | | | | | | | | |
| NM_005076 | | | | | | | | | | | |
| NM_021969 | 2.2 | | 1.7 | | 1.7 | 2.6 | 1.5 | | | | |
| NM_003272 | | | | | | | | | | | |
| NM_000108 | | | | | | | | | | | |
| NM_013283 | | | | | | | | | | | |
| NM_003714 | | | | | | | | | | | |
| NM_001631 | | | | | | | | | | | |
| NM_004769 | | | | | | | | | | | |
| NM_000717 | | | | | | | | | | | 1.5 |
| NM_000928 | | 4.2 | 4.7 | | 2.6 | 4.3 | | | | 7.4 | |
| NM_005242 | | | | | | | | | | | |
| NM_004969 | | | | | | 2.6 | | | | 2.8 | 4.9 |
| NM_005379 | 1.6 | | 1.7 | | | | | | 1.6 | | |
| NM_000775 | | | | | | | | 5.7 | | | |
| NM_006214 | | | | 1.6 | | | | | | 3.2 | |
| NM_001914 | | | | | | | | | | | |
| NM_001863 | | | 1.7 | | | | 2.1 | | | | |
| NM_030756 | | | | | | | | | 2.3 | | |
| | 2 | 1 | 4 | 1 | 2 | 3 | 3 | 0 | 2 | 4 | 2 |

Z score cut-off was 1.5 or above (p < 0.07).
“na” and “aa” refer to the nucleic acid and amino acid SEQ ID NO, respectively, for the associated markers

In addition to detecting and diagnosing HGD and developing a prognosis of esophageal adenocarcinoma, treatment of cancer, including, but not limited to adenocarcinoma, esophageal adenocarcioma, and colon cancer is also possible by administering to a patient a therapeutically effective amount of an antagonist of one or more of the following adenocarcinoma marker polypeptides: CAD17 (liver-intestine cadherin, NM_{—}004063) (SEQ ID NO:46), CLDN15 (claudin 15, NM_{—}014343) (SEQ ID NO:48), SLNAC1 (sodium channel, NM_{—}004769) (SEQ ID NO:24), CFTR (chloride channel, NM_{—}000492) (SEQ ID NO:50), H2R (histamine H2 receptor, NM_{—}022304) (SEQ ID NO:52), PRSS8 (serine protease, NM_{—}002773) (SEQ ID NO:8), PA21 (phospholipase A2 group IB, NM_{—}000928) (SEQ ID NO:28), AGR2 (anterior gradient 2 homolog, (NM_{—}006408) (SEQ ID NO:4), EGFR (NM_{—}005228) (SEQ ID NO:54), EPHB2 (NM_{—}004442) (SEQ ID NO:56), CRIPTO CR-1 (NM_{—}003212) (SEQ ID NO:58), Eprin B1 (NM_{—}004429) (SEQ ID NO:60), MMP-17/MT4-MMP (NM_{—}016155) (SEQ ID NO:62), MMP26 (NM_{—}021801) (SEQ ID NO:64), ADAM10 (NM_{—}001110) (SEQ ID NO:66), ADAM8 (NM_{—}001109) (SEQ ID NO:6), ADAM1 (XM_{—}132370) (SEQ ID NO:68), TIM1 (NM_{—}003254) (SEQ ID NO:70), MUC1 (XM_{—}053256) (SEQ ID NO:72), CEA (NM_{—}004363) (SEQ ID NO:74), NCA (NM_{—}002483) (SEQ ID NO:76), Follistatin (NM_{—}006350) (SEQ ID NO:78), Claudin 1 (NM_{—}021101) (SEQ ID NO:80), Claudin 14 (NM_{—}012130) (SEQ ID NO:82), tenascin-R (NM_{—}003285) (SEQ ID NO:84), CAD3 (NM_{—}001793) (SEQ ID NO:86), AXO1 (NM_{—}005076) (SEQ ID NO:10), CONT (NM_{—}001843) (SEQ ID NO:88), Osteopontin (NM_{—}000582) (SEQ ID NO:90), Galectin 8 (NM_{—}006499) (SEQ ID NO:92), PGS1 (bihlycan, NM_{—}001711) (SEQ ID NO:94), Frizzled 2 (NM_{—}001466) (SEQ ID NO:96), ISLR (NM_{—}005545) (SEQ ID NO:98), FLJ23399 (NM_{—}022763) (SEQ ID NO:100), TEM1 (NM_{—}020404) (SEQ ID NO:102), Tie2 ligand2 (NM_{—}001147) (SEQ ID NO:104), STC-2 (NM_{—}003714) (SEQ ID NO:20), VEGFC (NM_{—}005429) (SEQ ID NO:106), tPA (NM_{—}000930) (SEQ ID NO:108), Endothelin 1 (NM_{—}001955) (SEQ ID NO:2), Thrombomodulin (NM_{—}000361) (SEQ ID NO:110), TF (NM_{—}001993) (SEQ ID NO:112), GPR4 (NM_{—}005282) (SEQ ID NO:114), GPR66 (NM_{—}006056) (SEQ ID NO:116), SLC22A2 (NM_{—}003058) ((SEQ ID NO:118), MLSN1 (NM_{—}002420) (SEQ ID NO: 120), or ATN2 (Na/K transport, NM_{—}000702) (SEQ ID NO: 122). The antagonist is a small molecule that binds and inactivates the polypeptide; binds and inactivates a precursor of the polypeptide; prevents translation of the polypeptide; prevents its transcription; or the like. Alternatively, the antagonist is an antibody that specifically binds the polypeptide and inhibits or prevents its activity. Where the antagonist is an antibody, the antibody is optionally a monoclonal antibody, a humanized antibody, or a binding fragment thereof. The treatment involves contacting a cancer cell with an antagonist of at least one of the polypeptides encoded by the adenocarcinoma marker genes listed above, alternatively with an antagonist of at least three, alternatively with at least five, and alternatively with at least eight of the polypeptides encoded by the adenocarcinoma marker genes listed above.

Further, a method of screening for a compound that inhibits cancer cell growth or causes the death of a cancer cell, particularly an adenocarcinoma cell, an esophageal adenocarcinoma cell, or a colon cancer cell, is an aspect of the invention. Accordingly, the screening method involves contacting a cancer cell, such as one expressing at least one, three, five, eight or more of the adenocarcinoma gene markers selected from the group consisiting of CAD17 (liver-intestine cadherin, NM_{—}004063) (SEQ ID NO:45), CLDN15 (claudin 15, NM_{—}014343) (SEQ ID NO:47), SLNAC1 (sodium channel, NM_{—}004769) (SEQ ID NO:23), CFTR (chloride channel, NM_{—}000492) (SEQ ID NO:49), H2R (histamine H2 receptor, NM_{—}022304) (SEQ ID NO:51), PRSS8 (serine protease, NM_{—}002773) (SEQ ID NO:7), PA21 (phospholipase A2 group IB, NM_{—}000928) (SEQ ID NO:27), AGR2 (anterior gradient 2 homolog, (NM_{—}006408) (SEQ ID NO:3), EGFR (NM_{—}005228) (SEQ ID NO:53), EPHB2 (NM_{—}004442) (SEQ ID NO:55), CRIPTO CR-1 (NM_{—}003212) (SEQ ID NO:57), Eprin B1 (NM_{—}004429) (SEQ ID NO:59), MMP-17/MT4-MMP (NM_{—}016155) (SEQ ID NO:61), MMP26 (NM_{—}021801) (SEQ ID NO:63), ADAM10 (NM_{—}001110) (SEQ ID NO:65), ADAM8 (NM_{—}001109) (SEQ ID NO:5), ADAM1 (XM_{—}132370) (SEQ ID NO:67), TIM1 (NM_{—}003254) (SEQ ID NO:69), MUC1 (XM_{—}053256) (SEQ ID NO:71), CEA (NM_{—}004363) (SEQ ID NO:73), NCA (NM_{—}002483) (SEQ ID NO:75), Follistatin (NM_{—}006350) (SEQ ID NO:77), Claudin 1 (NM_{—}021101) (SEQ ID NO:79), Claudin 14 (NM_{—}012130) (SEQ ID NO:81), tenascin-R (NM_{—}003285) (SEQ ID NO:83), CAD3 (NM_{—}001793) (SEQ ID NO:85), AXO1 (NM_{—}005076) (SEQ ID NO:9), CONT (NM_{—}001843) (SEQ ID NO:87), Osteopontin (NM_{—}000582) (SEQ ID NO:89), Galectin 8 (NM_{—}006499) (SEQ ID NO:91), PGS1 (bihlycan, NM_{—}001711) (SEQ ID NO:93), Frizzled 2 (NM_{—}001466) (SEQ ID NO:95), ISLR (NM_{—}005545) (SEQ ID NO:97), FLJ23399 (NM 022763) (SEQ ID NO:99), TEM1 (NM_{—}020404) (SEQ ID NO:101), Tie2 ligand2 (NM_{—}001147) (SEQ ID NO:103), STC-2 (NM_{—}003714) (SEQ ID NO:19), VEGFC (NM_{—}005429) (SEQ ID NO:105), tPA (NM_{—}000930) (SEQ ID NO:107), Endothelin 1 (NM_{—}001955) (SEQ ID NO:1), Thrombomodulin (NM_{—}000361) (SEQ ID NO:109), TF (NM_{—}001993) (SEQ ID NO:111), GPR4 (NM_{—}005282) (SEQ ID NO:113), GPR66 (NM_{—}006056) (SEQ ID NO:115), SLC22A2 (NM_{—}003058) ((SEQ ID NO:117), MLSN1 (NM_{—}002420) (SEQ ID NO:119), and ATN2 (Na/K transport, NM_{—}000702) (SEQ ID NO:121), followed by determining cancer cell growth inhibition or cancer cell death.

### Example 5

### Nucleic Acid and Amino Acid Sequence Identity Determinations

As shown below, Table 5 provides the complete source code for the ALIGN-2 sequence comparison computer program. This source code may be routinely compiled for use on a UNIX operating system to provide the ALIGN-2 sequence comparison computer program.

In addition, disclosed herein are hypothetical exemplifications for using the below described method to determine % amino acid sequence identity and % nucleic acid sequence identity using the ALIGN-2 sequence comparison computer program, wherein “PRO” represents the amino acid sequence of a hypothetical HGD marker polypeptide of interest, “Comparison Protein” represents the amino acid sequence of a polypeptide against which the “PRO” polypeptide of interest is being compared, “PRO-DNA” represents a hypothetical HGD marker polypeptide-encoding nucleic acid sequence of interest, “Comparison DNA” represents the nucleotide sequence of a nucleic acid molecule against which the “PRO-DNA” nucleic acid molecule of interest is being compared, “X”, “Y”, and “Z” each represent different hypothetical amino acid residues and “N”, “L” and “V” each represent different hypothetical nucleotides.

Example calculations for determining % amino acid sequence identity and nucleic acid sequence identity: 1.

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity=

(the number of identically matching amino acid residues between the two polypeptide sequences as determined by ALIGN-2) divided by (the total number of amino acid residues of the PRO polypeptide)=

5 divided by 15=33.3% 2.

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity=

(the number of identically matching amino acid residues between the two polypeptide sequences as determined by ALIGN-2) divided by (the total number of amino acid residues of the PRO polypeptide)=

5 divided by 10=50% 3.

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity=

(the number of identically matching nucleotides between the two nucleic acid sequences as determined by ALIGN-2) divided by (the total number of nucleotides of the PRO-DNA nucleic acid sequence)=

6 divided by 14=42.9% 4.

| | | |
|---|---|---|
| PRO-DNA | NNNNNNNNNNNN | (Length = 12 nucleotides) |
| Comparison DNA | NNNNLLLVV | (Length = 9 nucleotides) |

% nucleic acid sequence identity=

(the number of identically matching nucleotides between the two nucleic acid sequences as determined by ALIGN-2) divided by (the total number of nucleotides of the PRO-DNA nucleic acid sequence)=

4 divided by 12=33.3%

Although the foregoing refers to particular embodiments, it will be understood that the present invention is not so limited. It will occur to those of ordinary skill in the art that various modifications may be made to the disclosed embodiments without diverting from the overall concept of the invention. All such modifications are intended to be within the scope of the present invention.

What is claimed is:

1. A method of detecting of high-grade dysplasia (HGD) in cells of a tissue sample, the method comprising:
(a) obtaining a test tissue sample suspected of comprising cells exhibiting HGD;
(b) establishing the level of expression in the test tissue sample of at least eight genes selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO: 13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43), or variants thereof having at least 80% nucleic acid sequence identity, wherein the tissue is from esophagus or colon; and
(c) comparing expression of the at least eight genes to a baseline expression of the genes in normal tissue controls of the same tissue type, wherein an increase of at least 1.5-fold in expression of the genes relative to the baseline expression indicates that cells of the test sample exhibit HGD.

2. The method of claim 1, wherein the tissue is human tissue.

3. A method of identifying a esophageal tissue susceptable to esophageal adenocarcoma, comprising detecting esophageal HGD in a test tissue sample according to claim 1.

4. A method according to claim 1, wherein an increase of at least 2-fold in expression of genes relative to the baseline is observed.

5. A method according to claim 1, wherein at least one of the at least eight genes is selected from the group consisting of AGR2 (SEQ ID NO:3), TM7SF 1 (SEQ ID NO:13), MAT2B (SEQ ID NO:17), SLNAC1 (SEQ ID NO:23), and TCF4 (SEQ ID NO:43), or variants thereof having at least 80% nucleic acid sequence identity.

6. A method for determining predisposition of a mammalian tissue to a neo-plastic transformation by detecting HGD in cells of the tissue, the method comprising determining in a cell from the tissue expression of a nucleic acid sequence of at least eight genes selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43), or variants thereof having at least 80% nucleic acid sequence identity, wherein the tissue of from esophagus or colon, and wherein the expression in the test sample is at least 1.5-fold above baseline expression in a normal tissue control of the same tissue type.

7. A method according to claim 6, wherein the tissue is human tissue.

8. A method according to claim 6, wherein at least one of the at least eight genes is selected from the group consisting of AGR2 (SEQ ID NO:3), TM7SF1 (SEQ ID NO:13), MAT2B (SEQ ID NO:17), SLNAC1 (SEQ ID NO:23), and TCF4 (SEQ ID NO:43), or variants thereof having at least 80% nucleic acid sequence identity.

9. A method of detecting high-grade dysplasia (HGD) in cells of a mammalian tissue sample, the method comprising:
(a) obtaining a test tissue sample suspected of comprising cells exhibiting HGD;
(b) establishing the level of expression in the test tissue sample of at least eight polypeptides encoded by genes selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43), or variants thereof having at least 80% nucleic acid sequence identity, wherein the tissue is from esophagus or colon; and
(c) comparing expression of the at least eight polypeptides in the test tissue sample to expression of the at least eight polypeptides in normal tissue controls of the same tissue type, wherein an increase of at least 1.5-fold in expression of the polypeptides in the test tissue sample relative to the normal tissue controls indicates that cells of the test sample exhibit HGD.

10. A method as according to claim 9 comprising contacting the test tissue sample with an antibody that specifically binds one of the at least eight polypeptides under conditions that permit the antibody to bind the polypeptide.

11. A method according to claim 9, wherein at least one of the at least eight polypeptides expressed by a gene selected from the group consisting of AGR2 (SEQ ID NO:3), TM7SF1 (SEQ ID NO:13), MAT2B (SEQ ID NO:17), SLNAC1 (SEQ ID NO:23), and TCF4 (SEQ ID NO:43), or variants thereof having at least 80% nucleic acid sequence entity.

12. The method of claim 1, wherein gene expression is determined by nucleic acid microarray analysis.

13. The method of claim 12, wherein analysis comprises contacting nucleic acid from a test tissue sample with a nucleic acid microarray comprising nucleic acid probe sequences, wherein at least eight of the nucleic acid probe sequences separately comprises at least 50 contiguous nucleotides from a gene selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43), or variants thereof having at least 80% nucleic acid sequence identity.

14. The method of claim 13, wherein the at least eight nucleic acid probe sequences comprise at least 60 contiguous nucleotides from a gene selected from the group.

15. The method of claim 14, wherein the at least eight nucleic acid probe sequences comprise at least 80 contiguous nucleotides from a gene selected from the group.

16. The method of claim 15, wherein the at least eight nucleic acid probe sequences comprise at least 100 contiguous nucleotides from a gene selected from the group.

17. The method of claim 16, wherein the at least eight nucleic acid probe sequences comprise at least 150 contiguous nucleotides from a gene selected from the group.

18. The method of claim 17, wherein the at least eight nucleic acid probe sequences comprise at least 200 contiguous nucleotides from a gene selected from the group.

19. The method of claim 13, wherein the nucleic acid microarray comprises nucleic acid probe sequences from at least ten genes selected from the group.

20. The method of claim 19, wherein the nucleic acid microarray comprises nucleic acid probe sequences from at least twelve genes selected from the group.

21. The method of claim 20, wherein the nucleic acid microarray comprises nucleic acid probe sequences from at least fifteen genes selected from the group.

22. The method of claim 21, wherein the nucleic acid microarray comprises nucleic acid probe sequences from at least eighteen genes selected from the group.

23. The method of claim 22, wherein the nucleic acid microarray comprises nucleic acid probe sequences from at least twenty genes selected from the group.

24. The method of claim 23, wherein the nucleic acid microarray comprises nucleic acid probe sequences from at least twenty two genes selected from the group.

25. The method of claim 1, wherein gene expression is determined by nucleic acid hybridization under high stringency conditions of a detectable probe comprising at least 50 contiguous nucleotides from a gene selected from the group to nucleic acid of cells of the test tissue sample relative to cells of the normal tissue control.

26. The method of claim 25, wherein the hybridization is in situ hybridization.

27. The method of claim 26, wherein the hybridization is fluorescent in situ hybridization.

28. The method of claim 1, wherein gene expression is determined by polymerase chain reaction (PCR) analysis.

29. The method of claim 1, wherein gene expression is determined by real-time polymerase chain reaction (RT-PCR) analysis.

30. The method of claim 1, wherein gene expression is determined by Taqman® polymerase chain reaction analysis.

31. A kit comprising a microarray, the microarray comprising nucleic acid probe sequences, wherein at least eight of the nucleic acid probe sequences each comprise at least 50 contiguous nucleotides from a gene selected from the group consisting of ET-1 (endothelin-1, NM_{—}001955) (SEQ ID NO:1); AGR2 (anterior gradient 2 (Xenepus laevis) homolog, NM_{—}006408) (SEQ ID NO:3); ADAM8 (NM_{—}001109) (SEQ ID NO:5); PRSS8 (Prostasin precursor, serine protease, NM_{—}002773) (SEQ ID NO:7); AXO1 (Axonin-1 precursor, NM_{—}005076) (SEQ ID NO:9); NROB2 (Nuclear hormone receptor, NM_{—}021969) (SEQ ID NO:11); TM7SF1 (NM_{—}003272) (SEQ ID NO:13); DLDH (dihydrolipamide dehydrogenase, NM_{—}000108) (SEQ ID NOS:15); MAT2B (methionine adenosyltransferase II, beta, NM_{—}013283) (SEQ ID NO:17); STC-2 (stanniocalcin-2, NM_{—}003714) (SEQ ID NO:19); PPBI (alkaline phosphatase, intestinal precursor, NM_{—}001631) (SEQ ID NO:21); SLNAC1 (sodium channel receptor SLNAC1, NM_{—}004769) (SEQ ID NO:23); CAH4 (carbonic anhydrase iv precursor, NM_{—}000717) (SEQ ID NO:25); PA21 (phopholipase a2 precursor, NM_{—}000928) (SEQ ID NO:27); PAR2 (proteinase activated receptor 2 precursor, NM_{—}005242) (SEQ ID NO:29); IDE (insulin-degrading enzyme, NM_{—}004969) (SEQ ID NO:31); MYO1A (myosin-1A, NM_{—}005379) (SEQ ID NO:33); CYP2J2 (cytochrome P450 monooxygenase, NM_{—}000775) (SEQ ID NO:35); PHYH (phytanoyl-CoA-hydroxylase (Refsum disease), NM_{—}006214) (SEQ ID NO:37); CYB5 (cytochrome b5, 3′ end, NM_{—}001914) (SEQ ID NO:39); COXVIb (coxVIb gene, last exon and flanking sequence, NM_{—}001863) (SEQ ID NO:41); and TCF4 (NM_{—}030756) (SEQ ID NO:43), or variants thereof having at least 80% nucleic acid sequence identity, and a package insert indicating that the microarray is for use in detecting HGD in a test tissue sample, wherein the tissue is from esophagus or colon, and wherein an increase in expression in the test tissue sample of at least 1.5-fold of the at least eight genes relative to a normal tissue control of the same tissue type indicates that cells of the test tissue exhibit HGD.

32. The kit of claim 31, wherein the nucleic acid probe sequences each comprise at least 60 contiguous nucleotides from a gene selected from the group.

33. The kit of claim 32, wherein the nucleic acid probe sequences each comprise at least 80 contiguous nucleotides from a gene selected from the group.

34. The kit of claim 33, wherein the nucleic acid probe sequences each comprise at least 100 contiguous nucleotides from a gene selected from the group.

35. The kit of claim 34, wherein the nucleic acid probe sequences each comprise at least 150 contiguous nucleotides from a gene selected from the group.

36. The kit of claim 35, wherein the nucleic acid probe sequences each comprise at least 200 contiguous nucleotides from a gene selected from the group.

37. A method of detecting cancer in a patient, the method comprising:
(a) obtaining a test tissue sample from the patient;
(b) establishing the level of expression of a gene selected from the group consisting of CAD17 (liver-intestine cadherin, NM_{—}004063) (SEQ ID NO:45), CLDN15 (claudin 15, NM_{—}014343) (SEQ ID NO:47), SLNAC1 (sodium channel, NM_{—}004769) (SEQ ID NO:23), CFTR (chloride channel, NM_{—}000492) (SEQ ID NO:49), H2R (histamine H2 receptor, NM_{—}022304) (SEQ ID NO:51), PRSS8 (serine protease, NM_{—}002773) (SEQ ID NO:7), PA21 (phospholipase A2 group IB, NM_{—}000928) (SEQ ID NO:27), AGR2 (anterior gradient 2 homolog, (NM_{—}006408) (SEQ ID NO:3), EGFR (NM_{—}005228) (SEQ ID NO:53), EPHB2 (NM_{—}004442) (SEQ ID NO:55), CRIPTO CR-1 (NM_{—}003212) (SEQ ID NO:57), Eprin B1 (NM_{—}004429) (SEQ ID NO:59), MMP-17/MT4-MMP (NM_{—}016155) (SEQ ID NO:61), MMP26 (NM_{—}021801) (SEQ ID NO:63), ADAM10 (NM_{—}001110) (SEQ ID NO:65), ADAM8 (NM_{—}001109) (SEQ ID NO:5), ADAM1 (XM_{—}132370) (SEQ ID NO:67), TIM1 (NM_{—}003254) (SEQ ID NO:69), MUC1 (XM_{—}053256) (SEQ ID NO:71), CEA (NM_{—}004363) (SEQ ID NO:73), NCA (NM_{—}002483) (SEQ ID NO:75), Follistatin (NM_{—}006350) (SEQ ID NO:77), Claudin 1 (NM_{—}021101) (SEQ ID NO:79), Claudin 14 (NM_{—}012130) (SEQ ID NO:81), tenascin-R (NM_{—}003285) (SEQ ID NO:83), CAD3 (NM_{—}001793) (SEQ ID NO:85), AXO1 (NM_{—}005076) (SEQ ID NO:9), CONT (NM_{—}001843) (SEQ ID NO:87), Osteopontin (NM_{—}000582) (SEQ ID NO:89), Galectin 8 (NM_{—}006499) (SEQ ID NO:91), PGS1 (bihlycan, NM_{—}001711) (SEQ ID NO:93), Frizzled 2 (NM_{—}001466) (SEQ ID NO.:95), ISLR (NM_{—}005545) (SEQ ID NO:97), FLJ23399 (NM_{—}022763) (SEQ ID NO:99), TEM1 (NM_{—}020404) (SEQ ID NO:101), Tie2 ligand2 (NM_{—}001147) (SEQ ID NO:103), STC-2 (NM_{—}003714) (SEQ ID NO:19), VEGFC (NM_{—}005429) (SEQ ID NO:105), tPA (NM_{—}000930) (SEQ ID NO:107), Endothelin 1 (NM_{—}001955) (SEQ ID NO:1), Thrombomodulin (NM_{—}000361) (SEQ ID NO:109), TF (NM_{—}001993) (SEQ ID NO:111), GPR4 (NM_{—}005282) (SEQ ID NO:113), GPR66 (NM_{—}006056) (SEQ ID NO:115), SLC22A2 (NML003058) ((SEQ ID NO:117), MLSN1 (NM_{—}002420) (SEQ ID NO:119), and ATN2 (Na/K transport, NM_{—}000702) (SEQ ID NO:121), or variants thereof having at least 80% nucleic acid sequence identity, wherein the test tissue is from esophagus or colon; and wherein the expressing in the test tissue is at a level at least 1.5-fold above expression of the gene in a normal tissue control of the same tissue type.

38. The method of claim 37, wherein inhibition of cell growth is cell death.

39. The method of claim 37, wherein at least two genes selected from the group are expressed at a level at least 1.5-fold above expression of the gene in a normal cell control.

40. The method of claim 39, wherein at least three genes selected from the group are expressed at a level at least 1.5-fold above expression of the gene in a normal cell control.

41. The method of claim 40, wherein at least 5 genes selected from the group are expressed at a level at least 1.5-fold above expression of the gene in a normal cell control.

42. The method of claim 41, wherein at least 8 genes selected from the group are expressed at a level at least 1.5-fold above expression of the gene in a normal cell control.

43. The method of claim 1, wherein the expression p value is less than 0.07.

44. The method of claim 6, wherein the expression p value is less than 0.07.

45. The method of claim 9, wherein the expression p value is less than 0.07.


SEQUENCE LISTING (123 sequences)

SEQ ID NO: 1 - DNA, 1334 bp, Homo sapien
cgccgcgtgcgcctgcagacgctccgctcgctgccttctctcctggcaggcgctgccttttctccccgttaaagggcacttgggctgaaggatcgctttgagatctgaggaacccgcagcgctttgagggacctgaagctgtttttcttcgttttcctttgggttcagtttgaacgggaggtttttgatccctttttttcagaatggattatttgctcatgattttctctctgctgtttgtggcttgccaaggagctccagaaacagcagtcttaggcgctgagctcagcgcggtgggtgagaacggcggggagaaacccactcccagtccaccctggcggctccgccggtccaagcgctgctcctgctcgtccctgatggataaagagtgtgtctacttctgccacctggacatcatttgggtcaacactcccgagcacgttgttccgtatggacttggaagccctaggtccaagagagccttggagaatttacttcccacaaaggcaacagaccgtgagaatagatgccaatgtgctagccaaaaagacaagaagtgctggaatttttgccaagcaggaaaagaactcagggctgaagacattatggagaaagactggaataatcataagaaaggaaaagactgttccaagcttgggaaaaagtgtatttatcagcagttagtgagaggaagaaaaatcagaagaagttcagaggaacacctaagacaaaccaggtcggagaccatgagaaacagcgtcaaatcatcttttcatgatcccaagctgaaaggcaatccctccagagagcgttatgtgacccacaaccgagcacattggtgacagaccttcggggcctgtctgaagccatagcctccacggagagccctgtggccgactctgcactctccaccctggctgggatcagagcaggagcatcctctgctggttcctgactggcaaaggaccagcgtcctcgttcaaaacattccaagaaaggttaaggagttcccccaaccatcttcactggcttccatcagtggtaactgctttggtctcttctttcatctggggatgacaatggacctctcagcagaaacacacagtcacattcgaattcgggtggcatcctccggagagagagagaggaaggagattccacacaggggtggagtttctgacgaaggtcctaagggagtgtttgtgtctgactcaggcgcctggcacatttcagggagaaactccaaagtccacacaaagattttctaaggaatgcacaaattgaaaacacactcaaaagacaaacatgcaagtaaagaaaaaaaaaaaaaaaaa

SEQ ID NO: 2 - protein, 212 aa, Homo sapien
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGNPSRERYVTHNRAHW

SEQ ID NO: 3 - DNA, 1701 bp, Homo sapien
ccgcatcctagccgccgactcacacaaggcaggtgggtgaggaaatccagagttgccatggagaaaattccagtgtcagcattcttgctccttgtggccctctcctacactctggccagagataccacagtcaaacctggagccaaaaaggacacaaaggactctcgacccaaactgccccagaccctctccagaggttggggtgaccaactcatctggactcagacatatgaagaagctctatataaatccaagacaagcaacaaacccttgatgattattcatcacttggatgagtgcccacacagtcaagctttaaagaaagtgtttgctgaaaataaagaaatccagaaattggcagagcagtttgtcctcctcaatctggtttatgaaacaactgacaaacacctttctcctgatggccagtatgtccccaggattatgtttgttgacccatctctgacagttagagccgatatcactggaagatattcaaatcgtctctatgcttacgaacctgcagatacagctctgttgcttgacaacatgaagaaagctctcaagttgctgaagactgaattgtaaagaaaaaaaatctccaagcccttctgtctgtcaggccttgagacttgaaaccagaagaagtgtgagaagactggctagtgtggaagcatagtgaacacactgattaggttatggtttaatgttacaacaactattttttaagaaaaacaagttttagaaatttggtttcaagtgtacatgtgtgaaaacaatattgtatactaccatagtgagccatgattttctaaaaaaaaaaataaatgttttgggggtgttctgttttctccaacttggtctttcacagtggttcgtttaccaaataggattaaacacacacaaaatgctcaaggaagggacaagacaaaaccaaaactagttcaaatgatgaagaccaaagaccaagttatcatctcaccacaccacaggttctcactagatgactgtaagtagacacgagcttaatcaacagaagtatcaagccatgtgctttagcataaaagaatatttagaaaaacatcccaagaaaatcacatcactacctagagtcaactctggccaggaactctaaggtacacactttcatttagtaattaaattttagtcagattttgcccaacctaatgctctcagggaaagcctctggcaagtagctttctccttcagaggtctaatttagtagaaaggtcatccaaagaacatctgcactcctgaacacaccctgaagaaatcctgggaattgaccttgtaatcgatttgtctgtcaaggtcctaaagtactggagtgaaataaattcagccaacatgtgactaattggaagaagagcaaagggtggtgacgtgttgatgaggcagatggagatcagaggttactagggtttaggaaacgtgaaaggctgtggcatcagggtaggggagcattctgcctaacagaaattagaattgtgtgttaatgtcttcactctatacttaatctcacattcattaatatatggaattcctctactgcccagcccctcctgatttctttggcccctggactatggtgctgtatataatgctttgcagtatctgttgcttgtcttgattaacttttttggataaaaccttttttgaacagaaaaaaaaaaaaaaaaaaaa

SEQ ID NO: 4 - protein, 175 aa, Homo sapien
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 5 - DNA, 3236 bp, Homo sapien
gacccggccatgcgcggcctcgggctctggctgctgggcgcgatgatgctgcctgcgattgcccccagccggccctgggccctcatggagcagtatgaggtcgtgttgccgcggcgtctgccaggcccccgagtccgccgagctctgccctcccacttgggcctgcacccagagagggtgagctacgtccttggggccacagggcacaacttcaccctccacctgcggaagaacagggacctgctgggttccggctacacagagacctatacggctgccaatggctccgaggtgacggagcagcctcgcgggcaggaccactgcttataccagggccacgtagaggggtacccggactcagccgccagcctcagcacctgtgccggcctcaggggtttcttccaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggactctctgccatcccgagagacccgctacgtggagctgtatgtggtcgtggacaatgcagagttccagatgctggggagcgaagcagccgtgcgtcatcgggtgctggaggtggtgaatcacgtggacaagctatatcagaaactcaacttccgtgtggtcctggtgggcctggagatttggaatagtcaggacaggttccacgtcagccccgaccccagtgtcacactggagaacctcctgacctggcaggcacggcaacggacacggcggcacctgcatgacaacgtacagctcatcacgggtgtcgacttcaccgggactactgtggggtttgccagggtgtccgccatgtgctcccacagctcaggggctgtgaaccaggaccacagcaagaaccccgtgggcgtggcctgcaccatggcccatgagatgggccacaacctgggcatggaccatgatgagaacgtccagggctgccgctgccaggaacgcttcgaggccggccgctgcatcatggcaggcagcattggctccagtttccccaggatgttcagtgactgcagccaggcctacctggagagctttttggagcggccgcagtcggtgtgcctcgccaacgcccctgacctcagccacctggtgggcggccccgtgtgtgggaacctgtttgtggagcgtggggagcagtgcgactgcggcccccccgaggactgccggaaccgctgctgcaactctaccacctgccagctggctgagggggcccagtgtgcgcacggtacctgctgccaggagtgcaaggtgaagccggctggtgagctgtgccgtcccaagaaggacatgtgtgacctcgaggagttctgtgacggccggcaccctgagtgcccggaagacgccttccaggagaacggcacgccctgctccgggggctactgctacaacggggcctgtcccacactggcccagcagtgccaggccttctgggggccaggtgggcaggctgccgaggagtcctgcttctcctatgacatcctaccaggctgcaaggccagccggtacagggctgacatgtgtggcgttctgcagtgcaagggtgggcagcagcccctggggcgtgccatctgcatcgtggatgtgtgccacgcgctcaccacagaggatggcactgcgtatgaaccagtgcccgagggcacccggtgtggaccagagaaggtttgctggaaaggacgttgccaggacttacacgtttacagatccagcaactgctctgcccagtgccacaaccatggggtgtgcaaccacaagcaggagtgccactgccacgcgggctgggccccgccccactgcgcgaagctgctgactgaggtgcacgcagcgtccgggagcctccccgtcctcgtggtggtggttctggtgctcctggcagttgtgctggtcaccctggcaggcatcatcgtctaccgcaaagcccggagccgcatcctgagcaggaacgtggctcccaagaccacaatggggcgctccaaccccctgttccaccaggctgccagccgcgtgccggccaagggcggggctccagccccatccaggggcccccaagagctggtccccaccacccacccgggccagcccgcccgacacccggcctcctcggtggctctgaagaggccgccccctgctcctccggtcactgtgtccagcccacccttcccagttcctgtctacacccggcaggcaccaaagcaggtcatcaagccaacgttcgcacccccagtgcccccagtcaaacccggggctggtgcggccaaccctggtccagctgagggtgctgttggcccaaaggttgccctgaagccccccatccagaggaagcaaggagccggagctcccacagcaccctaggggggcacctgcgcctgtgtggaaatttggagaagttgcggcagagaagccatgcgttccagccttccacggtccagctagtgccgctcagccctagaccctgactttgcaggctcagctgctgttctaacctcagtaatgcatctacctgagaggctcctgctgtccacgccctcagccaattccttctccccgccttggccacgtgtagccccagctgtctgcaggcaccaggctgggatgagctgtgtgcttgcgggtgcgtgtgtgtgtacgtgtctccaggtggccgctggtctcccgctgtgttcaggaggccacatatacagcccctcccagccacacctgcccctgctctggggcctgctgagccggctgccctgggcacccggttccaggcagcacagacgtggggcatccccagaaagactccatcccaggaccaggttcccctccgtgctcttcgagagggtgtcagtgagcagactgcaccccaagctcccgactccaggtcccctgatcttgggcctgtttcccatgggattcaagagggacagccccagctttgtgtgtgtttaagcttaggaatgccctttatggaaagggctatgtgggagagtcagctatcttgtctggttttcttgagacctcagatgtgtgttcagcagggctgaaagcttttattctttaataatgagaaatgtatattttactaataaattattgaccgagttctgtagattcttgttaga

SEQ ID NO: 6 - protein, 824 aa, Homo sapien
MRGLGLWLLGAMMLPAIAPSRPWALMEQYEVVLPRRLPGPRVRRALPSHLGLHPERVSYVLGATGHNFTLHLRKNRDLLGSGYTETYTAANGSEVTEQPRGQDHCLYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAASGSLPVLVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQVIKPTFAPPVPPVKPGAGAANPGPAEGAVGPKVALKPPIQRKQGAGAPTAP

SEQ ID NO: 7 - DNA, 1938 bp, Homo sapien
gactttggtggcaagaggagctggcggagcccagccagtgggcggggccaggggaggggcgggcaggtaggtgcagccactcctgggaggaccctgcgtggccagacggtgctggtgactcgtccacactgctcgcttcggatactccaggcgtctcccgttgcggccgctccctgccttagaggccagccttggacacttgctgcccctttccagcccggattctgggatccttccctctgagccaacatctgggtcctgccttcgacaccaccccaaggcttcctaccttgcgtgcctggagtctgccccaggggcccttgtcctgggccatggcccagaagggggtcctggggcctgggcagctgggggctgtggccattctgctctatcttggattactccggtcggggacaggagcggaaggggcagaagctccctgcggtgtggccccccaagcacgcatcacaggtggcagcagtgcagtcgccggtcagtggccctggcaggtcagcatcacctatgaaggcgtccatgtgtgtggtggctctctcgtgtctgagcagtgggtgctgtcagctgctcactgcttccccagcgagcaccacaaggaagcctatgaggtcaagctgggggcccaccagctagactcctactccgaggacgccaaggtcagcaccctgaaggacatcatcccccaccccagctacctccaggagggctcccagggcgacattgcactcctccaactcagcagacccatcaccttctcccgctacatccggcccatctgcctccctgcagccaacgcctccttccccaacggcctccactgcactgtcactggctggggtcatgtggccccctcagtgagcctcctgacgcccaagccactgcagcaactcgaggtgcctctgatcagtcgtgagacgtgtaactgcctgtacaacatcgacgccaagcctgaggagccgcactttgtccaagaggacatggtgtgtgctggctatgtggaggggggcaaggacgcctgccagggtgactctgggggcccactctcctgccctgtggagggtctctggtacctgacgggcattgtgagctggggagatgcctgtggggcccgcaacaggcctggtgtgtacactctggcctccagctatgcctcctggatccaaagcaaggtgacagaactccagcctcgtgtggtgccccaaacccaggagtcccagcccgacagcaacctctgtggcagccacctggccttcagctctgccccagcccagggcttgctgaggcccatccttttcctgcctctgggcctggctctgggcctcctctccccatggctcagcgagcactgagctggccctacttccaggatggatgcatcacactcaaggacaggagcctggtccttccctgatggcctttggacccagggcctgacttgagccactccttccttcaggactctgcgggaggctggggccccatcttgatctttgagcccattcttctgggtgtgctttttgggaccatcactgagagtcaggagttttactgcctgtagcaatggccagagcctctggcccctcacccaccatggaccagcccattggccgagctcctggggagctcctgggacccttggctatgaaaatgagccctggctcccacctgtttctggaagactgctcccggcccgcctgcccagactgatgagcacatctctctgccctctccctgtgttctgggctggggccacctttgtgcagcttcgaggacaggaaaggccccaatcttgcccactggccgctgagcgcccccgagccctgactcctggactccggaggactgagcccccaccggaactgggctggcgcttggatctggggtgggagtaacagggcagaaatgattaaaatgtttgagcac

SEQ ID NO: 8 - protein, 343 aa, Homo sapien
MAQKGVLGPGQLGAVAILLYLGLLRSGTGAEGAEAPCGVAPQARITGGSSAVAGQWPWQVSITYEGVHVCGGSLVSEQWVLSAAHCFPSEHHKEAYEVKLGAHQLDSYSEDAKVSTLKDIIPHPSYLQEGSQGDIALLQLSRPITFSRYIRPICLPAANASFPNGLHCTVTGWGHVAPSVSLLTPKPLQQLEVPLISRETCNCLYNIDAKPEEPHFVQEDMVCAGYVEGGKDACQGDSGGPLSCPVEGLWYLTGIVSWGDACGARNRPGVYTLASSYASWIQSKVTELQPRVVPQTQESQPDSNLCGSHLAFSSAPAQGLLRPILFLPLGLALGLLSPWLSEH

SEQ ID NO: 9 - DNA, 7650 bp, Homo sapien
acacacacgcgccctcacccgccaccgccgccgcggccgccgccgcacccggacagcgagcggctgaggccgccagggcccaaaggacagcggcccagacaggggctggcggcccggccggccccggctcaccgactcgggcagcatccacctgccccagccaacacccttctctcgccccaggtcctttctcagcctccagctgggctgtccccaagctgagctgaggctcttctcctccgatccccacctctgcccggacatccaccatggggacagccaccaggaggaagccacacctgctgctggtagctgctgtggcccttgtctcctcttcagcttggagttcagccctgggatcccaaaccaccttcgggcctgtctttgaagaccagcccctcagtgtgctattcccagaggagtccacggaggagcaggtgttgctggcatgccgcgcccgggccagccctccagccacctatcggtggaagatgaatggtaccgagatgaagctggagccaggttcccgtcaccagctggtggggggcaacctggtcatcatgaaccccaccaaggcacaggatgccggggtctaccagtgcctggcctccaacccagtgggcaccgttgtcagcagggaggccatcctccgcttcggctttctgcaggaattctccaaggaggagcgagacccagtgaaagctcatgaaggctggggggtgatgttgccctgtaacccacctgcccactacccaggcttgtcctaccgctggctcctcaacgagttccccaacttcatcccgacggacgggcgtcacttcgtgtcccagaccacagggaacctgtacattgcccgaaccaatgcctcagacctgggcaactactcctgtttggccaccagccacatggacttctccaccaagagcgtcttcagcaagtttgctcagctcaacctggctgctgaagatacccggctctttgcacccagcatcaaggcccggttcccagcagagacctatgcactggtggggcagcaggtcaccctggagtgcttcgcctttgggaaccctgtcccccggatcaagtggcgcaaagtggacggctccctgtccccgcagtggaccacagctgagcccaccctgcagatccccagcgtcagctttgaggatgagggcacctacgagtgtgaggcggagaactccaagggccgagacaccgtgcagggccgcatcatcgtgcaggctcagcctgagtggctaaaagtgatctcggacacagaggctgacattggctccaacctgcgttggggctgtgcagccgccggcaagccccggcctacagtgcgctggctgcggaacggggagcctctggcctcccagaaccgggtggaggtgttggctggggacctgcggttctccaagctgagcctggaagactcgggcatgtaccagtgtgtggcagagaataagcacggtaccatctacgccagcgccgagctagccgtgcaagcactcgcccctgacttcaggctgaatcccgtgaggcgtctgatccccgcggcccgcgggggagagatccttatcccctgccagccccgggcagctccaaaggccgtggtgctctggagcaaaggcacggagattttggtcaacagcagcagagtgactgtaactccagatggcaccttgatcataagaaacatcagccggtcagatgaaggcaaatacacctgctttgctgagaacttcatgggcaaagccaacagcactggaatcctatctgtgcgagatgcaaccaaaatcactctagccccctcaagtgccgacatcaacttgggtgacaacctgaccctacagtgccatgcctcccacgaccccaccatggacctcaccttcacctggaccctggacgacttccccatcgactttgataagcctggagggcactaccggagaactaatgtgaaggagaccattggggatctgaccatcctgaacgcccagctgcgccatggggggaagtacacgtgcatggcccagacggtggtggacagcgcgtccaaggaggccacagtcctggtccgaggtccgccaggtcccccaggaggtgtggtggtgagggacattggcgacaccaccatccagctcagctggagccgtggcttcgacaaccacagccccatcgctaagtacaccctgcaagctcgcactccacctgcagggaagtggaagcaggttcggaccaatcctgcaaacatcgagggcaatgccgagactgcacaggtgctgggcctcaccccctggatggactatgagttccgggtcatagccagcaacattctgggcactggggagcctagtgggccctccagcaaaatccggaccagggaagcagccccctcggtggcaccctcaggactcagcggaggaggtggagcccccggagagctcatcgtcaactggacgcccatgtcacgggagtaccagaacggagacggcttcggctacctgctgtccttccgcaggcagggcagcactcactggcagaccgcccgggtgcctggcgccgatgcccagtactttgtctacagcaacgagagcgtccggccctacacgccctttgaggtcaagatccgcagctacaaccgccgcggggatgggcccgagagcctcactgcactcgtgtactcagctgaggaagagcccagggtggcccctaccaaggtgtgggccaaaggggtctcatcctcagagatgaacgtgacctgggaacccgtgcagcaggacatgaatggtatcctcctggggtatgagatccgctactggaaagctggggacaaagaagcagctgcggaccgagtgaggacagcagggctggacaccagtgcccgagtcagcggcctgcatcccaacaccaagtaccatgtgaccgtgagggcctacaaccgggctggcactgggcctgccagcccttctgccaacgccacgaccatgaagccccctccgcggcgacctcctggcaacatctcctggactttctcaagctctagtcttagcattaagtgggaccctgtggtccctttccgaaatgagtctgcagtcaccggctataagatgctgtaccagaatgacttacacctgactcccacgctccacctcaccggcaagaactggatagaaatcccagtgcctgaagacattggccatgccctggtacaaattcggaccacagggcccggaggggatgggatccctgcagaagtccacatcgtgaggaatggaggcacaagcatgatggtggagaacatggcagtccgcccagcaccacaccctggcaccgtcatttcccactccgtggcgatgctgatcctcataggctccctggagctctgatcctggaacccctccctctgcgccgcagctggacgccacctccgacggacacagccagccccttcctgctgccaaggtggcctgacactgtgccagagagtggctggttttaaatacctactttaaacagtgccctttttgtaggaggtaggatattttatattctgccgcaggatagaacccacgcaaggattttctttaaattgagaggcaccaggcagtaacttccatgatgacactgacgcctatacctgagctctaggctgcctggagggaaggaacaggcccatgggaagaagggggttttaaaaacatgtcttcaactcagcagagatggccctctgggaccctatacggactccgccacttgagagcagtcctaggcccggcaggaacaccagacatgaacaggttgaagaactggagcgaagtgcacacctcaccatccttcagtctaaggaagaagggcaagccctgggaccaagagctctcccgccttctccctcgagcagcagcaaggaccctgacgctgtccccgataactccctaggggctcctgcctgcccaagcggctgagaaccagcgccccgatgcctgaggctgggagcctgagccccttcagctttgaggggggtgatactccaggctgtttggggtgggagccaaaaagagttgagaggccagggcccttggtggaaaggggcaccagccttggtctgagatagtcacaacccaggtgacgatgccctctcagccaacactgccaacctgaccctgtcatcccgattgacagcgccacttcaggtggctgggtgactaaagggcttgtcttggtggggtctcccacccctccaagacccattctgcacagtccctccagggtttgggcaggagatggccaatcatgcgcccacctctccagtgctgcctgcagtcagctcggcctccccgacctgcagccccagactctgctctcccagcactgactcactcctgcctgggaggggaatgcagcattcatgctgtgtgtcctggtattgggaggtttctgggaagggcagaggataaatgtggccctgcctgctcccaggtatacctaggaccacctggccagatccgctcccagacggccttggactgcttgcatttccccggagaaaaaggggttaataaatgggccatcctttcctgagctctgggtatactaccagtcacagaacgtcagagctggaagaagccttagagctcaacttcttcaagcccctcactttacagatgaggaaatggaggtggtccagagagggtctgggattcccaaggtcacacagcccagaagagatggggctgggttaagaactcgagtcttccacctttctgttcaaggctgtttgtctacccagaggaaggaggcactgctgaatggctatggcctggctaagaaggtgattagtcagtagggtgtgaaaattctacttcaaggggttcggattggtgatcatggggattggcatggctgggttcccgtccaaggtgtgggcagagcttctaccaaacttcaacatggagggctgacttgaagctccctgtccccctcactcttgccccaagaaaagaggccaaagcaagagcagattccctaggcaagagcagcagcacaactaggaaaccccaaagcccatgctccgacaggtggcccttcacagggggcagcgggacaggcatcttgaagggcatatgtcctcggaagctccgagcctgttttctgtagtttatagttagagctctattttgttatggttttttaaacttttaagtcctgctctattttcctgggcaggtttatgttgatgtttacccactacaattttttaaaaatataagctcacatgccttttccctgccacagccaaacccccactgcaccctacccacccacccctagcccaggtcagctttcctggagctggctaatgaaagcctcctcacctcttcccaacccttacaagcaagggtgctaggggctcagctatacgaccattctccctgacagggagtccaaacttggcctagcatccctcctggcccccctctggccacgacttggcctgtgcctggttctctatcagaaaggggatgctgaacaaaacctccttccaagttttatccaattcgttcctcattgcctcgggctgcgtcaggggaagcaggggacaggtgtccagttgctgggccgagggaggagctggtttggcataggacctaaccagtgaagctagaggctacagccactaaacttgcttcaggccaacgatagttactcacaagtaagtaccttaatgctaatgaggtccactaaaaaggggaggaaggcagacctcctgggagacccacgaagggtttttagccagggaaaactgagccccaggaaaacctaaccactgggcaggcagaatttgtttgagggatagaacgacaacaaaataaatgttcctgcagcctgagatttcaggtagagtactgactaaggtttaataagacaataggtgacctgaggacatgcaagcttgtaaaatgcaacagcctcctgctagagtgacttgtacatgagcttgcttgcagaagactagattagatgtttctcaggatcccctcctgcgcaggggttctctgattttcgtgttctctgcccagatgggctgggggagttgagagtgtgcttattttcactgcgatcatgagaccacagttctgggttatctcctctcatacatcaagccccagaggaggcggcaagaggaacagccacaaacaagtactttaccccacagcttagtggccagtaaacaccctggggactaggaaaaggaaccaactgtaggcacctctccagggcctagggagacaagtgtcctctcttctgcatacatttgggctccccttacagagccctttgccctggctctctggtccttgttgctctaacagtccagatgtacacccagcctcagggggaaggcagctctctccagacagagtctcagggcccagcaaggtcaggttatctgctttcattcagggcaacaaatgatacaaatggtgccagggagtggcaaggccatgggggtaggtgggggtgtctttttcttttcataaagtaacaacagacgagactgaggttaaacatcagaaaaaaacctctggaatgaccttcctcattccaggaggccctggaataaggaagaggcttctttctgagggagctttgaggaattttgacagctgttgacatgggatttgggaaaggtgaagctgtgactggaggggcaggagatggtccaagtgtccatccagagatgagactcttagaatcaaagtgttcagcccaggaagtcttggagatcccaccttctgtggccctgcaccttatgggaagccattaagggggctcatctaggaattctggttacagcccagtgctcatcccagcgtatgctgcctctttagggcagccccaagggccagccagcctgtactctgggcaagagcccaaaatggctaggaatgtttgactcccttaatctcttccccagctacagaggaatcttttctctgcctggtctcagaatgggactgccaactggctcattggtgggagacacagtatcctcaaacctgtggccactggcatgacagtggtgctctgtctccctgggtgacacccaccctaggcttcctcctggatgtgatggggattgccagagaggctcttagcataaaaggcattaggtgggcatttttctgtgtgcccccaaaaagctccatggaaacaggcacctggtagctgcggaacacccgtggacttgtgtatatggtcataggctttgggaagacaggacgtaaaggaaaatgagagaaacaaaatgggtcagatagctttggccacagccccaggcagcctttggggcctatgacacttagtgcccttagatgggatacatcttgcctcggccccaagactcctccaacttacccgtcccatccagggcctgcacagcttagagaggctcacagcttggcaaatgctagggcttcatcagaccactgacttgactcagtgtttgttaaaatggaaccactcccgttggcctactgtttctctcctgtacttcttgtaatgatagttatttattgactctggtagcaggcagttcttaaataaagatggtttctcaacctgttggggaaaaaaaaaaaaaaa

SEQ ID NO: 10 - protein, 1040 aa, Homo sapien
MGTATRRKPHLLLVAAVALVSSSAWSSALGSQTTFGPVFEDQPLSVLFPEESTEEQVLLACRARASPPATYRWKMNGTEMKLEPGSRHQLVGGNLVIMNPTKAQDAGVYQCLASNPVGTVVSREAILRFGFLQEFSKEERDPVKAHEGWGVMLPCNPPAHYPGLSYRWLLNEFPNFIPTDGRHFVSQTTGNLYIARTNASDLGNYSCLATSHMDFSTKSVFSKFAQLNLAAEDTRLFAPSIKARFPAETYALVGQQVTLECFAFGNPVPRIKWRKVDGSLSPQWTTAEPTLQIPSVSFEDEGTYECEAENSKGRDTVQGRIIVQAQPEWLKVISDTEADIGSNLRWGCAAAGKPRPTVRWLRNGEPLASQNRVEVLAGDLRFSKLSLEDSGMYQCVAENKHGTIYASAELAVQALAPDFRLNPVRRLIPAARGGEILIPCQPRAAPKAVVLWSKGTEILVNSSRVTVTPDGTLIIRNISRSDEGKYTCFAENFMGKANSTGILSVRDATKITLAPSSADINLGDNLTLQCHASHDPTMDLTFTWTLDDFPIDFDKPGGHYRRTNVKETIGDLTILNAQLRHGGKYTCMAQTVVDSASKEATVLVRGPPGPPGGVVVRDIGDTTIQLSWSRGFDNHSPIAKYTLQARTPPAGKWKQVRTNPANIEGNAETAQVLGLTPWMDYEFRVIASNILGTGEPSGPSSKIRTREAAPSVAPSGLSGGGGAPGELIVNWTPMSREYQNGDGFGYLLSFRRQGSTHWQTARVPGADAQYFVYSNESVRPYTPFEVKIRSYNRRGDGPESLTALVYSAEEEPRVAPTKVWAKGVSSSEMNVTWEPVQQDMNGILLGYEIRYWKAGDKEAAADRVRTAGLDTSARVSGLHPNTKYHVTVRAYNRAGTGPASPSANATTMKPPPRRPPGNISWTFSSSSLSIKWDPVVPFRNESAVTGYKMLYQNDLHLTPTLHLTGKNWIEIPVPEDIGHALVQIRTTGPGGDGIPAEVHIVRNGGTSMMVENMAVRPAPHPGTVISHSVAMLILIGSLEL

SEQ ID NO: 11 - DNA, 1168 bp, Homo sapien
gagctggaagtgagagcagatccctaaccatgagcaccagccaaccaggggcctgcccatgccagggagctgcaagccgccccgccattctctacgcacttctgagctccagcctcaaggctgtcccccgaccccgtagccgctgcctatgtaggcagcaccggcccgtccagctatgtgcacctcatcgcacctgccgggaggccttggatgttctggccaagacagtggccttcctcaggaacctgccatccttctggcagctgcctccccaggaccagcggcggctgctgcagggttgctggggccccctcttcctgcttgggttggcccaagatgctgtgacctttgaggtggctgaggccccggtgcccagcatactcaagaagattctgctggaggagcccagcagcagtggaggcagtggccaactgccagacagaccccagccctccctggctgcggtgcagtggcttcaatgctgtctggagtccttctggagcctggagcttagccccaaggaatatgcctgcctgaaagggaccatcctcttcaaccccgatgtgccaggcctccaagccgcctcccacattgggcacctgcagcaggaggctcactgggtgctgtgtgaagtcctggaaccctggtgcccagcagcccaaggccgcctgacccgtgtcctcctcacggcctccaccctcaagtccattccgaccagcctgcttggggacctcttctttcgccctatcattggagatgttgacatcgctggccttcttggggacatgcttttgctcaggtgacctgttccagcccaggcagagatcaggtgggcagaggctggcagtgctgattcagcctggccatccccagaggtgacccaatgctcctggaggggcaagcctgtatagacagcacttggctccttaggaacagctcttcactcagccacaccccacattggacttccttggtttggacacagtgctccagctgcctgggaggcttttggtggtccccacagcctctgggccaagactcctgtcccttcttgggatgagaatgaaagcttaggctgcttattggaccagaagtcctatcgactttatacagaactgaattaagttattgatttttgtaataaaaggtatgaaacactaaaaaaaaa

SEQ ID NO: 12 - protein, 257 aa, Homo sapien
MSTSQPGACPCQGAASRPAILYALLSSSLKAVPRPRSRCLCRQHRPVQLCAPHRTCREALDVLAKTVAFLRNLPSFWQLPPQDQRRLLQGCWGPLFLLGLAQDAVTFEVAEAPVPSILKKILLEEPSSSGGSGQLPDRPQPSLAAVQWLQCCLESFWSLELSPKEYACLKGTILFNPDVPGLQAASHIGHLQQEAHWVLCEVLEPWCPAAQGRLTRVLLTASTLKSIPTSLLGDLFFRPIIGDVDIAGLLGDMLLLR

SEQ ID NO: 13 - DNA, 1998 bp, Homo sapien
cggcgcgatgcgcggagacccccgcgggggcggcggcggccgtgagccccgatgaggcccgagcgtccccggccgcgcggcagcgcccccggcccgatggagaccccgccgtgggacccagcccgcaacgactcgctgccgcccacgctgaccccggccgtgcccccctacgtgaagcttggcctcaccgtcgtctacaccgtgttctacgcgctgctcttcgtgttcatctacgtgcagctctggctggtgctgcgttaccgccacaagcggctcagctaccagagcgtcttcctctttctctgcctcttctgggcctccctgcggaccgtcctcttctccttctacttcaaagacttcgtggcggccaattcgctcagccccttcgtcttctggctgctctactgcttccctgtgtgcctgcagtttttcaccctcacgctgatgaacttgtacttcacgcaggtgattttcaaagccaagtcaaaatattctccagaattactcaaataccggttgcccctctacctggcctccctcttcatcagccttgttttcctgttggtgaatttaacctgtgctgtgctggtaaagacgggaaattgggagaggaaggttatcgtctctgtgcgagtggccattaatgacacgctcttcgtgctgtgtgccgtctctctctccatctgtctctacaaaatctctaagatgtccttagccaacatttacttggagtccaagggctcctccgtgtgtcaagtgactgccatcggtgtcaccgtgatactgctttacacctctcgggcctgctacaacctgttcatcctgtcattttctcagaacaagagcgtccattcctttgattatgactggtacaatgtatcagaccaggcagatttgaagaatcagctgggagatgctggatacgtattatttggagtggtgttatttgtttgggaactcttacctaccaccttagtcgtttatttcttccgagttagaaatcctacaaaggaccttaccaaccctggaatggtccccagccatggattcagtcccagatcttatttctttgacaaccctcgaagatatgacagtgatgatgaccttgcctggaacattgcccctcagggacttcagggaggttttgctccagattactatgattggggacaacaaactaacagcttcctggcacaagcaggaactttgcaagactcaactttggatcctgacaaaccaagccttgggtagcatcagttaacagttttatggacgattcctcagatgaaaagcttcagaaaagcatagtgacagctgaatttttagggcacttttccttaagaaatagaacttgatttttatttgttacaggtttccaatggccccataggaataagcaataatgtagactgataaacccttattttagtactaaagagggagccttgctatttcagtgggtataatttaaactttttaaagaaaatctgtacttttataaagatgtattttgtataacttaaataataatgctaaagtatactagggtttttttttcttgagaatgttactgcaatcatgttgtagtttgcacagacttttatgcataattcactttaaaaatatagaatatatggtctaatagttttttaaagcttttggactaaagtattccacaaatcttacctctttaggtcactgatggtcactccgattctgagtgccacattggtagactcctaaaatacagttgacaacttagccaattgcaactccagtgttgataattaaaatgaaatggtaaagcagcagactgtaaggtctttagagatttttttttaaggttcaggccgtaggttcctcaaggaatctcttaagttttgcccaaagactggtacttcctttcagtagggcgctaatgtatacacattaatgataagttgataacattaaaaatgtagctgacttatcctattaaacctcctctgctatgttcac

SEQ ID NO: 14 - protein, 399 aa, Homo sapien
MRPERPRPRGSAPGPMETPPWDPARNDSLPPTLTPAVPPYVKLGLTVVYTVFYALLFVFIYVQLWLVLRYRHKRLSYQSVFLFLCLFWASLRTVLFSFYFKDFVAANSLSPFVFWLLYCFPVCLQFFTLTLMNLYFTQVIFKAKSKYSPELLKYRLPLYLASLFISLVFLLVNLTCAVLVKTGNWERKVIVSVRVAINDTLFVLCAVSLSICLYKISKMSLANIYLESKGSSVCQVTAIGVTVILLYTSRACYNLFILSFSQNKSVHSFDYDWYNVSDQADLKNQLGDAGYVLFGVVLFVWELLPTTLVVYFFRVRNPTKDLTNPGMVPSHGFSPRSYFFDNPRRYDSDDDLAWNIAPQGLQGGFAPDYYDWGQQTNSFLAQAGTLQDSTLDPDKPSLG

SEQ ID NO: 15 - DNA, 2320 bp, Homo sapien
gcgcagggaggggagaccttggcggacggcggagccccagcggaggtgaaagtattggcggaaaggaaaatacagcggaaaaatgcagagctggagtcgtgtgtactgctccttggccaagagaggccatttcaatcgaatatctcatggcctacagggactttctgcagtgcctctgagaacttacgcagatcagccgattgatgctgatgtaacagttataggttctggtcctggaggatatgttgctgctattaaagctgcccagttaggcttcaagacagtctgcattgagaaaaatgaaacacttggtggaacatgcttgaatgttggttgtattccttctaaggctttattgaacaactctcattattaccatatggcccatggaacagattttgcatctagaggaattgaaatgtccgaagttcgcttgaatttagacaagatgatggagcagaagagtactgcagtaaaagctttaacaggtggaattgcccacttattcaaacagaataaggttgttcatgtcaatggatatggaaagataactggcaaaaatcaagtcactgctacgaaagctgatggcggcactcaggttattgatacaaagaacattcttatagccacgggttcagaagttactccttttcctggaatcacgatagatgaagatacaatagtgtcatctacaggtgctttatctttaaaaaaagttccagaaaagatggttgttattggtgcaggagtaataggtgtagaattgggttcagtttggcaaagacttggtgcagatgtgacagcagttgaatttttaggtcatgtaggtggagttggaattgatatggagatatctaaaaactttcaacgcatccttcaaaaacaggggtttaaatttaaattgaatacaaaggttactggtgctaccaagaagtcagatggaaaaattgatgtttctattgaagctgcttctggtggtaaagctgaagttatcacttgtgatgtactcttggtttgcattggccgacgaccctttactaagaatttgggactagaagagctgggaattgaactagatcctagaggtagaattccagtcaataccagatttcaaactaaaattccaaatatctatgccattggtgatgtagttgctggtccaatgctggctcacaaagcagaggatgaaggcattatctgtgttgaaggaatggctggtggtgctgtgcacattgactacaattgtgtgccatcagtgatttacacacaccctgaagttgcttgggttggcaaatcagaagagcagttgaaagaagagggtattgagtacaaagttgggaaattcccatttgctgctaacagcagagctaagacaaatgctgacacagatggcatggtgaagatccttgggcagaaatcgacagacagagtactgggagcacatattcttggaccaggtgctggagaaatggtaaatgaagctgctcttgctttggaatatggagcatcctgtgaagatatagctagagtctgtcatgcacatccgaccttatcagaagcttttagagaagcaaatcttgctgcgtcatttggcaaatcaatcaacttttgaattagaagattatatatttttttttctgaaatttcctgggagcttttgtagaagtcacattcctgaacaggatattctcacagctccaagaatttctaggactgaattatgaaacttttggaaggtatttaataggtttggacaaaatggaatactcttatatctatattttacataaatttagtattttgtttcagtgcactaatatgtaagacaaaaaggactacttattgtagtcatcctggaatatctccgtcaactcatattttcatgctgttcatgaaagattcaatgcccctgaatttaaatagctcttttctctgatacagaaaagttgaattttacatggctggagctagaatttgatatgtgaacagttgtgtttgaagcacagtgatcaagttatttttaatttggttttcacattggaaacaagtcagtcattcagatatgattcaaatgtctataaaccaaactgatgtaagtaaatggtctctcacttgttttatttaacctctaaattctttcattttaggggtagcatttgtgttgaagaggttttaaagcttccattgttgtctgcaactctgaagggtaattatatagttacccaaattaagagagtctatttacggaactcaaatacgtgggcattcaaatgtattacagtggggaatgaagatactgaaataaacgtcttaaatattc

SEQ ID NO: 16 - protein, 509 aa, Homo sapien
MQSWSRVYCSLAKRGHFNRISHGLQGLSAVPLRTYADQPIDADVTVIGSGPGGYVAAIKAAQLGFKTVCIEKNETLGGTCLNVGCIPSKALLNNSHYYHMAHGTDFASRGIEMSEVRLNLDKMMEQKSTAVKALTGGIAHLFKQNKVVHVNGYGKITGKNQVTATKADGGTQVIDTKNILIATGSEVTPFPGITIDEDTIVSSTGALSLKKVPEKMVVIGAGVIGVELGSVWQRLGADVTAVEFLGHVGGVGIDMEISKNFQRILQKQGFKFKLNTKVTGATKKSDGKIDVSIEAASGGKAEVITCDVLLVCIGRRPFTKNLGLEELGIELDPRGRIPVNTRFQTKIPNIYAIGDVVAGPMLAHKAEDEGIICVEGMAGGAVHIDYNCVPSVIYTHPEVAWVGKSEEQLKEEGIEYKVGKFPFAANSRAKTNADTDGMVKILGQKSTDRVLGAHILGPGAGEMVNEAALALEYGASCEDIARVCHAHPTLSEAFREANLAASFGKSINF

SEQ ID NO: 17 - DNA, 2090 bp, Homo sapien
gttctgggcctaggggaggcgggccgagggcgtctgagctgaggcccgcgtcgatcctgggttggaggaggtggcggccgctgaggctgcggcgtgaagacggcgggcatggtggggcgggagaaagagctctctatacactttgttcccgggagctgtcggctggtggaggaggaagttaacatccctaataggagggttctggttactggtgccactgggcttcttggcagagctgtacacaaagaatttcagcagaataattggcatgcagttggctgtggtttcagaagagcaagaccaaaatttgaacaggttaatctgttggattctaatgcagttcatcacatcattcatgattttcagccccatgttatagtacattgtgcagcagagagaagaccagatgttgtagaaaatcagccagatgctgcctctcaacttaatgtggatgcttctgggaatttagcaaaggaagcagctgctgttggagcatttctcatctacattagctcagattatgtatttgatggaacaaatccaccttacagagaggaagacataccagctcccctaaatttgtatggcaaaacaaaattagatggagaaaaggctgtcctggagaacaatctaggagctgctgttttgaggattcctattctgtatggggaagttgaaaagctcgaagaaagtgctgtgactgttatgtttgataaagtgcagttcagcaacaagtcagcaaacatggatcactggcagcagaggttccccacacatgtcaaagatgtggccactgtgtgccggcagctagcagagaagagaatgctggatccatcaattaagggaacctttcactggtctggcaatgaacagatgactaagtatgaaatggcatgtgcaattgcagatgccttcaacctccccagcagtcacttaagacctattactgacagccctgtcctaggagcacaacgtccgagaaatgctcagcttgactgctccaaattggagaccttgggcattggccaacgaacaccatttcgaattggaatcaaagaatcactttggcctttcctcattgacaagagatggagacaaacggtctttcattagtttatttgtgttgggttcttttttttttttaaatgaaaagtatagtatgtggcactttttaaagaacaaaggaaatagttttgtatgagtactttaattgtgactcttaggatctttcaggtaaatgatgctcttgcactagtgaaattgtctaaagaaactaaagggcagtcatgccctgtttgcagtaatttttctttttatcattttgtttgtcctggctaaacttggagtttgagtatagtaaattatgatccttaaatatttgagagtcaggatgaagcagatctgctgtagacttttcagatgaaattgttcattctcgtaacctccatattttcaggatttttgaagctgttgaccttttcatgttgattattttaaattgtgtgaaatagtataaaaatcattggtgttcattatttgctttgcctgagctcagatcaaaatgtttgaagaaaggaactttatttttgcaagttacgtacagtttttatgcttgagatatttcaacatgttatgtatattggaacttctacagcttgatgcctcctgcttttatagcagtttatggggagcacttgaaagagcgtgtgtacatgtattttttttctaggcaaacattgaatgcaaacgtgtatttttttaatataaatatataactgtccttttcatcccatgttgccgctaagtgatatttcatatgtgtggttatactcataataatgggccttgtaagtcttttcaccattcatgaataataataaatatgtactgctggcatgtaatgcttagttttcttgtatttacttctttttttaaatgtaaggaccaaacttctaaactaattgttcttttgttgctttaatttttaaaaattacattcttctgatgtaacatgtgatacatacaaaagaatatagtttaatatgtattgaaataaaacacaataaaattaaaaaaaaaaaaaaaaaaaa

SEQ ID NO: 18 - protein, 334 aa, Homo sapien
MVGREKELSIHFVPGSCRLVEEEVNIPNRRVLVTGATGLLGRAVHKEFQQNNWHAVGCGFRRARPKFEQVNLLDSNAVHHIIHDFQPHVIVHCAAERRPDVVENQPDAASQLNVDASGNLAKEAAAVGAFLIYISSDYVFDGTNPPYREEDIPAPLNLYGKTKLDGEKAVLENNLGAAVLRIPILYGEVEKLEESAVTVMFDKVQFSNKSANMDHWQQRFPTHVKDVATVCRQLAEKRMLDPSIKGTFHWSGNEQMTKYEMACAIADAFNLPSSHLRPITDSPVLGAQRPRNAQLDCSKLETLGIGQRTPFRIGIKESLWPFLIDKRWRQTVFH

SEQ ID NO: 19 - DNA, 2380 bp, Homo sapien
gaggaggagggaaaaggcgagcaaaaaggaagagtgggaggaggaggggaagcggcgaaggaggaagaggaggaggaggaagaggggagcacaaaggatccaggtctcccgacgggaggttaataccaagaaccatgtgtgccgagcggctgggccagttcatgaccctggctttggtgttggccacctttgacccggcgcgggggaccgacgccaccaacccacccgagggtccccaagacaggagctcccagcagaaaggccgcctgtccctgcagaatacagcggagatccagcactgtttggtcaacgctggcgatgtggggtgtggcgtgtttgaatgtttcgagaacaactcttgtgagattcggggcttacatgggatttgcatgacttttctgcacaacgctggaaaatttgatgcccagggcaagtcattcatcaaagacgccttgaaatgtaaggcccacgctctgcggcacaggttcggctgcataagccggaagtgcccggccatcagggaaatggtgtcccagttgcagcgggaatgctacctcaagcacgacctgtgcgcggctgcccaggagaacacccgggtgatagtggagatgatccatttcaaggacttgctgctgcacgaaccctacgtggacctcgtgaacttgctgctgacctgtggggaggaggtgaaggaggccatcacccacagcgtgcaggttcagtgtgagcagaactggggaagcctgtgctccatcttgagcttctgcacctcggccatccagaagcctcccacggcgccccccgagcgccagccccaggtggacagaaccaagctctccagggcccaccacggggaagcaggacatcacctcccagagcccagcagtagggagactggccgaggtgccaagggtgagcgaggtagcaagagccacccaaacgcccatgcccgaggcagagtcgggggccttggggctcagggaccttccggaagcagcgagtgggaagacgaacagtctgagtattctgatatccggaggtgaaatgaaaggcctggccacgaaatctttcctccacgccgtccattttcttatctatggacattccaaaacatttaccattagagaggggggatgtcacacgcaggattctgtggggactgtggacttcatcgaggtgtgtgttcgcggaacggacaggtgagatggagacccctggggccgtggggtctcaggggtgcctggtgaattctgcacttacacgtactcaagggagcgcgcccgcgttatcctcgtacctttgtcttctttccatctgtggagtcagtgggtgtcggccgctctgttgtgggggaggtgaaccagggaggggcagggcaaggcagggcccccagagctgggccacacagtgggtgctgggcctcgccccgaagcttctggtgcagcagcctctggtgctgtctccgcggaagtcagggcggctggattccaggacaggagtgaatgtaaaaataaatatcgcttagaatgcaggagaagggtggagaggaggcaggggccgagggggtgcttggtgccaaactgaaattcagtttcttgtgtggggccttgcggttcagagctcttggcgagggtggagggaggagtgtcatttctatgtgtaatttctgagccattgtactgtctgggctgggggggacactgtccaagggagtggcccctatgagtttatattttaaccactgcttcaaatctcgatttcactttttttatttatccagttatatctacatatctgtcatctaaataaatggctttcaaacaaagcaactgggtcattaaaaccagctcaaagggggtttaaaaaaaaaaaaaccagcccatcctttgaggctgatttttcttttttttaagttctattttaaaagctatcaaacagcgacatagccatacatctgactgcctgacatggactcctgcccacttgggggaaaccttatacccagaggaaaatacacacctggggagtacatttgacaaatttcccttaggatttcgttatctcaccttgaccctcagccaagattggtaaagctgcgtcctggcgattccaggagacccagctggaaacctggcttctccatgtgaggggatgggaaaggaaagaagagaatgaagactacttagtaattcccatcaggaaatgctgaccttttacataaaatcaaggagactgctgaaaatctctaagggacaggattttccagatcctaattggaaatttagcaataaggagaggagtccaaggggacaaataaaggcagagagagagagagagagagggagaggaagaaaagagagagagaaaagagcctcgtgcc

SEQ ID NO: 20 - protein, 302 aa, Homo sapien
MCAERLGQFMTLALVLATFDPARGTDATNPPEGPQDRSSQQKGRLSLQNTAEIQHCLVNAGDVGCGVFECFENNSCEIRGLHGICMTFLHNAGKFDAQGKSFIKDALKCKAHALRHRFGCISRKCPAIREMVSQLQRECYLKHDLCAAAQENTRVIVEMIHFKDLLLHEPYVDLVNLLLTCGEEVKEAITHSVQVQCEQNWGSLCSILSFCTSAIQKPPTAPPERQPQVDRTKLSRAHHGEAGHHLPEPSSRETGRGAKGERGSKSHPNAHARGRVGGLGAQGPSGSSEWEDEQSEYSDIRR

SEQ ID NO: 21 - DNA, 2516 bp, Homo sapien
gttcctggtgtccccacttcgcctccctcctgctgcccccaagacatgcaggggccctgggtgctgctgctgctgggcctgaggctacagctctccctgggcgtcatcccagctgaggaggagaacccggccttctggaaccgccaggcagctgaggccctggatgctgccaagaagctgcagcccatccagaaggtcgccaagaacctcatcctcttcctgggcgatgggttgggggtgcccacggtgacagccaccaggatcctaaaggggcagaagaatggcaaactggggcctgagacgcccctggccatggaccgcttcccatacctggctctgtccaagacatacaatgtggacagacaggtgccagacagcgcagccacagccacggcctacctgtgcggggtcaaggccaacttccagaccatcggcttgagtgcagccgcccgctttaaccagtgcaacacgacacgcggcaatgaggtcatctccgtgatgaaccgggccaagcaagcaggaaagtcagtaggagtggtgaccaccacacgggtgcagcacgcctcgccagccggcacctacgcacacacagtgaaccgcaactggtactcagatgctgacatgcctgcctcagcccgccaggaggggtgccaggacatcgccactcagctcatctccaacatggacattgacgtgatccttggcggaggccgcaagtacatgtttcccatggggaccccagaccctgagtacccagctgatgccagccagaatggaatcaggctggacgggaagaacctggtgcaggaatggctggcaaagcaccagggtgcctggtatgtgtggaaccgcactgagctcatgcaggcgtccctggaccagtctgtgacccatctcatgggcctctttgagcccggagacacgaaatatgagatcctccgagaccccacactggacccctccctgatggagatgacagaggctgccctgcgcctgctgagcaggaacccccgcggcttctacctctttgtggagggcggccgcatcgaccatggtcatcatgagggtgtggcttaccaggcagtcactgaggcggtcatgttcgacgacgccattgagagggcgggccagctcaccagcgaggaggacacgctgaccctcgtcaccgctgaccactcccatgtcttctcctttggtggctacaccttgcgagggagctccatcttcgggttggcccccagcaaggctcaggacagcaaagcctacacgtccatcctgtacggcaatggcccgggctacgtgttcaactcaggcgtgcgaccagacgtgaatgagagcgagagcgggagccccgattaccagcagcaggcggcggtgcccctgtcgtccgagacccacggaggcgaagacgtggcggtgtttgcgcgcggcccgcaggcgcacctggtgcatggtgtgcaggagcagagcttcgtagcgcatgtcatggccttcgctgcctgtctggagccctacacggcctgcgacctggcgctccccgcctgcaccaccgacgccgcgcacccagttgccgcgtcgctgccactgctggccgggaccctgctgctgctgggggcgtccgctgctccctgagtgccccactccggagttatcctgctccccacctccgggcgtcctgccctgttccccgtcctgagccgccacttccagcgaacacacacaggtgtcctgccgttggaccttcacctcctagagataaaccagcctcagctggcgcagcggggcccttcttccctccgcatccccttcagggagcaggagcccagggcgccctgggagctgagcctgggacttccaggacctcccctcaggttgttctctgattcttcctcccaaccccagagactgcagatttgtgccatgcggctgcctgcaccccagacaataaagggaccaaaaccacccaacccccaccctgcctctatcctaaggaagaccaagcaggcctggacccagagacgtcccccatcgtgggacacgacacacccagaccgcgtgccccaccgtcttagcttcaatcctggcagcacctggtagacccaaggacttgggtggatcaggacacctgaagaagagaagcttccggcaaccctgcaacccacccaaggaggctactggatcggggattcccaggggggctttgacacagtcctctgctgtctccccactaggatcattccacacccctgcacctgaccaagggaccaatgaggcagaggcttgccccaagtcacagccactcagatgcttcctgccccccagtgcccattccaggtcaccagatccaaggagcgcttgaggagctctgggtacagggcagcaacccagagcccatgggccctcccgggacatctggatgctgggcatagatttctcaacaaggaagactcccctgcctcctcaaggtctccattctcctaggagacaaagcaataataaaaggtgttagacaatgt

SEQ ID NO: 22 - protein, 528 aa, Homo sapien
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEILRDPTLDPSLMEMTEAALRLLSRNPRGFYLFVEGGRIDHGHHEGVAYQAVTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLALPACTTDAAHPVAASLPLLAGTLLLLGASAAP

SEQ ID NO: 23 - DNA, 1746 bp, Homo sapien
agaattcggcacgacggggttctggccatgaagcccacctcaggcccagaggaggcccggcggccagcctcggacatccgcgtgttcgccagcaactgctcgatgcacgggctgggccacgtcttcgggccaggcagcctgagcctgcgccgggggatgtgggcagcggccgtggtcctgtcagtggccaccttcctctaccaggtggctgagagggtgcgctactacagggagttccaccaccagactgccctggatgagcgagaaagccaccggctcatcttcccggctgtcaccctgtgcaacatcaacccactgcgccgctcgcgcctaacgcccaacgacctgcactgggctgggtctgcgctgctgggcctggatcccgcagagcacgccgccttcctgcgcgccctgggccggccccctgcaccgcccggcttcatgcccagtcccacctttgacatggcgcaactctatgcccgtgctgggcactccctggatgacatgctgctggactgtcgcttccgtggccaaccttgtgggcctgagaacttcaccacgatcttcacccggatgggaaagtgctacacatttaactctggcgctgatggggcagagctgctcaccactactaggggtggcatgggcaatgggctggacatcatgctggacgtgcagcaggaggaatatctacctgtgtggagggacaatgaggagaccccgtttgaggtggggatccgagtgcagatccacagccaggaggagccgcccatcatcgatcagctgggcttgggggtgtccccgggctaccagacctttgtttcttgccagcagcagcagctgagcttcctgccaccgccctggggcgattgcagttcagcatctctgaaccccaactatgagccagagccctctgatcccctaggctcccccagccccagccccagccctccctatacccttatggggtgtcgcctggcctgcgaaacccgctacgtggctcggaagtgcggctgccgaatggtgtacatgccaggcgacgtgccagtgtgcagcccccagcagtacaagaactgtgcccacccggccatagatgccatgcttcgcaaggactcgtgcgcctgccccaacccgtgcgccagcacgcgctacgccaaggagctctccatggtgcggatcccgagccgcgccgccgcgcgcttcctggcccggaagctcaaccgcagcgaggcctacatcgcggagaacgtgctggccctggacatcttctttgaggccctcaactatgagaccgtggagcagaagaaggcctatgagatgtcagagctgcttggtgacattgggggccagatggggctgttcatcggggccagcctgctcaccatcctcgagatcctagactacctctgtgaggtgttccgagacaaggtcctgggatatttctggaaccgacagcactcccaaaggcactccagcaccaatctgcttcaggaagggctgggcagccatcgaacccaagttccccacctcagcctgggccccagacctcccacccctccctgtgccgtcaccaagactctctccgcctcccaccgcacctgctaccttgtcacacagctctagacctgctgtctgtgtcctcggagccccgccctgacatcctggacatgcctagcctgcacgtagcttttccgtcttcaccccaaataaagtcctaatgcatcaaaaaaaaaaaaaaaaaaaaaa

SEQ ID NO: 24 - protein, 531 aa, Homo sapien
MKPTSGPEEARRPASDIRVFASNCSMHGLGHVFGPGSLSLRRGMWAAAVVLSVATFLYQVAERVRYYREFHHQTALDERESHRLIFPAVTLCNINPLRRSRLTPNDLHWAGSALLGLDPAEHAAFLRALGRPPAPPGFMPSPTFDMAQLYARAGHSLDDMLLDCRFRGQPCGPENFTTIFTRMGKCYTFNSGADGAELLTTTRGGMGNGLDIMLDVQQEEYLPVWRDNEETPFEVGIRVQIHSQEEPPIIDQLGLGVSPGYQTFVSCQQQQLSFLPPPWGDCSSASLNPNYEPEPSDPLGSPSPSPSPPYTLMGCRLACETRYVARKCGCRMVYMPGDVPVCSPQQYKNCAHPAIDAMLRKDSCACPNPCASTRYAKELSMVRIPSRAAARFLARKLNRSEAYIAENVLALDIFFEALNYETVEQKKAYEMSELLGDIGGQMGLFIGASLLTILEILDYLCEVFRDKVLGYFWNRQHSQRHSSTNLLQEGLGSHRTQVPHLSLGPRPPTPPCAVTKTLSASHRTCYLVTQL

SEQ ID NO: 25 - DNA, 1104 bp, Homo sapien
ctcggtgcgcgaccccggctcagaggactctttgctgtcccgcaagatgcggatgctgctggcgctcctggccctctccgcggcgcggccatcggccagtgcagagtcacactggtgctacgaggttcaagccgagtcctccaactacccctgcttggtgccagtcaagtggggtggaaactgccagaaggaccgccagtcccccatcaacatcgtcaccaccaaggcaaaggtggacaaaaaactgggacgcttcttcttctctggctacgataagaagcaaacgtggactgtccaaaataacgggcactcagtgatgatgttgctggagaacaaggccagcatttctggaggaggactgcctgccccataccaggccaaacagttgcacctgcactggtccgacttgccatataagggctcggagcacagcctcgatggggagcactttgccatggagatgcacatagtacatgagaaagagaaggggacatcgaggaatgtgaaagaggcccaggaccctgaagacgaaattgcggtgctggcctttctggtggaggctggaacccaggtgaacgagggcttccagccactggtggaggcactgtctaatatccccaaacctgagatgagcactacgatggcagagagcagcctgttggacctgctccccaaggaggagaaactgaggcactacttccgctacctgggctcactcaccacaccgacctgcgatgagaaggtcgtctggactgtgttccgggagcccattcagcttcacagagaacagatcctggcattctctcagaagctgtactacgacaaggaacagacagtgagcatgaaggacaatgtcaggcccctgcagcagctggggcagcgcacggtgataaagtccggggccccgggtcggccgctgccctgggccctgcctgccctgctgggccccatgctggcctgcctgctggccggcttcctgcgatgatggctcacttctgcacgcagcctctctgttgcctcagctctccaagttccaggcttccggtccttagccttcccaggtgggactttaggcatgattaaaatatggacatatttttggag

SEQ ID NO: 26 - protein, 312 aa, Homo sapien
MRMLLALLALSAARPSASAESHWCYEVQAESSNYPCLVPVKWGGNCQKDRQSPINIVTTKAKVDKKLGRFFFSGYDKKQTWTVQNNGHSVMMLLENKASISGGGLPAPYQAKQLHLHWSDLPYKGSEHSLDGEHFAMEMHIVHEKEKGTSRNVKEAQDPEDEIAVLAFLVEAGTQVNEGFQPLVEALSNIPKPEMSTTMAESSLLDLLPKEEKLRHYFRYLGSLTTPTCDEKVVWTVFREPIQLHREQILAFSQKLYYDKEQTVSMKDNVRPLQQLGQRTVIKSGAPGRPLPWALPALLGPMLACLLAGFLR

SEQ ID NO: 27 - DNA, 585 bp, Homo sapien
tggtcatctcagttcttttctcaccttgactgcaagatgaaactccttgtgctagctgtgctgctcacagtggccgccgccgacagcggcatcagccctcgggccgtgtggcagttccgcaaaatgatcaagtgcgtgatcccggggagtgaccccttcttggaatacaacaactacggctgctactgtggcttggggggctcaggcacccccgtggatgaactggacaagtgctgccagacacatgacaactgctatgaccaggccaagaagctggacagctgtaaatttctgctggacaacccgtacacccacacctattcatactcgtgctctggctcggcaatcacctgtagcagcaaaaacaaagagtgtgaggccttcatttgcaactgcgaccgcaacgctgccatctgcttttcaaaagctccatataacaaggcacacaagaacctggacaccaagaagtattgtcagagttgaatatcacctctcaaaagcatcacctctatctgcctcatctcacactgtactctccaataaagcaccttgttgaaagacctcaaaaaaaaaaaaaaaaaaaaa

SEQ ID NO: 28 - protein, 148 aa, Homo sapien
MKLLVLAVLLTVAAADSGISPRAVWQFRKMIKCVIPGSDPFLEYNNYGCYCGLGGSGTPVDELDKCCQTHDNCYDQAKKLDSCKFLLDNPYTHTYSYSCSGSAITCSSKNKECEAFICNCDRNAAICFSKAPYNKAHKNLDTKKYCQS

SEQ ID NO: 29 - DNA, 2876 bp, Homo sapien
tgaaacctaacccgccctggggaggcgcgcagcagaggctccgattcggggcaggtgagaggctgactttctctcggtgcgtccagtggagctctgagtttcgaatcggcggcggcggattccccgcgcgcccggcgtcggggcttccaggaggatgcggagccccagcgcggcgtggctgctgggggccgccatcctgctagcagcctctctctcctgcagtggcaccatccaaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccatccctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattgagttttccaggtcctcagatgggaattgcacagtaggatgtggaacctgtttaatgttatgaggacgtgtctgttatttcctaatcaaaaaggtctcaccacataccatgtggatgcagcacctctcaggattgctaggagctcccctgtttgcatgagaaaagtagtcccccaaattaacatcagtgtctgtttcagaatctctctactcagatgaccccagaaactgaaccaacagaagcagacttttcagaagatggtgaagacagaaacccagtaacttgcaaaaagtagacttggtgtgaagactcacttctcagctgaaattatatatatacacatatatatattttacatctgggatcatgatagacttgttagggcttcaaggccctcagagatgatcagtccaactgaacgaccttacaaatgaggaaaccaagataaatgagctgccagaatcaggtttccaatcaacagcagtgagttgggattggacagtagaatttcaatgtccagtgagtgaggttcttgtaccacttcatcaaaatcatggatcttggctgggtgcggtgcctcatgcctgtaatcctagcactttgggaggctgaggcaggcaatcacttgaggtcaggagttcgagaccagcctggccatcatggcgaaacctcatctctactaaaaatacaaaagttaaccaggtgtgtggtgcacgtttgtaatcccagttactcaggaggctgaggcacaagaattgagtatcactttaactcaggaggcagaggttgcagtgagccgagattgcaccactgcactccagcttgggtgataaaataaaataaaatagtcgtgaatcttgttcaaaatgcagattcctcagattcaataatgagagctcagactgggaacagggcccaggaatctgtgtggtacaaacctgcatggtgtttatgcacacagagatttgagaaccattgttctgaatgctgcttccatttgacaaagtgccgtgataatttttgaaaagagaagcaaacaatggtgtctcttttatgttcagcttataatgaaatctgtttgttgacttattaggactttgaattatttctttattaaccctctgagtttttgtatgtattattattaaagaaaaatgcaatcaggattttaaacatgtaaatacaaattttgtataacttttgatgacttcagtgaaattttcaggtagtctgagtaatagattgttttgccacttagaatagcatttgccacttagtattttaaaaaataattgttggagtatttattgtcagttttgttcacttgttatctaatacaaaattataaagccttcagagggtttggaccacatctctttggaaaatagtttgcaacatatttaagagatacttgatgccaaaatgactttatacaacgattgtatttgtgacttttaaaaataattattttattgtgtaattgatttataaataacaaaattttttttacaacttaaaaaaaaaaaaaaa

SEQ ID NO: 30 - protein, 397 aa, Homo sapien
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 31 - DNA, 3279 bp, Homo sapien
ccggctcgaagcgcaacgaggaagcgtttgcggtgatcccggcgactgcgctggctaatgcggtaccggctagcgtggcttctgcaccccgcactgcccagcaccttccgctcagtcctcggcgcccgcctgccgcctccggagcgcctgtgtggtttccaaaaaaagacttacagcaaaatgaataatccagccatcaagagaataggaaatcacattaccaagtctcctgaagacaagcgagaatatcgagggctagagctggccaatggtatcaaagtacttcttatgagtgatcccaccacggataagtcatcagcagcacttgatgtgcacataggttcattgtcggatcctccaaatattgctggcttaagtcatttttgtgaacatatgctttttttgggaacaaagaaataccctaaagaaaatgaatacagccagtttctcagtgagcatgcaggaagttcaaatgcctttactagtggagagcataccaattactattttgatgtttctcatgaacacctagaaggtgccctagacaggtttgcacagttttttctgtgccccttgttcgatgaaagttgcaaagacagagaggtgaatgcagttgattcagaacatgagaagaatgtgatgaatgatgcctggagactctttcaattggaaaaagctacagggaatcctaaacaccccttcagtaaatttgggacaggtaacaaatatactctggagactagaccaaaccaagaaggcattgatgtaagacaagagctactgaaattccattctgcttactattcatccaacttaatggctgtttgtgttttaggtcgagaatctttagatgacttgactaatctggtggtaaagttattttctgaagtagagaacaaaaatgttccattgccagaatttcctgaacaccctttccaagaagaacatcttaaacaactttacaaaatagtacccattaaagatattaggaatctctatgtgacatttcccatacctgaccttcagaaatactacaaatcaaatcctggtcattatcttggtcatctcattgggcatgaaggtcctggaagtctgttatcagaacttaagtcaaagggctgggttaatactcttgttggtgggcagaaggaaggagcccgaggttttatgttttttatcattaatgtggacttgaccgaggaaggattattacatgttgaagatataattttgcacatgtttcaatacattcagaagttacgtgcagaaggacctcaagaatgggttttccaagagtgcaaggacttgaatgctgttgcttttaggtttaaagacaaagagaggccacggggctatacatctaagattgcaggaatattgcattattatcccctagaagaggtgctcacagcggaatatttactggaagaatttagacctgacttaatagagatggttctcgataaactcagaccagaaaatgtccgggttgccatagtttctaaatcttttgaaggaaaaactgatcgcacagaagagtggtatggaacccagtacaaacaagaagctataccggatgaagtcatcaagaaatggcaaaatgctgacctgaatgggaaatttaaacttcctacaaagaatgaatttattcctacgaattttgagattttaccgttagaaaaagaggcgacaccataccctgctcttattaaggatacagtcatgagcaaactttggttcaaacaagatgataagaaaaaaaagccgaaggcttgtctcaactttgaatttttcagcccatttgcttatgtggaccccttgcactgtaacatggcctatttgtaccttgagctcctcaaagactcactcaacgagtatgcatatgcagcagagctagcaggcttgagctatgatctccaaaataccatctatgggatgtatctttcagtgaaaggttacaatgacaagcagccaattttactaaagaagattattgagaaaatggctacctttgagattgatgaaaaaagatttgaaattatcaaagaagcatatatgcgatctcttaacaatttccgggctgaacagcctcaccagcatgccatgtactacctccgcttgctgatgactgaagtggcctggactaaagatgagttaaaagaagctctggatgatgtaacccttcctcgccttaaggccttcatacctcagctcctgtcacggctgcacattgaagcccttctccatggaaacataacaaagcaggctgcattaggaattatgcagatggttgaagacaccctcattgaacatgctcataccaaacctctccttccaagtcagctggttcggtatagagaagttcagctccctgacagaggatggtttgtttatcagcagagaaatgaagttcacaataactgtggcatcgagatatactaccaaacagacatgcaaagcacctcagagaatatgtttctggagctcttctgtcagattatctcggaaccttgcttcaacaccctgcgcaccaaggagcagttgggctatatcgtcttcagcgggccacgtcgagctaatggcatacagagcttgagattcatcatccagtcagaaaagccacctcactacctagaaagcagagtggaagctttcttaattaccatggaaaagtccatagaggacatgacagaagaggccttccaaaaacacattcaggcattagcaattcgtcgactagacaaaccaaagaagctatctgctgagtgtgctaaatactggggagaaatcatctcccagcaatataattttgacagagataacactgaggttgcatatttaaagacacttaccaaggaagatatcatcaaattctacaaggaaatgttggcagtagatgctccaaggagacataaggtatccgtccatgttcttgccagggaaatggattcttgtcctgttgttggagagttcccatgtcaaaatgacataaatttgtcacaagcaccagccttgccacaacctgaagtgattcagaacatgaccgaattcaagcgtggtctgccactgtttccccttgtgaaaccacatattaacttcatggctgcaaaactctgaagattccccatgcatgggaaagtgcaagtggatgcattcctgagtcttccagagcctaagaaaatcatcttggccactttaatagtttctgattcactattagagaaacaaacaaaaaattgtcaaatgtcattatgtagaaatattataaatccaaagtaa

SEQ ID NO: 32 - protein, 1019 aa, Homo sapien
MRYRLAWLLHPALPSTFRSVLGARLPPPERLCGFQKKTYSKMNNPAIKRIGNHITKSPEDKREYRGLELANGIKVLLMSDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTVMSKLWFKQDDKKKKPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQSLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 33 - DNA, 3624 bp, Homo sapien
cagggagcctgggctggaagaggcagcaaaagggaaaatcagaagagtggacactggcaagaggagggcagcctttttcccagcttccttgcaccatggacagctcccattaagccacctctccatcctggggccaggactcttatgccccattcctgtcaaattgagatttcatccaccattctccaaggacagtgaagttataccctagttccagtgttgggatcagtggcccctctggacatgcctctcctggaaggttctgtgggggtggaggatcttgtcctcctggaacccttggtggaggagtcactgctcaagaatcttcagcttcgctatgaaaacaaggagatttatacctacattgggaatgtggtgatctcagtgaatccctatcaacagcttcccatctatgggccagagttcattgccaaatatcaagactatactttctatgagctgaagccccatatctacgcattggcaaatgtggcgtaccagtcactgagggacagggaccgagaccagtgtatcctcatcacaggcgagagtggatcagggaagactgaggccagcaagctggtgatgtcttatgtggctgccgtctgtgggaaaggagagcaggtgaactctgtgaaggagcagctgctacagtctaacccagtgctggaggcttttggcaatgccaagaccattcgcaacaacaattcctcccgatttggaaaatacatggatattgaatttgacttcaagggatcccccctcggtggtgtcatcacaaactatctgcttgagaaatcccgattagtgaagcagctcaaaggagaaaggaacttccacatcttctatcagctgctggctggagcagatgaacagctgctgaaggccctgaagcttgagcgggatacaactggctatgcctatctgaatcatgaagtatccagagtggatggcatggacgacgcctccagcttcagggctgtacagagtgcaatggcagtgattgggttctcggaggaggagattcgacaagtgctagaggtgacatccatggtgctaaagctggggaacgtgttggtggctgatgagttccaggccagtgggataccagcaagtggcatccgtgatgggagaggtgttcgggagattggggagatggtgggcttgaattcagaagaagtagagagagctttgtgctcgaggaccatggaaacagccaaggaaaaggtggtcactgcactgaatgttatgcaggctcagtatgctcgggacgccctggctaagaacatctacagccgcctctttgactggatagtgaatcgaatcaatgagagcatcaaggtgggcatcggggaaaagaagaaggtaatgggagtccttgatatctacggttttgagatattagaggataatagctttgagcaatttgtgatcaactactgcaatgagaagctgcagcaggtgttcatagagatgaccctgaaagaagagcaagaggaatataagagagaaggcataccgtggacaaaggtggactactttgataatggcatcatttgtaagctcattgagcataatcagcgaggtatcctggccatgttggatgaggagtgcctgcggcctggggtggtcagtgactccactttcctagcaaagctgaaccagctcttctccaagcatggccactacgagagcaaagtcacccagaatgcccagcgtcagtatgaccacaccatgggcctcagctgcttccgcatctgccactatgcgggcaaggtgacatacaacgtgaccagctttattgacaagaataatgacctactcttccgagacctgttgcaggccatgtggaaggcccagcaccccctccttcggtccttgtttcctgagggcaatcctaagcaggcatctctcaaacgccccccgactgctggggcccagttcaagagttctgtggccatcctcatgaagaatctgtattccaagagccccaactacatcaggtgcataaagcccaatgagcatcagcagcgaggtcagttctcttcagacctggtggcaacccaggctcggtacctgggactgctggagaacgtacgggtgcgacgggcaggctatgcccaccgccagggttatgggcccttcctggaaaggtaccgattgctgagccggagcacctggcctcactggaatgggggagaccgggaaggtgttgagaaggtcctgggggagctgagcatgtcctcgggggagctggcctttggcaagacaaagatcttcattagaagccccaagactcttttctacctcgaagaacagaggcgcctgagactccagcagctggccacactcatacagaagatttaccgaggctggcgctgccgcacccactaccaactgatgcgaaagagtcagatcctcatctcctcttggtttcggggaaacatgcaaaagaaatgctatgggaagataaaggcatccgtgttattgatccaggcttttgtgagagggtggaaggcccgaaagaattatcgcaaatatttccggtcagaggctgccctcaccttggcagatttcatctacaagagcatggtacagaaattcctactggggctgaagaacaatttgccatccacaaacgtcttagacaagacatggccagccgccccctacaagtgcctcagcacagcaaatcaggagctgcagcagctcttctaccagtggaagtgcaagaggttccgggatcagctgtccccgaagcaggtagagatcctgagggaaaagctctgtgccagtgaactgttcaagggcaagaaggcttcatatccccagagtgtccccattccattctgtggtgactacattgggctgcaagggaaccccaagctgcagaagctgaaaggcggggaggaggggcctgttctgatggcagaggccgtgaagaaggtcaatcgtggcaatggcaagacttcttctcggattctcctcctgaccaagggccatgtgattctcacagacaccaagaagtcccaggccaaaattgtcattgggctagacaatgtggctggggtgtcagtcaccagcctcaaggatgggctctttagcttgcatctgagtgagatgtcatcggtgggctccaagggggacttcctgctggtcagcgagcatgtgattgaactgctgaccaaaatgtaccgggctgtgctggatgccacgcagaggcagcttacagtcaccgtgactgagaagttctcagtgaggttcaaggagaacagtgtggctgtcaaggtcgtccagggccctgcaggtggtgacaacagcaagctacgctacaaaaaaaaggggagtcattgcttggaggtgactgtgcagtgaggagggggcaccatgcagagatggcagttgcttcctcctgaaccagcactaatccccctctgccctcctgtgtgggaggatctctaacccctctgatcgtggcgcatggcttggggattaaactacccttgaagaggacccttgtcccaaacccttcttgttctctcctccaaaagtagcttcctccaacccgcagcctctctgcacactaataaaacatgtggcttggaaaggttcaaaaaaaaaaaa

SEQ ID NO: 34 - protein, 1043 aa, Homo sapien
MPLLEGSVGVEDLVLLEPLVEESLLKNLQLRYENKEIYTYIGNVVISVNPYQQLPIYGPEFIAKYQDYTFYELKPHIYALANVAYQSLRDRDRDQCILITGESGSGKTEASKLVMSYVAAVCGKGEQVNSVKEQLLQSNPVLEAFGNAKTIRNNNSSRFGKYMDIEFDFKGSPLGGVITNYLLEKSRLVKQLKGERNFHIFYQLLAGADEQLLKALKLERDTTGYAYLNHEVSRVDGMDDASSFRAVQSAMAVIGFSEEEIRQVLEVTSMVLKLGNVLVADEFQASGIPASGIRDGRGVREIGEMVGLNSEEVERALCSRTMETAKEKVVTALNVMQAQYARDALAKNIYSRLFDWIVNRINESIKVGIGEKKKVMGVLDIYGFEILEDNSFEQFVINYCNEKLQQVFIEMTLKEEQEEYKREGIPWTKVDYFDNGIICKLIEHNQRGILAMLDEECLRPGVVSDSTFLAKLNQLFSKHGHYESKVTQNAQRQYDHTMGLSCFRICHYAGKVTYNVTSFIDKNNDLLFRDLLQAMWKAQHPLLRSLFPEGNPKQASLKRPPTAGAQFKSSVAILMKNLYSKSPNYIRCIKPNEHQQRGQFSSDLVATQARYLGLLENVRVRRAGYAHRQGYGPFLERYRLLSRSTWPHWNGGDREGVEKVLGELSMSSGELAFGKTKIFIRSPKTLFYLEEQRRLRLQQLATLIQKIYRGWRCRTHYQLMRKSQILISSWFRGNMQKKCYGKIKASVLLIQAFVRGWKARKNYRKYFRSEAALTLADFIYKSMVQKFLLGLKNNLPSTNVLDKTWPAAPYKCLSTANQELQQLFYQWKCKRFRDQLSPKQVEILREKLCASELFKGKKASYPQSVPIPFCGDYIGLQGNPKLQKLKGGEEGPVLMAEAVKKVNRGNGKTSSRILLLTKGHVILTDTKKSQAKIVIGLDNVAGVSVTSLKDGLFSLHLSEMSSVGSKGDFLLVSEHVIELLTKMYRAVLDATQRQLTVTVTEKFSVRFKENSVAVKVVQGPAGGDNSKLRYKKKGSHCLEVTVQ

SEQ ID NO: 35 - DNA, 1876 bp, Homo sapien
gagccatgctcgcggcgatgggctctctggcggctgccctctgggcagtggtccatcctcggactctcctactgggcactgtcgcctttctgctcgctgctgactttctcaaaagacggcgcccaaagaactacccgccggggccctggcgcctgcccttccttggcaacttcttccttgtggacttcgagcagtcgcacctggaggttcagctgtttgtgaagaaatatgggaacctttttagcttggagcttggtgacatatctgcagttcttattactggcttgcccttaatcaaagaagcccttatccacatggaccaaaactttgggaaccgccccgtgacccctatgcgagaacatatctttaagaaaaatggattgattatgtcaagtggccaggcatggaaggagcaaagaaggttcactctgacagcactaaggaactttggtttaggaaagaagagcttagaggaacgcattcaggaggaggcccaacacctcactgaagcaataaaagaggagaacggacagccttttgaccctcatttcaagatcaacaatgcagtttccaatatcatttgctccatcaccttcggagaacgctttgagtaccaggatagttggtttcagcagctgctgaagttactagatgaagtcacatacttggaggcttcaaagacatgccagctctacaatgtctttccatggataatgaaattcctgcctggaccccaccaaactctcttcagcaactggaaaaaactgaaattgtttgtttctcatatgattgacaaacacagaaaggattggaatcctgcagaaacaagagactttattgatgcttaccttaaagaaatgtcaaagcacacaggcaatcctacttcaagtttccatgaagaaaacctcatctgcagcaccctggacctcttctttgccggaaccgagacaacttccacaactctgcgatgggctctgctttatatggccctctacccagaaatccaagaaaaagtacaagctgagattgacagagtgattggccaggggcagcagccgagcacagccgcccgggagtccatgccctacaccaatgctgtcatccatgaggtgcagagaatgggcaacatcatccccctgaacgttcccagggaagtgacagttgataccactttggctgggtaccacctgcccaagggtaccatgatcctgaccaatttgacggcgctgcacagggaccccacagagtgggccacccctgacacattcaatccggaccattttctggagaatggacagtttaagaaaagggaagcctttatgcctttctcaataggaaagcgggcatgcctcggagaacagttggccaggactgagctgtttattttcttcacttcccttatgcaaaaatttaccttcaggcccccaaacaatgagaagctgagcctgaagtttagaatgggtatcaccatttccccagtcagtcaccgcctctgcgctgttcctcaggtgtaatattgttaagaaagaaaggggcaaggaaagtaagaagacatggcacgtgttctgaaaccactggtgtctgctcagatgtgttgggacaaaatgaaagtgactttcaagaaagatcagaggaatttgactcagagaaaactagatccaaatcccagctctactgtctcgtccgaattagccttgggaaaatcatttatatgctaaataatttacctttttatctaggagatgaaaagaggataatgtttccttccataaagaaagttcttgtaagaatcaaaagaaatggtgagctttaagtggtttgtaaaccataaaacacatcataaaagttctatctataaaaaaaaaaaaaaaaaaaa

SEQ ID NO: 36 - protein, 502 aa, Homo sapien
MLAAMGSLAAALWAVVHPRTLLLGTVAFLLAADFLKRRRPKNYPPGPWRLPFLGNFFLVDFEQSHLEVQLFVKKYGNLFSLELGDISAVLITGLPLIKEALIHMDQNFGNRPVTPMREHIFKKNGLIMSSGQAWKEQRRFTLTALRNFGLGKKSLEERIQEEAQHLTEAIKEENGQPFDPHFKINNAVSNIICSITFGERFEYQDSWFQQLLKLLDEVTYLEASKTCQLYNVFPWIMKFLPGPHQTLFSNWKKLKLFVSHMIDKHRKDWNPAETRDFIDAYLKEMSKHTGNPTSSFHEENLICSTLDLFFAGTETTSTTLRWALLYMALYPEIQEKVQAEIDRVIGQGQQPSTAARESMPYTNAVIHEVQRMGNIIPLNVPREVTVDTTLAGYHLPKGTMILTNLTALHRDPTEWATPDTFNPDHFLENGQFKKREAFMPFSIGKRACLGEQLARTELFIFFTSLMQKFTFRPPNNEKLSLKFRMGITISPVSHRLCAVPQV

SEQ ID NO: 37 - DNA, 1577 bp, Homo sapien
gcccgctgcggtaaatggggcagaggccgggaggggtgggggttccccgcgccgcagccatggagcagcttcgcgccgccgcccgtctgcagattgttctgggccacctcggccgcccctcggccggggctgtcgtagctcatcccacttcagggactatttcctctgccagtttccatcctcaacaattccagtatactctggataataatgttctaaccctggaacagagaaaattttatgaagaaaatgggtttctagtaatcaaaaatcttgtacctgatgccgatattcaacgctttcggaatgagtttgaaaaaatctgcagaaaggaggtgaaaccattaggattaacagtaatgagagatgtgaccatttcgaaatccgaatatgctccaagtgagaagatgatcacgaaggtccaggatttccaggaagataaggagctcttcagatactgcactctccccgagattctgaaatatgtggagtgcttcactggacctaatattatggccatgcacacaatgttgataaacaaacctccagattctggcaagaagacgtcccgtcaccccctgcaccaggacctgcactatttccccttcaggcccagcgatctcatcgtttgcgcctggacggcgatggagcacatcagccggaacaacggctgtctggttgtgctcccaggcacacacaagggctccctgaagccccacgattaccccaagtgggaggggggagttaacaaaatgttccacgggatccaggactacgaggaaaacaaggcccgggtgcacctggtgatggagaagggcgacactgttttcttccatcctttgctcatccacggatctggtcagaataaaacccagggattccggaaggcaatttcctgccatttcgccagtgccgattgccactacattgacgtgaagggcaccagtcaagaaaacatcgagaaggaagttgtaggaatagcacataaattctttggagctgaaaatagcgtgaacttgaaggatatttggatgtttcgagctcgacttgtgaaaggagaaagaaccaatctttgaaatagccatctgctataactctttcaacagaaaaccaaaaccaaacgaaatgtctaaggaaaatgttttcttaatgagatgatgtaaccttttctatcacttgttaaaagcagaaaacatgtatcaggtacttaattgcatagagttagttttgcagcacaatggtgttgctttaatggaaaaaaaaaacagtaaaagtgaaatattactgttttaaggaaaactaatttagggtggcagccaataaaggtggttggtgtctaatttaagtgttaaatcaatttctttcattcagttagctctttacccaagaagaagtgaatgatttggagcttagggtatgttttgtatcccctttctgataaacccattccctaccaattttatgtcataagagatttttttcccccaaatctagaacaatgtataatacattcacatctagtcaagggcataggaacggtgtcatggagtccaaataaagtggatattcctgctcgg

SEQ ID NO: 38 - protein, 338 aa, Homo sapien
MEQLRAAARLQIVLGHLGRPSAGAVVAHPTSGTISSASFHPQQFQYTLDNNVLTLEQRKFYEENGFLVIKNLVPDADIQRFRNEFEKICRKEVKPLGLTVMRDVTISKSEYAPSEKMITKVQDFQEDKELFRYCTLPEILKYVECFTGPNIMAMHTMLINKPPDSGKKTSRHPLHQDLHYFPFRPSDLIVCAWTAMEHISRNNGCLVVLPGTHKGSLKPHDYPKWEGGVNKMFHGIQDYEENKARVHLVMEKGDTVFFHPLLIHGSGQNKTQGFRKAISCHFASADCHYIDVKGTSQENIEKEVVGIAHKFFGAENSVNLKDIWMFRARLVKGERTNL

SEQ ID NO: 39 - DNA, 716 bp, Homo sapien
atggcagagcagtcggacgaggccgtgaagtactacaccctagaggagattcagaagcacaaccacagcaagagcacctggctgatcctgcaccacaaggtgtacgatttgaccaaatttctggaagagcatcctggtggggaagaagttttaagggaacaagctggaggtgacgctactgagaactttgaggatgtcgggcactctacagatgccagggaaatgtccaaaacattcatcattggggagctccatccagatgacagaccaaagttaaacaagcctccagaaccttaaaggcggtgtttcaaggaaactcttatcactactattgattctagttccagttggtggaccaactgggtgatccctgccatctctgcagtggccgtcgccttgatgtatcgcctatacatggcagaggactgaacacctcctcagaagtcagcgcaggaagagcctgctttggacacgggagaaaagaagccattgctaactacttcaactgacagaaaccttcacttgaaaacaatgattttaatatatctctttctttttcttccgacattagaaacaaaacaaaaagaactgtcctttctgcgctcaaatttttcgagtgtgcctttttattcatctactttattttgatgtttccttaatgtgtaatttacttattataagcatgatcttttaaaaatatatttggcttttaaagt

SEQ ID NO: 40 - protein, 98 aa, Homo sapien
MAEQSDEAVKYYTLEEIQKHNHSKSTWLILHHKVYDLTKFLEEHPGGEEVLREQAGGDATENFEDVGHSTDAREMSKTFIIGELHPDDRPKLNKPPEP

SEQ ID NO: 41 - DNA, 578 bp, Homo sapien
cctcctgggagggagctgaagccgctcgcaagactcccgtagtccccacctctctcagcttccggctggtagtagttccgcttcctgtccgactgtggtgtctttgctgagggtcacattgagctgcaggttgaatccggggtgcctttaggattcagcaccatggcggaagacatggagaccaaaatcaagaactacaagaccgccccttttgacagccgcttccccaaccagaaccagactagaaactgctggcagaactacctggacttccaccgctgtcagaaggcaatgaccgctaaaggaggcgatatctctgtgtgcgaatggtaccagcgtgtgtaccagtccctctgccccacatcctgggtcacagactgggatgagcaacgggctgaaggcacgtttcccgggaagatctgaactggctgcatctccctttcctctgtcctccatccttctcccaggatggtgaagggggacctggtacccagtgatccccaccccaggatcctaaatcatgacttacctgctaataaaaactcattggaaaagtgaaaaaaaaaaaaaaaaaaaaa

SEQ ID NO: 42 - protein, 86 aa, Homo sapien
MAEDMETKIKNYKTAPFDSRFPNQNQTRNCWQNYLDFHRCQKAMTAKGGDISVCEWYQRVYQSLCPTSWVTDWDEQRAEGTFPGKI

SEQ ID NO: 43 - DNA, 2444 bp, Homo sapien
ggttttttttttttaccccccttttttatttattatttttttgcacattgagcggatccttgggaacgagagaaaaaagaaacccaaactcacgcgtgcagaagatctccccccccttcccctcccctcctccctcttttcccctccccaggagaaaaagacccccaagcagaaaaaagttcaccttggactcgtctttttcttgcaatattttttgggggggcaaaactttgagggggtgattttttttggcttttcttcctccttcatttttcttccaaaattgctgctggtgggtgaaaaaaaaatgccgcagctgaacggcggtggaggggatgacctaggcgccaacgacgaactgatttccttcaaagacgagggcgaacaggaggagaagagctccgaaaactcctcggcagagagggatttagctgatgtcaaatcgtctctagtcaatgaatcagaaacgaatcaaaacagctcctccgattccgaggcggaaagacggcctccgcctcgctccgaaagtttccgagacaaatcccgggaaagtttggaagaagcggccaagaggcaagatggagggctctttaaggggccaccgtatcccggctaccccttcatcatgatccccgacctgacgagcccctacctccccaacggatcgctctcgcccaccgcccgaacctatctccagatgaaatggccactgcttgatgtccaggcagggagcctccagagtagacaagccctcaaggatgcccggtccccatcaccggcacacattgtctctaacaaagtgccagtggtgcagcaccctcaccatgtccaccccctcacgcctcttatcacgtacagcaatgaacacttcacgccgggaaacccacctccacacttaccagccgacgtagaccccaaaacaggaatcccacggcctccgcaccctccagatatatccccgtattacccactatcgcctggcaccgtaggacaaatcccccatccgctaggatggttagtaccacagcaaggtcaaccagtgtacccaatcacgacaggaggattcagacacccctaccccacagctctgaccgtcaatgcttccgtgtccaggttccctccccatatggtcccaccacatcatacgctacacacgacgggcattccgcatccggccatagtcacaccaacagtcaaacaggaatcgtcccagagtgatgtcggctcactccatagttcaaagcatcaggactccaaaaaggaagaagaaaagaagaagccccacataaagaaacctcttaatgcattcatgttgtatatgaaggaaatgagagcaaaggtcgtagctgagtgcacgttgaaagaaagcgcggccatcaaccagatccttgggcggaggtggcatgcactgtccagagaagagcaagcgaaatactacgagctggcccggaaggagcgacagcttcatatgcaactgtaccccggctggtccgcgcgggataactatggaaagaagaagaagaggaaaagggacaagcagccgggagagaccaatgaacacagcgaatgtttcctaaatccttgcctttcacttcctccgattacagacctcagcgctcctaagaaatgccgagcgcgctttggccttgatcaacagaataactggtgcggcccttgcaggagaaaaaaaaagtgcgttcgctacatacaaggtgaaggcagctgcctcagcccaccctcttcagatggaagcttactagattcgcctcccccctccccgaacctgctaggctcccctccccgagacgccaagtcacagactgagcagacccagcctctgtcgctgtccctgaagcccgaccccctggcccacctgtccatgatgcctccgccacccgccctcctgctcgctgaggccacccacaaggcctccgccctctgtcccaacggggccctggacctgcccccagccgctttgcagcctgccgccccctcctcatcaattgcacagccgtcgacttcttggttacattcccacagctccctggccgggacccagccccagccgctgtcgctcgtcaccaagtctttagaatagctttagcgtcgtgaaccccgctgctttgtttatggttttgtttcacttttcttaatttgccccccacccccaccttgaaaggttttgttttgtactctcttaattttgtgccatgtggctacattagttgatgtttatcgagttcattggtcaatatttgacccattcttatttcaatttctccttttaaatatgtagatgagagaagaacctcatgattggtaccaaaatttttatcaacagctgtttaaagtctttgtagcgtttaaaaaatatatatatatacataactgttatgtagttcggatagcttagttttaaaagactgattaaaaaacaaaaaaaa

SEQ ID NO: 44 - protein, 596 aa, Homo sapien
MPQLNGGGGDDLGANDELISFKDEGEQEEKSSENSSAERDLADVKSSLVNESETNQNSSSDSEAERRPPPRSESFRDKSRESLEEAAKRQDGGLFKGPPYPGYPFIMIPDLTSPYLPNGSLSPTARTYLQMKWPLLDVQAGSLQSRQALKDARSPSPAHIVSNKVPVVQHPHHVHPLTPLITYSNEHFTPGNPPPHLPADVDPKTGIPRPPHPPDISPYYPLSPGTVGQIPHPLGWLVPQQGQPVYPITTGGFRHPYPTALTVNASVSRFPPHMVPPHHTLHTTGIPHPAIVTPTVKQESSQSDVGSLHSSKHQDSKKEEEKKKPHIKKPLNAFMLYMKEMRAKVVAECTLKESAAINQILGRRWHALSREEQAKYYELARKERQLHMQLYPGWSARDNYGKKKKRKRDKQPGETNEHSECFLNPCLSLPPITDLSAPKKCRARFGLDQQNNWCGPCRRKKKCVRYIQGEGSCLSPPSSDGSLLDSPPPSPNLLGSPPRDAKSQTEQTQPLSLSLKPDPLAHLSMMPPPPALLLAEATHKASALCPNGALDLPPAALQPAAPSSSIAQPSTSWLHSHSSLAGTQPQPLSLVTKSLE

SEQ ID NO: 45 - DNA, 3697 bp, Homo sapien
agggagtgttcccgggggagatactccagtcgtagcaagagtctcgaccactgaatggaagaaaaggacttttaaccaccattttgtgacttacagaaaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccatagaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttgagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagagtttgaggagagggagtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaacgtgaggactcaggaaaaataaataaataaaagaacagccttttcccttagtattaacagaaatgtttctgtgtcattaaccatctttaatcaatgtgacatgttgctctttggctgaaattcttcaacttggaaatgacacagacccacagaaggtgttcaaacacaacctactctgcaaaccttggtaaaggaaccagtcagctggccagatttcctcactacctgccatgcatacatgctgcgcatgttttcttcattcgtatgttagtaaagttttggttattatatatttaacatgtggaagaaaacaagacatgaaaagagtggtgacaaatcaagaataaacactggttgtagtcagttttgtttgttaa

SEQ ID NO: 46 - protein, 832 aa, Homo sapien
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIEVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 47 - DNA, 1258 bp, Homo sapien
ctcgtcaacagctgccgcgcgcaggcttagctcattcctctgacctgccaggaagcagagagacccacagagcaggagggaggcagaaagtggagacggacctgagcccgaggaagaggcaggcagaggctgaggctgattccaccccagcctgcctggacaaccctccttagccgcagccccttccagttccctaggggttctgcccctccccctctctggggcaccagccccccagggtcctgcatcccaccatgtcgatggctgtggaaacctttggcttcttcatggcaactgtggggctgctgatgctgggggtgactctgccaaacagctactggcgagtgtccactgtgcacgggaacgtcatcaccaccaacaccatcttcgagaacctctggtttagctgtgccaccgactccctgggcgtctacaactgctgggagttcccgtccatgctggccctctctgggtatattcaggcctgccgggcactcatgatcaccgccatcctcctgggcttcctcggcctcttgctaggcatagcgggcctgcgctgcaccaacattgggggcctggagctctccaggaaagccaagctggcggccaccgcaggggccctccacattctggccggtatctgcgggatggtggccatctcctggtacgccttcaacatcacccgggacttcttcgaccccttgtaccccggaaccaagtacgagctgggccccgccctctacctggggtggagcgcctcactgatctccatcctgggtggcctctgcctctgctccgcctgctgctgcggctctgacgaggacccagccgccagcgcccggcggccctaccaggctcccgtgtccgtgatgcccgtcgccacctcggaccaagaaggcgacagcagctttggcaaatacggcagaaacgcctacgtgtagcagctctggcccgtgggccccgctgtcttcccactgccccaaggagaggggacctggccggggcccattcccctatagtaacctcaggggccggccacgccccgctcccgtagccccgccccggccacggccccgtgtcttgcactctcatggcccctccaggccaagaactgctcttgggaagtcgcatatctcccctctgaggctggatccctcatcttctgaccctgggttctgggctgtgaaggggacggtgtccccgcacgtttgtattgtgtataaatacattcattaataaatgcatattgtgaccgttc

SEQ ID NO: 48 - protein, 1258 aa, Homo sapien
CTCGTCAACAGCTGCCGCGCGCAGGCTTAGCTCATTCCTCTGACCTGCCAGGAAGCAGAGAGACCCACAGAGCAGGAGGGAGGCAGAAAGTGGAGACGGACCTGAGCCCGAGGAAGAGGCAGGCAGAGGCTGAGGCTGATTCCACCCCAGCCTGCCTGGACAACCCTCCTTAGCCGCAGCCCCTTCCAGTTCCCTAGGGGTTCTGCCCCTCCCCCTCTCTGGGGCACCAGCCCCCCAGGGTCCTGCATCCCACCATGTCGATGGCTGTGGAAACCTTTGGCTTCTTCATGGCAACTGTGGGGCTGCTGATGCTGGGGGTGACTCTGCCAAACAGCTACTGGCGAGTGTCCACTGTGCACGGGAACGTCATCACCACCAACACCATCTTCGAGAACCTCTGGTTTAGCTGTGCCACCGACTCCCTGGGCGTCTACAACTGCTGGGAGTTCCCGTCCATGCTGGCCCTCTCTGGGTATATTCAGGCCTGCCGGGCACTCATGATCACCGCCATCCTCCTGGGCTTCCTCGGCCTCTTGCTAGGCATAGCGGGCCTGCGCTGCACCAACATTGGGGGCCTGGAGCTCTCCAGGAAAGCCAAGCTGGCGGCCACCGCAGGGGCCCTCCACATTCTGGCCGGTATCTGCGGGATGGTGGCCATCTCCTGGTACGCCTTCAACATCACCCGGGACTTCTTCGACCCCTTGTACCCCGGAACCAAGTACGAGCTGGGCCCCGCCCTCTACCTGGGGTGGAGCGCCTCACTGATCTCCATCCTGGGTGGCCTCTGCCTCTGCTCCGCCTGCTGCTGCGGCTCTGACGAGGACCCAGCCGCCAGCGCCCGGCGGCCCTACCAGGCTCCCGTGTCCGTGATGCCCGTCGCCACCTCGGACCAAGAAGGCGACAGCAGCTTTGGCAAATACGGCAGAAACGCCTACGTGTAGCAGCTCTGGCCCGTGGGCCCCGCTGTCTTCCCACTGCCCCAAGGAGAGGGGACCTGGCCGGGGCCCATTCCCCTATAGTAACCTCAGGGGCCGGCCACGCCCCGCTCCCGTAGCCCCGCCCCGGCCACGGCCCCGTGTCTTGCACTCTCATGGCCCCTCCAGGCCAAGAACTGCTCTTGGGAAGTCGCATATCTCCCCTCTGAGGCTGGATCCCTCATCTTCTGACCCTGGGTTCTGGGCTGTGAAGGGGACGGTGTCCCCGCACGTTTGTATTGTGTATAAATACATTCATTAATAAATGCATATTGTGACCGTTC

SEQ ID NO: 49 - DNA, 6129 bp, Homo sapien
aattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatattacaatgctgtattttaaaagaatgattatgaattacatttgtataaaataatttttatatttgaaatattgactttttatggcactagtatttttatgaaatattatgttaaaactgggacaggggagaacctagggtgatattaaccaggggccatgaatcaccttttggtctggagggaagccttggggctgatcgagttgttgcccacagctgtatgattcccagccagacacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgt

SEQ ID NO: 50 - protein, 1480 aa, Homo sapien
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMMIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILNEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECLFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 51 - DNA, 1847 bp, Homo sapien
ctcctgccctccactgactccagagagggagatccccagtacttgactccatcacgcagatgggagcaggcaccagctatggagagggatacagctgcgtctccacatgacccatcctgcatgacaccaaagccaccgccagacagtgcctcggattctatgcaaaacctgggaagcggagacctaccccagccccgggaggaagctagctcttcaggggaccgtctgaggactggagtttgatccatgaacctggcttcgaggccttgcttttctctcttcttcattcatattcattcccaacaccttagaaggtgttgcttaatttatttctagaaaagcagcccagagtcagtcattgaagccttccccaccccctggccaaaaaaaaaaaaaaaaaaaaactggacacattttggatctgttgggagcttggagtccagtggttggcatagttgtcacattgggagcagagaagaagcaaccaggggccctgatcaggggactgagccgtagagtcccaggatggcacccaatggcacagcctcttccttttgcctggactctaccgcatgcaagatcaccatcaccgtggtccttgcggtcctcatcctcatcaccgttgctggcaatgtggtcgtctgtctggccgtgggcttgaaccgccggctccgcaacctgaccaattgtttcatcgtgtccttggctatcactgacctgctcctcggcctcctggtgctgcccttctctgccatctaccagctgtcctgcaagtggagctttggcaaggtcttctgcaatatctacaccagcctggatgtgatgctctgcacagcctccattcttaacctcttcatgatcagcctcgaccggtactgcgctgtcatggacccactgcggtaccctgtgctggtcaccccagttcgggtcgccatctctctggtcttaatttgggtcatctccattaccctgtcctttctgtctatccacctggggtggaacagcaggaacgagaccagcaagggcaatcataccacctctaagtgcaaagtccaggtcaatgaagtgtacgggctggtggatgggctggtcaccttctacctcccgctactgatcatgtgcatcacctactaccgcatcttcaaggtcgcccgggatcaggccaagaggatcaatcacattagctcctggaaggcagccaccatcagggagcacaaagccacagtgacactggccgccgtcatgggggccttcatcatctgctggtttccctacttcaccgcgtttgtgtaccgtgggctgagaggggatgatgccatcaatgaggtgttagaagccatcgttctgtggctgggctatgccaactcagccctgaaccccatcctgtatgctgcgctgaacagagacttccgcaccgggtaccaacagctcttctgctgcaggctggccaaccgcaactcccacaaaacttctctgaggtccaacgcctctcagctgtccaggacccaaagccgagaacccaggcaacaggaagagaaacccctgaagctccaggtgtggagtgggacagaagtcacggccccccagggagccacagacaggtaatagccctagccattggtgcacaggatgggggcaatgggaggggatgctactgatgggaatgattaagggagctgctgtttaggtggtgctggtttatgttctaggaactcttcatgagcactttgtaaacaccctcttgcttaatcctcccaacggcccccaaaggtagaacttagctcccttttaaaaggagcacattaaaattctcagaggacttggcaagggccgcacagctggggcat

SEQ ID NO: 52 - protein, 359 aa, Homo sapien
MAPNGTASSFCLDSTACKITITVVLAVLILITVAGNVVVCLAVGLNRRLRNLTNCFIVSLAITDLLLGLLVLPFSAIYQLSCKWSFGKVFCNIYTSLDVMLCTASILNLFMISLDRYCAVMDPLRYPVLVTPVRVAISLVLIWVISITLSFLSIHLGWNSRNETSKGNHTTSKCKVQVNEVYGLVDGLVTFYLPLLIMCITYYRIFKVARDQAKRINHISSWKAATIREHKATVTLAAVMGAFIICWFPYFTAFVYRGLRGDDAINEVLEAIVLWLGYANSALNPILYAALNRDFRTGYQQLFCCRLANRNSHKTSLRSNASQLSRTQSREPRQQEEKPLKLQVWSGTEVTAPQGATDR

SEQ ID NO: 53 - DNA, 5512 bp, Homo sapien
gagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatgaattcttccttagacttacttttgtaaaaatgtccccacggtacttactccccactgatggaccagtggtttccagtcatgagcgttagactgacttgtttgtcttccattccattgttttgaaactcagtatgccgcccctgtcttgctgtcatgaaatcagcaagagaggatgacacatcaaataataactcggattccagcccacattggattcatcagcatttggaccaatagcccacagctgagaatgtggaatacctaaggataacaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtgaattcaggtagtaaatatgaaactagggtttgaaattgataatgctttcacaacatttgcagatgttttagaaggaaaaaagttccttcctaaaataatttctctacaattggaagattggaagattcagctagttaggagcccattttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttcc

SEQ ID NO: 54 - protein, 1210 aa, Homo sapien [sequence omitted]

SEQ ID NO: 55 - DNA, 4711 bp, Homo sapien
gccccgggaagcgcagccatggctctgcggaggctgggggccgcgctgctgctgctgccgctgctcgccgccgtggaagaaacgctaatggactccactacagcgactgctgagctgggctggatggtgcatcctccatcagggtgggaagaggtgagtggctacgatgagaacatgaacacgatccgcacgtaccaggtgtgcaacgtgtttgagtcaagccagaacaactggctacggaccaagtttatccggcgccgtggcgcccaccgcatccacgtggagatgaagttttcggtgcgtgactgcagcagcatccccagcgtgcctggctcctgcaaggagaccttcaacctctattactatgaggctgactttgactcggccaccaagaccttccccaactggatggagaatccatgggtgaaggtggataccattgcagccgacgagagcttctcccaggtggacctgggtggccgcgtcatgaaaatcaacaccgaggtgcggagcttcggacctgtgtcccgcagcggcttctacctggccttccaggactatggcggctgcatgtccctcatcgccgtgcgtgtcttctaccgcaagtgcccccgcatcatccagaatggcgccatcttccaggaaaccctgtcgggggctgagagcacatcgctggtggctgcccggggcagctgcatcgccaatgcggaagaggtggatgtacccatcaagctctactgtaacggggacggcgagtggctggtgcccatcgggcgctgcatgtgcaaagcaggcttcgaggccgttgagaatggcaccgtctgccgaggttgtccatctgggactttcaaggccaaccaaggggatgaggcctgtacccactgtcccatcaacagccggaccacttctgaaggggccaccaactgtgtctgccgcaatggctactacagagcagacctggaccccctggacatgccctgcacaaccatcccctccgcgccccaggctgtgatttccagtgtcaatgagacctccctcatgctggagtggacccctccccgcgactccggaggccgagaggacctcgtctacaacatcatctgcaagagctgtggctcgggccggggtgcctgcacccgctgcggggacaatgtacagtacgcaccacgccagctaggcctgaccgagccacgcatttacatcagtgacctgctggcccacacccagtacaccttcgagatccaggctgtgaacggcgttactgaccagagccccttctcgcctcagttcgcctctgtgaacatcaccaccaaccaggcagctccatcggcagtgtccatcatgcatcaggtgagccgcaccgtggacagcattaccctgtcgtggtcccagccggaccagcccaatggcgtgatcctggactatgagctgcagtactatgagaaggagctcagtgagtacaacgccacagccataaaaagccccaccaacacggtcaccgtgcagggcctcaaagccggcgccatctatgtcttccaggtgcgggcacgcaccgtggcaggctacgggcgctacagcggcaagatgtacttccagaccatgacagaagccgagtaccagacaagcatccaggagaagttgccactcatcatcggctcctcggccgctggcctggtcttcctcattgctgtggttgtcatcgccatcgtgtgtaacagaagacgggggtttgagcgtgctgactcggagtacacggacaagctgcaacactacaccagtggccacatgaccccaggcatgaagatctacatcgatcctttcacctacgaggaccccaacgaggcagtgcgggagtttgccaaggaaattgacatctcctgtgtcaaaattgagcaggtgatcggagcaggggagtttggcgaggtctgcagtggccacctgaagctgccaggcaagagagagatctttgtggccatcaagacgctcaagtcgggctacacggagaagcagcgccgggacttcctgagcgaagcctccatcatgggccagttcgaccatcccaacgtcatccacctggagggtgtcgtgaccaagagcacacctgtgatgatcatcaccgagttcatggagaatggctccctggactcctttctccggcaaaacgatgggcagttcacagtcatccagctggtgggcatgcttcggggcatcgcagctggcatgaagtacctggcagacatgaactatgttcaccgtgacctggctgcccgcaacatcctcgtcaacagcaacctggtctgcaaggtgtcggactttgggctctcacgctttctagaggacgatacctcagaccccacctacaccagtgccctgggcggaaagatccccatccgctggacagccccggaagccatccagtaccggaagttcacctcggccagtgatgtgtggagctacggcattgtcatgtgggaggtgatgtcctatggggagcggccctactgggacatgaccaaccaggatgtaatcaatgccattgagcaggactatcggctgccaccgcccatggactgcccgagcgccctgcaccaactcatgctggactgttggcagaaggaccgcaaccaccggcccaagttcggccaaattgtcaacacgctagacaagatgatccgcaatcccaacagcctcaaagccatggcgcccctctcctctggcatcaacctgccgctgctggaccgcacgatccccgactacaccagctttaacacggtggacgagtggctggaggccatcaagatggggcagtacaaggagagcttcgccaatgccggcttcacctcctttgacgtcgtgtctcagatgatgatggaggacattctccgggttggggtcactttggctggccaccagaaaaaaatcctgaacagtatccaggtgatgcgggcgcagatgaaccagattcagtctgtggaggtttgacattcacctgcctcggctcacctcttcctccaagccccgccccctctgccccacgtgccggccctcctggtgctctatccactgcagggccagccactcgccaggaggccacgggccacgggaagaaccaagcggtgccagccacgagacgtcaccaagaaaacatgcaactcaaacgacggaaaaaaaaagggaatgggaaaaaagaaaacagatcctgggagggggcgggaaatacaaggaatattttttaaagaggattctcataaggaaagcaatgactgttcttgcgggggataaaaaagggcttgggagattcatgcgatgtgtccaatcggagacaaaagcagtttctctccaactccctctgggaaggtgacctggccagagccaagaaacactttcagaaaaacaaatgtgaaggggagagacaggggccgcccttggctcctgtccctgctgctcctctaggcctcactcaacaaccaagcgcctggaggacgggacagatggacagacagccaccctgagaacccctctgggaaaatctattcctgccaccactgggcaaacagaagaatttttctgtctttggagagtattttagaaactccaatgaaagacactgtttctcctgttggctcacagggctgaaaggggcttttgtcctcctgggtcagggagaacgcggggaccccagaaaggtcagccttcctgaggatgggcaacccccaggtctgcagctccaggtacatatcacgcgcacagcctggcagcctggccctcctggtgcccactcccgccagcccctgcctcgaggactgatactgcagtgactgccgtcagctccgactgccgctgagaagggttgatcctgcatctgggtttgtttacagcaattcctggactcgggggtattttggtcacagggtggttttggtttagggggtttgtttgttgggttgttttttgttttttggttttttttaatgacaatgaagtgacactttgacatttcctaccttttgaggacttgatccttctccaggaagaaggtgctttctgcttactgacttaggcaatacaccaagggcgagattttatatgcacatttctggatttttttatacggttttcattgacactcttccctcctcccacctgccaccaggcctcaccaaagcccactgccatggggccatctgggccattcagagactggagtgagatttgggtgtggagggggaggcgccaaggtggaggagcttcccactccaggactgttgatgaaagggacagattgaggaggaagtgggctctgaggctgcagggctggaagtccttgcccacttcccactctcctgccccaatctatctagtacttcccaggcaaataggcccctttgaggctcctgagtgccctcagatggtcaaaacccagttttccctctgggagcctaaaccaggctgcatcggaggccaggacccggatcattcactgtgataccctgccctccagagggtgcgctcagagacacgggcaagcatgcctcttcccttccctggagagaaagtgtgtgatttctctcccacctccttccccccaccagacctttgctgggcctaaaggtcttggccatggggacgccctcagtctagggatctggccacagactccctcctgtgaaccaacacagacacccaagcagagcaatcagttagtgaattg

SEQ ID NO: 56 - protein, 987 aa, Homo sapien
MALRRLGAALLLLPLLAAVEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPRIIQNGAIFQETLSGAESTSLVAARGSCIANAEEVDVPIKLYCNGDGEWLVPIGRCMCKAGFEAVENGTVCRGCPSGTFKANQGDEACTHCPINSRTTSEGATNCVCRNGYYRADLDPLDMPCTTIPSAPQAVISSVNETSLMLEWTPPRDSGGREDLVYNIICKSCGSGRGACTRCGDNVQYAPRQLGLTEPRIYISDLLAHTQYTFEIQAVNGVTDQSPFSPQFASVNITTNQAAPSAVSIMHQVSRTVDSITLSWSQPDQPNGVILDYELQYYEKELSEYNATAIKSPTNTVTVQGLKAGAIYVFQVRARTVAGYGRYSGKMYFQTMTEAEYQTSIQEKLPLIIGSSAAGLVFLIAVVVIAIVCNRRRGFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSSGINLPLLDRTIPDYTSFNTVDEWLEAIKMGQYKESFANAGFTSFDVVSQMMMEDILRVGVTLAGHQKKILNSIQVMRAQMNQIQSVEV

SEQ ID NO: 57 - DNA, 2033 bp, Homo sapien
ggagaatccccggaaaggctgagtctccagctcaaggtcaaaacgtccaaggccgaaagccctccagtttcccctggacgccttgctcctgcttctgctacgaccttctggggaaaacgaatttctcattttcttcttaaattgccattttcgctttaggagatgaatgttttcctttggctgttttggcaatgactctgaattaaagcgatgctaacgcctcttttccccctaattgttaaaagctatggactgcaggaagatggcccgcttctcttacagtgtgatttggatcatggccatttctaaagtctttgaactgggattagttgccgggctgggccatcaggaatttgctcgtccatctcggggatacctggccttcagagatgacagcatttggccccaggaggagcctgcaattcggcctcggtcttcccagcgtgtgccgcccatggggatacagcacagtaaggagctaaacagaacctgctgcctgaatgggggaacctgcatgctggggtccttttgtgcctgccctccctccttctacggacggaactgtgagcacgatgtgcgcaaagagaactgtgggtctgtgccccatgacacctggctgcccaagaagtgttccctgtgtaaatgctggcacggtcagctccgctgctttcctcaggcatttctacccggctgtgatggccttgtgatggatgagcacctcgtggcttccaggactccagaactaccaccgtctgcacgtactaccacttttatgctagttggcatctgcctttctatacaaagctactattaatcgacattgacctatttccagaaatacaattttagatatcatgcaaatttcatgaccagtaaaggctgctgctacaatgtcctaactgaaagatgatcatttgtagttgccttaaaataatgaatacaatttccaaaatggtctctaacatttccttacagaactacttcttacttctttgccctgccctctcccaaaaaactacttcttttttcaaaagaaagtcagccatatctccattgtgcctaagtccagtgtttctttttttttttttttttgagacggagtctcactctgtcacccaggctggactgcaatgacgcgatcttggttcactgcaacctccgcatccggggttcaagccattctcctgcctaagcctcccaagtaactgggattacaggcatgtgtcaccatgcccagctaatttttttgtattttagtagagatgggggtttcaccatattggccagtctggtctcgaactctgaccttgtgatccatcgatcagcctctcgagtgctgagattacacacgtgagcaactgtgcaaggcctggtgtttcttgatacatgtaattctaccaaggtcttcttaatatgttcttttaaatgattgaattatatgttcagattattggagactaattctaatgtggaccttagaatacagttttgagtagagttgatcaaaatcaattaaaatagtctctttaaaaggaaagaaaacatctttaaggggaggaaccagagtgctgaaggaatggaagtccatctgcgtgtgtgcagggagactgggtaggaaagaggaagcaaatagaagagagaggttgaaaaacaaaatgggttacttgattggtgattaggtggtggtagagaagcaagtaaaaaggctaaatggaagggcaagtttccatcatctatagaaagctatataagacaagaactcccctttttttcccaaaggcattataaaaagaatgaagcctccttagaaaaaaaattatacctcaatgtccccaacaagattgcttaataaattgtgtttcctccaagctattcaattcttttaactgttgtagaagacaaaatgttcacaatatatttagttgtaaaccaagtgatcaaactacatattgtaaagcccatttttaaaatacattgtatatatgtgtatgcacagtaaaaatggaaactatattgacctaaaaaaaaaaaaa

SEQ ID NO: 58 - protein, 188 aa, Homo sapien
MDCRKMARFSYSVIWIMAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 59 - DNA, 3346 bp, Homo sapien
gagtagacagcacagcggcagcggagggagtctatgcgagctggacagcagtgggaggtttgtgaggctcgcactggccgcagaccctcgggctcgatcgcccgggagccaggactcggcgacgcgaggctgccgggctacccggccgaggcttcgggggcgcaaactaatgggactggctcgctcggcagcatctccccgctcttctaagtacactgagcagggcccgcgctgaagtagaagctgtccgggggcgcgtagcccggagtcccagtgtggcccggaggaacggagcccgtgccagggcggcccagtcgggagcccggggaccgagcttgtgctgtggggaaacccccacttcttccaagggacagcgatcccgggacggtcgaggcgtcggggcggtcaccgagacctctgcgggaagaccccgtcggggagagggcgcgcagccccgaagcgtctcgggaagtcgagcggaatcgggcgggatcacccgggggcgcagagcccccgtcgcgcctcgtgcggcagcggagagcccaggagaacgagccctcgggggccgaagcccatgcccgggttgggggcggctgcccagtgagtcctcctggccggccgggcggagaagagcgacaccgaagccggcgggaggggagcacttcaaggccggcggctgcggaggatgggcgcctgagcggctccgagcgcagcgcggcagaggaaggcgaggcgagctttggtgaggaggcgccaagggatcccgaagtgcagtctgcccccgggaagatggctcggcctgggcagcgttggctcggcaagtggcttgtggcgatggtcgtgtgggcgctgtgccggctcgccacaccgctggccaagaacctggagcccgtatcctggagctccctcaaccccaagttcctgagtgggaagggcttggtgatctatccgaaaattggagacaagctggacatcatctgcccccgagcagaagcagggcggccctatgagtactacaagctgtacctggtgcggcctgagcaggcagctgcctgtagcacagttctcgaccccaacgtgttggtcacctgcaataggccagagcaggaaatacgctttaccatcaagttccaggagttcagccccaactacatgggcctggagttcaagaagcaccatgattactacattacctcaacatccaatggaagcctggaggggctggaaaaccgggagggcggtgtgtgccgcacacgcaccatgaagatcatcatgaaggttgggcaagatcccaatgctgtgacgcctgagcagctgactaccagcaggcccagcaaggaggcagacaacactgtcaagatggccacacaggcccctggtagtcggggctccctgggtgactctgatggcaagcatgagactgtgaaccaggaagagaagagtggcccaggtgcaagtgggggcagcagcggggaccctgatggcttcttcaactccaaggtggcattgttcgcggctgtcggtgccggttgcgtcatcttcctgctcatcatcatcttcctgacggtcctactactgaagctacgcaagcggcaccgcaagcacacacagcagcgggcggctgccctctcgctcagtaccctggccagtcccaaggggggcagtggcacagcgggcaccgagcccagcgacatcatcattcccttacggactacagagaacaactactgcccccactatgagaaggtgagtggggactacgggcaccctgtctacatcgtccaagagatgccgccccagagcccggcgaacatctactacaaggtctgagtgcccggcacggcctcaggcccccgagggacagtcggcctggaccggacctctcctttcgcccccacaccccctccccttgccagctgtgcccacctttgtatttagttttgtagtttcttggcttttataatccccctttttccctgccccctgggcttcggaggggggtgcttgtgcccctaacccccatgctcttgtgccttccccctctggccaggcctctgggctccgtgggggcgccccttcttggaaggcagggctggacactgatggacagcaggcagggagacagtcccctggccctgcccctccctcgccccccttgccaccttcccaggactgcttgtccgctatcatcactgtttttaatgcttttgtgttcattttttagctgtcaactcattttcatctgttttttgaagaaaaatggaaaaatgtaaaaggcagcccctccccaggctttgtgagcctggcccaagccagtacaagagggcctggggcacgatgtggtcagccaggaagcataggatgccatttcttttatagattccttggtatttctggtggggtaaggggcaggccagggctgttcacgcccatgagggaagaggaaagtgccactgggcaaggtgtcccaccctcccctcctgaccctcctacgaggcttatcctggcaatggggtagtcactgccacccttccacacacacacacacacacacacacacaaaaaaaaatcccttccttgtgggattcttgggcatctcctgcctccctcactctcacggtaattaatgtcttaattggctgttgcctggggaacaggagagctgctgcaggcagatgacctcatggggggtggagggaggtgaggtgcccaggtggctatttgccctgcagagctgggagtttcacccccaccccccaccctgttctctccttacctttggcatcctttggcctggtggggaaacagaggcccagggtggagacctaagcgggtataagaccaggtggcctgctccttttctgggccctagcacaggtgggtaacccccacccaacccagctcctgctgctgtcccagtcttgggctggggcctggaaagaggaagaggctgcctggggctgggccagcccgctgtgcactttgaccccagttccttgccagcacggctgctaacagactgccacttgagtgcgccttgcaggcactcccagagcagccatggaaggagctggccctcacaccatccacctccacactgcctcctggccagctgcccaccccagtgccaggtgggagagggagcagaacagccagccccttccaggtggcagtcggaagggtttttgtttttgtttctgttgccatttgtgtaaatactagtctttttggaaaaaaaataatgtaaagatgttttgtataaactctgaattattttcttgttgcttttttcttagaaaaaaatgagaactaaaaaaaaaaaattaaccacatggaaaaaaaaaaa

SEQ ID NO: 60 - protein, 346 aa, Homo sapien
MARPGQRWLGKWLVAMVVWALCRLATPLAKNLEPVSWSSLNPKFLSGKGLVIYPKIGDKLDIICPRAEAGRPYEYYKLYLVRPEQAAACSTVLDPNVLVTCNRPEQEIRFTIKFQEFSPNYMGLEFKKHHDYYITSTSNGSLEGLENREGGVCRTRTMKIIMKVGQDPNAVTPEQLTTSRPSKEADNTVKMATQAPGSRGSLGDSDGKHETVNQEEKSGPGASGGSSGDPDGFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 61 - DNA, 2438 bp, Homo sapien
ccggcgggggcgccgcggagagcggagggcgccgggctgcggaacgcgaagcggagggcgcgggaccctgcacgccgcccgcgggcccatgtgagcgccatgcggcgccgcgcagcccggggacccggcccgccgcccccagggcccggactctcgcggttgccgctgctgccgctgccgctgctgctgctgctggcgctggggacccgcgggggctgcgccgcgcccgcacccgcgccgcgcgccgaggacctcagcctgggagtggagtggctaagcaggttcggttacctgcccccggctgaccccacaacagggcagctgcagacgcaagaggagctgtctaaggccatcacagccatgcagcagtttggtggcctggaggccaccggcatcctggacgaggccaccctggccctgatgaaaaccccacgctgctccctgccagacctccctgtcctgacccaggctcgcaggagacgccaggctccagcccccaccaagtggaacaagaggaacctgtcgtggagggtccggacgttcccacgggactcaccactggggcacgacacggtgcgtgcactcatgtactacgccctcaaggtctggagcgacattgcgcccctgaacttccacgaggtggcgggcagcaccgccgacatccagatcgacttctccaaggccgaccataacgacggctaccccttcgacggccccggcggcaccgtggcccacgccttcttccccggccaccaccacaccgccggggacacccactttgacgatgacgaggcctggaccttccgctcctcggatgcccacgggatggacctgtttgcagtggctgtccacgagtttggccacgccattgggttaagccatgtggccgctgcacactccatcatgcggccgtactaccagggcccggtgggtgacccgctgcgctacgggctcccctacgaggacaaggtgcgcgtctggcagctgtacggtgtgcgggagtctgtgtctcccacggcgcagcccgaggagcctcccctgctgccggagcccccagacaaccggtccagcgccccgcccaggaaggacgtgccccacagatgcagcactcactttgacgcggtggcccagatccgcggtgaagctttcttcttcaaaggcaagtacttctggcggctgacgcgggaccggcacctggtgtccctgcagccggcacagatgcaccgcttctggcggggcctgccgctgcacctggacagcgtggacgccgtgtacgagcgcaccagcgaccacaagatcgtcttctttaaaggagacaggtactgggtgttcaaggacaataacgtagaggaaggatacccgcgccccgtctccgacttcagcctcccgcctggcggcatcgacgctgccttctcctgggcccacaatgacaggacttatttctttaaggaccagctgtactggcgctacgatgaccacacgaggcacatggaccccggctaccccgcccagagccccctgtggaggggtgtccccagcacgctggacgacgccatgcgctggtccgacggtgcctcctacttcttccgtggccaggagtactggaaagtgctggatggcgagctggaggtggcacccgggtacccacagtccacggcccgggactggctggtgtgtggagactcacaggccgatggatctgtggctgcgggcgtggacgcggcagaggggccccgcgcccctccaggacaacatgaccagagccgctcggaggacggttacgaggtctgctcatgcacctctggggcatcctctcccccgggggccccaggcccactggtggctgccaccatgctgctgctgctgccgccactgtcaccaggcgccctgtggacagcggcccaggccctgacgctatgacacacagcgcgagcccatgagaggacagaggcggtgggacagcctggccacagagggcaaggactgtgccggagtccctgggggaggtgctggcgcgggatgaggacgggccaccctggcaccggaaggccagcagagggcacggcccgccagggctgggcaggctcaggtggcaaggacggagctgtcccctagtgagggactgtgttgactgacgagccgaggggtggccgctccagaagggtgcccagtcaggccgcaccgccgccagcctcctccggccctggagggagcatctcgggctgggggcccacccctctctgtgccggcgccaccaaccccacccacactgctgcctggtgctcccgccggcccacagggcctccgtccccaggtccccagtggggcagccctccccacagacgagccccccacatggtgccgcggcacgtcccccctgtgacgcgttccagaccaacatgacctctccctgctttgtaaaaaaaaaaaaaaaaaaa

SEQ ID NO: 62 - protein, 606 aa, Homo sapien
MRRRAARGPGPPPPGPGLSRLPLLPLPLLLLLALGTRGGCAAPAPAPRAEDLSLGVEWLSRFGYLPPADPTTGQLQTQEELSKAITAMQQFGGLEATGILDEATLALMKTPRCSLPDLPVLTQARRRRQAPAPTKWNKRNLSWRVRTFPRDSPLGHDTVRALMYYALKVWSDIAPLNFHEVAGSTADIQIDFSKADHNDGYPFDGPGGTVAHAFFPGHHHTAGDTHFDDDEAWTFRSSDAHGMDLFAVAVHEFGHAIGLSHVAAAHSIMRPYYQGPVGDPLRYGLPYEDKVRVWQLYGVRESVSPTAQPEEPPLLPEPPDNRSSAPPRKDVPHRCSTHFDAVAQIRGEAFFFKGKYFWRLTRDRHLVSLQPAQMHRFWRGLPLHLDSVDAVYERTSDHKIVFFKGDRYWVFKDNNVEEGYPRPVSDFSLPPGGIDAAFSWAHNDRTYFFKDQLYWRYDDHTRHMDPGYPAQSPLWRGVPSTLDDAMRWSDGASYFFRGQEYWKVLDGELEVAPGYPQSTARDWLVCGDSQADGSVAAGVDAAEGPRAPPGQHDQSRSEDGYEVCSCTSGASSPPGAPGPLVAATMLLLLPPLSPGALWTAAQALTL

SEQ ID NO: 63 - DNA, 1009 bp, Homo sapien
gacaaatgagggtttggcatgcagctcgtcatcttaagagttactatcttcttgccctggtgtttcgccgttccagtgccccctgctgcagaccataaaggatgggactttgttgagggctatttccatcaatttttcctgaccgagaaggagtcgccactccttacccaggagacacaaacacagctcctgcaacaattccatcggaatgggacagacctacttgacatgcagatgcatgctctgctacaccagccccactgtggggtgcctgatgggtccgacacctccatctcgccaggaagatgcaagtggaataagcacactctaacttacaggattatcaattacccacatgatatgaagccatccgcagtgaaagacagtatatataatgcagtttccatctggagcaatgtgacccctttgatattccagcaagtgcagaatggagatgcagacatcaaggtttctttctggcagtgggcccatgaagatggttggccctttgatgggccaggtggtatcttaggccatgcctttttaccaaattctggaaatcctggagttgtccattttgacaagaatgaacactggtcagcttcagacactggatataatctgttcctggttgcaactcatgagattgggcattctttgggcctgcagcactctgggaatcagagctccataatgtaccccacttactggtatcacgaccctagaaccttccagctcagtgccgatgatatccaaaggatccagcatttgtatggagaaaaatgttcatctgacataccttaatgttagcacagaggacttattcaacctgtcctttcagggagtttattggaggatcaaagaactgaaagcactagagcagccttggggactgctaggatgaagccctaaagaatgcaacctagtcaggttagctgaaccgacactcaaaacgctactgagtcacaataaagattgttttaaagagtaaaaaaaaaaaaaaaaaaaa

SEQ ID NO: 64 - protein, 261 aa, Homo sapien
MQLVILRVTIFLPWCFAVPVPPAADHKGWDFVEGYFHQFFLTEKESPLLTQETQTQLLQQFHRNGTDLLDMQMHALLHQPHCGVPDGSDTSISPGRCKWNKHTLTYRIINYPHDMKPSAVKDSIYNAVSIWSNVTPLIFQQVQNGDADIKVSFWQWAHEDGWPFDGPGGILGHAFLPNSGNPGVVHFDKNEHWSASDTGYNLFLVATHEIGHSLGLQHSGNQSSIMYPTYWYHDPRTFQLSADDIQRIQHLYGEKCSSDIP

SEQ ID NO: 65 - DNA, 3410 bp, Homo sapien
gaattcgaggatccgggtaccatgggcggcggcaggcctagcagcacgggaaccgtcccccgcgcgcatgcgcgcgcccctgaagcgcctgggggacgggtatgggcgggaggtaggggcgcggctccgcgtgccagttgggtgcccgcgcgtcacgtggtgaggaaggaggcggaggtctgagtttcgagggagggggggagagaagagggaacgagcaagggaaggaaagcggggaaaggaggaaggaaacgaacgagggggagggaggtccctgttttggaggagctaggagcgttgccggcccctgaagtggagcgagagggaggtgcttcgccgtttctcctgccaggggaggtcccggcttcccgtggaggctccggaccaagccccttcagcttctccctccggatcgatgtgctgctgttaacccgtgaggaggcggcggcggcggcagcggcagcggaagatggtgttgctgagagtgttaattctgctcctctcctgggcggcggggatgggaggtcagtatgggaatcctttaaataaatatatcagacattatgaaggattatcttacaatgtggattcattacaccaaaaacaccagcgtgccaaaagagcagtctcacatgaagaccaatttttacgtctagatttccatgcccatggaagacatttcaacctacgaatgaagagggacacttcccttttcagtgatgaatttaaagtagaaacatcaaataaagtacttgattatgatacctctcatatttacactggacatatttatggtgaagaaggaagttttagccatgggtctgttattgatggaagatttgaaggattcatccagactcgtggtggcacattttatgttgagccagcagagagatatattaaagaccgaactctgccatttcactctgtcatttatcatgaagatgatattaactatccccataaatacggtcctcaggggggctgtgcagatcattcagtatttgaaagaatgaggaaataccagatgactggtgtagaggaagtaacacagatacctcaagaagaacatgctgctaatggtccagaacttctgaggaaaaaacgtacaacttcagctgaaaaaaatacttgtcagctttatattcagactgatcatttgttctttaaatattacggaacacgagaagctgtgattgcccagatatccagtcatgttaaagcgattgatacaatttaccagaccacagacttctccggaatccgtaacatcagtttcatggtgaaacgcataagaatcaatacaactgctgatgagaaggaccctacaaatcctttccgtttcccaaatattggtgtggagaagtttctggaattgaattctgagcagaatcatgatgactactgtttggcctatgtcttcacagaccgagattttgatgatggcgtacttggtctggcttgggttggagcaccttcaggaagctctggaggaatatgtgaaaaaagtaaactctattcagatggtaagaagaagtccttaaacactggaattattactgttcagaactatgggtctcatgtacctcccaaagtctctcacattacttttgctcacgaagttggacataactttggatccccacatgattctggaacagagtgcacaccaggagaatctaagaatttgggtcaaaaagaaaatggcaattacatcatgtatgcaagagcaacatctggggacaaacttaacaacaataaattctcactctgtagtattagaaatataagccaagttcttgagaagaagagaaacaactgttttgttgaatctggccaacctatttgtggaaatggaatggtagaacaaggtgaagaatgtgattgtggctatagtgaccagtgtaaagatgaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagactgtgcaagggaaggaatatgtaatggcttcacagctctctgcccagcatctgaccctaaaccaaacttcacagactgtaataggcatacacaagtgtgcattaatgggcaatgtgcaggttctatctgtgagaaatatggcttagaggagtgtacgtgtgccagttctgatggcaaagatgataaagaattatgccatgtatgctgtatgaagaaaatggacccatcaacttgtgccagtacagggtctgtgcagtggagtaggcacttcagtggtcgaaccatcaccctgcaacctggatccccttgcaacgattttagaggttactgtgatgttttcatgcggtgcagattagtagatgctgatggtcctctagctaggcttaaaaaagcaatttttagtccagagctctatgaaaacattgctgaatggattgtggctcattggtgggcagtattacttatgggaattgctctgatcatgctaatggctggatttattaagatatgcagtgttcatactccaagtagtaatccaaagttgcctcctcctaaaccacttccaggcactttaaagaggaggagacctccacagcccattcagcaaccccagcgtcagcggccccgagagagttatcaaatgggacacatgagacgctaactgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgcattttcttcacttgcaggcaaacttggctctcaataaacttttaccacaaattgaaataaatatatttttttcaactgccaatcaaggctaggaggctcgaccacctcaacattggagacatcacttgccaatgtacataccttgttatatgcagacatgtatttcttacgtacactgtacttctgtgtgcaattgtaaacagaaattgcaatatggatgtttctttgtattataaaatttttccgctcttaattaaaaattactgtttaattgacatactcaggataacagagaatggtggtattcagtggtccaggattctgtaatgctttacacaggcagttttgaaatgaaaatcaatttaccccatggtacccggatcctcgaattc

SEQ ID NO: 66 - protein, 748 aa, Homo sapien
MVLLRVLILLLSWAAGMGGQYGNPLNKYIRHYEGLSYNVDSLHQKHQRAKRAVSHEDQFLRLDFHAHGRHFNLRMKRDTSLFSDEFKVETSNKVLDYDTSHIYTGHIYGEEGSFSHGSVIDGRFEGFIQTRGGTFYVEPAERYIKDRTLPFHSVIYHEDDINYPHKYGPQGGCADHSVFERMRKYQMTGVEEVTQIPQEEHAANGPELLRKKRTTSAEKNTCQLYIQTDHLFFKYYGTREAVIAQISSHVKAIDTIYQTTDFSGIRNISFMVKRIRINTTADEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPICGNGMVEQGEECDCGYSDQCKDECCFDANQPEGRKCKLKPGKQCSPSQGPCCTAQCAFKSKSEKCRDDSDCAREGICNGFTALCPASDPKPNFTDCNRHTQVCINGQCAGSICEKYGLEECTCASSDGKDDKELCHVCCMKKMDPSTCASTGSVQWSRHFSGRTITLQPGSPCNDFRGYCDVFMRCRLVDADGPLARLKKAIFSPELYENIAEWIVAHWWAVLLMGIALIMLMAGFIKICSVHTPSSNPKLPPPKPLPGTLKRRRPPQPIQQPQRQRPRESYQMGHMRR

SEQ ID NO: 67 - DNA, 13497 bp, Homo sapien
atgctgatggctgttggcgatcggatctacgatctcgttatgcaaattggcctgcccgccgatggcttccgcctcttccggcgtggagcggcctggcaaacgctcttcctgctctgcgctctcgcctattgcataaatgaagccagcagcgagggtcgcgtggtctgctactatacgaactggagtgtctatcggccgggcacagccaaattcaatccgcagaacataaatccatacctgtgtacacatttggtgtacgcgttcggtggattcaccaaggataaccaaatgaagccctttgacaagtaccaggacatcgaacagggtggctatgccaagttcactggactcaaaacgtacaacaaacagctgaagaccatgattgctattggcggatggaacgaggcgagttccagattctctccattggtggctagtaacgagcgtcggcagcagttcatcaagaacatcttgaaattcctgcggcagaaccattttgatggcatcgacctggattgggaatatccagcccatcgagagggcggcaagtcccgggatcgtgataactatgcccagtttgtccaagagcttagggccgaattcgaaagggaagcggagaagaccggacgcactcgtctcctattgaccatggcagttcctgctggcatcgagtacattgacaagggttacgatgtgcccaagttgaacaagtatttggattggttcaatgtgctgacctatgatttccattcttcccacgagccatcggttaaccatcatgctccgctttattcgctggaggaagactccgagtacaattacgatgccgagttaaatattgactactccatcaaatactatctgaaagcgggtgcagatcgtgacaagcttgttctgggcatacccacctatggccgatcctatactttgatcaacgaggagagcaccgaactgggagcaccggcagagggtccgggagaacagggtgatgccaccagggaaaagggatacctggcttactatgagatctgccagaccctcaaggacgaccccgagtggactgtggtgcagccgaatgccaatgttatgggtccctatgcctacagacgcaatcagtgggtgggctacgatgatgaggctatcgtgcgcaagaaggccgaatatgtggtggcccagggactgggcggcattatgttctgggccatcgacaacgatgatttccgcggcacctgcaacggaaagccatatcctcttatcgaggctgccaaggaagccatggtggaggcattgggactgggcatcaacgaggtggccaagccaagtggaccacagaagccatcaagatctcgcagtcgcgataatgccagcaataggaaccgtctaaatggcaagaccgaagctccactcagctccaggagacccagtgccacaaggagaccagctgtgagctctactcaggcaccaccgccaagcaccaccttcaaactgaccgaagccgagggatcatctctttacattggaggcagggcatccaccacgccgccaccaccaacgactcccgatccaggttcggacttcaagtgcgaggaggagggattcttccagcatccaagagattgcaaaaagtactactggtgcctggatagtggaccgtctggtctaggaattgtggctcacatgttcacttgtccatcgggactttactttaatccggctgccgattcctgcgactttgctcgcaatgtgccctgcaagaccaaaaaatctacaacggcggcaccagtgacatccacaaccccggcaaccaccacagtgcgttccaatcgagtaacagctgctcccacgtctcgtccagtatatccacgcaccaccaccacaacaagcactacgacaaccactacgacaactccatccactgttgatgaagacttggaatatgaagaagatactgacgagctgtcgccaagcaaatccacggatgctgaggaggatccacaggtgattaaggaactaatcgatttgattcgcaaagtgggcggcgtggagcaactggagaagcatcttttgcgtaacaaggatggatcgattacattgaaggaaaattctgccactggtgcagccactactccgtcgactataagcaaatctctgtatgatcgtgtgctaagtcgacctggaacattgaattccttcagccgcaatcgtttcaagatcagcgaagcttcagagacaagcacagaacccactacttcgagcagttcgtcaaggggttcatcgactctcacctccaacaccaactccaaatattcctcagttctgaggggcaacagtcgccaaggtccacagaacgagggcattgaaaaattggctgagttcgatggtttcctgaaggaacgcaaacaatatgtgaccatcaatcgccatcgatctgcgagtcagggagatgaggaggagcacgcagatcagcaggaagaggaggagaacctggccgaagttgagacaaccacccgtcgtcctttgagctctatcactccgtcttacacaagtctgagacgctctaggcccaccacggtggcaccgccagcagaagaatcccacgaagaggcagagcaacagacccagacacaggtgaaatcctatgccaccttgagccgtactcgtggacgcaccacgtcatctccagaagtcacggaagcggcgcccagttcgacaaccaatcgttacaaatacttcgagcgtacacgacccactaagagtgccactgccgaagattccgaagatccaacagaagatgaagaggaagagtacgaggacgagcaaaaggacattgtcacggtacagagcaaacaatccacaaacacacgtaaatatgcgagcatcggccgcagaacaaccaccaccacaacagcaacaccagaaactacaactacaacaacaacaacaaccgccggcactgagactgccaaggccagcaccaccaccaacaacaacaacaacaacaacagccactacaacagcagcaataacaacaacaatgtgaaactgaataaccaattaccaacagaagagaacatcacaacaacacccagcaccaccgcccaatccgaaaccacaaccacaactaacgaaaccactgaaccaaatgaaagcacctcaaccaccaccacatccataactaataatctgcataccaccaccaccacacccacaccgattgtagcatccactgtcccaacaacaaccgctaatggcattagctcagactccctgctagccaccgagttgagtgaagcctcacccacccacctttctcccagtcccgactcagaaacatcaacaccgacaacaacaagcacaacaacaacggaacaacccgaactggacaccacaacaacgacaccgaaaaccacaacaacaaccaccacgggcaacaacgaactgaatgacgttaacaacgtggacgaggacagtgaggtaacgaaaaccaagactcaatacaagtatgcaaccaccaaccgtcgacgcataaccacaacaacaacaacagccaccaagaacagcaacaacaacaacaatgcagaagctgcgaacgatgctagtccaacaacaaacggtttgagtagtttaaatagcattagaaccaacccgggcagaaggcaaccccaaccagagcagacccaaacaacaacctccgaaccaaacctttctagtcccagaccctttggttatccccgacgtcgcacacgacccacagttagcaccacaaccaccactatttcccaaaccgataatgataacaataccgataataacgataacgaaaccgatgcagttgctcaagtagtgaagaagacacgactatctccaggggataggcccaaggtgagtgccagtcttccaacagcaaccgcaatcaacacacgaaccaatacctcctcactgcaccaccaagaatcccaagtagaagtagctggtaacggtggcaatgatagcttaaggcatgatgtagttagctctagtttaagtcaatcccaatccaacaaaatcgataccgatgacctcagtaccacacagcagcataccaagtacacttggcgtgcggtgcgacgtcctgcatcgcagcgcacagtggttcccaattctttagcgggcgacgacaaggactcccgacgctttgccggcaagcagttgaacaccgaatcaattgtggatgacgagcttcaaaccaccaccaagttccgcagtcgccgcctaaattccgccgaagatgagtccgaagtggctctcgaggtggcaaccgcgaccccaacgcacggttcgcgcagctaccagagcatccaacgttctgctagcaaagcgagcttagatgactctcaaatccattacaaagccatcattcgcgattccgagggcggtgctcatctaaccgccggacgcagttccagctttgtgaggaatttcggcgatgcagccaaaccaactccaccgcaccaacctattagcagaggtggacagattgtggagagcaccaccgaagatgaaaatgtggctgctgaaataatcgatgatgagaagaggggtgagacgaaggcgcctgctggaagcgagaatactgatgacagcaacacggcgaccgaacaggaatctcccgaaattgtaactgaagctgcacaaccacagctcgagattaccactttgccatcagaaacttcagatgtcagcagttctacggaacaatctgtaagcagcaccactgaagaaagctccagcagcaccgcagacttggatatcgtcgctgaagaagccagtctaggcgctgaaaccgacaaaaagtctacttctgagaatgataatggagaatcttcaacggaaatcagctcttctgaagccccaattagcagcacaactggacaatctgaagatgtaagcagcaccactgaaactaattcagaagccattgaaaaagaaattgcctctgattctaacgatggatccagtgatgatcctgccagcagtacggaatttatagaaatcactaacacaactagcagcccagtcagtctccaggaagattcttcgacaacgacagaaaaactcacacggcgagcatttaaccgctttagctcaactactcctgcagttgtgccagaagatgaaaccacatccaccgtaaaccaaaggcgtcgagttattgtgcgtaacagaatcagcaccactgaagcagagagtgaggctcagacaaccacagaagaaccaaaacgacgtagtttttacagaaccagcaccacagctgaaccaagcagctcaactgaagcagacagtgatgcccagatatctactgaaactacaacacgtcgttctttctttagaactaggaccactgaagcagctagcagcaccactgaagaacctagcagccccacagaaccagagattgaagtcgagacaaccaccgagggtccaacgcgccgttcgttcttcagacgtagcaccactgtggctcctagtagcaccactgaagaaatcagtagcagctccgtagatgatgatgctgaagcgaatattatcaccactcgtcgttcgctattcactacgccagcgccaagttcgacagaagccacaaccacagcaactgcagaagattccgaagttagtagctccaccaggcgtagtttcttccgtaccagcactaccacagaaggcacaacaagtactacagaagaggcaaaggatattgaacatgagagtgagaccaccgctgctttgcctaaacgtcgagttatagtcagaggcaattttaggccccgcaaggagggagatctctcctctcttctagcggcagatgccaacaagcgagtgagaaacaaccacagcaccacaagcactgaaacacctgctaatagtcaatcaactacatccaatgaagaagatacggtggcacaaccaccacaagctgaggtgaaggcaaccactggtcgggtttccttaaatgctgttcgaaaccgcaccaccacaaaaaccgaaagtctaggcaatggaatcacccgcactcgcaccacctacgtccgcactctcgacgctggtcaaaagattgtgaaacgcattcacactaaaaccatagaagagaaacccgcggagtatgagtacattatcgatgaagtgacccacccaccagcggcgagcaccacgcctcgcacagtgacacgcaatcgtggatccgtgcgattccagagtaatgatctttcttcgctgctggccttggactttgcctcacgtagcacccgcaagaaacaggcccaaacagagacaacagttaccaaaacgcgaagacgcctacttaagaaacccaaggaaaccatcgaacatgaagaggtggaagaatacgagtatgaagcaggtcaggaagctgggaacgaagttgaggaggcgccacgtgtgagcacgacagccaggactataatccgaagaactagaccgacaacaatcagaacaacaactacggaaacacctcaaaatattgaggctagcacccgcagggctagcttcgctttcaagcgtccctccaaagttagcacaaccactgaggaaccaactacaagctctacggaacccacaatatctgcagaagctaccaccagaagagttctcaacttcagaagaccagtgagcacgacaagcaccccagcttcagatgagtccactgaggaggcaactgctgcccccattgaggccaccaccagaagagttcttgcgttcaaaagacctgtcagcacaacaaccaccccagctccagtagacgaagagtccacagaagaatccacccctacttctatagagggcaacaccagaaggattctcgcttatagaaggcccgtgagcacaacaactaccacaccagttcctgttgaagatgaatcttcaacagatcagttggcagcggccaagcaaaagtttataaatcgtctcaaatctagtaccacaacgacaacatcaattcctgaaacaacaaccaccgaagaagacttaagcgatcttaaggtacagcttagcaatgccataaaccgtttgcaaacggaaaataaactagaagtccagacaatcaccaagggtagtgaagccgcagaagatgaaggcgatgataagctgtctttgccaatttaccacagaagaaagtactatcagtatgtaaaggattcaccaattacctatatcgacaagtcccccgcaccaccggatatcgaaagtgtgactgtaaatattaagcaacaaattcatgatgtgttcaatgtgagtgaaaatgagacgccacataattctctgggcgatgacgaagagaccgaaggacatcgcgtggctatggcgcaggccaaggaaatcaatgccgagctcgaagaaaaggaaagaggcgaagatgaagccagggctttacgcacctacaccagattaaaccgcacccgcttgaccctgtccaccaggttacaggaaaagacccaaagtgaaccgctagacaccacaaccaggaggagctacagcgtccctcagcgtttccgtattcgctcaacgacgccgataccatctaaaatagaaaacagcgaagaggacgatgaagaaactaaagacaatgaaggaccatcgccgagcacaacaaccgtgacaccaccatccataaaactacctaccagacgactttttacacccagaaggcccgttaatgctgtagaggactcagattcttctgatatacgtaaagacaatgaagaagagttaaaggtggaatcaaccacaaaacgcctatatgccggcttaaatagattgagaggcaggggaagcaccaccacaaccaccgaagaagcaactgattcaaccaccgagacagcaaccacaacagccaaaagtacaagacaaccttatgtgggtattagtcgcagagtaacaactacaacgaccactgagaagtcagccgaaagtagtactgaatataatggaaatgaagatgaagaaacggagtccaccacagtaactcctgaacaggagataagcgatgatgctgaagaaaataaggttgcaatcaaagaaatagatgaccaagttagcaagaaagctcccgaagaggctgaagacaccagcactgaggagcccgaactggaagcctttatagacgatgacaatgaaatccccctcgaagaaagcggacctaaaactgaaaccacatcaacaacaacaacaacaacatcaacgaccaccactacaccagcaagcaccacatcacgtcgtcagttggtaattcgtcgccgttttaatggcaccataacaacaaccactacggttgcccctgtcgctgatgagaatctcgaaaatgagatcgatccaagtgatacggagagttcaacaccaaaagcggcaactacaacttcaccaagacggcaacttttaatacgccgacgtttcaatgcgaccagcagcgggtcaacaacaacgacaactgcaaatcctagtgccgacaatgaaatagatcagggcgagacaaaaagaacaacgcgtcgaccaattctgtcacgtcggcgttttaatgcgacttccatcacagcaaccacgacgggatcaacgaacggcgacgagataagcacacgacgtccgtatgcggccttgaaccgctcacgtaatcgctttacaacaccgcaaacaacaaccaccgacggtggggcaaacggcgatgatgatgattacgacggagaggaagaggagcagctagcaccgccgcgagctgtcttccttcaaacaaaccgccatcgcgctctgaaacccactcctgaagacgaggaggagggtgcagctgcggtgccaggacgtaggccactcaattttgccgcacgtcgcaccaccgctgcaccactgagagttagctctagtacgcgacgaaatctggtggcaatcaatagaaatctttaccaccgaccagaggaagataacgaggaagaacccgaagaggagtacgacgaaaacgaggatggggatgatgatcaagaagaatctgtagatcctcaagtcacaagcactacaacacgttcacgactaaatcaattgctagccaacaggcaaagacaaccactcaggacaaccactgaaaaacagacagagacagatagcaacgatacagaaactgactcggacaacggagatgaaaacgatgatgatgaagataacgatagctccgtcgaggtatcgaacagtaaccacacactaaaacacagcactatattcggggttggcaccactaactttaacaatcttactaaccgtagcactgcactgaatgtagctagtcaacgtagtaacagcaccgtagccaattatattaatcgattcaagtcaaacagttacacaaacaaaaacaaaccagtcacagtcacagctaatattaaggcagatagtacagatgataaagataatgataatgatgaagatgatgatgatgatgataacgatgatgataattatgctagtctagagaatgaggggaaggagaaaacgagtggggctggtttgaatgccttaggtaatgatgttaattccacacgacgctttcagaatcgttatcaattgagccgcaccagaggcagcaccaccaccaacaccaacccaacaacaacacaacaaccccaaacaacaagcaccgccagacgattggcgtttgggggccgtcagcgcgcccaggtaactaaactaaccctagtcgatgagcagactgaagagaccgagactaagggtgattcccgcgaagaggagaaggaggaggaggaggaagaagactccaacgcaaccacaacaacaacaaccacaaccaccagcagaccaacaccaaagcgcatacgtgtgcttaaattccgcagacccttaaatagcaatagcaatagtaccatcaatgtagatagcaccactaatagcgccactgacactaacccagacaccaccacagccacaccaacaacagcaggtcaaagcaccaccagcaacagcaacaacaacaacaacaacaccacaagcaccaccggcaataagcgtttccgcaagattgttcgcaaattgaggcctgtggactcaagtactgcggctagtgtggataattccgatgagaccacccgtaaacctttcgttcccagccacaccagattcgctgatcaggataatgaccttgttaatctccgccagcgaatcaaggagcagcaagcacgaggtgagccgcaggatggagttattagcaatcgatttaagaccctgggccaaaaggacgatcaggatgtgtcggagttgcaaaagttgagggacaaagtaaaggcagagcaggcgaggggagaaggagaacagggcgtgatcaacgatcgtctgaagaagcttctggccgagaagggctcgagcatctcaagccaaagagaagaatcttcaacagatgatgactcttcatcggtttcctcggccagacccttctttaaacgaaaactggtggcacgtagaccctacacaccaccctcggccagtggtggcaccacaaaggcaccactgacctttagcacctcccgacctacggctaaattcgtgcgcaggaagaacggaagatttgatcccttcaactcgagtgttcgcaatcgtggagagggctttgtgagaagtgatccgagaggatccagattgcctggaaccgatcgcttcaagtctcagggaaatagcgaagacgatgacgaagtggaagagcgccatgagcaaccactgcagaatcagtttgcaaccacattgcgaagaccttttgtgccgaagacccgaccagttttggataagtccaagcctgagcaggaagatggtgcagaagagagtgaggaggaagacgaggaggaggattccgaggaagaaggtgacgaagaagaggatgaagaagaagaggacgtgaagccaggtggagaagaggacaatgcacaagaggataacaaaccgaagtttaattccccttacaaacccaaagataatcgagctccaccgggtagtcgtcccacatttggaaccaccggaagtggatcaccacctaccgcatccggaaatgtaccatacaatcctcgcaatcgcccatcaaactcggcaaatggaaatagcacaccatccaaccgttttggcaccacaaagcgacctcgtgtggttaaccgaccaccgggagtagcctcaccaaatttgaccttgaaacccgtggccagtgattatgaacgcaccacaccgctgacaccactgaaaccggcgccatttatacccagcaataacaggagttacgagaggaagtactcgggtccatccacagaggccgcggagacggccagcgagaattccctgatagaagatttgaatatcgatgcactgaatgcgagaaacaagaagatctttgacaaacacagtaagaaacatccggctctgaagccaaaggtggttaaggtggaatccgagacgggattagaggtggaagccggtacagaggtggcggtggaagatgaaaccaccgaggagcagcagcaggagcagggctttgtgaccaccacacccagtacaccaccatcaccagcaccacccagcacacagtcagacacagccaccaccacagacacgccaccagaaactgaaactgaaactgaaactgaaactgaaacagaaactgaaactgaaaatgtaaccgaaattgaaacagcgaccaatgctaatgaagccacttccatcaattcacaggatcaaaccatctcgagcactactcaagcaccaccgccggccacaaccttgttgcatgtgttcacccttctcgagggtgagggtcaagaagaggagcccacgaccagaaagccaacggtacgcctctatcccaccatacaaacggaagtggtgcccaagcacaagttaatcgagattaatcgcattgtcgagattaattccaaacaggccaaggctgctcagaggaagtccaaggcgaatcatgactttagcaccttaatggtggagtccctgccccacgtggagcagctgggcgagatcagtgtggtgaaatatgtccacttggtggatggcagtgatattcagatcaacgatggtcacagcacagtggcagattacacgcccaccgaacccacatcagcggctgaacgtcctgtctcgctgcccgttaggaattctctgccagaaaccgagggcgctgacaccgatcgcagtggcaagtccttggtgcctgaagttctaactgccgcattggagacctctaccatttcgctggagggactatttgattcggccagaaagggcaagcaactgagcagtaatacaatcattggcgaaacggaggagagcacaaccattggcagtagcagtagtttggccagcgaaaccggagagaccaccacaccggcacccacttacgtaagacccattgtacccctcctaagacccgagtccaatgagtcctcaccactggtcatttcgatagccaacctcgatcaggtgatactgagcaaggtgcaaaagtcgctggccgagaatagccaaaccacagtggcccccgaagctgccagcgactcgaattccgccttctctgtgcgccaaccgctggtggtgcaggcgcccattagcaatggtgctcaggaaatcgatcaggatacacttaacacacaagatcagacaatcaatggtgctattagtgtgaaaactaatccaattatacaaacgacaaccaacaggccaaatgacgatcaggtggctgaagaaaccacaatctttagcattgaaacggccactgagccagagcttaacacacaaacaacaattcccaaaacagaagccaatagcgaaacagtaacagccatgccaattggtgctgttattatgggtcagtttggtctaaacacacaatcaacaaccgctgttgacaatgacaaccaactaaatgctcaaacaacaagcacaattagcagtggcgctgttagttccgtagctattggtggtaacacacaaacagcaaacgcggacaatgctcgccaagaaaatacacaatcaacaggcacaataacaagtgagattagcagtggcgctattagttccgacaaccacaatcacataggcacacaaacaacagccaccatcgatagcagttcagaaacaacacccacccaaatatcaaccacaattagcagtggtgcaattagtggtcatattgatggtagcatcaatctgaacacacaaacaaacaccaccatcagcactaacaacaccaccaccagcaccaccgatgtgggcagcaaagttagcgaggcagttagctttagttccgagacgcatgtggtgcatcgcaaaaagatgggtcgcaaggggcgtggccgacgcctgcgcaatcgcaaaacgacaacaacgacaacaaccacagaaacgccaaccacgacggaggcgacatttgatgacaccaccacagtggtgccagaatag

SEQ ID NO: 68 - protein, 4498 aa, Homo sapien
MLMAVGDRIYDLVMQIGLPADGFRLFRRGAAWQTLFLLCALAYCINEASSEGRVVCYYTNWSVYRPGTAKFNPQNINPYLCTHLVYAFGGFTKDNQMKPFDKYQDIEQGGYAKFTGLKTYNKQLKTMIAIGGWNEASSRFSPLVASNERRQQFIKNILKFLRQNHFDGIDLDWEYPAHREGGKSRDRDNYAQFVQELRAEFEREAEKTGRTRLLLTMAVPAGIEYIDKGYDVPKLNKYLDWFNVLTYDFHSSHEPSVNHHAPLYSLEEDSEYNYDAELNIDYSIKYYLKAGADRDKLVLGIPTYGRSYTLINEESTELGAPAEGPGEQGDATREKGYLAYYEICQTLKDDPEWTVVQPNANVMGPYAYRRNQWVGYDDEAIVRKKAEYVVAQGLGGIMFWAIDNDDFRGTCNGKPYPLIEAAKEAMVEALGLGINEVAKPSGPQKPSRSRSRDNASNRNRLNGKTEAPLSSRRPSATRRPAVSSTQAPPPSTTFKLTEAEGSSLYIGGRASTTPPPPTTPDPGSDFKCEEEGFFQHPRDCKKYYWCLDSGPSGLGIVAHMFTCPSGLYFNPAADSCDFARNVPCKTKKSTTAAPVTSTTPATTTVRSNRVTAAPTSRPVYPRTTTTTSTTTTTTTTPSTVDEDLEYEEDTDELSPSKSTDAEEDPQVIKELIDLIRKVGGVEQLEKHLLRNKDGSITLKENSATGAATTPSTISKSLYDRVLSRPGTLNSFSRNRFKISEASETSTEPTTSSSSSRGSSTLTSNTNSKYSSVLRGNSRQGPQNEGIEKLAEFDGFLKERKQYVTINRHRSASQGDEEEHADQQEEEENLAEVETTTRRPLSSITPSYTSLRRSRPTTVAPPAEESHEEAEQQTQTQVKSYATLSRTRGRTTSSPEVTEAAPSSTTNRYKYFERTRPTKSATAEDSEDPTEDEEEEYEDEQKDIVTVQSKQSTNTRKYASIGRRTTTTTTATPETTTTTTTTTAGTETAKASTTTNNNNNNNSHYNSSNNNNNVKLNNQLPTEENITTTPSTTAQSETTTTTNETTEPNESTSTTTTSITNNLHTTTTTPTPIVASTVPTTTANGISSDSLLATELSEASPTHLSPSPDSETSTPTTTSTTTTEQPELDTTTTTPKTTTTTTTGNNELNDVNNVDEDSEVTKTKTQYKYATTNRRRITTTTTTATKNSNNNNNAEAANDASPTTNGLSSLNSIRTNPGRRQPQPEQTQTTTSEPNLSSPRPFGYPRRRTRPTVSTTTTTISQTDNDNNTDNNDNETDAVAQVVKKTRLSPGDRPKVSASLPTATAINTRTNTSSLHHQESQVEVAGNGGNDSLRHDVVSSSLSQSQSNKIDTDDLSTTQQHTKYTWRAVRRPASQRTVVPNSLAGDDKDSRRFAGKQLNTESIVDDELQTTTKFRSRRLNSAEDESEVALEVATATPTHGSRSYQSIQRSASKASLDDSQIHYKAIIRDSEGGAHLTAGRSSSFVRNFGDAAKPTPPHQPISRGGQIVESTTEDENVAAEIIDDEKRGETKAPAGSENTDDSNTATEQESPEIVTEAAQPQLEITTLPSETSDVSSSTEQSVSSTTEESSSSTADLDIVAEEASLGAETDKKSTSENDNGESSTEISSSEAPISSTTGQSEDVSSTTETNSEAIEKEIASDSNDGSSDDPASSTEFIEITNTTSSPVSLQEDSSTTTEKLTRRAFNRFSSTTPAVVPEDETTSTVNQRRRVIVRNRISTTEAESEAQTTTEEPKRRSFYRTSTTAEPSSSTEADSDAQISTETTTRRSFFRTRTTEAASSTTEEPSSPTEPEIEVETTTEGPTRRSFFRRSTTVAPSSTTEEISSSSVDDDAEANIITTRRSLFTTPAPSSTEATTTATAEDSEVSSSTRRSFFRTSTTTEGTTSTTEEAKDIEHESETTAALPKRRVIVRGNFRPRKEGDLSSLLAADANKRVRNNHSTTSTETPANSQSTTSNEEDTVAQPPQAEVKATTGRVSLNAVRNRTTTKTESLGNGITRTRTTYVRTLDAGQKIVKRIHTKTIEEKPAEYEYIIDEVTHPPAASTTPRTVTRNRGSVRFQSNDLSSLLALDFASRSTRKKQAQTETTVTKTRRRLLKKPKETIEHEEVEEYEYEAGQEAGNEVEEAPRVSTTARTIIRRTRPTTIRTTTTETPQNIEASTRRASFAFKRPSKVSTTTEEPTTSSTEPTISAEATTRRVLNFRRPVSTTSTPASDESTEEATAAPIEATTRRVLAFKRPVSTTTTPAPVDEESTEESTPTSIEGNTRRILAYRRPVSTTTTTPVPVEDESSTDQLAAAKQKFINRLKSSTTTTTSIPETTTTEEDLSDLKVQLSNAINRLQTENKLEVQTITKGSEAAEDEGDDKLSLPIYHRRKYYQYVKDSPITYIDKSPAPPDIESVTVNIKQQIHDVFNVSENETPHNSLGDDEETEGHRVAMAQAKEINAELEEKERGEDEARALRTYTRLNRTRLTLSTRLQEKTQSEPLDTTTRRSYSVPQRFRIRSTTPIPSKIENSEEDDEETKDNEGPSPSTTTVTPPSIKLPTRRLFTPRRPVNAVEDSDSSDIRKDNEEELKVESTTKRLYAGLNRLRGRGSTTTTTEEATDSTTETATTTAKSTRQPYVGISRRVTTTTTTEKSAESSTEYNGNEDEETESTTVTPEQEISDDAEENKVAIKEIDDQVSKKAPEEAEDTSTEEPELEAFIDDDNEIPLEESGPKTETTSTTTTTTSTTTTTPASTTSRRQLVIRRRFNGTITTTTTVAPVADENLENEIDPSDTESSTPKAATTTSPRRQLLIRRRFNATSSGSTTTTTANPSADNEIDQGETKRTTRRPILSRRRFNATSITATTTGSTNGDEISTRRPYAALNRSRNRFTTPQTTTTDGGANGDDDDYDGEEEEQLAPPRAVFLQTNRHRALKPTPEDEEEGAAAVPGRRPLNFAARRTTAAPLRVSSSTRRNLVAINRNLYHRPEEDNEEEPEEEYDENEDGDDDQEESVDPQVTSTTTRSRLNQLLANRQRQPLRTTTEKQTETDSNDTETDSDNGDENDDDEDNDSSVEVSNSNHTLKHSTIFGVGTTNFNNLTNRSTALNVASQRSNSTVANYINRFKSNSYTNKNKPVTVTANIKADSTDDKDNDNDEDDDDDDNDDDNYASLENEGKEKTSGAGLNALGNDVNSTRRFQNRYQLSRTRGSTTTNTNPTTTQQPQTTSTARRLAFGGRQRAQVTKLTLVDEQTEETETKGDSREEEKEEEEEEDSNATTTTTTTTTSRPTPKRIRVLKFRRPLNSNSNSTINVDSTTNSATDTNPDTTTATPTTAGQSTTSNSNNNNNNTTSTTGNKRFRKIVRKLRPVDSSTAASVDNSDETTRKPFVPSHTRFADQDNDLVNLRQRIKEQQARGEPQDGVISNRFKTLGQKDDQDVSELQKLRDKVKAEQARGEGEQGVINDRLKKLLAEKGSSISSQREESSTDDDSSSVSSARPFFKRKLVARRPYTPPSASGGTTKAPLTFSTSRPTAKFVRRKNGRFDPFNSSVRNRGEGFVRSDPRGSRLPGTDRFKSQGNSEDDDEVEERHEQPLQNQFATTLRRPFVPKTRPVLDKSKPEQEDGAEESEEEDEEEDSEEEGDEEEDEEEEDVKPGGEEDNAQEDNKPKFNSPYKPKDNRAPPGSRPTFGTTGSGSPPTASGNVPYNPRNRPSNSANGNSTPSNRFGTTKRPRVVNRPPGVASPNLTLKPVASDYERTTPLTPLKPAPFIPSNNRSYERKYSGPSTEAAETASENSLIEDLNIDALNARNKKIFDKHSKKHPALKPKVVKVESETGLEVEAGTEVAVEDETTEEQQQEQGFVTTTPSTPPSPAPPSTQSDTATTTDTPPETETETETETETETETENVTEIETATNANEATSINSQDQTISSTTQAPPPATTLLHVFTLLEGEGQEEEPTTRKPTVRLYPTIQTEVVPKHKLIEINRIVEINSKQAKAAQRKSKANHDFSTLMVESLPHVEQLGEISVVKYVHLVDGSDIQINDGHSTVADYTPTEPTSAAERPVSLPVRNSLPETEGADTDRSGKSLVPEVLTAALETSTISLEGLFDSARKGKQLSSNTIIGETEESTTIGSSSSLASETGETTTPAPTYVRPIVPLLRPESNESSPLVISIANLDQVILSKVQKSLAENSQTTVAPEAASDSNSAFSVRQPLVVQAPISNGAQEIDQDTLNTQDQTINGAISVKTNPIIQTTTNRPNDDQVAEETTIFSIETATEPELNTQTTIPKTEANSETVTAMPIGAVIMGQFGLNTQSTTAVDNDNQLNAQTTSTISSGAVSSVAIGGNTQTANADNARQENTQSTGTITSEISSGAISSDNHNHIGTQTTATIDSSSETTPTQISTTISSGAISGHIDGSINLNTQTNTTISTNNTTTSTTDVGSKVSEAVSFSSETHVVHRKKMGRKGRGRRLRNRKTTTTTTTTETPTTTEATFDDTTTVVPE

SEQ ID NO: 69 - DNA, 782 bp, Homo sapien
aggggccttagcgtgccgcatcgccgagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagtttcgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaactgaagcctgcacagtgtccaccctgttcccactcccatctttcttccggacaatgaaataaagagttaccacccagc

SEQ ID NO: 70 - protein, 207 aa, Homo sapien
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 71 - DNA, 481 bp, Homo sapien
ccactgcacggtagggggtcctgtaggaggctggtggcagggttggattgtgggccctaggcttctgggcgggatgatgacattgagattctggcccctgtatccacaggtgatggagacctgccagatgtccaggagcccccgagagcggctgttgctgcttttgctgctgctactgcttgtgccctggggcactggccctgcctcaggtgttgccctgcccctcgctggtgtgttcagcctccgcgccccgggtcgtgcctgggcgggcttgggtagccccctgtctcggcgcagcctggcgctagctgacgacgcggcctttcgggagcgcgcgcgcctgctggccgccctggagcgccgccgctggctggactcttacatgcagaagctgttgctactggacgcgccctgagcctaataaagagcctgtcgcactgcgactgcgcctctttgctgcgccactctcttgtgggtgtgt

SEQ ID NO: 72 - protein, 100 aa, Homo sapien
METCQMSRSPRERLLLLLLLLLLVPWGTGPASGVALPLAGVFSLRAPGRAWAGLGSPLSRRSLALADDAAFRERARLLAALERRRWLDSYMQKLLLLDAP

SEQ ID NO: 73 - DNA, 2974 bp, Homo sapien
ctcagggcagagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggattgctgattctttaaatgtcttgtttcccagatttcaggaaactttttttcttttaagctatccactcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtataacagaaaaa

SEQ ID NO: 74 - protein, 702 aa, Homo sapien
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 75 - DNA, 2249 bp, Homo sapien
ctcctctacaaagaggtggacagagaagacagcagagaccatgggacccccctcagcccctccctgcagattgcatgtcccctggaaggaggtcctgctcacagcctcacttctaaccttctggaacccacccaccactgccaagctcactattgaatccacgccattcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtggctctgatatagcagccctggtgtattttcgatatttcaggaagactggcagattggaccagaccctgaattcttctagctcctccaatcccattttatcccatggaaccactaaaaacaaggtctgctctgctcctgaagccctatatgctggagatggacaactcaatgaaaatttaaagggaaaaccctcaggcctgaggtgtgtgccactcagagacttcacctaactagagacagtcaaactgcaaaccatggtgagaaattgacgacttcacactatggacagcttttcccaagatgtcaaaacaagactcctcatcatgataaggctcttacccccttttaatttgtccttgcttatgcctgcctctttcgcttggcaggatgatgctgtcattagtatttcacaagaagtagcttcagagggtaacttaacagagtgtcagatctatcttgtcaatcccaacgttttacataaaataagagatcctttagtgcacccagtgactgacattagcagcatctttaacacagccgtgtgttcaaatgtacagtggtccttttcagagttggacttctagactcacctgttctcactccctgttttaattcaacccagccatgcaatgccaaataatagaattgctccctaccagctgaacagggaggagtctgtgcagtttctgacacttgttgttgaacatggctaaatacaatgggtatcgctgagactaagttgtagaaattaacaaatgtgctgcttggttaaaatggctacactcatctgactcattctttattctattttagttggtttgtatcttgcctaaggtgcgtagtccaactcttggtattaccctcctaatagtcatactagtagtcatactccctggtgtagtgtattctctaaaagctttaaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgctttaagatttggtcacactctcacctaggtgagcgcattgagccagtggtgctaaatgctacatactccaactgaaatgttaaggaagaagatagatccaaaaaaaaaaaaaaaaaa

SEQ ID NO: 76 - protein, 344 aa, Homo sapien
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 77 - DNA, 1386 bp, Homo sapien
gctcctcgccccgcgcctgcccccaggatggtccgcgcgaggcaccagccgggtgggctttgcctcctgctgctgctgctctgccagttcatggaggaccgcagtgcccaggctgggaactgctggctccgtcaagcgaagaacggccgctgccaggtcctgtacaagaccgaactgagcaaggaggagtgctgcagcaccggccggctgagcacctcgtggaccgaggaggacgtgaatgacaacacactcttcaagtggatgattttcaacgggggcgcccccaactgcatcccctgtaaagaaacgtgtgagaacgtggactgtggacctgggaaaaaatgccgaatgaacaagaagaacaaaccccgctgcgtctgcgccccggattgttccaacatcacctggaagggtccagtctgcgggctggatgggaaaacctaccgcaatgaatgtgcactcctaaaggcaagatgtaaagagcagccagaactggaagtccagtaccaaggcagatgtaaaaagacttgtcgggatgttttctgtccaggcagctccacatgtgtggtggaccagaccaataatgcctactgtgtgacctgtaatcggatttgcccagagcctgcttcctctgagcaatatctctgtgggaatgatggagtcacctactccagtgcctgccacctgagaaaggctacctgcctgctgggcagatctattggattagcctatgagggaaagtgtatcaaagcaaagtcctgtgaagatatccagtgcactggtgggaaaaaatgtttatgggatttcaaggttgggagaggccggtgttccctctgtgatgagctgtgccctgacagtaagtcggatgagcctgtctgtgccagtgacaatgccacttatgccagcgagtgtgccatgaaggaagctgcctgctcctcaggtgtgctactggaagtaaagcactccggatcttgcaactgaatctgcccgtaaaacctgagccattgattcttcagaactttctgcagtttttgacttcatagattatgctttaaaaaattttttttaacttattgcataacagcagatgccaaaaacaaaaaaagcatctcactgcaagtcacataaaaatgcaacgctgtaatatggctgtatcagagggctttgaaaacatacactgagctgcttctgcgctgttgttgtccgtatttaaacaacagctcccctgtattcccccatctagccatttcggaagacaccgaggaagaggaggaagatgaagaccaggactacagctttcctatatcttctattctagagtggtaaactctctataagtgttcagtgttcacatagcctttgtgcaaaaaaaaaaaaaaaaaaaaa

SEQ ID NO: 78 - protein, 317 aa, Homo sapien
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 79 - DNA, 3445 bp, Homo sapien
gagcaaccgcagcttctagtatccagactccagcgccgccccgggcgcggaccccaaccccgacccagagcttctccagcggcggcgcagcgagcagggctccccgccttaacttcctccgcggggcccagccaccttcgggagtccgggttgcccacctgcaaactctccgccttctgcacctgccacccctgagccagcgcgggcgcccgagcgagtcatggccaacgcggggctgcagctgttgggcttcattctcgccttcctgggatggatcggcgccatcgtcagcactgccctgccccagtggaggatttactcctatgccggcgacaacatcgtgaccgcccaggccatgtacgaggggctgtggatgtcctgcgtgtcgcagagcaccgggcagatccagtgcaaagtctttgactccttgctgaatctgagcagcacattgcaagcaacccgtgccttgatggtggttggcatcctcctgggagtgatagcaatctttgtggccaccgttggcatgaagtgtatgaagtgcttggaagacgatgaggtgcagaagatgaggatggctgtcattgggggtgcgatatttcttcttgcaggtctggctattttagttgccacagcatggtatggcaatagaatcgttcaagaattctatgaccctatgaccccagtcaatgccaggtacgaatttggtcaggctctcttcactggctgggctgctgcttctctctgccttctgggaggtgccctactttgctgttcctgtccccgaaaaacaacctcttacccaacaccaaggccctatccaaaacctgcaccttccagcgggaaagactacgtgtgacacagaggcaaaaggagaaaatcatgttgaaacaaaccgaaaatggacattgagatactatcattaacattaggaccttagaattttgggtattgtaatctgaagtatggtattacaaaacaaacaaacaaacaaaaaacccatgtgttaaaatactcagtgctaaacatggcttaatcttattttatcttctttcctcaatataggagggaagatttttccatttgtattactgcttcccattgagtaatcatactcaattgggggaaggggtgctccttaaatatatatagatatgtatatatacatgtttttctattaaaaatagacagtaaaatactattctcattatgttgatactagcatacttaaaatatctctaaaataggtaaatgtatttaattccatattgatgaagatgtttattggtatattttctttttcgtctatatatacatatgtaacagtcaaatatcatttactcttcttcattagctttgggtgcctttgccacaagacctagcctaatttaccaaggatgaattctttcaattcttcatgcgtgcccttttcatatacttattttattttttaccataatcttatagcacttgcatcgttattaagcccttatttgttttgtgtttcattggtctctatctcctgaatctaacacatttcatagcctacattttagtttctaaagccaagaagaatttattacaaatcagaactttggaggcaaatctttctgcatgaccaaagtgataaattcctgttgaccttcccacacaatccctgtactctgacccatagcactcttgtttgctttgaaaatatttgtccaattgagtagctgcatgctgttcccccaggtgttgtaacacaactttattgattgaatttttaagctacttattcatagttttatatccccctaaactacctttttgttccccattccttaattgtattgttttcccaagtgtaattatcatgcgttttatatcttcctaataaggtgtggtctgtttgtctgaacaaagtgctagactttctggagtgataatctggtgacaaatattctctctgtagctgtaagcaagtcacttaatctttctacctcttttttctatctgccaaattgagataatgatacttaaccagttagaagaggtagtgtgaatattaattagtttatattactctcattctttgaacatgaactatgcctatgtagtgtctttatttgctcagctggctgagacactgaagaagtcactgaacaaaacctacacacgtaccttcatgtgattcactgccttcctctctctaccagtctatttccactgaacaaaacctacacacataccttcatgtggttcagtgccttcctctctctaccagtctatttccactgaacaaaacctacgcacataccttcatgtggctcagtgccttcctctctctaccagtctatttccattctttcagctgtgtctgacatgtttgtgctctgttccattttaacaactgctcttacttttccagtctgtacagaatgctatttcacttgagcaagatgatgtaatggaaagggtgttggcattggtgtctggagacctggatttgagtcttggtgctatcaatcaccgtctgtgtttgagcaaggcatttggctgctgtaagcttattgcttcatctgtaagcggtggtttgtaattcctgatcttcccacctcacagtgatgttgtggggatccagtgagatagaatacatgtaagtgtggttttgtaatttaaaaagtgctatactaagggaaagaattgaggaattaactgcatacgttttggtgttgcttttcaaatgtttgaaaacaaaaaaaatgttaagaaatgggtttcttgccttaaccagtctctcaagtgatgagacagtgaagtaaaattgagtgcactaaacaaataagattctgaggaagtcttatcttctgcagtgagtatggcccgatgctttctgtggctaaacagatgtaatgggaagaaataaaagcctacgtgttggtaaatccaacagcaagggagatttttgaatcataataactcataaggtgctatctgttcagtgatgccctcagagctcttgctgttagctggcagctgacgctgctaggatagttagtttggaaatggtacttcataataaactacacaaggaaagtcagccactgtgtcttatgaggaattggacctaataaattttagtgtgccttccaaacctgagaatatatgcttttggaagttaaaatttaaatggcttttgccacatacatagatcttcatgatgtgtgagtgtaattccatgtggatatcagttaccaaacattacaaaaaaattttatggcccaaaatgaccaacgaaattgttacaatagaatttatccaattttgatctttttatattcttctaccacacctggaaacagaccaatagacattttggggttttataataggaatttgtataaagcattactctttttcaataaattgttttttaatttaaaaaaaggaaaaaaaaaaaaaaaaa

SEQ ID NO: 80 - protein, 211 aa, Homo sapien
MANAGLQLLGFILAFLGWIGAIVSTALPQWRIYSYAGDNIVTAQAMYEGLWMSCVSQSTGQIQCKVFDSLLNLSSTLQATRALMVVGILLGVIAIFVATVGMKCMKCLEDDEVQKMRMAVIGGAIFLLAGLAILVATAWYGNRIVQEFYDPMTPVNARYEFGQALFTGWAAASLCLLGGALLCCSCPRKTTSYPTPRPYPKPAPSSGKDYV

SEQ ID NO: 81 - DNA, 1233 bp, Homo sapien
gtttgcttcaccttctgccaggattgtaagtttcctgaggcctccccagtcctgcggaactggctccggctggcacctgaggagcggcgtgaccccgagggcccagggagctgcccggctggcctaggcaggcagccgcaccatggccagcacggccgtgcagcttctgggcttcctgctcagcttcctgggcatggtgggcacgttgatcaccaccatcctgccgcactggcggaggacagcgcacgtgggcaccaacatcctcacggccgtgtcctacctgaaagggctctggatggagtgtgtgtggcacagcacaggcatctaccagtgccagatctaccgatccctgctggcgctgccccaagacctccaggctgcccgcgccctcatggtcatctcctgcctgctctcgggcatagcctgcgcctgcgccgtcatcgggatgaagtgcacgcgctgcgccaagggcacacccgccaagaccacctttgccatcctcggcggcaccctcttcatcctggccggcctcctgtgcatggtggccgtctcctggaccaccaacgacgtggtgcagaacttctacaacccgctgctgcccagcggcatgaagtttgagattggccaggccctgtacctgggcttcatctcctcgtccctctcgctcattggtggcaccctgctttgcctgtcctgccaggacgaggcaccctacaggccctaccaggccccgcccagggccaccacgaccactgcaaacaccgcacctgcctaccagccaccagctgcctacaaagacaatcgggccccctcagtgacctcggccacgcacagcgggtacaggctgaacgactacgtgtgagtccccacagcctgcttctcccctgggctgctgtgggctgggtccccggcgggactgtcaatggaggcaggggttccagcacaaagtttacttctgggcaatttttgtatccaaggaaataatgtgaatgcgaggaaatgtctttagagcacagggacagagggggaaataagaggaggagaaagctctctataccaaagactgaaaaaaaaaatcctgtctgtttttgtatttattatatatatttatgtgggtgatttgataacaagtttaatataaagtgacttgggagtttggtcagtggggttggtttgtgatccaggaataaaccttgcggatgtggctgtttatgaaaaaaaaaaaaaaaaaaa

SEQ ID NO: 82 - protein, 239 aa, Homo sapien
MASTAVQLLGFLLSFLGMVGTLITTILPHWRRTAHVGTNILTAVSYLKGLWMECVWHSTGIYQCQIYRSLLALPQDLQAARALMVISCLLSGIACACAVIGMKCTRCAKGTPAKTTFAILGGTLFILAGLLCMVAVSWTTNDVVQNFYNPLLPSGMKFEIGQALYLGFISSSLSLIGGTLLCLSCQDEAPYRPYQAPPRATTTTANTAPAYQPPAAYKDNRAPSVTSATHSGYRLNDYV

SEQ ID NO: 83 - DNA, 4716 bp, Homo sapien
ccttggtttccgttgcagattcccacaactccatgctgtgtgctgcaggctggtcctgaacccagatctctggctgagaggatgggggcagatggggaaacagtggttctgaagaacatgctcattggcgtcaacctgatccttctgggctccatgatcaagccttcagagtgtcagctggaggtcaccacagaaagggtccagagacagtcagtggaggaggagggaggcattgccaactacaacacgtccagcaaagagcagcctgtggtcttcaaccacgtgtacaacattaacgtgcccttggacaacctctgctcctcagggctagaggcctctgctgagcaggaggtgagtgcagaagacgagactctggcagagtacatgggccagacctcagaccacgagagccaggtcacctttacacacaggatcaacttccccaaaaaggcctgtccatgtgccagttcagcccaggtgctgcaggagctgctgagccggatcgagatgctggagagggaggtgtcggtgctgcgagaccagtgcaacgccaactgctgccaagaaagtgctgccacaggacaactggactatatccctcactgcagtggccacggcaactttagctttgagtcctgtggctgcatctgcaacgaaggctggtttggcaagaattgctcggagccctactgcccgctgggttgctccagccggggggtgtgtgtggatggccagtgcatctgtgacagcgaatacagcggggatgactgttccgaactccggtgcccaacagactgcagctcccgggggctctgcgtggacggggagtgtgtctgtgaagagccctacactggcgaggactgcagggaactgaggtgccctggggactgttcggggaaggggagatgtgccaacggtacctgtttatgcgaggagggctacgttggtgaggactgcggccagcggcagtgtctgaatgcctgcagtgggcgaggacaatgtgaggaggggctctgcgtctgtgaagagggctaccagggccctgactgctcagcagttgcccctccagaggacttgcgagtggctggtatcagcgacaggtccattgagctggaatgggacgggccgatggcagtgacggaatatgtgatctcttaccagccgacggccctggggggcctccagctccagcagcgggtgcctggagattggagtggtgtcaccatcacggagctggagccaggtctcacctacaacatcagcgtctacgctgtcattagcaacatcctcagccttcccatcactgccaaggtggccacccatctctccactcctcaagggctacaatttaagacgatcacagagaccaccgtggaggtgcagtgggagcccttctcattttccttcgatgggtgggaaatcagcttcattccaaagaacaatgaagggggagtgattgctcaggtccccagcgatgttacgtcctttaaccagacaggactaaagcctggggaggaatacattgtcaatgtggtggctctgaaagaacaggcccgcagcccccctacctcggccagcgtctccacagtcattgacggccccacgcagatcctggttcgcgatgtctcggacaccgtggcttttgtggagtggattccccctcgagccaaagtcgatttcattcttttgaaatatggcctggtgggcggggaaggtgggaggaccaccttccggctgcagcctcccctgagccaatactcagtgcaggccctgcggcctggctcccgatacgaggtgtcagtcagtgccgtccgagggaccaacgagagcgattctgccaccactcagttcacaacagagatcgatgcccccaagaacttgcgagttggttctcgcacagcaaccagccttgacctcgagtgggataacagtgaagccgaagttcaggagtacaaggttgtgtacagcaccctggcgggtgagcaatatcatgaggtactggtccccaggggcattggtccaaccaccagggccaccctgacagatctggtacctggcactgagtatggagttggaatatctgccgtcatgaactcacagcaaagcgtgccagccaccatgaatgccaggactgaacttgacagtccccgagacctcatggtgacagcctcctcggagacctccatctccctcatctggaccaaggccagtggccccattgaccactaccgaattacctttaccccatcctctgggattgcctcagaagtcaccgtacccaaggacaggacctcatacacactaacagatctagagcctggggcagagtacatcatttccgtcactgctgagaggggtcggcagcagagcttggagtccactgtggatgctttcacaggcttccgtcccatctctcatctgcacttttctcatgtgacctcctccagtgtgaacatcacttggagtgatccatctcccccagcagacagactcattcttaactacagccccagggatgaggaggaagagatgatggaggtctccctggatgccaccaagaggcatgctgtcctgatgggcctgcaaccagccacagagtatattgtgaaccttgtggctgtccatggcacagtgacctctgagcccattgtgggctccatcaccacaggaattgatcccccaaaagacatcacaattagcaatgtgaccaaggactcagtgatggtctcctggagccctcctgttgcatctttcgattactaccgagtatcatatcgacccacccaagtgggacgactagacagctcagtggtgcccaacactgtgacagaattcaccatcaccagactgaacccagctaccgaatacgaaatcagcctcaacagcgtgcggggcagggaggaaagcgagcgcatctgtactcttgtgcacacagccatggacaaccctgtggatctgattgctaccaatatcactccaacagaagccctgctgcagtggaaggcaccagtgggtgaggtggagaactacgtcattgttcttacacactttgcagtcgctggagagaccatccttgttgacggagtcagtgaggaatttcggcttgttgacctgcttcctagcacccactatactgccaccatgtatgccaccaatggacctctcaccagtggcaccatcagcaccaacttttctactctcctggaccctccggcaaacctgacagccagtgaagtcaccagacaaagtgccctgatctcctggcagcctcccagggcagagattgaaaattatgtcttgacctacaaatccaccgacggaagccgcaaggagctgattgtggatgcagaagacacctggattcgactggagggcctgttggagaacacagactacacggtgctcctgcaggcagcacaggacaccacgtggagcagcatcacctccaccgctttcaccacaggaggccgggtgttccctcatccccaagactgtgcccagcatttgatgaatggagacactttgagtggggtttaccccatcttcctcaatggggagctgagccagaaattacaagtgtactgtgatatgaccaccgacgggggcggctggattgtattccagaggcggcagaatggccaaactgattttttccggaaatgggctgattaccgtgttggcttcgggaacgtggaggatgagttctggctggggctggacaatatacacaggatcacatcccagggccgctatgagctgcgcgtggacatgcgggatggccaggaggccgccttcgcctcctacgacaggttctctgtcgaggacagcagaaacctgtacaaactccgcataggaagctacaacggcactgcgggggactccctcagctatcatcaaggacgccctttctccacagaggatagagacaatgatgttgcagtgactaactgtgccatgtcgtacaagggagcatggtggtataagaactgccaccggaccaacctcaatgggaagtacggggagtccaggcacagtcagggcatcaactggtaccattggaaaggccatgagttctccatcccctttgtggaaatgaagatgcgcccctacaaccaccgtctcatggcagggagaaaacggcagtccttacagttctgagcagtgggcggctgcaagccaaccaatattttctgtcatttgtttgtattttataatatgaaacaaggggggagggtaatagcaatgtgttttgcaacatattaagagtatgtgaaggaagcagggatgtcgcaggaatccgctggctaacatctgctcttggtttctgctgccctggagcctgaccctcagtctccattctccctcctacccaggcctcctcaaccttcacctcctttcccaccaaggaggagaagtaggaagttttcttaaagggccaattcaaagccaagtcgtggggtgcagattgttatggtgacaggcacacacatttttctacccttcttctgagatgtcctctgccttccaggtatttgtgattttgtcacagcctgacatggccaggttctcacactggcccagagaaaagagcctcagcaagagagttttgccaacaattccccttaaaaggaaacagatcaactacaccgcatcccaacaacccaggttcttttccttccttccttccttcctcccttccttctttcctgccttccc

SEQ ID NO: 84 - protein, 1358 aa, Homo sapien
MGADGETVVLKNMLIGVNLILLGSMIKPSECQLEVTTERVQRQSVEEEGGIANYNTSSKEQPVVFNHVYNINVPLDNLCSSGLEASAEQEVSAEDETLAEYMGQTSDHESQVTFTHRINFPKKACPCASSAQVLQELLSRIEMLEREVSVLRDQCNANCCQESAATGQLDYIPHCSGHGNFSFESCGCICNEGWFGKNCSEPYCPLGCSSRGVCVDGQCICDSEYSGDDCSELRCPTDCSSRGLCVDGECVCEEPYTGEDCRELRCPGDCSGKGRCANGTCLCEEGYVGEDCGQRQCLNACSGRGQCEEGLCVCEEGYQGPDCSAVAPPEDLRVAGISDRSIELEWDGPMAVTEYVISYQPTALGGLQLQQRVPGDWSGVTITELEPGLTYNISVYAVISNILSLPITAKVATHLSTPQGLQFKTITETTVEVQWEPFSFSFDGWEISFIPKNNEGGVIAQVPSDVTSFNQTGLKPGEEYIVNVVALKEQARSPPTSASVSTVIDGPTQILVRDVSDTVAFVEWIPPRAKVDFILLKYGLVGGEGGRTTFRLQPPLSQYSVQALRPGSRYEVSVSAVRGTNESDSATTQFTTEIDAPKNLRVGSRTATSLDLEWDNSEAEVQEYKVVYSTLAGEQYHEVLVPRGIGPTTRATLTDLVPGTEYGVGISAVMNSQQSVPATMNARTELDSPRDLMVTASSETSISLIWTKASGPIDHYRITFTPSSGIASEVTVPKDRTSYTLTDLEPGAEYIISVTAERGRQQSLESTVDAFTGFRPISHLHFSHVTSSSVNITWSDPSPPADRLILNYSPRDEEEEMMEVSLDATKRHAVLMGLQPATEYIVNLVAVHGTVTSEPIVGSITTGIDPPKDITISNVTKDSVMVSWSPPVASFDYYRVSYRPTQVGRLDSSVVPNTVTEFTITRLNPATEYEISLNSVRGREESERICTLVHTAMDNPVDLIATNITPTEALLQWKAPVGEVENYVIVLTHFAVAGETILVDGVSEEFRLVDLLPSTHYTATMYATNGPLTSGTISTNFSTLLDPPANLTASEVTRQSALISWQPPRAEIENYVLTYKSTDGSRKELIVDAEDTWIRLEGLLENTDYTVLLQAAQDTTWSSITSTAFTTGGRVFPHPQDCAQHLMNGDTLSGVYPIFLNGELSQKLQVYCDMTTDGGGWIVFQRRQNGQTDFFRKWADYRVGFGNVEDEFWLGLDNIHRITSQGRYELRVDMRDGQEAAFASYDRFSVEDSRNLYKLRIGSYNGTAGDSLSYHQGRPFSTEDRDNDVAVTNCAMSYKGAWWYKNCHRTNLNGKYGESRHSQGINWYHWKGHEFSIPFVEMKMRPYNHRLMAGRKRQSLQF

SEQ ID NO: 85 - DNA, 3205 bp, Homo sapien
aaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacggatgaggatgatgccatctacacctacaatggggtggttgcttactccatccatagccaagaaccaaaggacccacacgacctcatgttcaccattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaatgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaagggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctcggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttcccagaaaaaaa

SEQ ID NO: 86 - protein, 829 aa, Homo sapien
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 87 - DNA, 3427 bp, Homo sapien
gctgtgccgcaccgaggcgagcaggagcagggaacaggtgtttaaaattatccaactgccatagagctaaattcttttttggaaaattgaaccgaacttctactgaatacaagatgaaaatgtggttgctggtcagtcatcttgtgataatatctattactacctgtttagcagagtttacatggtatagaagatatggtcatggagtttctgaggaagacaaaggatttggaccaatttttgaagagcagccaatcaataccatttatccagaggaatcactggaaggaaaagtctcactcaactgtagggcacgagccagccctttcccggtttacaaatggagaatgaataatggggacgttgatctcacaagtgatcgatacagtatggtaggaggaaaccttgttatcaacaaccctgacaaacagaaagatgctggaatatactactgtttagcatctaataactacgggatggtcagaagcactgaagcaaccctgagctttggatatcttgatcctttcccacctgaggaacgtcctgaggtcagagtaaaagaagggaaaggaatggtgcttctctgtgaccccccataccattttccagatgatcttagctatcgctggcttctaaatgaatttcctgtatttatcacaatggataaacggcgatttgtgtctcagacaaatggcaatctctacattgcaaatgttgaggcttccgacaaaggcaattattcctgctttgtttccagtccttctattacaaagagcgtgttcagcaaattcatcccactcattccaatacctgaacgaacaacaaaaccatatcctgctgatattgtagttcagttcaaggatgtatatgcattgatgggccaaaatgtgaccttagaatgttttgcacttggaaatcctgttccggatatccgatggcggaaggttctagaaccaatgccaagcactgctgagattagcacctctggggctgttcttaagatcttcaatattcagctagaagatgaaggcatctatgaatgtgaggctgagaacattagaggaaaggataaacatcaagcaagaatttatgttcaagcattccctgagtgggtagaacacatcaatgacacagaggtggacataggcagtgatctctactggccttgtgtggccacaggaaagcccatccctacaatccgatggttgaaaaatggatatgcgtatcataaaggggaattaagactgtatgatgtgacttttgaaaatgccggaatgtatcagtgcatagctgaaaacacatatggagccatttatgcaaatgctgagttgaagatcttggcgttggctccaacttttgaaatgaatcctatgaagaaaaagatcctggctgctaaaggtggaagggtgataattgaatgcaaacctaaagctgcaccgaaaccaaagttttcatggagtaaagggacagagtggcttgtcaatagcagcagaatactcatttgggaagatggtagcttggaaatcaacaacattacaaggaatgatggaggtatctatacatgctttgcagaaaataacagagggaaagctaatagcactggaacccttgttatcacagatcctacgcgaattatattggccccaattaatgccgatatcacagttggagaaaacgccaccatgcagtgtgctgcgtcctttgatcctgccttggatctcacatttgtttggtccttcaatggctatgtgatcgattttaacaaagagaatattcactaccagaggaattttatgctggattccaatggggaattactaatccgaaatgcgcagctgaaacatgctggaagatacacatgcactgcccagacaattgtggacaattcttcagcttcagctgaccttgtagtgagaggccctccaggccctccaggtggtctgagaatagaagacattagagccacttctgtggcacttacttggagccgtggttcagacaatcatagtcctatttctaaatacactatccagaccaagactattctttcagatgactggaaagatgcaaagacagatcccccaattattgaaggaaatatggaggcagcaagagcagtggacttaatcccatggatggagtatgaattccgcgtggtagcaaccaatacactgggtagaggagagcccagtataccatctaacagaattaaaacagacggtgctgcaccaaatgtggctccttcagatgtaggaggtggaggtggaagaaacagagagctgaccataacatgggcgcctttgtcaagagaataccactatggcaacaattttggttacatagtggcatttaagccatttgatggagaagaatggaaaaaagtcacagttactaatcctgatactggccgatatgtccataaagatgaaaccatgagcccttccactgcatttcaagttaaagtcaaggccttcaacaacaaaggagatggaccttacagcctagtagcagtcattaattcagcacaagacgctcccagtgaagccccaacagaagtaggtgtaaaagtcttatcatcttctgagatatctgttcattgggaacatgttttagaaaaaatagtggaaagctatcagattcggtattgggctgcccatgacaaagaagaagctgcaaacagagttcaagtcaccagccaagagtactcggccaggctcgagaaccttctgccagacacccagtattttatagaagtcggggcctgcaatagtgcagggtgtggacctccaagtgacatgattgaggctttcaccaagaaagcacctcctagccagcctccaaggatcatcagttcagtaaggtctggttcacgctatataatcacctgggatcatgtcgttgcactatcaaatgaatctacagtgacgggatataaggtactctacagacctgatggccagcatgatggcaagctgtattcaactcacaaacactccatagaagtcccaatccccagagatggagaatacgttgtggaggttcgcgcgcacagtgatggaggagatggagtggtgtctcaagtcaaaatttcaggtgcacccaccctatccccaagtcttctcggcttactgctgcctgcctttggcatccttgtctacttggaattctgaatgtgttgtgacagctgctgttcccatcccagctcagaagacacccttcaaccctgggatgaccacaattccttccaatttctgcggctccatcctaagccaaataaattatactttaacaaactattcaactgatttacaacacacatgatgactgaggcattcgggaaccccttcatccaaaagaataaacttttaaatggatataaatgatttttaactcgttccaatatgccttataaaccacttaacctgat

SEQ ID NO: 88 - protein, 1018 aa, Homo sapien
MKMWLLVSHLVIISITTCLAEFTWYRRYGHGVSEEDKGFGPIFEEQPINTIYPEESLEGKVSLNCRARASPFPVYKWRMNNGDVDLTSDRYSMVGGNLVINNPDKQKDAGIYYCLASNNYGMVRSTEATLSFGYLDPFPPEERPEVRVKEGKGMVLLCDPPYHFPDDLSYRWLLNEFPVFITMDKRRFVSQTNGNLYIANVEASDKGNYSCFVSSPSITKSVFSKFIPLIPIPERTTKPYPADIVVQFKDVYALMGQNVTLECFALGNPVPDIRWRKVLEPMPSTAEISTSGAVLKIFNIQLEDEGIYECEAENIRGKDKHQARIYVQAFPEWVEHINDTEVDIGSDLYWPCVATGKPIPTIRWLKNGYAYHKGELRLYDVTFENAGMYQCIAENTYGAIYANAELKILALAPTFEMNPMKKKILAAKGGRVIIECKPKAAPKPKFSWSKGTEWLVNSSRILIWEDGSLEINNITRNDGGIYTCFAENNRGKANSTGTLVITDPTRIILAPINADITVGENATMQCAASFDPALDLTFVWSFNGYVIDFNKENIHYQRNFMLDSNGELLIRNAQLKHAGRYTCTAQTIVDNSSASADLVVRGPPGPPGGLRIEDIRATSVALTWSRGSDNHSPISKYTIQTKTILSDDWKDAKTDPPIIEGNMEAARAVDLIPWMEYEFRVVATNTLGRGEPSIPSNRIKTDGAAPNVAPSDVGGGGGRNRELTITWAPLSREYHYGNNFGYIVAFKPFDGEEWKKVTVTNPDTGRYVHKDETMSPSTAFQVKVKAFNNKGDGPYSLVAVINSAQDAPSEAPTEVGVKVLSSSEISVHWEHVLEKIVESYQIRYWAAHDKEEAANRVQVTSQEYSARLENLLPDTQYFIEVGACNSAGCGPPSDMIEAFTKKAPPSQPPRIISSVRSGSRYIITWDHVVALSNESTVTGYKVLYRPDGQHDGKLYSTHKHSIEVPIPRDGEYVVEVRAHSDGGDGVVSQVKISGAPTLSPSLLGLLLPAFGILVYLEF

SEQ ID NO: 89 - DNA, 1616 bp, Homo sapien
ctccctgtgttggtggaggatgtctgcagcagcatttaaattctgggagggcttggttgtcagcagcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaaaaaaaaaaaaaaaaaaa

SEQ ID NO: 90 - protein, 300 aa, Homo sapien
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 91 - DNA, 2593 bp, Homo sapien
tggacttggatccgaggcagacgaggaagctgagaaaaccctggcgttgaccccgtggacctgggcgccccgggaaggtccagcgcttggtccaggcaggcggggatgtgcggtgaccaccctggtcctgaaaagtccagccccgaatctccctccctcctagacctggaggcctggaacagccagccgcccacggacgccagagccgggaaccctgacggcacttagctgctgacaaacaacctgctccgtggacgcctgaaacaccagtctttggggccagtgcctcagtttcaatccaggtaacctttaaatgaaacttgcctaaaatcttaggtcatacacagaagagactccaatcgacaagaagctggaaaagaatgatgttgtccttaaacaacctacagaatatcatctataacccggtaatcccgtatgttggcaccattcccgatcagctggatcctggaactttgattgtgatatgtgggcatgttcctagtgacgcagacagattccaggtggatctgcagaatggcagcagtgtgaaacctcgagccgatgtggcctttcatttcaatcctcgtttcaaaagggccggctgcattgtttgcaatactttgataaatgaaaaatggggacgggaagagatcacctatgacacgcctttcaaaagagaaaagtcttttgagatcgtgattatggtgctaaaggacaaattccaggtggctgtaaatggaaaacatactctgctctatggccacaggatcggcccagagaaaatagacactctgggcatttatggcaaagtgaatattcactcaattggttttagcttcagctcggacttacaaagtacccaagcatctagtctggaactgacagagataagtagagaaaatgttccaaagtctggcacgccccagcttcagactgtctctccctcctgggatttacagggtcatggctctgaaacattctgtagtgttctttggacacgagttttcctggagatcgctttctgcaggcctattggtctgactgtggcttcttttcagagcctgccattcgctgcaaggttgaacacccccatgggccctggacgaactgtcgtcgttaaaggagaagtgaatgcaaatgccaaaagctttaatgttgacctactagcaggaaaatcaaaggatattgctctacacttgaacccacgcctgaatattaaagcatttgtaagaaattcttttcttcaggagtcctggggagaagaagagagaaatattacctctttcccatttagtcctgggatgtactttgagatgataatttactgtgatgttagagaattcaaggttgcagtaaatggcgtacacagcctggagtacaaacacagatttaaagagctcagcagtattgacacgctggaaattaatggagacatccacttactggaagtaaggagctggtagcctacctacacagctgctacaaaaaccaaaatacagaatggcttctgtgatactggccttgctgaaacgcatctcactgtcattctattgtttatattgttaaaatgagcttgtgcaccattagatcctgctgggtgttctcagtccttgccatgaagtatggtggtgtctagcactgaatggggaaactgggggcagcaacacttatagccagttaaagccactctgccctctctcctactttggctgactcttcaagaatgccattcaacaagtatttatggagtacctactataatacagtagctaacatgtattgagcacagattttttttggtaaaactgtgaggagctaggatatatacttggtgaaacaaaccagtatgttccctgttctcttgagcttcgactcttctgtgctctattgctgcgcactgctttttctacaggcattacatcaactcctaaggggtcctctgggattagttaagcagctattaaatcacccgaagacactaatttacagaagacacaactccttccccagtgatcactgtcataaccagtgctctaccgtatcccatcactgaggactgatgttgactgacatcattttatcgtaataaacatgtggctctattagctgcaagctttaccaagtaattggcatgacatctgagcacagaaattaaggcaaaaaaccaaagcaaaacaaatacatggtgctgaaattaacttgatgccaagcccaaggcagctgatttctgtgtatttgaacttagggcaaatcagagtctacacagacgcctacagaaagtttcaggaagaggcaagatgcattcaatttgaaagatatttatgggcaacaaagtaaggtcaggattagacttcaggcattcataaggcaggcactatcagaaagtgtacgccaactaagggacccacaaagcaggcagaggtaatgcagaaatctgttttgttcccatgaaatcaccaatcaaggcctccgttcttctaaagattagtccatcatcattagcaactgagatcaaagcactcttccactttacgtgattaaaatcaaacctgtatcagcaaaaaaaaaaaaaaaaaaaaaaaaaaaaaaaaaa

SEQ ID NO: 92 - protein, 359 aa, Homo sapien
MLSLNNLQNIIYNPVIPYVGTIPDQLDPGTLIVICGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSDLQSTQASSLELTEISRENVPKSGTPQLQTVSPSWDLQGHGSETFCSVLWTRVFLEIAFCRPIGLTVASFQSLPFAARLNTPMGPGRTVVVKGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 93 - DNA, 2401 bp, Homo sapien
agcctcccgcccgccgcctctgtctccctctctccacaaactgcccaggagtgagtagctgctttcggtccgccggacacaccggacagatagacgtgcggacggcccaccaccccagcccgccaactagtcagcctgcgcctggcgcctcccctctccaggtccatccgccatgtggcccctgtggcgcctcgtgtctctgctggccctgagccaggccctgccctttgagcagagaggcttctgggacttcaccctggacgatgggccattcatgatgaacgatgaggaagcttcgggcgctgacacctcgggcgtcctggacccggactctgtcacacccacctacagcgccatgtgtcctttcggctgccactgccacctgcgggtggttcagtgctccgacctgggtctgaagtctgtgcccaaagagatctcccctgacaccacgctgctggacctgcagaacaacgacatctccgagctccgcaaggatgacttcaagggtctccagcacctctacgccctcgtcctggtgaacaacaagatctccaagatccatgagaaggccttcagcccactgcggaagctgcagaagctctacatctccaagaaccacctggtggagatcccgcccaacctacccagctccctggtggagctccgcatccacgacaaccgcatccgcaaggtgcccaagggagtgttcagcgggctccggaacatgaactgcatcgagatgggcgggaacccactggagaacagtggctttgaacctggagccttcgatggcctgaagctcaactacctgcgcatctcagaggccaagctgactggcatccccaaagacctccctgagaccctgaatgaactccacctagaccacaacaaaatccaggccatcgaactggaggacctgcttcgctactccaagctgtacaggctgggcctaggccacaaccagatcaggatgatcgagaacgggagcctgagcttcctgcccaccctccgggagctccacttggacaacaacaagttggccagggtgccctcagggctcccagacctcaagctcctccaggtggtctatctgcactccaacaacatcaccaaagtgggtgtcaacgacttctgtcccatgggcttcggggtgaagcgggcctactacaacggcatcagcctcttcaacaaccccgtgccctactgggaggtgcagccggccactttccgctgcgtcactgaccgcctggccatccagtttggcaactacaaaaagtagaggcagctgcagccaccgcggggcctcagtgggggtctctggggaacacagccagacatcctgatggggaggcagagccaggaagctaagccagggcccagctgcgtccaacccagccccccacctcgggtccctgaccccagctcgatgccccatcaccgcctctccctggctcccaagggtgcaggtgggcgcaaggcccggcccccatcacatgttcccttggcctcagagctgcccctgctctcccaccacagccacccagaggcaccccatgaagcttttttctcgttcactcccaaacccaagtgtccaaggctccagtcctaggagaacagtccctgggtcagcagccaggaggcggtccataagaatggggacagtgggctctgccagggctgccgcacctgtccagacacacatgttctgttcctcctcctcatgcatttccagcctttcaaccctccccgactctgcggctcccctcagcccccttgcaagttcatggcctgtccctcccagacccctgctccactggcccttcgaccagtcctcccttctgttctctctttccccgtccttcctctctctctctctctctctctctctctctttctgtgtgtgtgtgtgtgtgtgtgtgtgtgtgtgtgtgtgtgtgtgtcttgtgcttcctcagacctttctcgcttctgagcttggtggcctgttccctccatctctccgaacctggcttcgcctgtccctttcactccacaccctctggccttctgccttgagctgggactgctttctgtctgtccggcctgcacccagcccctgcccacaaaaccccagggacagcggtctccccagcctgccctgctcaggccttgcccccaaacctgtactgtcccggaggaggttgggaggtggaggcccagcatcccgcgcagatgacaccatcaaccgccagagtcccagacaccggttttcctagaagcccctcacccccactggcccactggtggctaggtctccccttatccttctggtccagcgcaaggaggggctgcttctgaggtcggtggctgtctttccattaaagaaacaccgtgcaacgtgaaaaaaaaaaaaaaaaaaaaa

SEQ ID NO: 94 - protein, 368 aa, Homo sapien
MWPLWRLVSLLALSQALPFEQRGFWDFTLDDGPFMMNDEEASGADTSGVLDPDSVTPTYSAMCPFGCHCHLRVVQCSDLGLKSVPKEISPDTTLLDLQNNDISELRKDDFKGLQHLYALVLVNNKISKIHEKAFSPLRKLQKLYISKNHLVEIPPNLPSSLVELRIHDNRIRKVPKGVFSGLRNMNCIEMGGNPLENSGFEPGAFDGLKLNYLRISEAKLTGIPKDLPETLNELHLDHNKIQAIELEDLLRYSKLYRLGLGHNQIRMIENGSLSFLPTLRELHLDNNKLARVPSGLPDLKLLQVVYLHSNNITKVGVNDFCPMGFGVKRAYYNGISLFNNPVPYWEVQPATFRCVTDRLAIQFGNYKK

SEQ ID NO: 95 - DNA, 1983 bp, Homo sapien
cgagtaaagtttgcaaagaggcgcgggaggcggcagccgcagcgaggaggcggcggggaagaagcgcagtctccgggttgggggcgggggcggggggggcgccaaggagccgggtggggggcggcggccagcatgcggccccgcagcgccctgccccgcctgctgctgccgctgctgctgctgcccgccgccgggccggcccagttccacggggagaagggcatctccatcccggaccacggcttctgccagcccatctccatcccgctgtgcacggacatcgcctacaaccagaccatcatgcccaaccttctgggccacacgaaccaggaggacgcaggcctagaggtgcaccagttctatccgctggtgaaggtgcagtgctcgcccgaactgcgcttcttcctgtgctccatgtacgcacccgtgtgcaccgtgctggaacaggccatcccgccgtgccgctctatctgtgagcgcgcgcgccagggctgcgaagccctcatgaacaagttcggttttcagtggcccgagcgcctgcgctgcgagcacttcccgcgccacggcgccgagcagatctgcgtcggccagaaccactccgaggacggagctcccgcgctactcaccaccgcgccgccgccgggactgcagccgggtgccgggggcaccccgggtggcccgggcggcggcggcgctcccccgcgctacgccacgctggagcaccccttccactgcccgcgcgtcctcaaggtgccatcctatctcagctacaagtttctgggcgagcgtgattgtgctgcgccctgcgaacctgcgcggcccgatggttccatgttcttctcacaggaggagacgcgtttcgcgcgcctctggatcctcacctggtcggtgctgtgctgcgcttccaccttcttcactgtcaccacgtacttggtagacatgcagcgcttccgctacccagagcggcctatcatttttctgtcgggctgctacaccatggtgtcggtggcctacatcgcgggcttcgtgctccaggagcgcgtggtgtgcaacgagcgcttctccgaggacggttaccgcacggtggtgcagggcaccaagaaggagggctgcaccatcctcttcatgatgctctacttcttcagcatggccagctccatctggtgggtcatcctgtcgctcacctggttcctggcagccggcatgaagtggggccacgaggccatcgaggccaactctcagtacttccacctggccgcctgggccgtgccggccgtcaagaccatcaccatcctggccatgggccagatcgacggcgacctgctgagcggcgtgtgcttcgtaggcctcaacagcctggacccgctgcggggcttcgtgctagcgccgctcttcgtgtacctgttcatcggcacgtccttcctcctggccggcttcgtgtcgctcttccgcatccgcaccatcatgaagcacgacggcaccaagaccgaaaagctggagcggctcatggtgcgcatcggcgtcttctccgtgctctacacagtgcccgccaccatcgtcatcgcttgctacttctacgagcaggccttccgcgagcactgggagcgctcgtgggtgagccagcactgcaagagcctggccatcccgtgcccggcgcactacacgccgcgcatgtcgcccgacttcacggtctacatgatcaaatacctcatgacgctcatcgtgggcatcacgtcgggcttctggatctggtcgggcaagacgctgcactcgtggaggaagttctacactcgcctcaccaacagccgacacggtgagaccaccgtgtgagggacgcccccaggccggaaccgcgcggcgctttcctccgcccggggtggggcccctacagactccgtattttatttttttaaataaaaaacgatcgaaaccatttcacttttaggttgctttttaaaagagaactctctgcccaacaccccc

SEQ ID NO: 96 - protein, 565 aa, Homo sapien
MRPRSALPRLLLPLLLLPAAGPAQFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPPGLQPGAGGTPGGPGGGGAPPRYATLEHPFHCPRVLKVPSYLSYKFLGERDCAAPCEPARPDGSMFFSQEETRFARLWILTWSVLCCASTFFTVTTYLVDMQRFRYPERPIIFLSGCYTMVSVAYIAGFVLQERVVCNERFSEDGYRTVVQGTKKEGCTILFMMLYFFSMASSIWWVILSLTWFLAAGMKWGHEAIEANSQYFHLAAWAVPAVKTITILAMGQIDGDLLSGVCFVGLNSLDPLRGFVLAPLFVYLFIGTSFLLAGFVSLFRIRTIMKHDGTKTEKLERLMVRIGVFSVLYTVPATIVIACYFYEQAFREHWERSWVSQHCKSLAIPCPAHYTPRMSPDFTVYMIKYLMTLIVGITSGFWIWSGKTLHSWRKFYTRLTNSRHGETTV

SEQ ID NO: 97 - DNA, 2294 bp, Homo sapien
aagcagttgttttgctggaaggagggagtgcgcgggctgccccgggctcctccctgccgcctcctctcagtggatggttccaggcaccctgtctggggcagggagggcacaggcctgcacatcgaaggtggggtgggaccaggctgcccctcgccccagcatccaagtcctcccttgggcgcccgtggccctgcagactctcagggctaaggtcctctgttgctttttggttccaccttagaagaggctccgcttgactaagagtagcttgaaggaggcaccatgcaggagctgcatctgctctggtgggcgcttctcctgggcctggctcaggcctgccctgagccctgcgactgtggggaaaagtatggcttccagatcgccgactgtgcctaccgcgacctagaatccgtgccgcctggcttcccggccaatgtgactacactgagcctgtcagccaaccggctgccaggcttgccggagggtgccttcagggaggtgcccctgctgcagtcgctgtggctggcacacaatgagatccgcacggtggccgccggagccctggcctctctgagccatctcaagagcctggacctcagccacaatctcatctctgactttgcctggagcgacctgcacaacctcagtgccctccaattgctcaagatggacagcaacgagctgaccttcatcccccgcgacgccttccgcagcctccgtgctctgcgctcgctgcaactcaaccacaaccgcttgcacacattggccgagggcaccttcaccccgctcaccgcgctgtcccacctgcagatcaacgagaaccccttcgactgcacctgcggcatcgtgtggctcaagacatgggccctgaccacggccgtgtccatcccggagcaggacaacatcgcctgcacctcaccccatgtgctcaagggtacgccgctgagccgcctgccgccactgccatgctcggcgccctcagtgcagctcagctaccaacccagccaggatggtgccgagctgcggcctggttttgtgctggcactgcactgtgatgtggacgggcagccggcccctcagcttcactggcacatccagatacccagtggcattgtggagatcaccagccccaacgtgggcactgatgggcgtgccctgcctggcacccctgtggccagctcccagccgcgcttccaggcctttgccaatggcagcctgcttatccccgactttggcaagctggaggaaggcacctacagctgcctggccaccaatgagctgggcagtgctgagagctcagtggacgtggcactggccacgcccggtgagggtggtgaggacacactggggcgcaggttccatggcaaagcggttgagggaaagggctgctatacggttgacaacgaggtgcagccatcagggccggaggacaatgtggtcatcatctacctcagccgtgctgggaaccctgaggctgcagtcgcagaaggggtccctgggcagctgcccccaggcctgctcctgctgggccaaagcctcctcctcttcttcttcctcacctccttctagccccacccagggcttccctaactcctccccttgcccctaccaatgcccctttaagtgctgcaggggtctggggttggcaactcctgaggcctgcatgggtgacttcacattttcctacctctccttctaatctcttctagagcacctgctatccccaacttctagacctgctccaaactagtgactaggatagaatttgatcccctaactcactgtctgcggtgctcattgctgctaacagcattgcctgtgctctcctctcaggggcagcatgctaacggggcgacgtcctaatccaactgggagaagcctcagtggtggaattccaggcactgtgactgtcaagctggcaagggccaggattgggggaatggagctggggcttagctgggaggtggtctgaagcagacagggaatgggagaggaggatgggaagtagacagtggctggtatggctctgaggctccctggggcctgctcaagctcctcctgctccttgctgttttctgatgatttgggggcttgggagtccctttgtcctcatctgagactgaaatgtggggatccaggatggcttccttcctcttacccttcctccctcagcctgcaacctctatcctggaacctgtcctccctttctccccaactatgcatctgttgtctgctcctctgcaaaggccagccagcttgggagcagcagagaaataaacagcatttctgatgcc

SEQ ID NO: 98 - protein, 428 aa, Homo sapien
MQELHLLWWALLLGLAQACPEPCDCGEKYGFQIADCAYRDLESVPPGFPANVTTLSLSANRLPGLPEGAFREVPLLQSLWLAHNEIRTVAAGALASLSHLKSLDLSHNLISDFAWSDLHNLSALQLLKMDSNELTFIPRDAFRSLRALRSLQLNHNRLHTLAEGTFTPLTALSHLQINENPFDCTCGIVWLKTWALTTAVSIPEQDNIACTSPHVLKGTPLSRLPPLPCSAPSVQLSYQPSQDGAELRPGFVLALHCDVDGQPAPQLHWHIQIPSGIVEITSPNVGTDGRALPGTPVASSQPRFQAFANGSLLIPDFGKLEEGTYSCLATNELGSAESSVDVALATPGEGGEDTLGRRFHGKAVEGKGCYTVDNEVQPSGPEDNVVIIYLSRAGNPEAAVAEGVPGQLPPGLLLLGQSLLLFFFLTSF

SEQ ID NO: 99 - DNA, 6894 bp, Homo sapien
tgacccggtccgtgtgggccagcgggaaggaagccagttgagggaagttctccatgaatgtacgtcacaatgatgatgaccgaccaaatccctctggaactgccaccattgctgaacggagaggtagccatgatgccccacttggtgaatggagatgcagctcagcaggttattctcgttcaagttaatccaggtgagactttcacaataagagcagaggatggaacacttcagtgcattcaaggacctgctgaagttcccatgatgtcacccaatggatccattcctcccattcatgtgcctccaggttatatctcacaggtgattgaagatagtactggagtccgccgggtggtggtcacaccccagtctcctgagtgttatcccccaagctacccctcagccatgtctccaacccatcatctccctccctatctgactcaccatccacattttattcataactcacacacggcttactacccacctgttaccggacctggagatatgccgcctcagttttttccccagcatcatcttccccacacaatatatggtgagcaagaaattataccattttatggaatgtcaagctacatcacccgagaagaccagtacagcaagcctccgcacaaaaaactgaaagaccgccagatcgatcgccagaaccgactcaacagacctccttctgctatctacaaaagcagctgcacaacagtatacaatggctatgggaagggccatagtggtggaagtggcggaggcggcagcggtagtggtcccggaattaagaaaacagagcgacgagcaagaagcagcccaaagtcgaatgattcagacttgcaagaatatgagttggaagtaaagagggtgcaagacattctttcgggaatagagaaaccacaggtttctaatattcaggcaagagcagttgtgttgtcctgggctccccctgttggactttcctgtggaccccacagtggtctttccttcccctacagttacgaggtggccttatcagacaaaggacgagatggaaaatacaagataatttacagtggagaagaattagaatgtaacctgaaagatcttagaccagcaacagattatcatgtgagggtgtatgccatgtacaattccgtaaagggatcctgctccgagcctgttagcttcaccacccacagctgtgcacccgagtgtcctttcccccctaagctggcacataggagcaaaagttcactaaccctgcagtggaaggcaccaattgacaacggttcaaaaatcaccaactaccttttagagtgggatgagggaaaaagaaatagtggtttcagacagtgcttcttcgggagccagaagcactgcaagttgacaaagctttgtccggcaatggggtacacattcaggctggccgctcgaaacgacattggtaccagtggttatagccaagaggtggtgtgctacacattaggaaatatccctcagatgccttctgcaccaaggctggttcgagctggcatcacatgggtcacgttgcagtggagtaagccagaaggctgttcacccgaggaagtgatcacctacaccttggaaattcaggaggatgaaaatgataaccttttccacccaaaatacactggagaggatttaacctgtactgtgaaaaatctcaaaagaagcacacagtatacattcaggctgactgcttctaatacggaaggaaaaagctgtccaagcgaagttcttgtttgtacgacgagtcctgacaggcctggacctcctaccagaccgcttgtcaaaggcccagttacatctcatggctttagtgtcaaatgggatccccctaaggacaatggtggttcagaaatcctcaagtacttgctagagattactgatggaaattctgaagcgaatcagtgggaagtggcctacagtgggtcggctaccgaatacaccttcacccacttgaaaccaggcactttgtacaaactccgagcatgctgcatcagtaccggcggacacagccagtgttctgaaagtctccctgttcgcacactaagcattgcaccaggtcaatgtcgaccaccgagggttttgggtagaccaaagcacaaagaagtccacttagagtgggatgttcctgcatcggaaagtggctgtgaggtctcagagtacagcgtggagatgacggagcccgaagacgtagcctcggaagtgtaccatggcccagagctggagtgcaccgtcggcaacctgcttcctggaaccgtgtatcgcttccgggtgagggctctgaatgatggagggtatggtccctattctgatgtctcagaaattaccactgctgcagggcctcctggacaatgcaaagcaccttgtatttcttgtacacctgatggatgtgtcttagtgggttgggagagtcctgatagttctggtgctgacatctcagagtacaggttggaatggggagaagatgaagaatccttagaactcatttatcatgggacagacacccgttttgaaataagagacctgttgcctgctgcacagtattgctgtagactacaggccttcaatcaagcaggggcagggccgtacagtgaacttgtcctttgccagacgccagcgtctgcccctgaccccgtctccactctctgtgtcctggaggaggagccccttgatgcctaccctgattcaccttctgcgtgccttgtactgaactgggaagagccgtgcaataacggatctgaaatccttgcttacaccattgatctaggagacactagcattaccgtgggcaacaccaccatgcatgttatgaaagatctccttccagaaaccacctaccggatcagaattcaggctataaatgaaattggagctggaccatttagtcagttcattaaagcaaaaactcggccattaccacccttgcctcctaggctagaatgtgctgctgctggtcctcagagcctgaagctaaaatggggagacagtaactccaagacacatgctgctgaggacattgtgtacacactacagctggaggacagaaacaagaggtttatttcaatctacagaggacccagccacacctacaaggtccagagactgacggaattcacatgctactccttcagaatccaggcagcaagcgaggctggagaagggcccttctcagaaacctataccttcagcacaaccaaaagtgtcccccccaccatcaaagcacctcgagtaacacagttagaaggaaattcatgtgaaattttatgggagacggtaccatcaatgaaaggtgaccctgttaactacattctgcaggtattggttggaagagaatctgagtacaaacaggtgtacaagggagaagaagccacattccaaatctcaggcctccagaccaacacagactacaggttccgcgtatgtgcgtgtcgtcgctgtttagacacctctcaggagctaagcggagccttcagcccctctgcggcttttgtattacaacgaagtgaggtcatgcttacaggggacatggggagcttagatgatcccaaaatgaagagcatgatgcctactgatgaacagtttgcagccatcattgtgcttggctttgcaactttgtccattttatttgcctttatattacagtacttcttaatgaagtaaacccaacaaaactagaggtatgaattaatgctacacattttaatacacacatttattcagatactcccctttttaaagcccttttgttttttgatttatatactctgttttacagatttagctagaaaaaaaatgtcagtgttttggtgcacctttttgaaatgcaaaactaggaaaaggttaaactggatttttttttttaaaaaaaagaaaaaaaaagaagaaaagtataccagataccaaaagctagctttcttatgttttcctttaaattttcagatttaccttcattctgttttcactgatgtcttttgcaagcctttgatttttttttttttgttacagtttagtaatttatattcaccagtcacttcatatgtcttgaacatctgtatctgtaaacatgaatcaccgtgtgtgtacttacagggctaggatttcagtgttgtcagagtattaccacacagcaacagcaacatacagaagatatgttcactcagataagactgccctaaacaaccattttgtcactcagttatttaactgtgtttagctcatttaaatcaaaatgtgtactttaatctaaaatgttttaataatctgtatttcttataattttaacactatgagctgcctgtataagaaatcaagtaaccagaatgcacctataaattatggagcattgtagattttaccacatcaattcatagcagtaactttaagagggcattgtgcaatagttagttgttttcttgttcagctattttaaaggctgctttaacttgtttgtttgtctttgtatataactacttctaatctaatcactagagttattatattctgttatgtttgaccagaattatatgacaagaactggtgacagtttagtgcctctgcccattgtccatgatttacactaattgtgagcagtcttcttatgtgtcagctcattatttttgaaacatttgcctttaggctgttctttgaggtatcaatgaagtgattgaatttcaataccttaattcagtgcacataatactaatgtaacagcagatgaaaattgataaaacccaaaagagagtcatctaaatttgtagttcctatttctgtgggtttgcctggccatggttggagagggaatggtgtttgatggtaaacacagggtgtttggggatcaaggagcctagattctctccctggatctgtcactaacttgctgcgtgacctgaacacgtcactttacctctctgtgcctcagttttcccatgcatgaaaaataaaataaaataaaacggggattctaatgtttgtaagtgctttgagatctttgaccaacaggtgctattggagtgcaaagtgttactcttacgtgtttattttgagtcatgagataatcaattttaacccaaagtcattggattatttatatgaagtccataatgttcgagtacctcagggacatttaagagttggaggtgcaaatatattccaaaagggtgcaacagacacagtgtatccccctgcttctgtttttgtatatttttgctacttggtttttcttgatcatagctattttgtgcttgatctttattgtctaagatgcagtatcctgtactagcttataatattcccataccaaagtcatggggaaacaaacattattttgtttttggtttatttatactatattctgcatacagtactttaaatgccaattacagtgcaatctttatttattgtaaaattttttaagtgtacttatgtactaattttcccttgtagcatgttatatttttgtgttttatacttttgtaattttaggtcagtcttgttccttggcaacatctgtagtattattaatcttctgacattttcttatgtttttaaaaagataagagcatctagtgcattaaatgccaaaaaaaaaatacattatcagtgattgaaacgtttacatgtacccaaaaaccataatcatctcttggaagaaaatgctgagatcaatgaattattctgtgtgcctatattgacgtagtgagtactagagagttctgtattttattattgactataataattagtttaattagctttgcaaactgatggcatcaaggtaaatatatttttgccaaagttctggccttccaaaactcacccccttatttaaatgtgtgctatgacccactatgaccacagcatctgcattttctaaaaaattccatgcaggtgttttggggagaggtattttttaagcaatgaaaattcaactgagtacaaagccccctcttggggggttggggaagtctcttttttgaaacacttcagaactgctgctataaagaaattctctaattggttgaattttttttttaagtaaatagtactttaggccaaaatttatatgaatatttgatcttcttgagattttcatactatcatttaaccaccaggaagctgaagtgtgtgaagtacaaagctgacagcactttattttattgctctccattatttggtattcattatattccttcagtcagaaaattattactctctatggcactgttttttatcacaaatatgtatatgtgatattgatatataactatatatattgccatcacacacgaacaataaaataaagtgttctattaacctgatctctttgcccttttgctatgtgaggagtgaatgagtggccttctgatgctctgactcttctctgtatgtcaaactcatccctggcacaagaaattccagtcatgtgaagcaaactgccctttgtcctcaaagaaattgttgaaaaagaaaactttttaaagagattttttgcatattctctgccttgttcttatcaacttgaaatgttggcattttctaaccttgttttgttggctacaataattcagtattcatgtcaaaattgagaagtgccctaattgaatgtgtttgaatgttatccttgcacaattctttaaattgaaagataaaatgttttacctcactgttggacatacattccaagcttttcaactctaggagaaaaagaaaatcatgttttcctgtattgtaaattttagactatttcatatacattgtattaaaactgccatatcaattttaatgtatagattttgcaaatattatgctatatgtaatacctaactgtatctgtagtgtatatgtaatatatttatgcccaataaatgttttaattctttctga

SEQ ID NO: 100 - protein, 1204 aa, Homo sapien
MYVTMMMTDQIPLELPPLLNGEVAMMPHLVNGDAAQQVILVQVNPGETFTIRAEDGTLQCIQGPAEVPMMSPNGSIPPIHVPPGYISQVIEDSTGVRRVVVTPQSPECYPPSYPSAMSPTHHLPPYLTHHPHFIHNSHTAYYPPVTGPGDMPPQFFPQHHLPHTIYGEQEIIPFYGMSSYITREDQYSKPPHKKLKDRQIDRQNRLNRPPSAIYKSSCTTVYNGYGKGHSGGSGGGGSGSGPGIKKTERRARSSPKSNDSDLQEYELEVKRVQDILSGIEKPQVSNIQARAVVLSWAPPVGLSCGPHSGLSFPYSYEVALSDKGRDGKYKIIYSGEELECNLKDLRPATDYHVRVYAMYNSVKGSCSEPVSFTTHSCAPECPFPPKLAHRSKSSLTLQWKAPIDNGSKITNYLLEWDEGKRNSGFRQCFFGSQKHCKLTKLCPAMGYTFRLAARNDIGTSGYSQEVVCYTLGNIPQMPSAPRLVRAGITWVTLQWSKPEGCSPEEVITYTLEIQEDENDNLFHPKYTGEDLTCTVKNLKRSTQYTFRLTASNTEGKSCPSEVLVCTTSPDRPGPPTRPLVKGPVTSHGFSVKWDPPKDNGGSEILKYLLEITDGNSEANQWEVAYSGSATEYTFTHLKPGTLYKLRACCISTGGHSQCSESLPVRTLSIAPGQCRPPRVLGRPKHKEVHLEWDVPASESGCEVSEYSVEMTEPEDVASEVYHGPELECTVGNLLPGTVYRFRVRALNDGGYGPYSDVSEITTAAGPPGQCKAPCISCTPDGCVLVGWESPDSSGADISEYRLEWGEDEESLELIYHGTDTRFEIRDLLPAAQYCCRLQAFNQAGAGPYSELVLCQTPASAPDPVSTLCVLEEEPLDAYPDSPSACLVLNWEEPCNNGSEILAYTIDLGDTSITVGNTTMHVMKDLLPETTYRIRIQAINEIGAGPFSQFIKAKTRPLPPLPPRLECAAAGPQSLKLKWGDSNSKTHAAEDIVYTLQLEDRNKRFISIYRGPSHTYKVQRLTEFTCYSFRIQAASEAGEGPFSETYTFSTTKSVPPTIKAPRVTQLEGNSCEILWETVPSMKGDPVNYILQVLVGRESEYKQVYKGEEATFQISGLQTNTDYRFRVCACRRCLDTSQELSGAFSPSAAFVLQRSEVMLTGDMGSLDDPKMKSMMPTDEQFAAIIVLGFATLSILFAFILQYFLMK

SEQ ID NO: 101 - DNA, 2565 bp, Homo sapien
tcgcgatgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtgatggggtgcagacccccctcatggagtatggggcgctggacacatggccggggctgcaccagggacccatgggggctgcccagctggacagatggcttcctgctccccaggcccagccagggtcctctctcaaccactagacttggctctcaggaactctgcttcctggcccagcgctcgtgaccaaggatacaccaaagcccttaagacctcagggggcgggtgctggggtcttctccaataaatggggtgtcaaccttaaaaaaaaaaaaaaaaaaaaaaaaaaa

SEQ ID NO: 102 - protein, 757 aa, Homo sapien
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 103 - DNA, 2269 bp, Homo sapien
tgggttggtgtttatctcctcccagccttgagggagggaacaacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgtttcagacggcagcagctcgggactctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagctgagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcaaaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacaaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaaacatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggctgagaatcagactgacagtttacagacgctgctgtcacaaccaagaatgttatgtgcaagtttatcagtaaataactggaaaacagaacacttatgttatacaatacagatcatcttggaactgcattcttctgagcactgtttatacactgtgtaaatacccatatgtcct

SEQ ID NO: 104 - protein, 496 aa, Homo sapien
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 105 - DNA, 2076 bp, Homo sapien
cggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 106 - protein, 419 aa, Homo sapien
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 107 - DNA, 2653 bp, Homo sapien
atggccctgtccactgagcatcctcccgccacacagaaacccgcccagccggggccaccgaccccaccccctgcctggaaacttaaggaggccggagctgtggggagctcagagctgagatcctacaggagtccagggctggagagaaaacctctgcgaggaaagggaaggagcaagccgtgaatttaagggacgctgtgaagcaatcatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgaccaggaacacccgactcctcaaaagcaaatgagatcccgcctcttcttcttcagaagacactgcaaaggcgcagtgcttctctacagacttctccagacccaccacaccgcagaagcgggacgagaccctacaggagagggaagagtgcattttcccagatacttcccattttggaagttttcaggacttggtctgatttcaggatactctgtcagatgggaagacatgaatgcacactagcctctccaggaatgcctcctccctgggcagaaagtggccatgccaccctgttttcagctaaagcccaacctcctgacctgtcaccgtgagcagctttggaaacaggaccacaaaaatgaaagcatgtctcaatagtaaaagataacaagatctttcaggaaagacggattgcattagaaatagacagtatatttatagtcacaagagcccagcagggcctcaaagttggggcaggctggctggcccgtcatgttcctcaaaagcacccttgacgtcaagtctccttcccctttccccactccctggctctcagaaggtattccttttgtgtacagtgtgtaaagtgtaaatcctttttctttataaactttagagtagcatgagagaattgtatcatttgaacaactaggcttcagcatatttatagcaatccatgttagtttttactttctgttgccacaaccctgttttatactgtacttaataaattcagatatatttttcacagtttttcc

SEQ ID NO: 108 - protein, 562 aa, Homo sapien
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 109 - DNA, 4050 bp, Homo sapien
cttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggccttccctggcgcctgcccctgtcggccccgcccgagaacctccctgcgccagggcagggtttactcatcccggcgaggtgatcccatgcgcgagggcgggcgcaagggcggccagagaacccagcaatccgagtatgcggcatcagcccttcccaccaggcacttccttccttttcccgaacgtccagggagggagggccgggcacttataaactcgagccctggccgatccgcatgtcagaggctgcctcgcaggggctgcgcgcacggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcatctagctgtctgctcagacagaacccctacatgaaacagaaacaaaaacactaaaaataaaaatggccatttgctttttcaccagatttgctaatttatcctgaaatttcagattcccagagcaaaataattttaaacaaagggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtg

SEQ ID NO: 110 - protein, 575 aa, Homo sapien
MLGVLVLGALALAGLGFPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEFHFPATCRPLAVEPGAAAAAVSITYGTPFAARGADFQALPVGSSAAVAPLGLQLMCTAPPGAVQGHWAREAPGAWDCSVENGGCEHACNAIPGAPRCQCPAGAALQADGRSCTASATQSCNDLCEHFCVPNPDQPGSYSCMCETGYRLAADQHRCEDVDDCILEPSPCPQRCVNTQGGFECHCYPNYDLVDGECVEPVDPCFRANCEYQCQPLNQTSYLCVCAEGFAPIPHEPHRCQMFCNQTACPADCDPNTQASCECPEGYILDDGFICTDIDECENGGFCSGVCHNLPGTFECICGPDSALARHIGTDCDSGKVDGGDSGSGEPPPSPTPGSTLTPPAVGLVHSGLLIGISIASLCLVVALLALLCHLRKKQGAARAKMEYKCAAPSKEVVLQHVRTERTPQRL

SEQ ID NO: 111 - DNA, 2153 bp, Homo sapien
aagactgcgagctccccgcaccccctcgcactccctctggccggcccagggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgttggagctactgcaaatgctatattgcactgtgaccgagaacttttaagaggatagaatacatggaaacgcaaatgagtatttcggagcatgaagaccctggagttcaaaaaactcttgatatgacctgttattaccattagcattctggttttgacatcagcattagtcactttgaaatgtaacgaatggtactacaaccaattccaagttttaatttttaacaccatggcaccttttgcacataacatgctttagattatatattccgcacttaaggattaaccaggtcgtccaagcaaaaacaaatgggaaaatgtcttaaaaaatcctgggtggacttttgaaaagcttttttttttttttttttttgagacggagtcttgctctgttgcccaggctggagtgcagtagcacgatctcggctcacttgcaccctccgtctctcgggttcaagcaattgtctgcctcagcctcccgagtagctgggattacaggtgcgcactaccacgccaagctaatttttgtattttttagtagagatggggtttcaccatcttggccaggctggtcttgaattcctgacctcagtgatccacccaccttggcctcccaaagatgctagtattatgggcgtgaaccaccatgcccagccgaaaagcttttgaggggctgacttcaatccatgtaggaaagtaaaatggaaggaaattgggtgcatttctaggacttttctaacatatgtctataatatagtgtttaggttcttttttttttcaggaatacatttggaaattcaaaacaattgggcaaactttgtattaatgtgttaagtgcaggagacattggtattctgggcagcttcctaatatgctttacaatctgcactttaactgacttaagtggcattaaacatttgagagctaactatatttttataagactactatacaaactacagagtttatgatttaaggtacttaaagcttctatggttgacattgtatatataattttttaaaaaggtttttctatatggggattttctatttatgtaggtaatattgttctatttgtatatattgagataatttatttaatatactttaaataaaggtgactgggaattgtt

SEQ ID NO: 112 - protein, 295 aa, Homo sapien
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 113 - DNA, 2696 bp, Homo sapien
ctggtgaccttacttatctctgttgctttctggggtcctaggaaatgccagcactcccacccacattgcctgaactttccaacactccctagctgcgctgtgtcctatctcaacacttcctcatgtatttcttgtgtcttctagaacattcccccgccattattacttcaatatggctacacatacttcctaattgccctgcaaaccatctccttctcaccattgcccagcgatgctttcgtctcctccataaacactcccggagaccaatttttgtgtcacccccatactccctcgttgacacactgactccatacataacctccttgaaaaacctctttattaatctcaccatcctccagacttccctcctgtcataattccatccctcctccaacttttccctctcaagctctgcccttcccagcccagcccagcctacccaacctcatctcttccctgtagaccacatcccaccatgttcccctgagcctccaaggaaggggctcagggggccccatggcctcccgctccctgtggccccacagcccccgtgggccaggggaagcgccccagaagccgaagtgcccaccatgggcaaccacacgtgggagggctgccacgtggactcgcgcgtggaccacctctttccgccatccctctacatctttgtcatcggcgtggggctgcccaccaactgcctggctctgtgggcggcctaccgccaggtgcaacagcgcaacgagctgggcgtctacctgatgaacctcagcatcgccgacctgctgtacatctgcacgctgccgctgtgggtggactacttcctgcaccacgacaactggatccacggccccgggtcctgcaagctctttgggttcatcttctacaccaatatctacatcagcatcgccttcctgtgctgcatctcggtggaccgctacctggctgtggcccacccactccgcttcgcccgcctgcgccgcgtcaagaccgccgtggccgtgagctccgtggtctgggccacggagctgggcgccaactcggcgcccctgttccatgacgagctcttccgagaccgctacaaccacaccttctgctttgagaagttccccatggaaggctgggtggcctggatgaacctctatcgggtgttcgtgggcttcctcttcccgtgggcgctcatgctgctgtcgtaccggggcatcctgcgggccgtgcggggcagcgtgtccaccgagcgccaggagaaggccaagatcaagcggctggccctcagcctcatcgccatcgtgctggtctgctttgcgccctatcacgtgctcttgctgtcccgcagcgccatctacctgggccgcccctgggactgcggcttcgaggagcgcgtcttttctgcataccacagctcactggctttcaccagcctcaactgtgtggcggaccccatcctctactgcctggtcaacgagggcgcccgcagcgatgtggccaaggccctgcacaacctgctccgctttctggccagcgacaagccccaggagatggccaatgcctcgctcaccctggagaccccactcacctccaagaggaacagcacagccaaagccatgactggcagctgggcggccactccgccctcccagggggaccaggtgcagctgaagatgctgccgccagcacaatgaaccccgagtggcacagaatccccagttttcccctctcatcccacagtcccttctctcctggtctggtgtatgcaaattgtatggaaaaagggctgtgttaatattcataagaatacaagaacttaggaagagtgaggttggtgtgtcactggtcaacctttgtgctcccagatcccatcacagtttggcgattgtggagggcctcctgaaggaggagatgagtaaatatatttttttggagacagggtctcactgtgttgcccaggctggagtgcagtagtgcagtcgtggctcactgcagcctccacctcctgggctctccagcgatcttcccacatcagcctcccgagtagctgggaccacaaatgtgagcccacccatgcctggctaatttttgtactttttgtataaatggagtctcactatgtttccccaggctgatcttgaactcctgggctcaagagatcctcctgccttggcctcccaaagtgctcagattagagatgtgagccgccatgtctggccagataaattaagtcaaacatttggtttccagaaaataaagacaaatagagaaggttagatttttttttttccaacaagtggataaaagtctgtgactcgggggaaagtggaaggagaaatgcagccgatatagagtcattatgtttgcaaagcccctggtcatacaggccagggaacataagaccgcaattctaagtttctagataaacagcgatctccaagtcaagactgaggatgaagagggagaatgtcagaactcaagtgaagggcaatcagggcagactgcctggaggagtgatgccagaaggtttgggaagaaggtgtgggacaagaagaaagggtatttattcattcattcaacagaggtttatgtagggcactgtgctgggtggggctggggacacaacaatgactgaggcagcctggccttgccttcacagggctcaccatacacaagtaaataaaaaatatgtaatgtttggaattgct

SEQ ID NO: 114 - protein, 362 aa, Homo sapien
MGNHTWEGCHVDSRVDHLFPPSLYIFVIGVGLPTNCLALWAAYRQVQQRNELGVYLMNLSIADLLYICTLPLWVDYFLHHDNWIHGPGSCKLFGFIFYTNIYISIAFLCCISVDRYLAVAHPLRFARLRRVKTAVAVSSVVWATELGANSAPLFHDELFRDRYNHTFCFEKFPMEGWVAWMNLYRVFVGFLFPWALMLLSYRGILRAVRGSVSTERQEKAKIKRLALSLIAIVLVCFAPYHVLLLSRSAIYLGRPWDCGFEERVFSAYHSSLAFTSLNCVADPILYCLVNEGARSDVAKALHNLLRFLASDKPQEMANASLTLETPLTSKRNSTAKAMTGSWAATPPSQGDQVQLKMLPPAQ

SEQ ID NO: 115 - DNA, 3354 bp, Homo sapien
agcggggggttcccggccggacaggcggggcgtcggggcgcgggctggggccgctgtcagtcagtccactggctcccgcgccgcgtctgtgtccgtcgctcggagggtggaagccggggtctcgcgggccgcgggccgcatgactcctctctgcctcaattgctctgtcctccctggagacctgtacccagggggtgcaaggaaccccatggcttgcaatggcagtgcggccagggggcactttgaccctgaggacttgaacctgactgacgaggcactgagactcaagtacctggggccccagcagacagagctgttcatgcccatctgtgccacatacctgctgatcttcgtggtgggcgctgtgggcaatgggctgacctgtctggtcatcctgcgccacaaggccatgcgcacgcctaccaactactacctcttcagcctggccgtgtcggacctgctggtgctgctggtgggcctgcccctggagctctatgagatgtggcacaactaccccttcctgctgggcgttggtggctgctatttccgcacgctactgtttgagatggtctgcctggcctcagtgctcaacgtcactgccctgagcgtggaacgctatgtggccgtggtgcacccactccaggccaggtccatggtgacgcgggcccatgtgcgccgagtgcttggggccgtctggggtcttgccatgctctgctccctgcccaacaccagcctgcacggcatccagcagctgcacgtgccctgccggggcccagtgccagactcagctgtttgcatgctggtccgcccacgggccctctacaacatggtagtgcagaccaccgcgctgctcttcttctgcctgcccatggccatcatgagcgtgctctacctgctcattgggctgcgactgcggcgggagaggctgctgctcatgcaggaggccaagggcaggggctctgcagcagccaggtccagatacacctgcaggctccagcagcacgatcggggccggagacaagtgaccaagatgctgtttgtcctggtcgtggtgtttggcatctgctgggccccgttccacgccgaccgcgtcatgtggagcgtcgtgtcacagtggacagatggcctgcacctggccttccagcacgtgcacgtcatctccggcatcttcttctacctgggctcggcggccaaccccgtgctctatagcctcatgtccagccgcttccgagagaccttccaggaggccctgtgcctcggggcctgctgccatcgcctcagaccccgccacagctcccacagcctcagcaggatgaccacaggcagcaccctgtgtgatgtgggctccctgggcagctgggtccaccccctggctgggaacgatggcccagaggcgcagcaagagaccgatccatcctgagtggagccttaaagtggcttcacctggaggggccagagggtcacctggagctggggagacacatctgccttcctctgcagggatccttcacgtactgtccctagttcagcctagaaattctgaccagcacctcagtttccctcagagggaaacagcaggaggagggatccctgactgctgaggactcacactgaccagacgccacaccttgtgcttcttatctgtccactgccactcccccagttcaaatccttaccctgcagaaatatcacagttagctggggctcagcagtcctccctctggggactccctgccaccactgccagtttctgaaacggtcccactgggtcctcactgtccttcccagttcctgttcaggttctggcaggggcccagggatccaggggacctggttccaatctcagccctgctgtcaccaccttgtcatgcaccatcaagcatatcagtctacctttctttttttctgagacagagtctcactctgtcgcccaggctagagtgcagtggcgcgattttggctcactgcaacctccgcctccggggttcaagcgattctcctgcctcagcctcccgagttgctgggactacaggtgagccccagcatgcccagctaattttttttaatttttagtagagacggggtttcaccatgttggccaggctggtctcaaactcttgacctcaggtgatccgccgacctcggcctcccaaagtcctcggattacaggcatgagccaccacacccggccaatcagtccacctttctaggccttggttccttgcctgaaaaatgaaagaggcgctggctttccacagtgtcatgctttggcactttagctatggttttctttctgtgtgtgtgtaagccactgcttataataaaaccaacaataccctcagactgaaagggcggaagttattatctgcatctttatcaaccccaagccccacttcctccctgacctccccatgccctccccagcctctcccagcacaagtggggcaaagccagcatgcaagcagaccccaccaccacagcccacctccgtcctcacatacgtgcaggctggctcgggagtccagtgagcagagcattggacttggctggccagagggtctctgagggcaagagacatggccaaccaagggcaaggagtgaccctgtggagggttctgccgaactcaatgcagtgagaagagggacagggacaagtagtccttgaaactgagccccattctgaatccctgcaggccaagtcattgctcagccaggactcagttcatgggggaaacttgacctgctgcagtccctgagtcttgtcctcctgagaggaagccctggcttccaaggctgggagctggaggatgaccttcggtcggtctgtctgggttctccctgcagacagcttcctagctcatgcccatagctcatgctccctgccgagaaagtggaggacgtggtacagggttgcagatgtttagttttaaaaattcaattataaaaataataaatgctcatgatagaaaatttggaaagtgcaaataagcaaaaatgaaaacaattttaaaaatgtaaaacctctcttgccagggaatgggggaagggcaagtgaggagttctttaatgggtgaagagtttcagttttgcaaaatgaaaaagttctggagatcagttgtgcaacaatatgaatatacataacaatactgaactatacactgaaatggttaagatggtacattttatgttatgtgtattttaccacaatttttataaaaagaggattaaatctaaaggaaagaaaaaattaaaaccacccataactttactctgaagcagtaacagtggcatgtttcctcctaaaaaaaaaaaaaaaaaaagaagaaaaaaaaataaagaaaaaaaaaaaaaaaa

SEQ ID NO: 116 - protein, 426 aa, Homo sapien
MTPLCLNCSVLPGDLYPGGARNPMACNGSAARGHFDPEDLNLTDEALRLKYLGPQQTELFMPICATYLLIFVVGAVGNGLTCLVILRHKAMRTPTNYYLFSLAVSDLLVLLVGLPLELYEMWHNYPFLLGVGGCYFRTLLFEMVCLASVLNVTALSVERYVAVVHPLQARSMVTRAHVRRVLGAVWGLAMLCSLPNTSLHGIQQLHVPCRGPVPDSAVCMLVRPRALYNMVVQTTALLFFCLPMAIMSVLYLLIGLRLRRERLLLMQEAKGRGSAAARSRYTCRLQQHDRGRRQVTKMLFVLVVVFGICWAPFHADRVMWSVVSQWTDGLHLAFQHVHVISGIFFYLGSAANPVLYSLMSSRFRETFQEALCLGACCHRLRPRHSSHSLSRMTTGSTLCDVGSLGSWVHPLAGNDGPEAQQETDPS

SEQ ID NO: 117 - DNA, 2512 bp, Homo sapien
ctttgaagtcagctggaccaaggaaaggccctgccctgaaggctggtcacttgcagaggtaaactcccctctttgacttctggccagggtttgtgctgagctggctgcagccgctctcagcctcgctccgggcacgtcgggcagcctcgggccctcctgcctgcaggatcatgcccaccaccgtggacgatgtcctggagcatggaggggagtttcactttttccagaagcaaatgtttttcctcttggctctgctctcggctaccttcgcgcccatctacgtgggcatcgtcttcctgggcttcacccctgaccaccgctgccggagccccggagtggccgagctgagtctgcgctgcggctggagtcctgcagaggaactgaactacacggtgccgggcccaggacctgcgggcgaagcctccccaagacagtgtaggcgctacgaggtggactggaaccagagcaccttcgactgcgtggaccccctggccagcctggacaccaacaggagccgcctgccactgggcccctgccgggacggctgggtgtacgagacgcctggctcgtccatcgtcaccgagtttaacctggtatgtgccaactcctggatgttggacctattccagtcatcagtgaatgtaggattctttattggctctatgagtatcggctacatagcagacaggtttggccgtaagctctgcctcctaactacagtcctcataaatgctgcagctggagttctcatggccatttccccaacctatacgtggatgttaatttttcgcttaatccaaggactggtcagcaaagcaggctggttaataggctacatcctgattacagaatttgttgggcggagatatcggagaacagtggggattttttaccaagttgcctatacagttgggctcctggtgctagctggggtggcttacgcacttcctcactggaggtggttgcagttcacagttgctctgcccaacttcttcttcttgctctattactggtgcatacctgagtctcccaggtggctgatctcccagaataagaatgctgaagccatgagaatcattaagcacatcgcaaagaaaaatggaaaatctctacccgcctcccttcagcgcctgagacttgaagaggaaactggcaagaaattgaacccttcatttcttgacttggtcagaactcctcagataaggaaacatactatgatattgatgtacaactggttcacgagctctgtgctctaccagggcctcatcatgcacatgggccttgcaggtgacaatatctacctggatttcttctactctgccctggttgaattcccagctgccttcatgatcatcctcaccatcgaccgcatcggacgccgttacccttgggctgcatcaaatatggttgcaggggcagcctgtctggcctcagtttttatacctggtgatctacaatggctaaaaattattatctcatgcttgggaagaatggggatcacaatggcctatgagatagtctgcctggtcaatgctgagctgtaccccacattcattaggaatcttggcgtccacatctgttcctcaatgtgtgacattggtggcatcatcacgccattcctggtctaccggctcactaacatctggcttgagctcccgctgatggttttcggcgtgcttggcttggttgctggaggtctggtgctgttgcttccagaaactaaagggaaagctttgcctgagaccatcgaggaagccgaaaatatgcaaagaccaagaaaaaataaagaaaagatgatttacctccaagttcagaaactagacattccattgaactaagaagagagaccgttgctgctgtcatgacctagctttgatggcagcaagaccaaaagtagaaatccctgcactcatcacaaagcccatacaactcaaccaaacttacccctgagccctatcaacctaggtctacagccagtggagtctattgtacactgtggaaaaatacccatgggaccagatcctgccaaattcttccagctcactttattctcagcattcctaggacattggacattggttttctggagggttttttttccatctttgtatttttttaaatttgattcttttctttgcaatgctatctaaccagaatacataggggaactgtgggctaggcaaacaaaatagaaaaaagtgtgaaaaacagtaaagttgggagaggagcatctattttcttaaagaaataaaacacccaaaacaatataaagttgtccagaatgtatgtcaagaattttagataggcctttcagtaacacaggtgaagaaatttttaaaaatacattgattattatctaggttagacttaaagtgaatctcaaataaaagaatcaggaatacaacttaagtgatcatgaggtccttccatatttagattgggtaagcatgaatgtgtattttctacaaaagaccttgagaagagttcaataaaaaatgttagcattataaaa

SEQ ID NO: 118 - protein, 555 aa, Homo sapien
MPTTVDDVLEHGGEFHFFQKQMFFLLALLSATFAPIYVGIVFLGFTPDHRCRSPGVAELSLRCGWSPAEELNYTVPGPGPAGEASPRQCRRYEVDWNQSTFDCVDPLASLDTNRSRLPLGPCRDGWVYETPGSSIVTEFNLVCANSWMLDLFQSSVNVGFFIGSMSIGYIADRFGRKLCLLTTVLINAAAGVLMAISPTYTWMLIFRLIQGLVSKAGWLIGYILITEFVGRRYRRTVGIFYQVAYTVGLLVLAGVAYALPHWRWLQFTVALPNFFFLLYYWCIPESPRWLISQNKNAEAMRIIKHIAKKNGKSLPASLQRLRLEEETGKKLNPSFLDLVRTPQIRKHTMILMYNWFTSSVLYQGLIMHMGLAGDNIYLDFFYSALVEFPAAFMIILTIDRIGRRYPWAASNMVAGAACLASVFIPGDLQWLKIIISCLGRMGITMAYEIVCLVNAELYPTFIRNLGVHICSSMCDIGGIITPFLVYRLTNIWLELPLMVFGVLGLVAGGLVLLLPETKGKALPETIEEAENMQRPRKNKEKMIYLQVQKLDIPLN

SEQ ID NO: 119 - DNA, 5431 bp, Homo sapien
gccctggccaaggaggaggctgaaagagcctgagctgtgccctctccattccactgctgtggcagggtcagaaatcttggatagagaaaaccttttgcaaacgggaatgtatctttgtaattcctagcacgaaagactctaacaggtgttgctgtggccagttcaccaaccagcatatcccccctctgccaagtgcaacacccagcaaaaatgaagaggaaaacaaacaggtggagactcagcctgagaaatggtctgttgccaagcacacccagagctacccaacagattcctatggagttcttgaattccagggtggcggatattccaataaagccatgtatatccgtgtatcctatgacaccaagccagactcactgctccatctcatggtgaaagattggcagctggaactccccaagctcttaatatctgtgcatggaggcctccagaactttgagatgcagcccaagctgaaacaagtctttgggaaaggcctgatcaaggctgctatgaccaccggggcctggatcttcaccgggggtgtcagcacaggtgttatcagccacgtaggggatgccttgaaagaccactcctccaagtccagaggccgggtttgtgctataggaattgctccatggggcatcgtggagaataaggaagacctggttggaaaggatgtaacaagagtgtaccagaccatgtccaaccctctaagtaagctctctgtgctcaacaactcccacacccacttcatcctggctgacaatggcaccctgggcaagtatggcgccgaggtgaagctgcgaaggctgctggaaaagcacatctccctccagaagatcaacacaagactggggcagggcgtgcccctcgtgggtctcgtggtggaggggggccctaacgtggtgtccatcgtcttggaatacctgcaagaagagcctcccatccctgtggtgatttgtgatggcagcggacgtgcctcggacatcctgtcctttgcgcacaagtactgtgaagaaggcggaataataaatgagtccctcagggagcagcttctagttaccattcagaaaacatttaattataataaggcacaatcacatcagctgtttgcaattataatggagtgcatgaagaagaaagaactcgtcactgtgttcagaatgggttctgagggccagcaggacatcgagatggcaattttaactgccctgctgaaaggaacaaacgtatctgctccagatcagctgagcttggcactggcttggaaccgcgtggacatagcacgaagccagatctttgtctttgggccccactggccgcccctgggaagcctggcacccccgacggacagcaaagccacggagaaggagaagaagccacccatggccaccaccaagggaggaagaggaaaagggaaaggcaagaagaaagggaaagtgaaagaggaagtggaggaagaaactgacccccggaagatagagctgctgaactgggtgaatgctttggagcaagcgatgctagatgctttagtcttagatcgtgtcgactttgtgaagctcctgattgaaaacggagtgaacatgcaacactttctgaccattccgaggctggaggagctttataacacaagactgggtccaccaaacacacttcatctgctggtgagggatgtgaaaaagagcaaccttccgcctgattaccacatcagcctcatagacatcgggctcgtgctggagtacctcatgggaggagcctaccgctgcaactacactcggaaaaactttcggaccctttacaacaacttgtttggaccaaagaggcctaaagctcttaaacttctgggaatggaagatgatgagcctccagctaaagggaagaaaaaaaaaaaaaagaaaaaggaggaagagatcgacattgatgtggacgaccctgccgtgagtcggttccagtatcccttccacgagctgatggtgtgggcagtgctgatgaaacgccagaaaatggcagtgttcctctggcagcgaggggaagagagcatggccaaggccctggtggcctgcaagctctacaaggccatggcccacgagtcctccgagagtgatctggtggatgacatctcccaggacttggataacaattccaaagacttcggccagcttgctttggagttattagaccagtcctataagcatgacgagcagatcgctatgaaactcctgacctacgagctgaaaaactggagcaactcgacctgcctcaaactggccgtggcagccaaacaccgggacttcattgctcacacctgcagccagatgctgctgaccgatatgtggatgggaagactgcggatgcggaagaaccccggcctgaaggttatcatggggattcttctaccccccaccatcttgtttttggaatttcgcacatatgatgatttctcgtatcaaacatccaaggaaaacgaggatggcaaagaaaaagaagaggaaaatacggatgcaaatgcagatgctggctcaagaaagggggatgaggagaacgagcataaaaaacagagaagtattcccatcggaacaaagatctgtgaattctataacgcgcccattgtcaagttctggttttacacaatatcatacttgggctacctgctgctgtttaactacgtcatcctggtgcggatggatggctggccgtccctccaggagtggatcgtcatctcctacatcgtgagcctggcgttagagaagatacgagagatcctcatgtcagaaccaggcaaactcagccagaaaatcaaagtttggcttcaggagtactggaacatcacagatctcgtggccatttccacattcatgattggagcaattcttcgcctacagaaccagccctacatgggctatggccgggtgatctactgtgtggatatcatcttctggtacatccgtgtcctggacatctttggtgtcaacaagtatctggggccatacgtgatgatgattggaaagatgatgatcgacatgctgtactttgtggtcatcatgctggtcgtgctcatgagtttcggagtagcccgtcaagccattctgcatccagaggagaagccctcttggaaactggcccgaaacatcttctacatgccctactggatgatctatggagaggtgtttgcagaccagatagacctctacgccatggaaattaatcctccttgtggtgagaacctatatgatgaggagggcaagcggcttcctccctgtatccccggcgcctggctcactccagcactcatggcgtgctatctactggtcgccaacatcctgctggtgaacctgctgattgctgtgttcaacaataccttctttgaagtaaaatcaatatccaaccaggtgtggaagttccagcgatatcagctgattatgacatttcatgacaggccagtcctgcccccaccgatgatcattttaagccacatctacatcatcattatgcgtctcagcggccgctgcaggaaaaagagagaaggggaccaagaggaacgggatcgtggattgaagctcttccttagcgacgaggagctaaagaggctgcatgagttcgaggagcagtgcgtgcaggagcacttccgggagaaggaggatgagcagcagtcgtccagcgacgagcgcatccgggtcacttctgaaagagttgaaaatatgtcaatgaggttggaagaaatcaatgaaagagaaacttttatgaaaacttccctgcagactgttgaccttcgacttgctcagctagaagaattatctaacagaatggtgaatgctcttgaaaatcttgcgggaatcgacaggtctgacctgatccaggcacggtcccgggcttcttctgaatgtgaggcaacgtatcttctccggcaaagcagcatcaatagcgctgatggctacagcttgtatcgatatcattttaacggagaagagttattatttgaggatacatctctctccacgtcaccagggacaggagtcaggaaaaaaacctgttccttccgtataaaggaagagaaggacgtgaaaacgcacctagtcccagaatgtcagaacagtcttcacctttcactgggcacaagcacatcagcaaccccagatggcagtcaccttgcagtagatgacttaaagaacgctgaagagtcaaaattaggtccagatattgggatttcaaaggaagatgatgaaagacagacagactctaaaaaagaagaaactatttccccaagtttaaataaaacagatgtgatacatggacaggacaaatcagatgttcaaaacactcagctaacagtggaaacgacaaatatagaaggcactatttcctatcccctggaagaaaccaaaattacacgctatttccccgatgaaacgatcaatgcttgtaaaacaatgaagtccagaagcttcgtctattcccggggaagaaagctggtcggtggggttaaccaggatgtagagtacagttcaatcacggaccagcaattgacgacggaatggcaatgccaagttcaaaagatcacgcgctctcatagcacagatattccttacattgtgtcggaagctgcagtgcaagctgagcataaagagcagtttgcagatatgcaagatgaacaccatgtcgctgaagcaattcctcgaatccctcgcttgtccctaaccattactgacagaaatgggatggaaaacttactgtctgtgaagccagatcaaactttgggattcccatctctcaggtcaaaaagtttacatggacatcctaggaatgtgaaatccattcagggaaagttagacagatctggacatgccagtagtgtaagcagcttagtaattgtgtctggaatgacagcagaagaaaaaaaggttaagaaagagaaagcttccacagaaactgaatgctagtctgttttgtttctttaatttttttttttaacagtcagaaccactaatgggtgtcatcttggccatctaaacatcatcaatttctaaaaacattttcccttaaaaaattttggaaattcagacttgatttacaatttaatgcactaaaagtagtattttgttagcatatgttagtaggcttagttttttcagttgcagtagtatcaaatgaaagtgatgatactgtaacgaagataaattggctaatcagtatacaagattatacaatctctttattactgagggccaccaaatagcctaggaagtgccctcgagcactgaagtcaccattaggtcacttaagaagtaagcaactagctgggcacagtggctcatgcctgtaatcctagcactttgggaggccaaggcagaaagatagcttgagtccaggagtttgagaccagcctgggcaacatagtgataccccatctcttaaaaaaaaaaaaaaaaaa

SEQ ID NO: 120 - protein, 1533 aa, Homo sapien
MYIRVSYDTKPDSLLHLMVKDWQLELPKLLISVHGGLQNFEMQPKLKQVFGKGLIKAAMTTGAWIFTGGVSTGVISHVGDALKDHSSKSRGRVCAIGIAPWGIVENKEDLVGKDVTRVYQTMSNPLSKLSVLNNSHTHFILADNGTLGKYGAEVKLRRLLEKHISLQKINTRLGQGVPLVGLVVEGGPNVVSIVLEYLQEEPPIPVVICDGSGRASDILSFAHKYCEEGGIINESLREQLLVTIQKTFNYNKAQSHQLFAIIMECMKKKELVTVFRMGSEGQQDIEMAILTALLKGTNVSAPDQLSLALAWNRVDIARSQIFVFGPHWPPLGSLAPPTDSKATEKEKKPPMATTKGGRGKGKGKKKGKVKEEVEEETDPRKIELLNWVNALEQAMLDALVLDRVDFVKLLIENGVNMQHFLTIPRLEELYNTRLGPPNTLHLLVRDVKKSNLPPDYHISLIDIGLVLEYLMGGAYRCNYTRKNFRTLYNNLFGPKRPKALKLLGMEDDEPPAKGKKKKKKKKEEEIDIDVDDPAVSRFQYPFHELMVWAVLMKRQKMAVFLWQRGEESMAKALVACKLYKAMAHESSESDLVDDISQDLDNNSKDFGQLALELLDQSYKHDEQIAMKLLTYELKNWSNSTCLKLAVAAKHRDFIAHTCSQMLLTDMWMGRLRMRKNPGLKVIMGILLPPTILFLEFRTYDDFSYQTSKENEDGKEKEEENTDANADAGSRKGDEENEHKKQRSIPIGTKICEFYNAPIVKFWFYTISYLGYLLLFNYVILVRMDGWPSLQEWIVISYIVSLALEKIREILMSEPGKLSQKIKVWLQEYWNITDLVAISTFMIGAILRLQNQPYMGYGRVIYCVDIIFWYIRVLDIFGVNKYLGPYVMMIGKMMIDMLYFVVIMLVVLMSFGVARQAILHPEEKPSWKLARNIFYMPYWMIYGEVFADQIDLYAMEINPPCGENLYDEEGKRLPPCIPGAWLTPALMACYLLVANILLVNLLIAVFNNTFFEVKSISNQVWKFQRYQLIMTFHDRPVLPPPMIILSHIYIIIMRLSGRCRKKREGDQEERDRGLKLFLSDEELKRLHEFEEQCVQEHFREKEDEQQSSSDERIRVTSERVENMSMRLEEINERETFMKTSLQTVDLRLAQLEELSNRMVNALENLAGIDRSDLIQARSRASSECEATYLLRQSSINSADGYSLYRYHFNGEELLFEDTSLSTSPGTGVRKKTCSFRIKEEKDVKTHLVPECQNSLHLSLGTSTSATPDGSHLAVDDLKNAEESKLGPDIGISKEDDERQTDSKKEETISPSLNKTDVIHGQDKSDVQNTQLTVETTNIEGTISYPLEETKITRYFPDETINACKTMKSRSFVYSRGRKLVGGVNQDVEYSSITDQQLTTEWQCQVQKITRSHSTDIPYIVSEAAVQAEHKEQFADMQDEHHVAEAIPRIPRLSLTITDRNGMENLLSVKPDQTLGFPSLRSKSLHGHPRNVKSIQGKLDRSGHASSVSSLVIVSGMTAEEKKVKKEKASTETEC

SEQ ID NO: 121 - DNA, 5511 bp, Homo sapien
tctctgtctgccagggtctccgactgtcccagacgggctggtgtgggcttgggatcctcctggtgacctctcccgctaaggtccctcagccactctgccccaagatgggccgtggggctggccgtgagtactcacctgccgccaccacggcagagaatgggggcggcaagaagaaacagaaggagaaggaactggatgagctgaagaaggaggtggcaatggatgaccacaagctgtccttggatgagctgggccgcaaataccaagtggacctgtccaagggcctcaccaaccagcgggctcaggacgttctggctcgagatgggcccaacgccctcacaccacctcccacaacccctgagtgggtcaagttctgccgtcagcttttcggggggttctccatcctgctgtggattggggctatcctctgcttcctggcctacggcatccaggctgccatggaggatgaaccatccaacgacaatctatatctgggtgtggtgctggcagctgtggtcattgtcactggctgcttctcctactaccaggaggccaagagctccaagatcatggattccttcaagaacatggtacctcagcaagcccttgtgatccgggagggagagaagatgcagatcaacgcagaggaagtggtggtgggagacctggtggaggtgaagggtggagaccgcgtccctgctgacctccggatcatctcttctcatggctgtaaggtggataactcatccttaacaggagagtcggagccccagacccgctcccccgagttcacccatgagaaccccctggagacccgcaatatctgtttcttctccaccaactgtgttgaaggcactgccaggggcattgtgattgccacaggagaccggacggtgatgggccgcatagctactctcgcctcaggcctggaggttgggcggacacccatagcaatggagattgaacacttcatccagctgatcacaggggtcgctgtattcctgggggtctccttcttcgtgctctccctcatcctgggctacagctggctggaggcagtcatcttcctcatcggcatcatagtggccaacgtgcctgaggggcttctggccactgtcactgtgtgcctgaccctgacagccaagcgcatggcacggaagaactgcctggtgaagaacctggaggcggtggagacgctgggctccacgtccaccatctgctcggacaagacgggcaccctcacccagaaccgcatgaccgtcgcccacatgtggttcgacaaccaaatccatgaggctgacaccaccgaagatcagtctggggccacttttgacaaacgatcccctacgtggacggccctgtctcgaattgctggtctctgcaaccgcgccgtcttcaaggcaggacaggagaacatctccgtgtctaagcgggacacagctggtgatgcctctgagtcagctctgctcaagtgcattgagctctcctgtggctcagtgaggaaaatgagagacagaaaccccaaggtggcagagattcctttcaactctaccaacaagtaccagctgtctatccacgagcgagaagacagcccccagagccacgtgctggtgatgaagggggccccagagcgcattctggaccggtgctccaccatcctggtgcagggcaaggagatcccgctcgacaaggagatgcaagatgcctttcaaaatgcctacatggagctggggggacttggggagcgtgtgctgggattctgtcaactgaatctgccatctggaaagtttcctcggggcttcaaattcgacacggatgagctgaactttcccacggagaagctttgctttgtggggctcatgtctatgattgaccctccccgggctgctgtgccagatgctgtgggcaagtgccgaagcgcaggcatcaaggtgatcatggtaaccggggatcaccctatcacagccaaggccattgccaaaggcgtgggcatcatatcagagggtaacgagactgtggaggacattgcagcccggctcaacattcccatgagtcaagtcaaccccagagaagccaaggcatgcgtggtgcacggctctgacctgaaggacatgacatcggagcagctcgatgagatcctcaagaaccacacagagatcgtctttgctcgaacgtctccccagcagaagctcatcattgtggagggatgtcagaggcagggagccattgtggccgtgacgggtgacggggtgaacgactcccctgcattgaagaaggctgacattggcattgccatgggcatctctggctctgacgtctctaagcaggcagccgacatgatcctgctggatgacaactttgcctccatcgtcacgggggtggaggagggccgcctgatctttgacaacttgaagaaatccatcgcctacaccctgaccagcaacatccccgagatcacccccttcctgctgttcatcattgccaacatccccctacctctgggcactgtgaccatcctttgcattgacctgggcacagatatggtccctgccatctccttggcctatgaggcagctgagagtgatatcatgaagcggcagccacgaaactcccagacggacaagctggtgaatgagaggctcatcagcatggcctacggacagatcgggatgatccaggcactgggtggcttcttcacctactttgtgatcctggcagagaacggtttcctgccatcacggctactgggaatccgcctcgactgggatgaccggaccatgaatgatctggaggacagctatggacaggagtggacctatgagcagcggaaggtggtggagttcacgtgccacacggcattctttgccagcatcgtggtggtgcagtgggctgacctcatcatctgcaagacccgccgcaactcagtcttccagcagggcatgaagaacaagatcctgatttttgggctcctggaggagacggcgttggctgcctttctctcttactgcccaggcatgggtgtagccctccgcatgtacccgctcaaagtcacctggtggttctgcgccttcccctacagcctcctcatcttcatctatgatgaggtccgaaagctcatcctgcggcggtatcctggtggctgggtggagaaggagacatactactgaccccattggaagaagaaccaggcatggaaagatggggagctctggaggtgttgtggggatggtgatggagagggatggaaataacgggtggcattgggtggcaacatttggggagagataatgaggcaactcagcaggctaagttgcggggtatataaattggggtgatgaccccatagacctaactgtgaacaatcagattagacactatgtgttagagtccccccgaccagatccttttccatcccactccactatgttgtctattttttctgaggaattaagggttaccccaccctgcccactcccatcccttcaaccccacttcctactgtaatagatcagcatccaaaagcaggaacccatctaaaccagaaggaagccctctcagatcaccccagcctcactccatttcccacttccacccccgttagcttcctgcaggactctatccctggcttccccttcagaccttgcaatcacaaaaggttcttctggtgagtgcaagagcctgagactggaaaaggtggacttgtctcccagtcgaggctggtaagggaccttcagggagagctgggcagacaggtgggagatggaggtagggctggctggaggaaggaaacaacaaaggaagtgaggtagtgccaatgacaggacatttgacatgagtctccagatagatgtcgtggactccagctctacgtcccacattttagaataccccaccagcagaacaaactcagatctcatcagggtagcagcagaggcaggaccagaaggcaatcaagagcttccagaaatgccacacttgtgtgccacagagttccccgctgacccttggttaggggtcctcttagtccacaaggtccggatgtcactcatgtacttaataacacttcaccttctgtaatactaagtcctcagagctccatgctgttctgaaagggatggccacaagttctttcccagcctcttccattccctttcttttcatgcccatcccgatgaacctgcatcattccccgacactgccaagccaaccctggaaaaggagttcgctggccattggctagaatcagggtggagaagttccctgaaccttcctgtctcccagggacatgtatgcttccagggacaagcttaggtcatgaacatggtcagaacctttggacaagaggaaaaatactaagagatttgctttttctgggtgcggtggctcatgcctgtaatcccagcactttgggaggccgaggcaggtggatcatgaggtcaggagttcgaggcgagcctggccaacatggtgaaaccctgtctctactaaaagtacaaaaaattagccagtcatggtggcacacgcctgtaatctcagctactcaggaggctgaggcaggagaattgcttgaacctgtgaggaagaggttgcagtgagctgagatcgtgccattacactccagcctgggcgaaagggtgagactccatctcaaaaaaaaaaaaaatgatttgcttttgacgtcttaggtggcagggctgttccctccaggcaaatgcccttcaaaccgacgatcattgtgcccacttaccctgggctggagagttggtttcaggttcctacaggagatagctttctttcccttactccctatctaacacttttgctctgcaggcagccttgcccattctctaagcctggcttagaaggcactgggaatgtcctgtagagagagacctagataggtcatgcaagtgagaaagacatctgaggaaaatggaagacctaaggcagacaggaaggaagcacaaaagacaagcattgggtcagacccataaaccacctcccaaaggctgtcatttcattgcactggaattttgctttatcagaagcaaggaagtaagggagtcattgccttgggcctgggaatctaagtgggagacaatattaatttggatccgattaattggagattactaactgtggacaaaagtttatctttgcacaatcaataaaaatggcatttttttagtaaattaagagcataaacaatattgctagaggtggcatgtttagtctaccaaaaacaatacttttcaggcactttagaaatatccttttagaagcagcgagtgcatgggctaattatcatcaatctttatgtatttgttaaagaaacatctacaggatctttattggtgaccttttgtaagacattagtttgaggtactacctatctacttgaaaataataaagtggcatttctttatgaaaaaaaaagaaatctcttccataattcagatttctacactttatacttgcctccctcctaaatcgtgatattgaaatatggtg

SEQ ID NO: 122 - protein, 1020 aa, Homo sapien
MGRGAGREYSPAATTAENGGGKKKQKEKELDELKKEVAMDDHKLSLDELGRKYQVDLSKGLTNQRAQDVLARDGPNALTPPPTTPEWVKFCRQLFGGFSILLWIGAILCFLAYGIQAAMEDEPSNDNLYLGVVLAAVVIVTGCFSYYQEAKSSKIMDSFKNMVPQQALVIREGEKMQINAEEVVVGDLVEVKGGDRVPADLRIISSHGCKVDNSSLTGESEPQTRSPEFTHENPLETRNICFFSTNCVEGTARGIVIATGDRTVMGRIATLASGLEVGRTPIAMEIEHFIQLITGVAVFLGVSFFVLSLILGYSWLEAVIFLIGIIVANVPEGLLATVTVCLTLTAKRMARKNCLVKNLEAVETLGSTSTICSDKTGTLTQNRMTVAHMWFDNQIHEADTTEDQSGATFDKRSPTWTALSRIAGLCNRAVFKAGQENISVSKRDTAGDASESALLKCIELSCGSVRKMRDRNPKVAEIPFNSTNKYQLSIHEREDSPQSHVLVMKGAPERILDRCSTILVQGKEIPLDKEMQDAFQNAYMELGGLGERVLGFCQLNLPSGKFPRGFKFDTDELNFPTEKLCFVGLMSMIDPPRAAVPDAVGKCRSAGIKVIMVTGDHPITAKAIAKGVGIISEGNETVEDIAARLNIPMSQVNPREAKACVVHGSDLKDMTSEQLDEILKNHTEIVFARTSPQQKLIIVEGCQRQGAIVAVTGDGVNDSPALKKADIGIAMGISGSDVSKQAADMILLDDNFASIVTGVEEGRLIFDNLKKSIAYTLTSNIPEITPFLLFIIANIPLPLGTVTILCIDLGTDMVPAISLAYEAAESDIMKRQPRNSQTDKLVNERLISMAYGQIGMIQALGGFFTYFVILAENGFLPSRLLGIRLDWDDRTMNDLEDSYGQEWTYEQRKVVEFTCHTAFFASIVVVQWADLIICKTRRNSVFQQGMKNKILIFGLLEETALAAFLSYCPGMGVALRMYPLKVTWWFCAFPYSLLIFIYDEVRKLILRRYPGGWVEKETYY

SEQ ID NO: 123 - DNA, 2249 bp, Homo sapien; identical to SEQ ID NO: 75